(19) United States
(12) Patent Application Publication
(10) Pub. No.: US 2026/0263668 A1
(43) Pub. Date: Sep. 10, 2026

(54) BREAST PUMP AND BREAST PUMP ASSEMBLY

(71) Applicant: Shenzhen Lute Jiacheng Supply Chain Management Co., Ltd., Shenzhen (CN)

(72) Inventors: Wanyuan CHEN, Shenzhen (CN); Tianlei LI, Shenzhen (CN)

(73) Assignee: Shenzhen Lute Jiacheng Supply Chain Management Co., Ltd., Shenzhen (CN)

(21) Appl. No.: 19/663,212

(22) Filed: Apr. 29, 2026

Related U.S. Application Data
(63) Continuation of application No. PCT/CN2024/128630, filed on Oct. 30, 2024.
(63) Continuation of application No. PCT/CN2024/128631, filed on Oct. 30, 2024.
(63) Continuation of application No. PCT/CN2025/081564, filed on Mar. 10, 2025.
(63) Continuation of application No. PCT/CN2025/096991, filed on May 23, 2025.
(63) Continuation of application No. PCT/CN2025/115555, filed on Aug. 19, 2025.

(30) Foreign Application Priority Data
Oct. 31, 2023 (CN) 202322946626.9
Oct. 31, 2023 (CN) 202322946846.1
Oct. 31, 2023 (CN) 202322948544.8
Oct. 31, 2023 (CN) 202322950064.5
Oct. 31, 2023 (CN) 202322953512.7
Mar. 29, 2024 (CN) 202420650099.9
Mar. 29, 2024 (CN) 202420650116.9
Mar. 29, 2024 (CN) 202420650849.2
Mar. 29, 2024 (CN) 202420651895.4
Mar. 29, 2024 (CN) 202420651906.9
Mar. 29, 2024 (CN) 202420656414.9
Mar. 29, 2024 (CN) 202420664590.7
Mar. 29, 2024 (CN) 202420670059.0
Jun. 28, 2024 (CN) 202421521935.X
Jun. 28, 2024 (CN) 202421525787.9
Jun. 28, 2024 (CN) 202421527184.2
Jun. 28, 2024 (CN) 202421527406.0
Jun. 28, 2024 (CN) 202421528368.0
Jun. 28, 2024 (CN) 202421535341.4
Jun. 28, 2024 (CN) 202421535369.8
Sep. 30, 2024 (CN) 202422412905.1

(51) Int. Cl. A61M 1/06

(52) U.S. Cl. CPC A61M 1/066

(57) ABSTRACT

The present disclosure provides a breast pump and a breast pump assembly. The breast pump assembly includes a container body, a container lid, a negative pressure part, and a nipple tunnel cap element, where the container body and the container lid enclose a milk storage chamber; the negative pressure part is connected to the container lid and has a negative pressure chamber configured to communicate with a negative pressure suction port of the breast pump; the nipple tunnel cap element is connected to the container lid; the nipple tunnel cap element has a nipple tunnel; the nipple tunnel communicates with the negative pressure chamber; the nipple tunnel is at least configured to guide milk from a breast; the negative pressure part, the nipple tunnel cap element, and the container lid are integrally formed; and one of the nipple tunnel and the negative pressure chamber communicates with the milk storage chamber.

### RELATED APPLICATIONS

The present patent document is a continuation of International Application No. PCT/CN2024/128630, filed Oct. 30, 2024, and entitled “BREAST PUMP ASSEMBLY, BREAST PUMP, BREAST PUMP APPARATUS, AND DUST COVER,” which claims priority to Chinese Patent Application Nos. 202322953512.7, 202322948544.8, 202322946846.1, 202322946626.9, and 202322950064.5, each filed Oct. 31, 2023; International Application No. PCT/CN2024/128631, filed Oct. 30, 2024, and entitled “BREAST PUMP,” which claims priority to Chinese Patent Application Nos. 202322948544.8, 202322946846.1, and 202322946626.9, each filed Oct. 31, 2023; International Application No. PCT/CN2025/081564, filed Mar. 10, 2025, and entitled “BREAST PUMP,” which claims priority to Chinese Patent Application Nos. 202420650849.2, 202420664590.7, 202420651906.9, 202420651895.4, 202420650099.9, 202420656414.9, 202420670059.0, and 202420650116.9, each filed Mar. 29, 2024; International Application No. PCT/CN2025/096991, filed May 23, 2025, and entitled “BREAST PUMP AND DIAPHRAGM ASSEMBLY,” which claims priority to Chinese Patent Application Nos. 202421527184.2, 202421535341.4, 202421527406.0, 202421521935.X, 202421528368.0, 202421525787.9, and 202421535369.8, each filed Jun. 28, 2024; and International Application No. PCT/CN2025/115555, filed Aug. 19, 2025, and entitled “BREAST PUMP,” which claims priority to Chinese Patent Application No. 202422412905.1, filed Sep. 30, 2024. The entire contents of the foregoing applications are incorporated herein by reference.

### TECHNICAL FIELD

The present disclosure relates to the technical field of maternal and infant products, and in particular, to a breast pump and a breast pump assembly.

### BACKGROUND

Breast pumps are designed for postpartum women. When breastfeeding is not practical, lactating women can use the breast pump to express milk from the breast for storage and subsequent use. This prevents the waste of breast milk, and facilitates the work and lives of the lactating women. Therefore, the breast pumps have gained widespread popularity in the market.

Typically, a breast pump includes a nipple tunnel cap element having a nipple tunnel and a negative pressure part having a negative pressure chamber. Through the negative pressure chamber, a negative pressure is transferred to the nipple tunnel, thereby expressing the milk from the breast. In the related art, the nipple tunnel cap element and the negative pressure part are detachably assembled together. This results in an excessive number of components of the breast pump, increasing assembly difficulty, and causing more assembly errors and potential airtightness hazards.

Breastfeeding plays an irreplaceable role in the growth and development of newborns. For mothers who are unable to breastfeed directly or desire to store milk for later use, breast pumps have become indispensable auxiliary tools.

Although a wide variety of breast pumps are available on the market, users still face several challenges during use, particularly regarding improving the milk expression efficiency and addressing problems of residual milk (i.e., “milk retention”) and accumulated milk (i.e., “milk accumulation”).

With the growing social awareness of the importance of breastfeeding and the increasing demand of modern women for work-life balance, the market demand for breast pumps as auxiliary lactation tools continues to rise. Existing breast pumps come in a variety of designs. They are intended to provide lactating women with a convenient and comfortable milk expression experience, while ensuring the effective storage and hygienic safety of the milk. Most early breast pumps are structurally simple and functionally single. For example, manual breast pumps generate a negative pressure for milk expression by manually squeezing an air sac or a handle. Despite their low cost, such pumps are cumbersome to operate, and are limited in efficiency and comfort. With the technical advancement, electric breast pumps have gradually become the mainstream in the market. The negative pressure pump is driven by an electric motor to generate a stable suction force, thereby greatly improving the milk expression efficiency and the user comfort.

As science and technology advance, electric breast pumps have emerged and become popular with users. The negative pressure pump is electrically driven to generate a stable suction force, thereby significantly improving the milk expression efficiency and the user comfort. However, existing electric breast pumps still have certain limitations regarding their structural design, size and portability. Due to a complex internal structure and a large size, some of these pumps lack portability, making them unsuitable for on-the-go or working mothers.

As auxiliary breastfeeding tools, breast pumps have gained widespread popularity in the current maternal and infant product market because they can effectively alleviate milk stasis, facilitate milk collection, and support breastfeeding. With technological advancements and growing consumer demands, the design of breast pumps has been evolving toward greater user-friendliness, higher efficiency, and improved hygiene.

For the annular main pump module provided with a through hole, the layout of components in the main pump module is restricted. Some pumping power assemblies differ significantly from other components in terms of size and weight, resulting in an unstable center of gravity of the breast pump. Consequently, the breast pump is prone to falling off during wearing and offers poor wearing comfort.

With the accelerating pace of modern life, more and more mothers use breast pumps to collect breast milk, thereby providing nutrition for infants when direct breastfeeding is not practical. For the sake of expressing milk from the breast, a traditional breast pump typically includes one or more components, such as a breast shield and a milk pumping conduit. However, in actual use, it is difficult to visually observe the flow condition of the milk and the internal state of the breast pump device. This not only compromises user experience, but may also lead to improper operation or misjudgment of the device state.

For most existing breast pumps, the whole milk expression process is not visible to users. Particularly in nighttime or low-light environments, it is difficult to determine whether the milk flows out smoothly, or whether air bubbles are present in the breast shield. This causes inconvenience to users and may even affect the milk expression efficiency and comfort.

### BRIEF SUMMARY

According to a first aspect, a first objective of the present disclosure is to provide a breast pump assembly, to solve the technical problem that the existing breast pump has an excessive number of components to cause large assembly difficulty and potential airtightness hazards.

To achieve the above objective, the present disclosure provides the following solutions: A breast pump assembly is configured for a breast pump, and includes: a container body and a container lid, where the container body and the container lid enclose a milk storage chamber; a negative pressure part, where the negative pressure part is connected to the container lid, the negative pressure part has a negative pressure chamber, and the negative pressure chamber is configured to communicate with a negative pressure suction port of the breast pump; and a nipple tunnel cap element, where the nipple tunnel cap element is connected to the container lid, the nipple tunnel cap element has a nipple tunnel, the nipple tunnel communicates with the negative pressure chamber, and the nipple tunnel is at least configured to guide milk from a breast; the negative pressure part, the nipple tunnel cap element, and the container lid are integrally formed, and one of the nipple tunnel and the negative pressure chamber communicates with the milk storage chamber.

As an implementation, both the nipple tunnel cap element and the negative pressure part are connected to a side of the container lid facing the milk storage chamber.

As an implementation, a first perforation and a second perforation are formed in the container lid; a central axis of the first perforation is parallel to a central axis of the second perforation; the negative pressure part is connected to the first perforation; the first perforation is configured to communicate the negative pressure chamber with the negative pressure suction port; the nipple tunnel cap element is connected to the second perforation; and the second perforation communicates with the nipple tunnel, and is configured to allow the milk to pass through and flow to the nipple tunnel.

As an implementation, the central axis of the first perforation is coaxial with a central axis of the negative pressure chamber; and the central axis of the second perforation is coaxial with a central axis of the nipple tunnel.

As an implementation, the first perforation is closely adjacent to the second perforation; and/or when the breast pump is placed against the breast, the first perforation is located below the second perforation.

As an implementation, when the breast pump is placed against the breast, the first perforation is located directly under the second perforation, and the negative pressure chamber communicates with the milk storage chamber.

As an implementation, the container body is provided with a first mounting groove; and the container lid is accommodated in the first mounting groove, so as to enclose the milk storage chamber with the container body.

A second objective of the present disclosure is to provide a breast pump, including: a breast shield, where the breast shield is provided with a breast receptacle; a main pump module, where the main pump module is provided with a negative pressure suction port configured to transfer a negative pressure; and the above breast pump assembly, where the nipple tunnel communicates with the breast receptacle, and the negative pressure chamber communicates with the negative pressure suction port.

As an implementation, the first perforation is formed in the container lid; the negative pressure part is connected to the first perforation and located on the side of the container lid facing the milk storage chamber; the breast pump further includes a liquid separation device; the liquid separation device includes a liquid separation device body and a ring connected to the liquid separation device body; a circumferential direction of the main pump module around the negative pressure suction port and/or a circumferential direction of the container lid around the first perforation is provided with a second mounting groove; the ring is disposed in the second mounting groove; and the liquid separation device body is accommodated in the negative pressure chamber.

As an implementation, the negative pressure part further has a first through hole and a second through hole; the first through hole is configured to communicate the negative pressure chamber with the nipple tunnel; the second through hole is configured to communicate the negative pressure chamber with the milk storage chamber; when the breast pump is placed against the breast, the negative pressure chamber is located below the nipple tunnel; the liquid separation device has a contracted state and an expanded state; and when the liquid separation device is in the expanded state, at least a portion of the liquid separation device corresponding to the first through hole is spaced apart from a wall of the negative pressure chamber.

According to the breast pump assembly and the breast pump provided by the present disclosure, both the negative pressure part and the nipple tunnel cap element are connected to the container lid, and the negative pressure part, the nipple tunnel cap element, and the container lid are integrally formed. That is, the negative pressure part, the nipple tunnel cap element, and the container lid are designed into a whole. This reduces the number of components of the breast pump assembly, thereby lowering the assembly difficulty of the breast pump assembly, and improving the assembly efficiency. In addition, the operation of assembling the negative pressure part and the nipple tunnel cap element on the container lid is omitted, thereby preventing the problem of poor airtightness due to assembly errors of the negative pressure part and the nipple tunnel cap element.

A third objective of the present disclosure is to provide a breast pump, to solve the technical problem that the user cannot quickly and effectively get a problem of the breast pump in use.

To achieve the above objective, the present disclosure provides the following solutions: A breast pump includes: a nipple tunnel cap element, where the nipple tunnel cap element is made of an optically transparent material and has a nipple tunnel, and the nipple tunnel is at least configured to guide milk from a breast; a negative pressure part, where the negative pressure part has a negative pressure chamber, and the negative pressure chamber at least communicates with the nipple tunnel; and a main pump module, where the main pump module is provided with a negative pressure suction port, and the negative pressure suction port communicates with the negative pressure chamber; and when the breast pump is placed against the breast, the negative pressure part is located below the nipple tunnel cap element, and the main pump module is located at a position other than on the nipple tunnel cap element.

As an implementation, at least a portion of the main pump module is disposed on a side of the negative pressure part away from the nipple tunnel cap element.

As an implementation, the negative pressure part is made of the optically transparent material.

As an implementation, the breast pump further includes a milk storage container; both the nipple tunnel cap element and the negative pressure part are connected to an inner side of the milk storage container, and the main pump module is disposed on an outer side of the milk storage container; and when the breast pump is placed against the breast, at least a portion of the milk storage container located above the nipple tunnel cap element is made of the optically transparent material.

As an implementation, the milk storage container is entirely made of the optically transparent material.

As an implementation, the milk storage container includes a first container wall and a second container wall; the first container wall and the second container wall enclose a milk storage chamber; both the negative pressure part and the nipple tunnel cap element are connected to a side of the second container wall facing the milk storage chamber; when the breast pump is placed against the breast, the first container wall is located above the nipple tunnel cap element; and transparency of the first container wall is greater than or equal to transparency of the second container wall.

As an implementation, the breast pump further includes a breast shield; the breast shield includes a breast attachment portion and a channel portion connected to the breast attachment portion; the breast attachment portion is configured to be adsorbed to the breast; the channel portion is provided with a nipple channel; and at least a portion of the channel portion extends into the nipple tunnel cap element, such that the nipple channel communicates with the nipple tunnel.

As an implementation, the channel portion is made of the optically transparent material.

As an implementation, the channel portion is in an interference fit with the nipple tunnel cap element; or an outer surface of the channel portion is smooth; or the breast shield is detachably connected to the nipple tunnel cap element.

As an implementation, an end of the channel portion away from the breast attachment portion and an inner surface of the nipple tunnel cap element define a step, so as to prevent backflow of the milk.

According to the breast pump provided by the present disclosure, the nipple tunnel cap element having the nipple tunnel is provided, and configured to deliver the milk from the breast, and the nipple tunnel cap element is made of the optically transparent material, such that the user can directly view a condition in the nipple tunnel. Since the negative pressure part is disposed under the nipple tunnel cap element, and the main pump module is disposed at the position other than on the nipple tunnel cap element, when the breast pump is used, the user views the breast pump from the position above the breast pump, and neither the negative pressure part nor the main pump module shields the nipple tunnel cap element. Thus, the user can view a flowing condition of the milk in the nipple tunnel in real time. In case of an excessively fast or slow flow rate, an operation mode can be adjusted at any time, so as to slow down or speed up flowing of the milk. Therefore, when the breast pump provided by the present disclosure is used, the user can quickly and effectively get the problem of the breast pump to immediately adjust the operation mode, greatly improving the use effect of the breast pump.

A fourth objective of the present disclosure is to provide a breast pump assembly, to solve the technical problem of the excessively large size of the breast pump.

To achieve the above objective, the present disclosure provides the following solutions: A breast pump assembly is configured for a breast pump, and includes: a negative pressure part, where the negative pressure part has a negative pressure chamber, and the negative pressure chamber is configured to communicate with a negative pressure suction port of the breast pump; and a nipple tunnel cap element, where the nipple tunnel cap element has a nipple tunnel, the nipple tunnel communicates with the negative pressure chamber, and the nipple tunnel is at least configured to guide milk from a breast; and the nipple tunnel cap element and the negative pressure part are arranged side by side in the breast pump.

As an implementation, when the breast pump is placed against the breast, the negative pressure part is located below the nipple tunnel cap element.

As an implementation, the nipple tunnel cap element and the negative pressure part share a first side wall; two opposite sides of the first side wall face the negative pressure chamber and the nipple tunnel, respectively; and a first through hole configured to communicate the negative pressure chamber with the nipple tunnel is formed in the first side wall.

As an implementation, when the breast pump is placed against the breast, the first through hole is located at a lowest position of the nipple tunnel cap element.

As an implementation, the negative pressure part further includes a second side wall and a first bottom wall; and the first side wall, the second side wall, and the first bottom wall enclose the negative pressure chamber; and a second through hole configured to communicate with negative pressure chamber is formed in the second side wall; and when the breast pump is placed against the breast, the second through hole is located below the first through hole.

As an implementation, when the breast pump is placed against the breast, the second through hole is located at a lowest position of the negative pressure part.

As an implementation, when the breast pump is placed against the breast, the second through hole directly faces the first through hole along a vertical direction; or, the second through hole is formed in an end of the second side wall close to the first bottom wall.

As an implementation, a length of the nipple tunnel cap element is the same as a length of the negative pressure part; and/or, an opening of the negative pressure chamber for communicating with the negative pressure suction port and an opening of the nipple tunnel for receiving the milk are arranged side by side and have a same orientation.

As an implementation, the breast pump assembly includes a container body and a container lid; the container body and the container lid enclose a milk storage chamber; and the nipple tunnel cap element and the negative pressure part are connected side by side to a same surface of the container lid and extend toward one side of the milk storage chamber.

A fifth objective of the present disclosure is to provide a breast pump, including: a breast shield, where a breast receptacle is formed in the breast shield; a main pump module, where the main pump module is provided with a negative pressure suction port configured to transfer a negative pressure; and the breast pump assembly, where the nipple tunnel communicates with the breast receptacle, and the negative pressure chamber communicates with the negative pressure suction port.

According to the breast pump assembly and the breast pump provided by the present disclosure, the nipple tunnel cap element and the negative pressure part are arranged side by side in the breast pump, i.e., the nipple tunnel cap element and the negative pressure part have a same extension direction, so the breast pump only needs to provide an enough space in a direction same as the extension direction of the nipple tunnel cap element and the negative pressure part to accommodate the nipple tunnel cap element and the negative pressure part. In contrast to the arrangement where the nipple tunnel cap element and the negative pressure part are crossed, and the breast pump provides enough accommodating spaces in two different extension directions for the nipple tunnel cap element and the negative pressure part, the present disclosure effectively reduces the occupied space of the breast pump assembly. Therefore, the breast pump assembly provided by this embodiment has a small occupied space, thereby reducing the overall size of the breast pump.

A sixth objective of the present disclosure is to provide a breast pump, to solve the technical problems of unreasonable structural design and large occupied space of the existing main pump module.

To achieve the above objective, the present disclosure provides the following solutions: A breast pump includes: a main pump module housing, where the main pump module housing includes a first shell and a second shell extending relative to the first shell in a bending manner, so as to form a step on the main pump module housing, and an accommodating chamber is formed in the main pump module housing; a milk pumping assembly, where the milk pumping assembly is at least partially located on the step; and a pumping power device that is disposed in the accommodating chamber, where the pumping power device is configured to generate a negative pressure.

As an implementation, the milk pumping assembly includes a milk collection unit and a breast shield; the milk collection unit is disposed on one side of the first shell; the second shell is disposed between the breast shield and the milk collection unit; and at least one of the first shell and the second shell has a cavity, so as to form the accommodating chamber.

As an implementation, the first shell has a first cavity; the second shell has a second cavity; the accommodating chamber includes the first cavity and the second cavity; and at least a portion of the pumping power device is accommodated in the first cavity.

As an implementation, the pumping power device includes a negative pressure pump, a solenoid valve, a battery, and a circuit board; the negative pressure pump, the solenoid valve, and the battery are accommodated in the first cavity; and the circuit board is accommodated in the second cavity.

As an implementation, the negative pressure pump, the battery, and the solenoid valve are arranged side by side; and the battery is located between the negative pressure pump and the solenoid valve.

As an implementation, a negative pressure suction port is formed in a side of the second shell provided with the first shell; and the negative pressure suction port communicates with the second cavity.

As an implementation, the breast pump further includes a key unit; and the key unit is disposed on a circumferential side, other than an end close to the first shell, of the second shell.

As an implementation, the second shell is further provided with a through hole; the through hole extends from a side of the second shell away from the first shell to a side of the second shell close to the first shell; and the through hole is configured to allow the breast shield to penetrate through.

As an implementation, the milk collection unit is supported on the first shell; and/or, the main pump module housing is L-shaped; and an L-shaped step is formed on the main pump module housing.

As an implementation, the milk collection unit includes a milk storage container and a breast pump assembly extending into the milk storage container; and the pumping power device is configured to provide the negative pressure for the breast pump assembly.

According to the breast pump provided by the present disclosure, the second shell extends relative to the first shell in the bending manner, the step is formed on the main pump module housing, and the milk pumping assembly is at least partially located on the step, such that the main pump module housing and the milk pumping assembly are closely assembled together. This arrangement is reasonable, and greatly reduces the occupied space of the main pump module housing and the overall size of the breast pump, thereby making the breast pump convenient to carry and use, and enhancing the user experience.

A seventh objective of the present disclosure is to provide a breast pump, to solve the technical problems of the large size and weight of the existing breast pump.

To achieve the above objective, the present disclosure provides the following solutions: A breast pump includes: a main pump module, where the main pump module is configured to provide a negative pressure; and a milk storage container, where the milk storage container includes a support wall and an enclosure wall, the support wall and the enclosure wall enclose a milk storage chamber, and the support wall is configured to support milk flowing to the milk storage chamber; and at least a portion of the main pump module is attached to a side of the support wall away from the milk storage chamber.

As an implementation, the main pump module includes a first bottom shell, a first top shell, and a negative pressure pump; the first bottom shell and the first top shell enclose a first accommodating chamber; the negative pressure pump is accommodated in the first accommodating chamber; and the first top shell is attached to the side of the support wall away from the milk storage chamber.

As an implementation, a projected contour of the first top shell along a direction toward the support wall falls within a contour of the support wall.

As an implementation, a thickness of the first top shell is less than or equal to a thickness of the first bottom shell.

As an implementation, the first top shell includes at least two panels; any two adjacent ones of the panels are disposed at an included angle; and the included angle is less than 180°.

As an implementation, each of the panels is non-planar; and/or, two adjacent ones of the panels are in smooth transition.

As an implementation, the at least two panels define a limiting recess; and the limiting recess is configured to limit the milk storage container.

As an implementation, the negative pressure pump corresponds to a bottom wall of the limiting recess.

As an implementation, one of the first top shell and the support wall is provided with an anti-disengagement snap groove, while the other is provided with an anti-disengagement snap protrusion; and the anti-disengagement snap protrusion is snap-fitted into the anti-disengagement snap groove.

As an implementation, the main pump module further includes a solenoid valve; and the negative pressure pump and the solenoid valve are accommodated side by side in the first accommodating chamber.

According to the breast pump provided by the present disclosure, with the milk storage container, expressed milk can flow to the milk storage chamber, and is supported on the support wall. At least a portion of the main pump module is attached to the side of the support wall away from the milk storage chamber. When the milk is supported on the support wall, since the temperature of the milk is lower than the temperature of the main pump module, the milk can absorb heat of the main pump module, thereby achieving a heat dissipation effect for the main pump module. In contrast to the heat dissipation method using heat sinks, this arrangement omits the heat sinks, greatly reduces the overall size and weight of the device, and further enhances the user experience.

According to a second aspect, in view of shortcomings in the prior art, the present disclosure provides a breast pump, to solve the problems of milk accumulation and low milk expression efficiency.

A technical solution adopted by embodiments of the present disclosure is as follows: A breast pump is provided, including: a milk pumping conduit, where a nipple tunnel is formed in the milk pumping conduit, the nipple tunnel is configured to receive milk from a breast, a milk outlet is formed in the milk pumping conduit, and a negative pressure is transferred to the nipple tunnel through the milk outlet; and a milk storage container, where a milk storage chamber is formed in the milk storage container, and the milk in the nipple tunnel flows out of the nipple tunnel through the milk outlet and flows to the milk storage chamber.

Optionally, the milk storage container is a milk bowl assembly; and the milk pumping conduit is connected to the milk bowl assembly.

Optionally, the milk bowl assembly includes a bowl body and a cover plate; the bowl body and the cover plate enclose the milk storage chamber; and the milk pumping conduit is connected to the cover plate.

Optionally, the breast pump includes a breast shield; and the milk pumping conduit is connected to the breast shield.

Optionally, the breast pump further includes a diaphragm bracket; a negative pressure chamber is formed in the diaphragm bracket; and the negative pressure chamber communicates with the nipple tunnel through the milk outlet.

Optionally, the diaphragm bracket is provided with a milk flow port; and after flowing out of the nipple tunnel through the milk outlet, the milk in the nipple tunnel flows through the negative pressure chamber and flows to the milk storage chamber through the milk flow port.

Optionally, the breast pump further includes a main pump module; and the main pump module is configured to provide the negative pressure.

Optionally, the breast pump further includes a main pump module; the main pump module includes a negative pressure port; the main pump module communicates with the negative pressure chamber through the negative pressure port; the main pump module is configured to form the negative pressure in the negative pressure chamber; and the negative pressure is transferred to the nipple tunnel through the milk outlet.

Optionally, the negative pressure chamber is at least partially lower than the nipple tunnel when the breast pump is worn.

Optionally, the breast pump further includes a diaphragm; the diaphragm is disposed in the negative pressure chamber; and when the breast pump is worn, at least a portion of the diaphragm is actuated below the nipple tunnel.

Optionally, the milk outlet is located at an underside of the milk pumping conduit.

Optionally, the milk outlet is located at a lower position of an end of the milk pumping conduit.

Optionally, the milk outlet is located between an end of the milk pumping conduit and the cover plate.

Optionally, when the breast pump is worn, an end of the milk pumping conduit away from a human body is sealed.

Optionally, the breast pump further includes a breast shield; and one end of the breast shield at least partially overlaps with the milk pumping conduit.

Optionally, a position where the one end of the breast shield overlaps with the milk pumping conduit forms a double-layered structure; and the double-layered structure is at least partially made of a light-transmissive material, such that an interior of the nipple tunnel or the breast shield can be observed through the double-layered structure.

Optionally, the end of the breast shield is inserted into the milk pumping conduit or the milk pumping conduit is inserted into the end of the breast shield.

Optionally, the breast pump further includes a breast shield; a through hole is formed in a thickness direction of the main pump module; the breast shield has a narrow end and a wide end; the narrow end of the breast shield penetrates into the through hole; and the main pump module is at least partially located between the wide end of the breast shield and the milk bowl assembly.

Optionally, the milk storage container is the milk bowl assembly; the main pump module includes a transverse portion and a longitudinal portion connected to the transverse portion in a bending manner; a first side of the milk bowl assembly contacts the longitudinal portion of the main pump module; a second side of the milk bowl assembly contacts the transverse portion of the main pump module; and the first side and the second side are two adjacent sides of the milk bowl assembly.

The breast pump further includes a diaphragm; the diaphragm is configured to be placed in the negative pressure chamber; the diaphragm divides an internal space of a diaphragm bracket into a first subspace and a second subspace; the first subspace communicates with a negative pressure port of the main pump module; the second subspace communicates with an internal space of the milk pumping conduit; and driven by the negative pressure provided by the main pump module, the diaphragm can deform in the internal space of the diaphragm bracket to transfer the negative pressure.

According to this embodiment of the present disclosure, the breast pump includes the milk pumping conduit and the milk storage container. The nipple tunnel is formed in the milk pumping conduit. The nipple tunnel is configured to receive the milk from the breast. The milk outlet is formed in the milk pumping conduit. The negative pressure is transferred to the nipple tunnel through the milk outlet. The milk storage chamber is formed in the milk storage container. The milk in the nipple tunnel flows out of the nipple tunnel through the milk outlet and flows to the milk storage chamber. The transfer of the negative pressure and the outflow of the milk are realized through the milk outlet, preventing the milk from remaining in the nipple tunnel, and improving the milk expression efficiency.

In view of shortcomings in the prior art, the present disclosure provides a breast pump, to solve the problems of milk accumulation and low milk expression efficiency.

To solve the above technical problem, a technical solution adopted by the present disclosure is as follows: A breast pump is provided, including: a milk pumping conduit, where a nipple tunnel is formed in the milk pumping conduit, and the nipple tunnel is configured to receive milk from a breast; and a diaphragm bracket, where a negative pressure chamber is formed in the diaphragm bracket, the negative pressure chamber communicates with the nipple tunnel, and the negative pressure chamber is at least partially lower than the nipple tunnel when the breast pump is worn.

Optionally, the breast pump further includes a diaphragm; the diaphragm is disposed in the negative pressure chamber; and when the breast pump is worn, at least a portion of the diaphragm is actuated below the nipple tunnel.

Optionally, a milk outlet is formed in the milk pumping conduit; a negative pressure is transferred to the nipple tunnel through the milk outlet; and the milk in the nipple tunnel flows out of the nipple tunnel through the milk outlet under the action of the negative pressure.

Optionally, the milk outlet is located at an underside of the milk pumping conduit.

Optionally, the milk outlet is located at a lower position of an end of the milk pumping conduit.

Optionally, when the breast pump is worn, an end of the milk pumping conduit away from a human body is sealed to form a sealed end.

Optionally, the milk outlet is located between the sealed end and a cover plate.

Optionally, the breast pump further includes a milk bowl assembly; and the milk pumping conduit is connected to the milk bowl assembly.

Optionally, the milk bowl assembly includes a bowl body and a cover plate; the bowl body and the cover plate enclose a milk storage chamber; and the milk pumping conduit is connected to the cover plate.

Optionally, the breast pump further includes a breast shield; an internal space of the breast shield communicates with the nipple tunnel; and the milk pumping conduit is connected to the breast shield.

According to this embodiment of the present disclosure, the breast pump includes the milk pumping conduit and the diaphragm bracket. The nipple tunnel is formed in the milk pumping conduit. The nipple tunnel is configured to receive the milk from the breast. The negative pressure chamber is formed in the diaphragm bracket. The negative pressure chamber communicates with the nipple tunnel. The negative pressure chamber is at least partially lower than the nipple tunnel when the breast pump is worn. This prevents the milk from remaining in the nipple tunnel, and improves the milk expression efficiency.

In view of shortcomings in the prior art, an embodiment of the present disclosure provides a breast pump, to solve the problems of insufficiently compact structure and low milk expression efficiency of the breast pump.

To solve the above technical problem, a technical solution adopted by the present disclosure is as follows: A breast pump is provided, including: a milk bowl assembly, where the milk bowl assembly includes a bowl body and a cover plate; and the bowl body and the cover plate enclose a milk storage chamber; and a milk pumping conduit, where the milk pumping conduit is connected to the cover plate, a nipple tunnel is formed in the milk pumping conduit, the nipple tunnel is configured to receive milk from a breast, a milk outlet is formed in the milk pumping conduit, the milk in the nipple tunnel flows out of the milk pumping conduit through the milk outlet under the action of a negative pressure, the milk pumping conduit includes a distal end away from the cover plate, and the milk outlet is located between the cover plate and the distal end.

Optionally, the distal end of the milk pumping conduit is a sealed end.

Optionally, the distal end of the milk pumping conduit is an unsealed end; and the milk outlet is located at a lower position of the distal end.

Optionally, the negative pressure is transferred to the nipple tunnel through the milk outlet; and the milk in the nipple tunnel flows out of the nipple tunnel through the milk outlet.

Optionally, the breast pump further includes a diaphragm bracket; a negative pressure chamber is formed in the diaphragm bracket; and the negative pressure chamber communicates with the nipple tunnel through the milk outlet.

Optionally, the diaphragm bracket is provided with a milk flow port; and after flowing out of the nipple tunnel through the milk outlet, the milk in the nipple tunnel flows through the negative pressure chamber and flows to the milk storage chamber through the milk flow port.

Optionally, the breast pump further includes a main pump module; and the main pump module is configured to provide the negative pressure.

Optionally, the breast pump further includes a main pump module; the main pump module includes a negative pressure port; the main pump module communicates with the negative pressure chamber through the negative pressure port; the main pump module is configured to form the negative pressure in the negative pressure chamber; and the negative pressure is transferred to the nipple tunnel through the milk outlet.

Optionally, the breast pump further includes a breast shield; and one end of the breast shield at least partially overlaps with the milk pumping conduit.

Optionally, a position where the one end of the breast shield overlaps with the milk pumping conduit forms a double-layered structure that includes at least two layers; and the double-layered structure is at least partially transparent, such that an interior of the nipple tunnel or the breast shield can be observed through the double-layered structure.

According to this embodiment of the present disclosure, the breast pump includes the milk bowl assembly and the milk pumping conduit. The milk bowl assembly includes the bowl body and the cover plate. The bowl body and the cover plate enclose the milk storage chamber. The milk pumping conduit is connected to the cover plate. The nipple tunnel is formed in the milk pumping conduit. The nipple tunnel is configured to receive the milk from the breast. The milk outlet is formed in the milk pumping conduit. The milk in the nipple tunnel flows out of the milk pumping conduit through the milk outlet under the action of the negative pressure. The milk pumping conduit includes the distal end away from the cover plate. The milk outlet is located between the cover plate and the distal end. This can contribute to miniaturized structural design of the breast pump, and can improve the milk expression efficiency.

According to a third aspect, in view of shortcomings in the prior art, the present disclosure provides a breast pump, to solve the problems of the uncompact structure and large size of the electric breast pump.

A technical solution adopted by the present disclosure is as follows: A breast pump is provided, including: a milk bowl assembly, where a milk storage chamber is formed in the milk bowl assembly; a main pump module, where a through hole is formed in a thickness direction of the main pump module; a breast shield, where the breast shield has a narrow end and a wide end, the narrow end of the breast shield penetrates into the through hole, and the main pump module is at least partially located between the wide end of the breast shield and the milk bowl assembly; and a diaphragm bracket, where the diaphragm bracket is connected to the milk bowl assembly and extends into the milk storage chamber.

The breast pump further includes a milk pumping conduit connected to the milk bowl assembly and extending into the milk storage chamber; the milk pumping conduit communicates with an internal space of the breast shield; and a length of the milk pumping conduit extending into the milk storage chamber is less than a length of the diaphragm bracket.

An end of the milk pumping conduit away from the breast shield is provided with a milk outlet; and the milk outlet is configured to allow milk in the milk pumping conduit to flow to the milk storage chamber.

A sum of a length of the milk pumping conduit and a length of the milk outlet is less than or equal to the length of the diaphragm bracket.

The milk bowl assembly includes a bowl body and a cover plate; the bowl body and the cover plate enclose the milk storage chamber; and both the milk pumping conduit and the diaphragm bracket are connected to the cover plate.

The milk pumping conduit and the diaphragm bracket are integrally connected with the cover plate.

The milk pumping conduit and the diaphragm bracket are arranged side by side.

The milk pumping conduit is located on the diaphragm bracket.

The main pump module includes a transverse portion and a longitudinal portion connected to the transverse portion in a bending manner; the through hole is formed in the longitudinal portion; the longitudinal portion is located between the wide end of the breast shield and the milk bowl assembly; a first side of the milk bowl assembly comes into contact with the longitudinal portion of the main pump module; a second side of the milk bowl assembly comes into contact with the transverse portion of the main pump module; and the first side and the second side are two adjacent sides of the milk bowl assembly.

The breast pump further includes a diaphragm; the diaphragm is configured to be placed in the diaphragm bracket; the diaphragm divides an internal space of the diaphragm bracket into a first subspace and a second subspace; the first subspace communicates with a negative pressure port of the main pump module; the second subspace communicates with an internal space of the milk pumping conduit; and driven by a negative pressure provided by the main pump module, the diaphragm can deform in the internal space of the diaphragm bracket to transfer the negative pressure.

According to this embodiment of the present disclosure, the breast pump includes the milk bowl assembly, the main pump module, and the breast shield. The milk storage chamber is formed in the milk bowl assembly. The through hole is formed in the thickness direction of the main pump module. The breast shield has the narrow end and the wide end. The narrow end of the breast shield penetrates into the through hole. The main pump module is at least partially located between the wide end of the breast shield and the milk bowl assembly. The diaphragm bracket is connected to the milk bowl assembly and extends into the milk storage chamber. The above configuration can reduce the overall occupied thickness space of the main pump module, the milk bowl assembly, and the breast shield. Moreover, the diaphragm bracket is connected to the milk bowl assembly and extends into the internal milk storage chamber of the milk bowl assembly, such that the diaphragm bracket does not additionally occupy the thickness space, further reducing the overall size of the breast pump, and facilitating portability.

In view of shortcomings in the prior art, the present disclosure provides a breast pump, to solve the problems of the large size and unstable connection of the breast pump.

To solve the above technical problem, a technical solution adopted by the present disclosure is as follows: A breast pump is provided, including: a main pump module, where the main pump module is configured to provide a negative pressure for milk expression; a milk bowl assembly, where the milk bowl assembly is configured to accommodate milk, and the milk bowl assembly is disposed on a first side of the main pump module; and a breast shield, where the breast shield has a narrow end and a wide end, an inner diameter of the breast shield from the narrow end to the wide end gradually increases, the wide end of the breast shield is disposed on a second side of the main pump module away from the first side, and the breast shield is connected to the main pump module.

The breast shield is detachably connected to the main pump module.

The breast pump further includes a milk pumping conduit connected to the milk bowl assembly; the milk pumping conduit extends into a milk storage chamber of the milk bowl assembly; and the narrow end of the breast shield communicates with an end of the milk pumping conduit.

A through hole is formed in a thickness direction of the main pump module; the narrow end of the breast shield is inserted into the through hole; and the narrow end of the breast shield cooperates with the through hole to realize a connection between the breast shield and the main pump module.

One of an outer wall of the narrow end and an inner wall of the through hole is provided with a groove, while the other is provided with a ball plunger; and the groove cooperates with the ball plunger to realize the connection between the breast shield and the main pump module.

The narrow end is in an interference fit with the through hole to realize the connection between the breast shield and the main pump module.

One of an outer wall of the narrow end and an inner wall of the through hole is provided with a snap fastener, while the other is provided with a snap groove; and the snap fastener cooperates with the snap groove to realize the connection between the breast shield and the main pump module.

One of the main pump module and the breast shield is provided with a locating groove, while the other is provided with a locating protrusion.

The main pump module includes a transverse portion and a longitudinal portion connected to the transverse portion in a bending manner; the through hole is formed in the longitudinal portion; the longitudinal portion is located between the wide end of the breast shield and the milk bowl assembly; a first side of the milk bowl assembly comes into contact with the longitudinal portion of the main pump module; a second side of the milk bowl assembly comes into contact with the transverse portion of the main pump module; and the first side and the second side are two adjacent sides of the milk bowl assembly.

An end surface of the narrow end abuts against an end surface of the milk pumping conduit, such that the narrow end communicates with the milk pumping conduit in a sealing manner.

According to this embodiment of the present disclosure, the breast pump includes the main pump module, the milk bowl assembly, and the breast shield. The main pump module is configured to provide the negative pressure for the milk expression. The milk bowl assembly is configured to accommodate the milk. The milk bowl assembly is disposed on the first side of the main pump module. The breast shield has the narrow end and the wide end. The inner diameter of the breast shield from the narrow end to the wide end gradually increases. The wide end of the breast shield is disposed on the second side of the main pump module away from the first side. The breast shield is connected to the main pump module. The breast shield and the milk bowl assembly are respectively disposed on two opposite sides of the main pump module, and the breast shield is connected to the main pump module, such that the overall size of the breast pump can be smaller. Meanwhile, the breast shield is connected to the main pump module, such that the connecting structure therebetween can be further designed more stably and reliably.

In view of shortcomings in the prior art, the present disclosure provides a breast pump, to solve the problems of the non-uniform suction force and uncomfortable and unstable wearing of the breast pump.

To solve the above technical problem, a technical solution adopted by the present disclosure is as follows: A breast pump is provided, including: a breast shield, where the breast shield has a wide end and a narrow end, and an inner diameter of the breast shield from the narrow end to the wide end gradually increases; a milk bowl assembly, where a milk storage chamber is formed in the milk bowl assembly; a milk pumping conduit, where the milk pumping conduit is connected to the milk bowl assembly and extends into the milk storage chamber, the milk pumping conduit communicates with the breast shield, and the milk storage chamber is configured to receive milk flowing out of the milk pumping conduit; and a main pump module that is configured to provide the milk pumping conduit with a negative pressure for milk expression; and when the milk storage chamber is empty, a center of gravity of the breast pump is located within the milk pumping conduit or the narrow end of the breast shield.

A through hole is formed in the main pump module; the breast shield penetrates into the through hole; and the main pump module is located between the wide end of the breast shield and the milk bowl assembly.

A through hole is formed in the main pump module; and a center of gravity of the main pump module is located within the through hole.

The main pump module includes a housing as well as a negative pressure pump, a solenoid valve, a first battery, and a second battery that are disposed in the housing; the negative pressure pump and the solenoid valve are located below the through hole; and the first battery and the second battery are located at a left side and a right side of the through hole, respectively.

The main pump module further includes a circuit board located in the housing; and the circuit board is located above the through hole.

The main pump module includes a transverse portion and a longitudinal portion connected to the transverse portion in a bending manner; the through hole is formed in the longitudinal portion; the longitudinal portion is located between the wide end of the breast shield and the milk bowl assembly; a first side of the milk bowl assembly comes into contact with the longitudinal portion of the main pump module; a second side of the milk bowl assembly comes into contact with the transverse portion of the main pump module; and the first side and the second side are two adjacent sides of the milk bowl assembly.

The main pump module includes a transverse portion and a longitudinal portion connected to the transverse portion in a bending manner; the through hole is formed in the longitudinal portion; the longitudinal portion is located between the wide end of the breast shield and the milk bowl assembly; a first side of the milk bowl assembly comes into contact with the longitudinal portion of the main pump module; a second side of the milk bowl assembly comes into contact with the transverse portion of the main pump module; the first side and the second side are two adjacent sides of the milk bowl assembly; and the negative pressure pump and the solenoid valve are disposed in the transverse portion.

The negative pressure pump is a piezoelectric pump.

The milk bowl assembly includes a bowl body and a cover plate; the bowl body and the cover plate enclose the milk storage chamber; and the milk pumping conduit is connected to the cover plate.

The milk pumping conduit and the cover plate are integrally formed.

According to this embodiment of the present disclosure, the breast pump includes the breast shield, the milk bowl assembly, the milk pumping conduit, and the main pump module. The breast shield has the wide end and the narrow end. The inner diameter of the breast shield from the narrow end to the wide end gradually increases. The milk storage chamber is formed in the milk bowl assembly. The milk pumping conduit is connected to the milk bowl assembly and extends into the milk storage chamber. The milk pumping conduit communicates with the breast shield. The milk storage chamber is configured to receive the milk flowing out of the milk pumping conduit. The main pump module is configured to provide the milk pumping conduit with the negative pressure for the milk expression. When the milk storage chamber is empty, the center of gravity is located within the milk pumping conduit or the narrow end of the breast shield. By disposing the center of gravity of the breast pump within the milk pumping conduit or the narrow end of the breast shield, and close to a central position of a nipple, the breast pump is worn on the human body more stably and comfortably, with a more uniform suction force.

An objective of the present disclosure is to provide a breast pump assembly and a breast pump, to solve the technical problem that the milk easily flows from the nipple tunnel to the negative pressure chamber.

A first aspect of the present disclosure provides a breast pump assembly, applied to a breast pump, and including: a fixing member; a diaphragm bracket, where the diaphragm bracket is connected to the fixing member, and has a negative pressure chamber; an external nipple tunnel cap element that is connected to the fixing member and arranged side by side with the diaphragm bracket, where when the breast pump is placed against a breast, the external nipple tunnel cap element is located on the diaphragm bracket, and the external nipple tunnel cap element is provided with a first nipple tunnel, a milk flow hole configured to communicate the first nipple tunnel with a milk storage chamber of the breast pump, and a first air guide hole configured to communicate the first nipple tunnel with the negative pressure chamber; and an internal nipple tunnel cap element that at least partially extends into the first nipple tunnel, where the internal nipple tunnel cap element is provided with a second nipple tunnel and a second air guide hole communicating with the second nipple tunnel, and the second nipple tunnel communicates with the first nipple tunnel; the internal nipple tunnel cap element and an inner side wall of the external nipple tunnel cap element enclose an air guide channel; both the first air guide hole and the second air guide hole communicate with the air guide channel, and the second air guide hole is located above a central axis of the second nipple tunnel when the breast pump is placed against the breast.

As an implementation, when the breast pump is placed against the breast, a line connecting the second air guide hole and the central axis of the second nipple tunnel forms an included angle of 80-100° with a horizontal plane.

As an implementation, the internal nipple tunnel cap element includes a milk guide body and a soft rubber ring; the milk guide body extends into the first nipple tunnel, and is provided with the second nipple tunnel and a first perforation communicating with the second nipple tunnel; the soft rubber ring is sleeved on a periphery of the milk guide body and located between the milk guide body and the external nipple tunnel cap element; the soft rubber ring is provided with a first annular groove and a second perforation communicating with the first annular groove; the external nipple tunnel cap element is configured to seal the first annular groove to form the air guide channel; and the first perforation and the second perforation correspondingly form the second air guide hole.

As an implementation, an outer side wall of the milk guide body is provided with a second annular groove; and the soft rubber ring is nested in the second annular groove.

As an implementation, the second annular groove is formed in an end of the milk guide body extending into the first nipple tunnel.

As an implementation, the internal nipple tunnel cap element is detachably connected to the external nipple tunnel cap element.

As an implementation, the breast pump assembly further includes a liquid separation device and a negative pressure chamber cover; the liquid separation device is circumferentially connected to the diaphragm bracket and extends into the negative pressure chamber; and the negative pressure chamber cover is configured to cover an opening of the negative pressure chamber and seal the negative pressure chamber; and the negative pressure chamber cover is connected to the milk guide body, and integrally formed with the milk guide body.

As an implementation, the breast pump assembly further includes a handle; and the handle is connected to the milk guide body, and integrally formed with the milk guide body.

As an implementation, the breast pump assembly further includes a container body, a container lid, a main pump module housing, and a breast shield;
the container lid is configured to cover and seal the container body, and encloses a milk storage chamber with the container body; the main pump module housing is disposed between the container lid and the breast shield; and the breast shield is configured to be placed against the breast; and
the fixing member is the container lid or the main pump module housing or the breast shield; and both the external nipple tunnel cap element and the diaphragm bracket extend toward the milk storage chamber from the fixing member.

A second aspect of the present disclosure provides a breast pump, including the above breast pump assembly.

According to the breast pump assembly and the breast pump provided by the present disclosure, by forming the air guide channel, and providing the first air guide hole and the second air guide hole to communicate with the air guide channel, the negative pressure chamber communicates with the second nipple tunnel, and a negative pressure can be generated in the second nipple tunnel, thereby expressing the milk. By forming the second air guide hole above the central axis of the second nipple tunnel, the milk in the second nipple tunnel does not easily flow to the air guide channel and the negative pressure chamber through the second air guide hole, thereby preventing contamination to the negative pressure chamber, and degradation of suction performance of the breast pump.

An objective of the present disclosure is to provide a breast pump assembly and a breast pump, to solve the technical problem that the sealing member shields the user from viewing the nipple tunnel.

A first aspect of the present disclosure provides a breast pump assembly, applied to a breast pump, and including: a milk storage container, where the milk storage container includes a container body and a container lid, and the container lid and the container body enclose a milk storage chamber; and a nipple tunnel cap element, where the nipple tunnel cap element is connected to a side of the container lid facing the milk storage chamber or connected to a fixing member of the breast pump and extends into the milk storage chamber through the container lid, and the nipple tunnel cap element has a nipple tunnel; the container lid is provided with a milk pouring port communicating with the milk storage chamber; the breast pump assembly further includes a sealing member; and the sealing member is configured to seal the milk pouring port.

As an implementation, when the breast pump is placed against a breast, the milk pouring port is located above the nipple tunnel cap element.

As an implementation, the container lid includes a first plate and a second plate; and the second plate gradually extends toward the milk storage chamber from the first plate, and forms an included angle of greater than 90° and less than 180° with the first plate; and
the nipple tunnel cap element is connected to the first plate; and the milk pouring port is formed in the second plate.

As an implementation, the sealing member includes one end configured to detachably seal the milk pouring port, and another end connected to the container lid; and the sealing member and the container lid are integrally injection-molded.

As an implementation, the milk storage container further includes a sealing ring; the sealing ring is disposed between the container body and the container lid; the sealing member includes one end configured to detachably seal the milk pouring port, and another end connected to the sealing ring through the container lid; and the sealing member, the sealing ring, and the container lid are integrally injection-molded.

As an implementation, an end of the nipple tunnel cap element facing the milk storage chamber is provided with a milk flow port; and the milk flow port is configured to communicate the nipple tunnel with the milk storage chamber.

As an implementation, when the breast pump is placed against a breast, at least an upper half of the container body and at least an upper half of the nipple tunnel cap element are made of an optically transparent material.

As an implementation, the breast pump assembly further includes a diaphragm bracket; and the diaphragm bracket is connected to the side of the container lid facing the milk storage chamber or connected to the fixing member of the breast pump and extends into the milk storage chamber through the container lid; and
when the breast pump is placed against the breast, the diaphragm bracket is located under the nipple tunnel cap element.

As an implementation, the breast pump assembly further includes a main pump module housing and a breast shield; the main pump module housing is disposed between the container lid and the breast shield; the breast shield is configured to be placed against the breast; and the fixing member is the main pump module housing or the breast shield.

A second aspect of the present disclosure provides a breast pump, where the breast pump includes the above breast pump assembly.

According to the breast pump assembly and the breast pump provided by the present disclosure, by forming the milk pouring port in the container lid, since the container lid is not located on a path of the user for viewing the nipple tunnel during milk expression of the breast pump, the milk pouring port is not located on the path of the user for viewing the nipple tunnel, either. Therefore, the sealing member for sealing the milk pouring port does not shield a sight of the user, and the user can quickly and effectively know the problem of the breast pump in use according to a milk flow condition. Therefore, the breast pump assembly provided by the present disclosure prevents the sealing member from shielding the sight of the user for viewing the nipple tunnel, such that the user can quickly and effectively know the problem of the breast pump in use, improving the user experience.

An objective of the present disclosure is to provide a diaphragm assembly and a breast pump, to solve the technical problem of the excessive number of components of the breast pump.

A first aspect of the present disclosure provides a diaphragm assembly, applied to a breast pump, and including: a liquid separation device, where the liquid separation device has a cavity and an opening communicating with the cavity; a diaphragm cover, where the diaphragm cover is configured to detachably cover the opening, and provided with a first through hole; and a connector, where the connector includes one end fixedly connected to the liquid separation device, and another end fixedly connected to the diaphragm cover. As an implementation, the connector is configured to allow the diaphragm cover to pivotally cover the opening.

As an implementation, the connector includes a connecting portion, an embedding portion, and a bendable portion; the bendable portion is connected between the connecting portion and the embedding portion and is bendable; the connecting portion is fixedly connected to the liquid separation device; a side of the diaphragm cover away from the cavity is provided with a mounting groove; the embedding portion is nested in the mounting groove; and the first through hole is formed in a bottom wall of the mounting groove or in the diaphragm cover outside a region where the mounting groove is located.

As an implementation, both the connector and the liquid separation device are made of soft rubber; the diaphragm cover is made of hard rubber; and the connector and the liquid separation device are integrally formed and/or the connector and the diaphragm cover are integrally formed.

As an implementation, the liquid separation device includes a bottom wall, two long side walls, and two short side walls; the two short side walls are spaced-apart and opposite to each other, and are connected between the two long side walls; the bottom wall, the two long side walls, and the two short side walls enclose the cavity; ends of the two long side walls and the two short side walls away from the bottom wall enclose the opening; the bottom wall is opposite to the opening; and the connector is fixedly connected to an end of one of the two short side walls close to the opening.

As an implementation, one of the two long side walls is provided with an avoidance groove; the avoidance groove is configured to avoid a nipple tunnel of the breast pump; an outer contour of the diaphragm cover matches with a shape of the opening; and/or, the diaphragm assembly further includes a lug; and the lug is connected to an end of the other short side wall close to the opening.

As an implementation, the diaphragm cover includes a cover body and a limiting protrusion; the limiting protrusion is connected to one side of the cover body; the cover body is configured to cover the opening; the limiting protrusion extends into the cavity and abuts against the liquid separation device, so as to limit the cover body; and the connector is fixedly connected to a side of the cover body away from the limiting protrusion.

As an implementation, the limiting protrusion is annular, or the limiting protrusion includes a plurality of tabs that are arranged annularly at intervals.

A second aspect of the present disclosure provides a breast pump, including the above diaphragm assembly.

As an implementation, the breast pump further includes a breast pump assembly, a main pump module, and a breast shield; the breast pump assembly is provided with a negative pressure chamber and a nipple tunnel communicating with the negative pressure chamber; the liquid separation device is disposed in the negative pressure chamber; the nipple tunnel is configured to guide milk; the main pump module is disposed between the breast pump assembly and the breast shield; the breast shield is configured to be placed against a breast; and the first through hole is configured to communicate the cavity with a suction pump of the main pump module.

According to the diaphragm assembly and the breast pump according to the present disclosure, since the connector is provided, and the connector includes one end fixedly connected to the liquid separation device, and another end fixedly connected to the diaphragm cover, the diaphragm cover detached from the opening of the liquid separation device is still connected to the liquid separation device through the connector. Therefore, the diaphragm cover and the liquid separation device are not separated into two independent components, preventing the diaphragm cover from losing, and reducing the number of independent components of the breast pump. Therefore, the diaphragm assembly provided by the present disclosure reduces the number of components, accelerates the disassembly and assembly speeds, and lowers the probability of losing the components.

An objective of the present disclosure is to provide a breast pump assembly and a breast pump, to solve the technical problem of the excessive number of components of the breast pump.

A first aspect of the present disclosure provides a breast pump assembly, applied to a breast pump, and including: a milk storage container, where the milk storage container includes a container body and a container lid, and the container lid and the container body enclose a milk storage chamber; a nipple tunnel cap element and a negative pressure part, where both the nipple tunnel cap element and the negative pressure part are connected to a side of the container lid facing the milk storage chamber or penetrate through the container lid and extend into the milk storage chamber, the nipple tunnel cap element has a nipple tunnel, the negative pressure part has a negative pressure chamber communicating with the nipple tunnel, the nipple tunnel cap element or the negative pressure part is further provided with a milk flow port communicating with the milk storage chamber, and the milk flow port is configured to allow milk to flow to the milk storage chamber from the nipple tunnel or the negative pressure chamber; and a one-way valve, where the one-way valve is connected to at least one of the container lid, the nipple tunnel cap element, and the negative pressure part, extends to the milk flow port, and is configured to allow the milk in the nipple tunnel to unidirectionally flow to the milk storage chamber.

As an implementation, the one-way valve is made of a soft material; the container lid, the nipple tunnel cap element, and the negative pressure part are made of a hard material; and the one-way valve is fixedly connected to at least one of the container lid, the nipple tunnel cap element, and the negative pressure part via integrated injection molding.

As an implementation, the nipple tunnel cap element and the negative pressure part are arranged side by side and share a wall; the milk flow port is formed in a side of the negative pressure part away from the nipple tunnel cap element; when the breast pump is placed against a breast, the negative pressure part is located under the nipple tunnel cap element; and the one-way valve includes one end fixedly connected to a junction between the negative pressure part and the nipple tunnel cap element, and another end extending to the milk flow port.

As an implementation, the negative pressure part includes a body portion and a snap-fit portion; the body portion and the nipple tunnel cap element are arranged side by side and share a wall; the body portion is provided with the negative pressure chamber and the milk flow port; the snap-fit portion is connected to a side of the body portion facing away from the negative pressure chamber and disposed close to the milk flow port; and the snap-fit portion and the body portion enclose a snap groove, so as to allow the one-way valve to be snap-inserted for limiting.

As an implementation, the one-way valve includes a fixing portion, a shielding portion and a snap insertion portion; the snap insertion portion is connected between the fixing portion and the shielding portion, and configured to be inserted into a portion of the negative pressure part or the nipple tunnel cap element close to the milk flow port to limit the shielding portion; the fixing portion is fixedly connected to at least one of the container lid, the nipple tunnel cap element, and the negative pressure part; and the shielding portion is configured to shield the milk flow port.

As an implementation, the fixing portion includes two support arms; and the two support arms are connected to two ends of the snap insertion portion, respectively.

As an implementation, the one-way valve is a single-disc one-way valve.

As an implementation, the milk storage container further includes a sealing ring and a sealing plug; the sealing ring is disposed between the container body and the container lid, and configured to seal the milk storage chamber; the milk storage container is provided with a milk pouring port communicating with the milk storage chamber; the sealing plug includes one end configured to detachably seal the milk pouring port, and another end connected to the sealing ring through the container lid; and the sealing plug and the sealing ring are at least integrally injection-molded with the container lid.

As an implementation, the breast pump assembly further includes a main pump module housing and a breast shield; the breast shield includes a first side portion and a second side portion opposite to the first side portion; the first side portion is configured to be placed against the breast; and the main pump module housing is disposed between the container lid and the second side portion.

A second aspect of the present disclosure provides a breast pump, including the above breast pump assembly.

According to the breast pump assembly and the breast pump provided by the present disclosure, by connecting the one-way valve to at least one of the container lid, the nipple tunnel cap element, and the negative pressure part, when the breast pump assembly is disassembled (for example, various components of the breast pump are disassembled for cleaning), the one-way valve is still connected to at least one of the container lid, the nipple tunnel cap element, and the negative pressure part. This greatly reduces the probability of losing the one-way valve, reduces the number of independent components of the breast pump assembly, and can accelerate the disassembly and assembly speeds. Therefore, the breast pump assembly provided by the present disclosure reduces the number of components, accelerates the disassembly and assembly speeds, and lowers the probability of losing the components.

In view of shortcomings in the prior art, the present disclosure provides a breast pump, to solve the problem of heating of the main pump module of the breast pump.

To solve the above technical problem, a technical solution adopted by the present disclosure is as follows: A breast pump is provided, including: a milk bowl assembly, where a milk storage chamber is formed in the milk bowl assembly; a breast shield, where the breast shield has a wide end and a narrow end, and an inner diameter of the breast shield from the narrow end to the wide end gradually increases; a main pump module, where the main pump module includes a negative pressure pump, and the negative pressure pump is configured to provide the breast pump with a negative pressure for milk expression; and a heat sink, where the heat sink is at least partially disposed between the negative pressure pump and the milk bowl assembly.

The main pump module includes a transverse portion and a longitudinal portion connected to the transverse portion in a bending manner; a first side of the milk bowl assembly comes into contact with the longitudinal portion of the main pump module; a second side of the milk bowl assembly comes into contact with the transverse portion of the main pump module; and the first side and the second side are two adjacent sides of the milk bowl assembly.

The negative pressure pump is located in the transverse portion of the main pump module; a surface of a side of the transverse portion close to the milk bowl assembly is provided with a mounting hole; and the heat sink is at least partially disposed in the mounting hole.

The heat sink includes a first portion and a second portion connected to the first portion in a bending manner; the first portion is disposed between the negative pressure pump and the milk bowl assembly; and the first portion and the second portion are disposed on two adjacent sides of the negative pressure pump, respectively.

A side of the heat sink close to the milk bowl assembly is provided with an assembly step; and the assembly step cooperates with the mounting hole of the transverse portion to limit the heat sink in the transverse portion.

The heat sink is exposed through the mounting hole; and the heat sink comes into contact with the milk bowl assembly when the milk bowl assembly and the main pump module are well-assembled.

The heat sink is made of a metal material.

A through hole is formed in a thickness direction of the main pump module; the narrow end of the breast shield penetrates into the through hole; and the main pump module is at least partially located between the wide end of the breast shield and the milk bowl assembly.

The main pump module includes a transverse portion and a longitudinal portion connected to the transverse portion in a bending manner; the through hole is formed in the longitudinal portion; and the longitudinal portion is located between the wide end of the breast shield and the milk bowl assembly.

The negative pressure pump is a piezoelectric pump.

According to this embodiment of the present disclosure, the breast pump includes the milk bowl assembly, the breast shield, the main pump module, and the heat sink. The milk storage chamber is formed in the milk bowl assembly. The breast shield has the wide end and the narrow end. The inner diameter of the breast shield from the narrow end to the wide end gradually increases. The main pump module includes the negative pressure pump. The negative pressure pump is configured to provide the breast pump with the negative pressure for milk expression. The heat sink is at least partially disposed between the negative pressure pump and the milk bowl assembly. By disposing the heat sink between the negative pressure pump and the milk bowl assembly, heat of the negative pressure pump is conducted to a gap between the milk bowl assembly and the main pump module, thereby dispersing to the air. Alternatively, the heat is conducted to the milk bowl assembly, thereby effectively mitigating the problem of heating of the main pump module.

According to a fourth aspect, in view of shortcomings in the prior art, the present disclosure provides a breast pump, to solve the problem of the unstable center of gravity of the breast pump during wearing.

To solve the above technical problem, a technical solution adopted by the present disclosure is as follows: A breast pump is provided, including: a milk bowl assembly, where a milk storage chamber is formed in the milk bowl assembly; a main pump module, where a through hole is formed in a thickness direction of the main pump module; a breast shield, where the breast shield has a narrow end and a wide end, the narrow end of the breast shield penetrates into the through hole, and the main pump module is at least partially located between the wide end of the breast shield and the milk bowl assembly; and a milk pumping conduit, where the milk pumping conduit communicates with an internal space of the breast shield, and when the milk storage chamber is empty, a center of gravity of the breast pump is located in the through hole of the main pump module or the milk pumping conduit; the main pump module includes a housing as well as a first pumping power device and a second pumping power device that are located in the housing; and the first pumping power device and the second pumping power device are located at two opposite sides of the through hole, respectively.

The main pump module further includes a first battery and a second battery that are located in the housing; and the first battery and the second battery are located at the two opposite sides of the through hole, respectively.

The first pumping power device is located at a left upper side of the through hole; the second pumping power device is located at a right upper side of the through hole; the first battery is located at a left lower side of the through hole; and the second battery is located at a right lower side of the through hole.

The main pump module further includes a first circuit board; and the first circuit board is located above the through hole.

The main pump module further includes a second circuit board; and the second circuit board is located below the through hole.

The first pumping power device includes a first negative pressure pump and a first solenoid valve; and the second pumping power device includes a second negative pressure pump and a second solenoid valve.

The first negative pressure pump is an air diaphragm pump or a piezoelectric pump; and the second negative pressure pump is an air diaphragm pump or a piezoelectric pump.

The main pump module includes a transverse portion and a longitudinal portion connected to the transverse portion in a bending manner; the through hole is formed in the longitudinal portion; the longitudinal portion is located between the wide end of the breast shield and the milk bowl assembly; a first side of the milk bowl assembly contacts the longitudinal portion of the main pump module; a second side of the milk bowl assembly contacts the transverse portion of the main pump module; and the first side and the second side are two adjacent sides of the milk bowl assembly.

The main pump module includes a transverse portion and a longitudinal portion connected to the transverse portion in a bending manner; the through hole is formed in the longitudinal portion; the longitudinal portion is located between the wide end of the breast shield and the milk bowl assembly; a first side of the milk bowl assembly contacts the longitudinal portion of the main pump module; a second side of the milk bowl assembly contacts the transverse portion of the main pump module; the first side and the second side are two adjacent sides of the milk bowl assembly; and the second circuit board is disposed in the transverse portion.

The milk bowl assembly includes a bowl body and a cover plate; the bowl body and the cover plate enclose the milk storage chamber; and the milk pumping conduit is connected to the cover plate.

According to this embodiment of the present disclosure, the breast pump includes: the milk bowl assembly, the main pump module, the breast shield, and the milk pumping conduit. The milk storage chamber is formed in the milk bowl assembly. The through hole is formed in the thickness direction of the main pump module. The breast shield has the narrow end and the wide end. The narrow end of the breast shield penetrates into the through hole. The main pump module is at least partially located between the wide end of the breast shield and the milk bowl assembly. The milk pumping conduit communicates with the internal space of the breast shield. When the milk storage chamber is empty, the center of gravity of the breast pump is located in the through hole of the main pump module or the milk pumping conduit. The main pump module includes the housing as well as the first pumping power device and the second pumping power device that are located in the housing. The first pumping power device and the second pumping power device are located at the two opposite sides of the through hole, respectively. By respectively disposing two pumping power assemblies at the two sides of the through hole to form a balance, the center of gravity of the breast pump is located in the through hole of the main pump module or the milk pumping conduit. Therefore, the breast pump can be worn stably and more comfortably, and is not prone to falling off. Moreover, when placed on a support surface, the breast pump is not prone to tipping over.

In view of shortcomings in the prior art, the present disclosure provides a breast pump, to solve the problem of the small capacity of the milk bowl assembly.

To solve the above technical problem, a technical solution adopted by the present disclosure is as follows: A breast pump is provided, including: a main pump module, where the main pump module includes a transverse portion and a longitudinal portion connected to the transverse portion in a bending manner, and a thickness of an upper half of the longitudinal portion of the main pump module is greater than a thickness of a lower half of the longitudinal portion of the main pump module; and a milk bowl assembly, where a first side of the milk bowl assembly contacts the longitudinal portion of the main pump module, a second side of the milk bowl assembly contacts the transverse portion of the main pump module, and the first side and the second side are two adjacent sides of the milk bowl assembly.

A thickness of the longitudinal portion of the main pump module gradually decreases from top to bottom.

A thickness of an upper half of the milk bowl assembly is less than a thickness of a lower half of the milk bowl assembly.

A thickness of the milk bowl assembly gradually increases from top to bottom.

A thickness of the transverse portion of the main pump module gradually decreases along a direction toward a junction between the transverse portion and the longitudinal portion.

A surface of the transverse portion of the main pump module that contacts the milk bowl assembly is non-planar.

A through hole is formed in a thickness direction of the longitudinal portion of the main pump module; the breast pump further includes a breast shield; the breast shield has a narrow end and a wide end; the narrow end of the breast shield penetrates into the through hole; and the main pump module is at least partially located between the wide end of the breast shield and the milk bowl assembly.

The milk bowl assembly includes a bowl body and a cover plate; the bowl body and the cover plate enclose a milk storage chamber; the breast pump further includes a milk pumping conduit and a diaphragm bracket; a negative pressure chamber is formed in the diaphragm bracket; and both the milk pumping conduit and the diaphragm bracket are connected to the cover plate.

The milk pumping conduit is located above the diaphragm bracket.

The breast pump further includes a diaphragm; the diaphragm is configured to be placed in the negative pressure chamber; the diaphragm divides an internal space of the diaphragm bracket into a first subspace and a second subspace; the first subspace communicates with a negative pressure port of the main pump module; the second subspace communicates with an internal space of the milk pumping conduit; and driven by a negative pressure provided by the main pump module, the diaphragm can deform in the internal space of the diaphragm bracket to transmit the negative pressure.

According to this embodiment of the present disclosure, the breast pump includes the main pump module and the milk bowl assembly. The main pump module includes the transverse portion and the longitudinal portion connected to the transverse portion in the bending manner. The thickness of the upper half of the longitudinal portion of the main pump module is greater than the thickness of the lower half of the longitudinal portion of the main pump module. The first side of the milk bowl assembly contacts the longitudinal portion of the main pump module. The second side of the milk bowl assembly contacts the transverse portion of the main pump module. The first side and the second side are two adjacent sides of the milk bowl assembly. In this way, the present disclosure can increase a capacity of the lower half of the milk bowl, without additionally increasing the overall thickness of the breast pump. Moreover, the present disclosure is more convenient for layout of components in the main pump module.

In view of shortcomings in the prior art, the present disclosure provides a breast pump, to solve the problem of the unstable mounting or positioning of the milk bowl assembly and the main pump module.

To solve the above technical problem, a technical solution adopted by the present disclosure is as follows: A breast pump is provided, including: a main pump module, where the main pump module includes a transverse portion and a longitudinal portion connected to the transverse portion in a bending manner; and a milk bowl assembly, where a first side of the milk bowl assembly contacts the longitudinal portion of the main pump module, a second side of the milk bowl assembly contacts the transverse portion of the main pump module, and the first side and the second side are two adjacent sides of the milk bowl assembly; a first coupling structure is disposed on the transverse portion; a second coupling structure matching with the first coupling structure is disposed on the second side of the milk bowl assembly; and the first coupling structure and the second coupling structure are configured to be coupled with each other, so as to realize mounting or positioning of the main pump module and the milk bowl assembly.

A third coupling structure is disposed on the longitudinal portion; a fourth coupling structure matching with the third coupling structure is disposed on the first side of the milk bowl assembly; and the third coupling structure and the fourth coupling structure are configured to be coupled with each other, so as to realize mounting or positioning of the main pump module and the milk bowl assembly.

Two third coupling structures are disposed on the longitudinal portion; two fourth coupling structures are correspondingly disposed on the first side of the milk bowl assembly; and one first coupling structure is disposed on the transverse portion.

Lines connecting positions where the two third coupling structures and the one first coupling structure are disposed enclose a triangle.

One of the first coupling structure and the second coupling structure is an elastic snap fastener, while the other is a snap groove.

The first coupling structure is the elastic snap fastener; the second coupling structure is the snap groove; and a surface of the elastic snap fastener facing the milk bowl assembly has an oblique plane that inclines upward along a direction toward the longitudinal portion.

One of the third coupling structure and the fourth coupling structure is a protrusion, while the other is a recess.

The third coupling structure is the protrusion; and the fourth coupling structure is the recess.

A surface of the protrusion facing the transverse portion of the main pump module is provided with a ball plunger.

The milk bowl assembly includes a bowl body and a cover plate; the bowl body and the cover plate enclose a milk storage chamber; and the second coupling structure is disposed on the bowl body.

According to this embodiment of the present disclosure, the breast pump includes the main pump module and the milk bowl assembly. The main pump module includes the transverse portion and the longitudinal portion connected to the transverse portion in the bending manner. The first side of the milk bowl assembly contacts the longitudinal portion of the main pump module. The second side of the milk bowl assembly contacts the transverse portion of the main pump module. The first side and the second side are two adjacent sides of the milk bowl assembly. The first coupling structure is disposed on the transverse portion. The second coupling structure matching with the first coupling structure is disposed on the second side of the milk bowl assembly. The first coupling structure and the second coupling structure are configured to be coupled with each other, so as to realize mounting or positioning of the main pump module and the milk bowl assembly. This can improve the stability of mounting or positioning the milk bowl assembly and the main pump module.

In view of shortcomings in the prior art, the present disclosure provides a breast pump, to solve the problem of the large size of the breast pump.

To solve the above technical problem, a technical solution adopted by the present disclosure is as follows: A breast pump is provided, including: a diaphragm bracket, where a negative pressure chamber is formed in the diaphragm bracket; and a milk pumping conduit, where a nipple tunnel is formed in the milk pumping conduit, the milk pumping conduit communicates with the negative pressure chamber, and the nipple tunnel is at least configured to guide milk from a breast; the milk pumping conduit and the diaphragm bracket are arranged side by side in the breast pump; a notch configured to avoid the milk pumping conduit is formed in the diaphragm bracket, and the milk pumping conduit is at least partially embedded into the notch of the diaphragm bracket.

The notch is formed in an upper side or a lower side of the diaphragm bracket, such that spaces respectively occupied by the diaphragm bracket and the milk pumping conduit in a vertical direction at least partially overlap.

A milk flow port is formed in the diaphragm bracket; and the milk flow port is configured to flow the milk in the negative pressure chamber to a milk storage chamber of the breast pump.

A recess that at least partially accommodates the milk flow port is formed in the diaphragm bracket.

A one-way valve is disposed at the milk flow port; the one-way valve is at least partially accommodated in the recess; and the one-way valve is configured to allow the milk to flow to the milk storage chamber unidirectionally.

The breast pump includes a milk bowl assembly; the milk storage chamber is formed in the milk bowl assembly; and the diaphragm bracket is connected to the milk bowl assembly and extends into the milk storage chamber.

The milk pumping conduit is connected to the milk bowl assembly and extends into the milk storage chamber.

The milk bowl assembly includes a bowl body and a cover plate; the bowl body and the cover plate enclose the milk storage chamber; and both the milk pumping conduit and the diaphragm bracket are connected to the cover plate.

The milk pumping conduit and the diaphragm bracket are integrally connected with the cover plate.

The milk pumping conduit is located above the diaphragm bracket.

The breast pump further includes: a main pump module, where a through hole is formed in a thickness direction of the main pump module; and a breast shield, where the breast shield has a narrow end and a wide end, the narrow end of the breast shield penetrates into the through hole, and the main pump module is at least partially located between the wide end of the breast shield and the milk bowl assembly.

The main pump module includes a transverse portion and a longitudinal portion connected to the transverse portion in a bending manner; the through hole is formed in the longitudinal portion; the longitudinal portion is located between the wide end of the breast shield and the milk bowl assembly; a first side of the milk bowl assembly contacts the longitudinal portion of the main pump module; a second side of the milk bowl assembly contacts the transverse portion of the main pump module; and the first side and the second side are two adjacent sides of the milk bowl assembly.

The breast pump further includes a diaphragm; the diaphragm is configured to be placed in the negative pressure chamber; the diaphragm divides an internal space of the diaphragm bracket into a first subspace and a second subspace; the first subspace communicates with a negative pressure port of the main pump module; the second subspace communicates with an internal space of the milk pumping conduit; and driven by a negative pressure provided by the main pump module, the diaphragm can deform in the internal space of the diaphragm bracket to transmit the negative pressure.

According to this embodiment of the present disclosure, the breast pump includes the diaphragm bracket and the milk pumping conduit. The negative pressure chamber is formed in the diaphragm bracket. The nipple tunnel is formed in the milk pumping conduit. The milk pumping conduit communicates with the negative pressure chamber. The nipple tunnel is at least configured to guide the milk from the breast. The milk pumping conduit and the diaphragm bracket are arranged side by side in the breast pump. The notch configured to avoid the milk pumping conduit is formed in the diaphragm bracket. The milk pumping conduit is at least partially embedded into the notch of the diaphragm bracket. Since the diaphragm bracket and the milk pumping conduit are arranged side by side, and the notch configured to avoid the milk pumping conduit is formed in the diaphragm bracket, such that the milk pumping conduit is at least partially embedded into the notch of the diaphragm bracket, and spaces occupied by the milk pumping conduit in transverse and longitudinal directions overlap. This effectively reduces the overall size of the breast pump, such that the breast pump is carried and stored more conveniently, meeting requirements of modern mothers on miniaturization and portability of the breast pump.

In view of shortcomings in the prior art, the present disclosure provides a breast pump, to solve the problem of the limited visible area of the breast pump.

To solve the above technical problem, a technical solution adopted by the present disclosure is as follows: A breast pump is provided, including: a main pump module, where the main pump module includes a transverse portion and a longitudinal portion connected to the transverse portion in a bending manner; and a milk bowl assembly, where a first side of the milk bowl assembly contacts the longitudinal portion of the main pump module, a second side of the milk bowl assembly contacts the transverse portion of the main pump module, the first side and the second side are two adjacent sides of the milk bowl assembly, and a height of the milk bowl assembly accounts for 70% or more of a height of the breast pump.

As an implementation, at least a portion of the milk bowl assembly not shielded by the main pump module is made of a light-transmissive material.

As an implementation, an area of a portion of the milk bowl assembly not shielded by the main pump module accounts for 60% or more of a surface area of the breast pump.

As an implementation, the breast pump further includes a milk pumping conduit extending into the milk bowl assembly; and the milk pumping conduit is made of a light-transmissive material.

As an implementation, the breast pump further includes a diaphragm bracket extending into the milk bowl assembly; and the diaphragm bracket is located below the milk pumping conduit.

As an implementation, the milk bowl assembly includes a bowl body and a cover plate; the bowl body and the cover plate enclose a milk storage chamber; the breast pump further includes the milk pumping conduit and the diaphragm bracket; a negative pressure chamber is formed in the diaphragm bracket; and both the milk pumping conduit and the diaphragm bracket are connected to the cover plate.

As an implementation, the diaphragm bracket is made of a light-transmissive material.

As an implementation, a through hole is formed in a thickness direction of the longitudinal portion of the main pump module; and the breast pump further includes a breast shield; the breast shield has a narrow end and a wide end; the narrow end of the breast shield penetrates into the through hole; and the main pump module is at least partially located between the wide end of the breast shield and the milk bowl assembly.

As an implementation, the breast pump further includes a diaphragm; the diaphragm is configured to be placed in the negative pressure chamber; the diaphragm divides an internal space of the diaphragm bracket into a first subspace and a second subspace; the first subspace communicates with a negative pressure port of the main pump module; the second subspace communicates with an internal space of the milk pumping conduit; and driven by a negative pressure provided by the main pump module, the diaphragm can deform in the internal space of the diaphragm bracket to transmit the negative pressure.

To solve the above technical problem, another technical solution adopted by the present disclosure is as follows: A breast pump is provided, including: a main pump module, where the main pump module includes a transverse portion and a longitudinal portion connected to the transverse portion in a bending manner; and a milk bowl assembly, where a first side of the milk bowl assembly contacts the longitudinal portion of the main pump module; a second side of the milk bowl assembly contacts the transverse portion of the main pump module, the first side and the second side are two adjacent sides of the milk bowl assembly, and an area of a portion of the milk bowl assembly not shielded by the main pump module accounts for 60% or more of a surface area of the breast pump.

According to this embodiment of the present disclosure, the breast pump includes the main pump module and the milk bowl assembly. The main pump module includes the transverse portion and the longitudinal portion connected to the transverse portion in the bending manner. The first side of the milk bowl assembly contacts the longitudinal portion of the main pump module. The second side of the milk bowl assembly contacts the transverse portion of the main pump module. The first side and the second side are two adjacent sides of the milk bowl assembly. The height of the milk bowl assembly accounts for 70% or more of the height of the breast pump. The main pump module is approximately L-shaped, the milk bowl assembly is placed on an L-shaped step of the milk bowl assembly, the height of the milk bowl assembly accounts for 70% or more of the total height of the breast pump, and a height of the transverse portion of the main pump module accounts for 30% or less of the total height of the breast pump. This can greatly increase the visible area of the milk bowl assembly, and facilitate observation of the user on a milk condition in the milk bowl, improving the visualization degree, and enhancing expression feedback experience of the user.

In view of shortcomings in the prior art, the present disclosure provides a breast pump, to solve the problem of the poor airtightness of the milk pumping channel of the breast pump.

To solve the above technical problem, a technical solution adopted by the present disclosure is as follows: A breast pump is provided, including: a breast shield, where the breast shield has a narrow end and a wide end, and an inner diameter of the breast shield gradually increases from the narrow end to the wide end; a milk pumping conduit, where an end surface of one end of the milk pumping conduit abuts against an end surface of the narrow end of the breast shield, and the milk pumping conduit communicates with an internal space of the breast shield; and a sealing structure, where the sealing structure is disposed around a port of the narrow end or around a port of an end of the milk pumping conduit close to the breast shield.

The sealing structure is connected to the port of the narrow end of the breast shield.

The sealing structure is horn-shaped, with an inner diameter gradually increasing along a direction toward the milk pumping conduit.

The sealing structure is made of a soft material, and the narrow end of the breast shield is at least partially made of a rigid material.

More than 50% of the breast shield is made of the soft material.

The sealing structure and the breast shield are integrally formed.

The breast pump further includes a milk bowl assembly; a milk storage chamber is formed in the milk bowl assembly; and the milk pumping conduit is connected to the milk bowl assembly and extends into the milk storage chamber.

The breast pump further includes a main pump module; a through hole is formed in a thickness direction of the main pump module; the narrow end of the breast shield penetrates into the through hole; and the main pump module is at least partially located between the wide end of the breast shield and the milk bowl assembly.

The main pump module includes a transverse portion and a longitudinal portion connected to the transverse portion in a bending manner; and a first side of the milk bowl assembly contacts the longitudinal portion of the main pump module; a second side of the milk bowl assembly contacts the transverse portion of the main pump module; and the first side and the second side are two adjacent sides of the milk bowl assembly.

The breast shield is detachably connected to the main pump module.

According to this embodiment of the present disclosure, the breast pump includes the breast shield, the milk pumping conduit, and the sealing structure. The breast shield has the narrow end and the wide end. The inner diameter of the breast shield gradually increases from the narrow end to the wide end. The end surface of one end of the milk pumping conduit abuts against the end surface of the narrow end of the breast shield. The milk pumping conduit communicates with the internal space of the breast shield. The sealing structure is disposed around the port of the narrow end or around the port of the end of the milk pumping conduit close to the breast shield. By providing the sealing structure at a position where the milk pumping conduit communicates with and is docked with the narrow end of the breast shield, the present disclosure can improve the airtightness of the milk pumping channel of the breast pump.

In view of shortcomings in the prior art, the present disclosure provides a breast pump, to solve the problem of the poor airtightness between the diaphragm cover and the main pump module.

To solve the above technical problem, a technical solution adopted by the present disclosure is as follows: A diaphragm assembly for a breast pump is provided, including: a diaphragm, where the diaphragm has a cavity and an opening communicating with the cavity; a diaphragm cover, where the diaphragm cover is configured to cover the opening, and provided with a through hole; and a sealing structure, where the sealing structure is disposed around the through hole, and the sealing structure is configured to form a seal with the main pump module when the diaphragm cover abuts against the main pump module of the breast pump.

As an implementation, a sealing layer is disposed on a surface of the diaphragm cover facing away from the diaphragm.

As an implementation, the sealing layer and the sealing structure are integrally formed.

As an implementation, the diaphragm cover is made of a rigid material; and the sealing layer is made of a soft material.

As an implementation, the sealing structure is a horn-shaped suction cup; a narrow end of the suction cup communicates with the through hole; and a wide end of the suction cup is configured to form a seal with the main pump module when the diaphragm cover abuts against the main pump module of the breast pump.

As an implementation, a recess is formed in the diaphragm cover; and the recess is configured to facilitate removal of the diaphragm cover from the opening by the user.

As an implementation, there are two recesses; and the two recesses are located at two opposite sides of the through hole, respectively.

As an implementation, the diaphragm assembly further includes a connector; and the connector includes one end connected to the diaphragm, and another end connected to the diaphragm cover.

As an implementation, the connector is disposed on an edge of a long side of the opening of the diaphragm.

To solve the above technical problem, another technical solution adopted by the present disclosure is as follows: A breast pump is provided, including the diaphragm assembly.

According to this embodiment of the present disclosure, the diaphragm assembly includes the diaphragm, the diaphragm cover, and the sealing structure. The diaphragm has the cavity and the opening communicating with the cavity. The diaphragm cover is configured to cover the opening, and provided with the through hole. The sealing structure is disposed around the through hole. The sealing structure is configured to form the seal with the main pump module when the diaphragm cover abuts against the main pump module of the breast pump. By providing the sealing structure on the contact portion between the diaphragm cover and the main pump module of the breast pump, when the breast pump operates, the main pump module is closely attached to the diaphragm cover to form the reliable sealing effect, thereby effectively preventing leakage and contamination.

According to a fifth aspect, in view of shortcomings in the prior art, the present disclosure provides a breast pump, to solve the problem of low visibility.

To solve the above technical problem, a technical solution adopted by the embodiment of the present disclosure is as follows: A breast pump includes: a milk pumping conduit, where a nipple tunnel is formed in the milk pumping conduit, and the nipple tunnel is configured to receive milk from a breast; and a breast shield, where an end of the breast shield at least partially overlaps with the milk pumping conduit, a position where the end of the breast shield overlaps with the milk pumping conduit forms a double-layered structure that includes at least two layers, and the double-layered structure is at least partially made of a light-transmissive material, such that an interior of the nipple tunnel or the breast shield can be observed through the double-layered structure.

Optionally, the end of the breast shield is inserted into the milk pumping conduit or the milk pumping conduit is inserted into the end of the breast shield.

Optionally, a region of the double-layered structure made of the light-transmissive material is located on the nipple tunnel.

Optionally, the breast pump further includes a main pump module and a milk bowl assembly; a through hole is formed in a thickness direction of the main pump module; the breast shield has a narrow end and a wide end; the narrow end of the breast shield penetrates into the through hole; and the main pump module is at least partially located between the wide end of the breast shield and the milk bowl assembly.

Optionally, the narrow end at least partially overlaps with the milk pumping conduit.

Optionally, the breast shield is connected to the main pump module.

Optionally, the milk pumping conduit is connected to the milk bowl assembly.

Optionally, the milk bowl assembly includes a bowl body and a cover plate; the bowl body and the cover plate enclose a milk storage chamber; and the milk pumping conduit is connected to the milk bowl assembly.

Optionally, the milk pumping conduit extends into the milk storage chamber.

Optionally, the milk bowl assembly is at least partially made of the light-transmissive material, such that the interior of the nipple tunnel or the breast shield can be observed through the milk bowl assembly and the double-layered structure, or an interior of the milk storage chamber can be observed through the milk bowl assembly.

According to the embodiment of the present disclosure, the breast pump includes the milk pumping conduit and the breast shield. The nipple tunnel is formed in the milk pumping conduit. The nipple tunnel is configured to receive the milk from the breast. An end of the breast shield at least partially overlaps with the milk pumping conduit. The position where the end of the breast shield overlaps with the milk pumping conduit forms the double-layered structure. The double-layered structure is at least partially made of the light-transmissive material, such that the interior of the nipple tunnel or the breast shield can be observed through the double-layered structure. The present disclosure can enable the user to observe the internal condition of the milk pumping conduit or the breast shield.

### BRIEF DESCRIPTION OF THE DRAWINGS

FIG. 1 is a schematic structural view of a breast pump assembly from a viewing angle according to an embodiment of the present disclosure;

FIG. 2 is a rear view of a breast pump assembly according to an embodiment of the present disclosure;

FIG. 3 is a sectional view along a line A-A in FIG. 2;

FIG. 4 is an exploded view of a breast pump assembly according to an embodiment of the present disclosure;

FIG. 5 is a schematic view of an assembly structure of a container lid, a negative pressure part, and a nipple tunnel cap element according to an embodiment of the present disclosure;

FIG. 6 is a schematic structural view of a container body according to an embodiment of the present disclosure;

FIG. 7 is a schematic structural view of a breast pump from a viewing angle according to an embodiment of the present disclosure;

FIG. 8 is a rear view of a breast pump according to an embodiment of the present disclosure;

FIG. 9 is a sectional view along a line B-B in FIG. 8;

FIG. 10 is an exploded view of a breast pump according to an embodiment of the present disclosure;

FIG. 11 is a schematic structural view of a liquid separation device according to an embodiment of the present disclosure;

FIG. 12 is a schematic structural view of a breast pump from a viewing angle according to an embodiment of the present disclosure;

FIG. 13 is a rear view of a breast pump according to an embodiment of the present disclosure;

FIG. 14 is a sectional view along a line C-C in FIG. 13;

FIG. 15 is a schematic view of an assembly structure of a nipple tunnel cap element, a negative pressure part, and a milk storage container according to an embodiment of the present disclosure;

FIG. 16 is a schematic structural view of a breast shield according to an embodiment of the present disclosure;

FIG. 17 is a schematic structural view of a breast pump assembly from a viewing angle according to an embodiment of the present disclosure;

FIG. 18 is a rear view of a breast pump assembly according to an embodiment of the present disclosure;

FIG. 19 is a sectional view along a line D-D in FIG. 18;

FIG. 20 is an exploded view of a breast pump assembly according to an embodiment of the present disclosure;

FIG. 21 is a schematic view of an assembly structure of a container lid, a negative pressure part, and a nipple tunnel cap element according to an embodiment of the present disclosure;

FIG. 22 is a schematic structural view of a breast pump from a viewing angle according to an embodiment of the present disclosure;

FIG. 23 is a rear view of a breast pump according to an embodiment of the present disclosure;

FIG. 24 is a sectional view along a line E-E in FIG. 23;

FIG. 25 is an exploded view of a breast pump according to an embodiment of the present disclosure;

FIG. 26 is a schematic structural view of a breast pump from a viewing angle according to an embodiment of the present disclosure;

FIG. 27 is a rear view of FIG. 26;

FIG. 28 is a sectional view along a line F-F in FIG. 27;

FIG. 29 is an exploded view of FIG. 26;

FIG. 30 is a schematic view of an assembly structure of a main pump module housing and a pumping power device from a viewing angle according to an embodiment of the present disclosure;

FIG. 31 is a right view of FIG. 30;

FIG. 32 is a front view of FIG. 30;

FIG. 33 is a sectional view along a line G-G in FIG. 32;

FIG. 34 is an exploded view of FIG. 30;

FIG. 35 is a schematic structural view of a breast pump from a viewing angle according to an embodiment of the present disclosure;

FIG. 36 is a rear view of the breast pump shown in FIG. 35;

FIG. 37 is a schematic structural view of a milk storage container according to an embodiment of the present disclosure;

FIG. 38 is a schematic structural view of a main pump module from a viewing angle according to an embodiment of the present disclosure;

FIG. 39 is a front view of the main pump module shown in FIG. 38;

FIG. 40 is a sectional view along a line H-H in FIG. 39;

FIG. 41 is a schematic structural view of the main pump module shown in FIG. 38 from another viewing angle;

FIG. 42 is a sectional view of a panel according to an embodiment of the present disclosure;

FIG. 43 is a sectional view of another panel according to an embodiment of the present disclosure;

FIG. 44 is a sectional view of still another panel according to an embodiment of the present disclosure;

FIG. 45 is an exploded view of another main pump module according to an embodiment of the present disclosure;

FIG. 46 is a schematic structural view of a breast pump according to an embodiment of the present disclosure;

FIG. 47 is a first schematic exploded view of a breast pump according to an embodiment of the present disclosure;

FIG. 48 is a second schematic exploded view of a breast pump according to an embodiment of the present disclosure;

FIG. 49 is a schematic sectional view of a milk bowl assembly according to an embodiment of the present disclosure;

FIG. 50 is a schematic structural view illustrating that a milk pumping conduit is disposed on a breast shield according to another embodiment of the present disclosure;

FIG. 51 is a schematic structural view illustrating that a negative pressure chamber only has a portion located at a position lower than a nipple tunnel according to another embodiment of the present disclosure;

FIG. 52 is a schematic structural view illustrating that a milk outlet is located at a lower position of an end of a milk pumping conduit according to another embodiment of the present disclosure;

FIG. 53 is a schematic structural view of a double-layered structure formed by a breast shield and a milk pumping conduit according to an embodiment of the present disclosure;

FIG. 54 is a schematic structural view of a cover plate according to an embodiment of the present disclosure; and

FIG. 55 is a third schematic exploded view of a breast pump according to an embodiment of the present disclosure.

FIG. 56 is a schematic structural view of a breast pump according to an embodiment of the present disclosure;

FIG. 57 is a schematic exploded view of a breast pump according to an embodiment of the present disclosure;

FIG. 58 is a schematic sectional view of a breast pump after a breast shield and a main pump module are removed according to an embodiment of the present disclosure;

FIG. 59 is a schematic structural view of a cover plate, a milk pumping conduit, and a diaphragm bracket of a breast pump according to an embodiment of the present disclosure;

FIG. 60 is a schematic exploded view of a milk bowl assembly and a main pump module of a breast pump according to an embodiment of the present disclosure;

FIG. 61 is a schematic structural view of a breast pump according to an embodiment of the present disclosure;

FIG. 62 is a schematic exploded view of a breast pump according to an embodiment of the present disclosure;

FIG. 63 is a schematic exploded view of a main pump module and a breast shield according to an embodiment of the present disclosure;

FIG. 64 is a schematic front view of a main pump module according to an embodiment of the present disclosure;

FIG. 65 is a schematic structural view of a breast shield according to an embodiment of the present disclosure;

FIG. 66 is a schematic exploded view of a main pump module and a milk bowl assembly according to an embodiment of the present disclosure;

FIG. 67 is a schematic sectional view of a breast pump after a breast shield and a main pump module are removed according to an embodiment of the present disclosure;

FIG. 68 is a schematic structural view of a cover plate, a milk pumping conduit, and a diaphragm bracket according to an embodiment of the present disclosure;

FIG. 69 is a schematic structural view of a breast pump according to an embodiment of the present disclosure;

FIG. 70 is a schematic exploded view of a breast pump according to an embodiment of the present disclosure;

FIG. 71 is a schematic internal view of a main pump module of a breast pump after a housing is removed according to an embodiment of the present disclosure;

FIG. 72 is a schematic sectional view of a breast pump after a main pump module and a breast shield are removed according to an embodiment of the present disclosure;

FIG. 73 is a schematic structural view of a cover plate, a milk pumping conduit, and a diaphragm bracket according to an embodiment of the present disclosure;

FIG. 74 is a schematic exploded view of a main pump module and a milk bowl assembly according to an embodiment of the present disclosure;

FIG. 75 is a schematic structural view of a breast pump according to an embodiment of the present disclosure;

FIG. 76 is an exploded view of the breast pump shown in FIG. 75;

FIG. 77 is a sectional view of the breast pump shown in FIG. 75 after a main pump module housing and a breast shield are removed;

FIG. 78 is a schematic assembly view of a diaphragm bracket, an external nipple tunnel cap element, and a container lid from a viewing angle according to an embodiment of the present disclosure;

FIG. 79 is a schematic assembly view of a diaphragm bracket, an external nipple tunnel cap element, and a container lid from another viewing angle according to an embodiment of the present disclosure;

FIG. 80 is a schematic assembly view of an internal nipple tunnel cap element, a negative pressure chamber cover, and a handle from a viewing angle according to an embodiment of the present disclosure;

FIG. 81 is a schematic assembly view of an internal nipple tunnel cap element, a negative pressure chamber cover, and a handle from another viewing angle according to an embodiment of the present disclosure;

FIG. 82 is a schematic structural view of a breast pump according to an embodiment of the present disclosure;

FIG. 83 is an exploded view of the breast pump shown in FIG. 82;

FIG. 84 is a sectional view of the breast pump shown in FIG. 82 after a main pump module housing and a breast shield are removed;

FIG. 85 is a schematic assembly view of a container lid, a nipple tunnel cap element, and a diaphragm bracket from a viewing angle according to an embodiment of the present disclosure;

FIG. 86 is a schematic assembly view of a container lid, a nipple tunnel cap element, and a diaphragm bracket from another viewing angle according to an embodiment of the present disclosure;

FIG. 87 is a schematic structural view of a diaphragm assembly in a closed state of a diaphragm cover according to an embodiment of the present disclosure;

FIG. 88 is a schematic structural view of a diaphragm assembly in an open state of a diaphragm cover according to an embodiment of the present disclosure;

FIG. 89 is an exploded view of the diaphragm assembly shown in FIG. 88;

FIG. 90 is a schematic structural view of the diaphragm assembly shown in FIG. 88 after a diaphragm cover is removed;

FIG. 91 is a schematic structural view of a diaphragm cover from a viewing angle according to an embodiment of the present disclosure;

FIG. 92 is a schematic structural view of a diaphragm cover from another viewing angle according to an embodiment of the present disclosure;

FIG. 93 is a schematic structural view of a breast pump according to an embodiment of the present disclosure;

FIG. 94 is an exploded view of the breast pump shown in FIG. 93;

FIG. 95 is a schematic structural view of a breast pump according to an embodiment of the present disclosure;

FIG. 96 is a sectional view of a milk storage container, a nipple tunnel cap element, and a negative pressure part according to an embodiment of the present disclosure;

FIG. 97 is a schematic assembly view of a milk storage container without a container body, a nipple tunnel cap element, and a negative pressure part according to an embodiment of the present disclosure;

FIG. 98 is a schematic assembly view of a container lid, a nipple tunnel cap element, and a negative pressure part from a viewing angle according to an embodiment of the present disclosure;

FIG. 99 is a schematic assembly view of a container lid, a nipple tunnel cap element, and a negative pressure part from another viewing angle according to an embodiment of the present disclosure;

FIG. 100 is a schematic structural view of a one-way valve according to an embodiment of the present disclosure;

FIG. 101 is a schematic structural view of a breast shield according to an embodiment of the present disclosure;

FIG. 102 is a schematic structural view of a breast pump according to an embodiment of the present disclosure;

FIG. 103 is a schematic exploded view of a breast pump according to an embodiment of the present disclosure;

FIG. 104 is a schematic exploded view of a main pump module and a heat sink of a breast pump according to an embodiment of the present disclosure; and

FIG. 105 is a schematic sectional view of a transverse portion of a main pump module of a breast pump according to an embodiment of the present disclosure.

FIG. 106 is a schematic structural view of a breast pump according to a first embodiment of the present disclosure;

FIG. 107 is a schematic exploded view of a breast pump according to a first embodiment of the present disclosure;

FIG. 108 is a schematic view of an internal structure of a main pump module according to a first embodiment of the present disclosure;

FIG. 109 is a schematic structural view of a breast pump according to a second embodiment of the present disclosure;

FIG. 110 is a first schematic exploded view of a breast pump according to a second embodiment of the present disclosure;

FIG. 111 is a second schematic exploded view of a breast pump according to a second embodiment of the present disclosure;

FIG. 112 is a schematic structural view of a breast pump according to a third embodiment of the present disclosure;

FIG. 113 is a first schematic exploded view of a breast pump according to a third embodiment of the present disclosure;

FIG. 114 is a second schematic exploded view of a breast pump according to a third embodiment of the present disclosure;

FIG. 115 is a schematic structural view of a breast pump according to a fourth embodiment of the present disclosure;

FIG. 116 is a first schematic exploded view of a breast pump according to a fourth embodiment of the present disclosure;

FIG. 117 is a second schematic exploded view of a breast pump according to a fourth embodiment of the present disclosure;

FIG. 118 is a schematic structural view of a diaphragm bracket according to a fourth embodiment of the present disclosure;

FIG. 119 is a schematic structural view of a disc-type one-way valve disposed at a milk flow port according to a fourth embodiment of the present disclosure;

FIG. 120 is a schematic structural view of a breast pump from a first viewing angle according to a fifth embodiment of the present disclosure;

FIG. 121 is a schematic structural view of a breast pump from a second viewing angle according to a fifth embodiment of the present disclosure;

FIG. 122 is a first schematic exploded view of a breast pump according to a fifth embodiment of the present disclosure;

FIG. 123 is a second schematic exploded view of a breast pump according to a fifth embodiment of the present disclosure;

FIG. 124 is a schematic structural view of a breast pump according to a sixth embodiment of the present disclosure;

FIG. 125 is a schematic exploded view of a breast pump according to a sixth embodiment of the present disclosure;

FIG. 126 is a schematic structural view of a breast shield and a sealing structure from a first viewing angle according to a sixth embodiment of the present disclosure;

FIG. 127 is a schematic structural view of a breast shield and a sealing structure from a second viewing angle according to a sixth embodiment of the present disclosure;

FIG. 128 is a schematic structural view of a diaphragm assembly in a closed state of a diaphragm cover according to a seventh embodiment of the present disclosure;

FIG. 129 is a schematic structural view of a diaphragm assembly in an open state of a diaphragm cover according to a seventh embodiment of the present disclosure;

FIG. 130 is a schematic sectional view of a diaphragm cover, a sealing structure, and a sealing layer according to a seventh embodiment of the present disclosure;

FIG. 131 is a schematic structural view of a breast pump according to a seventh embodiment of the present disclosure; and

FIG. 132 is a schematic exploded view of a breast pump according to a seventh embodiment of the present disclosure.

FIG. 133 is a schematic structural view of a breast pump according to an embodiment of the present disclosure;

FIG. 134 is a first schematic exploded view of a breast pump according to an embodiment of the present disclosure;

FIG. 135 is a second schematic exploded view of a breast pump according to an embodiment of the present disclosure;

FIG. 136 is a schematic sectional view of a milk bowl assembly according to an embodiment of the present disclosure;

FIG. 137 is a schematic structural view illustrating that a milk pumping conduit is disposed on a breast shield according to another embodiment of the present disclosure;

FIG. 138 is a schematic structural view illustrating that a negative pressure chamber only has a portion located at a position lower than a nipple tunnel according to another embodiment of the present disclosure;

FIG. 139 is a schematic structural view illustrating that a milk outlet is formed at an end and lower position of a milk pumping conduit according to another embodiment of the present disclosure;

FIG. 140 is a schematic structural view of a double-layered structure formed by a breast shield and a milk pumping conduit according to an embodiment of the present disclosure;

FIG. 141 is a schematic structural view of a cover plate according to an embodiment of the present disclosure; and

FIG. 142 is a third schematic exploded view of a breast pump according to an embodiment of the present disclosure.

The implementations of the objectives, functional characteristics and advantages of the present disclosure will be further described below with reference to the embodiments and the drawings.

### DETAILED DESCRIPTION OF THE DRAWINGS AND THE PRESENTLY PREFERRED EMBODIMENTS

The technical solutions in the embodiments of the present application are clearly and completely described below with reference to the accompanying drawings in the embodiments of the present application. Apparently, the described embodiments are merely some rather than all of the embodiments of the present application. All other embodiments obtained by those of ordinary skill in the art based on the embodiments of the present application without creative efforts should fall within the protection scope of the present application.

It should be noted that all the directional indications (such as upper, lower, left, right, front, back, etc.) in the examples of the present application are merely used to explain a relative position relationship, motion situations, and the like of the components in a specific gesture. If the specific gesture changes, the directivity indication also changes accordingly.

It should be noted that when a component is “fixed” or “provided” on the other component, the component may be “fixed” to or “provided” on the other component directly or via an intermediate component. When a component is “connected” to the other component, the component may be “connected” to the other component directly or via an intermediate component.

Moreover, the terms such as “first”, “second”, and the like described in the present application are used herein only for the purpose of description and are not intended to indicate or imply relative importance, or implicitly indicate the number of the indicated technical features. Therefore, features defined by “first” and “second” may explicitly or implicitly include at least one of the features. Furthermore, the technical solutions between the various embodiments may be combined with each other, but must be on the basis that the combination thereof can be implemented by a person of ordinary skill in the art. In case of a contradiction with the combination of the technical solutions or a failure to implement the combination, it should be considered that the combination of the technical solutions does not exist, and is not within the protection scope of the present disclosure.

According to a first aspect, a first objective of the present disclosure is to provide a breast pump assembly, to solve the technical problem that the existing breast pump has an excessive number of components to cause large assembly difficulty and potential airtightness hazards.

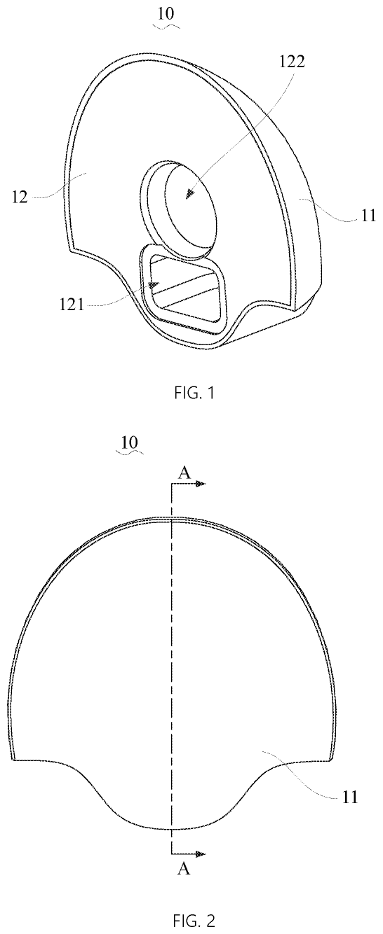

As shown in FIGS. 1-3 and FIG. 7, an embodiment of the present disclosure provides a breast pump assembly 10 for a breast pump 100, including a container body 11 and a container lid 12. The container body 11 and the container lid 12 enclose a milk storage chamber 111. In this way, the milk can be temporarily stored in the milk storage chamber 111 well.

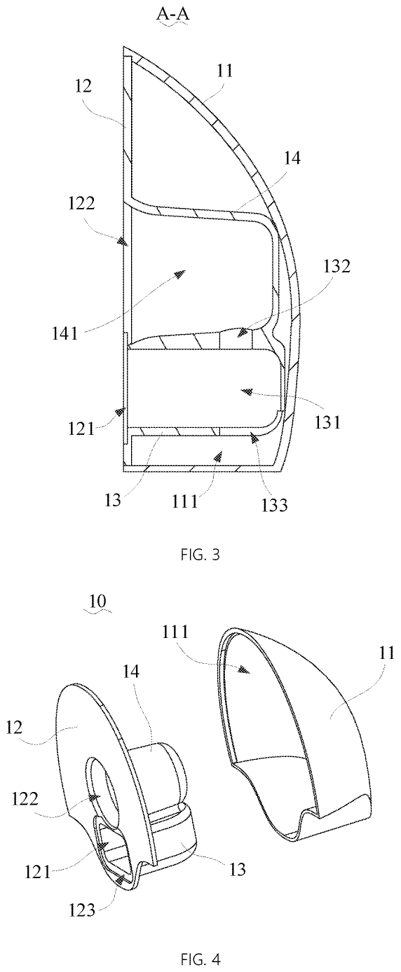

As an implementation, referring to FIGS. 3-5, FIG. 8, and FIG. 9, the breast pump assembly 10 further includes a negative pressure part 13 and a nipple tunnel cap element 14. Both the negative pressure part 13 and the nipple tunnel cap element 14 are connected to the container lid 12. The negative pressure part 13 has a negative pressure chamber 131. The negative pressure chamber 131 is configured to communicate with a negative pressure suction port 311 of the breast pump 100. The nipple tunnel cap element 14 has a nipple tunnel 141. The nipple tunnel 141 communicates with the negative pressure chamber 131. The nipple tunnel 141 is at least configured to guide milk from a breast. The negative pressure part 13, the nipple tunnel cap element 14, and the container lid 12 are integrally formed. One of the nipple tunnel 141 and the negative pressure chamber 131 communicates with the milk storage chamber 111.

According to the above technical solution, the nipple tunnel cap element 14 having the nipple tunnel 141 is provided. The nipple tunnel 141 communicates with a breast receptacle 21 that is configured to accommodate the breast, and can guide the milk expressed from the breast. The negative pressure part 13 having the negative pressure chamber 131 is provided. The negative pressure chamber 131 communicates with the nipple tunnel 141. The negative pressure chamber 131 can provide a negative pressure for the nipple tunnel 141, thereby providing the negative pressure for the breast receptacle 21 to express the milk. One of the negative pressure chamber 131 and the nipple tunnel 141 communicates with the milk storage chamber 111, so as to deliver the milk to the milk storage chamber 111. Both the negative pressure part 13 and the nipple tunnel cap element 14 are connected to the container lid 12, and the negative pressure part 13, the nipple tunnel cap element 14, and the container lid 12 are integrally formed. That is, the negative pressure part 13, the nipple tunnel cap element 14, and the container lid 12 are designed into a whole. This reduces the number of components of the breast pump assembly 10, thereby lowering the assembly difficulty of the breast pump assembly 10, and improving the assembly efficiency. In addition, the operation of assembling the negative pressure part 13 and the nipple tunnel cap element 14 on the container lid 12 is omitted, thereby preventing the problem of poor airtightness due to assembly errors of the negative pressure part 13 and the nipple tunnel cap element 14. Therefore, the breast pump assembly 10 provided by this embodiment of the present disclosure has low assembly difficulty and high assembly efficiency. It also prevents poor airtightness caused by the assembly errors of the negative pressure part 13 and the nipple tunnel cap element 14, thereby eliminating potential airtightness hazards.

As an implementation, both the nipple tunnel cap element 14 and the negative pressure part 13 are connected to a side of the container lid 12 facing the milk storage chamber 111. In this way, both the nipple tunnel cap element 14 and the negative pressure part 13 are accommodated in the milk storage chamber 111, i.e., both the nipple tunnel 141 and the negative pressure chamber 131 are accommodated in the milk storage chamber 111. This can effectively utilize the space of the milk storage chamber 111, reduce assembly of components outside the breast pump assembly 10, and facilitate communication of one of the nipple tunnel 141 and the negative pressure chamber 131 with the milk storage chamber 111. Certainly, in specific applications, as an alternative implementation, at least one of the nipple tunnel cap element 14 and the negative pressure part 13 may also be connected to a side of the container lid 12 away from the milk storage chamber 111.

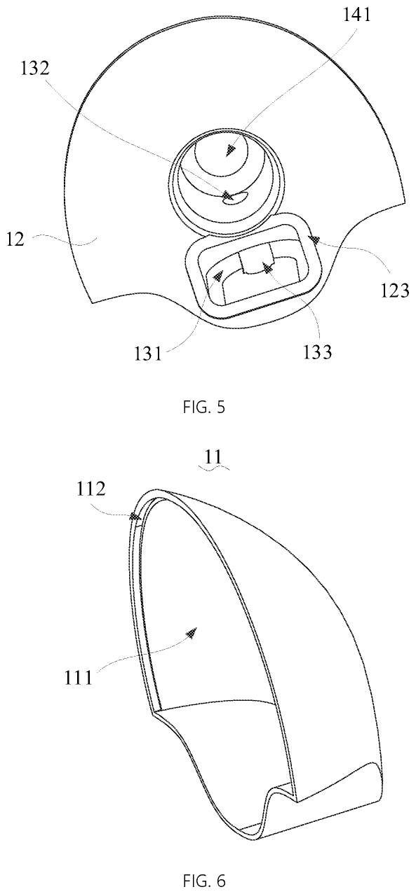

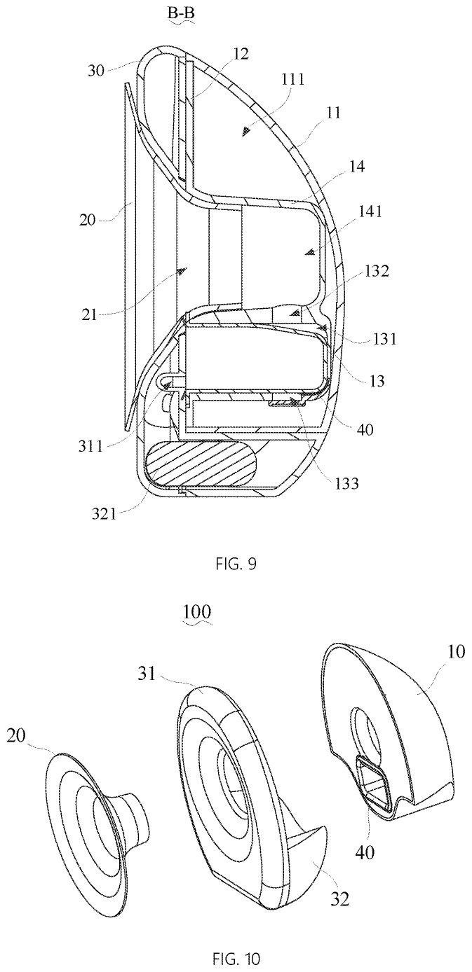

As an implementation, referring to FIG. 4, FIG. 5, and FIG. 9, a first perforation 121 and a second perforation 122 are formed in the container lid 12. A central axis of the first perforation 121 is parallel to a central axis of the second perforation 122. The negative pressure part 13 is connected to the first perforation 121. The first perforation 121 is configured to communicate the negative pressure chamber 131 with the negative pressure suction port 311. The nipple tunnel cap element 14 is connected to the second perforation 122. The second perforation 122 communicates with the nipple tunnel 141, and is configured to allow the milk to pass through and flow to the nipple tunnel 141. In this way, the negative pressure part 13 and the nipple tunnel cap element 14 are connected to the container lid 12, the milk can flow to the nipple tunnel 141, and the negative pressure suction port 311 communicates with the negative pressure chamber 131. In addition, the first perforation 121 communicates with the negative pressure chamber 131, and the second perforation 122 communicates with the nipple tunnel 141, facilitating cleaning of the negative pressure chamber 131 via the first perforation 121, and cleaning of the nipple tunnel 141 via the second perforation 122.

On the basis of the above implementation, the central axis of the first perforation 121 is coaxial with a central axis of the negative pressure chamber 131, and the central axis of the second perforation 122 is coaxial with a central axis of the nipple tunnel 141. That is, the central axis of the negative pressure chamber 131 is parallel to the central axis of the nipple tunnel 141, such that the negative pressure part 13 and the nipple tunnel cap element 14 are arranged side by side on the side of the container lid 12 facing the milk storage chamber 111. In contrast to the arrangement where the nipple tunnel cap element 14 and the negative pressure part 13 are crossed, this reduces the occupied space of the container body 11, thereby reducing the occupied space of the breast pump assembly 10. Specifically, both the central axis of the negative pressure chamber 131 and the central axis of the nipple tunnel 141 are perpendicular to a plane where the container lid 12 is located. Both the negative pressure part 13 and the nipple tunnel cap element 14 are perpendicular to the container lid 12.

As an implementation, the first perforation 121 is closely adjacent to the second perforation 122. Since the negative pressure part 13 is connected to the first perforation 121, and the nipple tunnel cap element 14 is connected to the second perforation 122, by making the first perforation 121 closely adjacent to the second perforation 122, the negative pressure part 13 and the nipple tunnel cap element 14 are arranged closely together, improving compactness of arrangement for the components of the breast pump assembly 10. It should be understood that in other embodiments, the first perforation 121 may also not be closely adjacent to the second perforation 122.

As an implementation, when the breast pump 100 is placed against the breast, the first perforation 121 is located below the second perforation 122. In this way, the negative pressure part 13 is disposed under the nipple tunnel cap element 14, i.e., the negative pressure chamber 131 is disposed under the nipple tunnel 141. In specific applications, the nipple tunnel 141 is located in a middle of the breast pump 100. By disposing the negative pressure chamber 131 under the nipple tunnel 141, the negative pressure chamber 131 is located on a middle lower portion of the breast pump 100. Therefore, the following pumping power device 321 is disposed on a lower portion of the breast pump 100, thereby lowering a center of gravity of the breast pump 100 and further enabling the breast pump 100 to be tightly worn on the breast. Certainly, in specific applications, when the breast pump 100 is placed against the breast, the first perforation 121 may also be located at other positions, such as a left side, a right side, and an upside, of the second perforation 122.

As an implementation, when the breast pump 100 is placed against the breast, the first perforation 121 is located directly under the second perforation 122, and the negative pressure chamber 131 communicates with the milk storage chamber 111. In this way, the negative pressure part 13 is disposed directly under the nipple tunnel cap element 14, thereby reducing the occupied space for left and right sides of the container body 11, and making an external contour of the container body 11 more aesthetically-pleasing. By this time, the negative pressure part 13 serves as a three-way structure, which communicates with the negative pressure suction port 311, the nipple tunnel 141, and the milk storage chamber 111.

As an implementation, referring to FIGS. 5-7, the container body 11 is provided with a first mounting groove 112. The container lid 12 is accommodated in the first mounting groove 112, so as to enclose the milk storage chamber 111 with the container body 11. In this way, the container lid 12 is not convexly disposed at an opening of the container body 11, reducing a thickness of the breast pump assembly 10, and thus facilitating thinning of the breast pump 100. Certainly, in specific applications, as an alternative implementation, the container body 11 may also not be provided with the first mounting groove 112.

As an implementation, the breast pump assembly 10 further includes a sealing member (not shown in the figure). The sealing member is disposed between the container lid 12 and the container body 11, so as to seal the milk storage chamber 111.

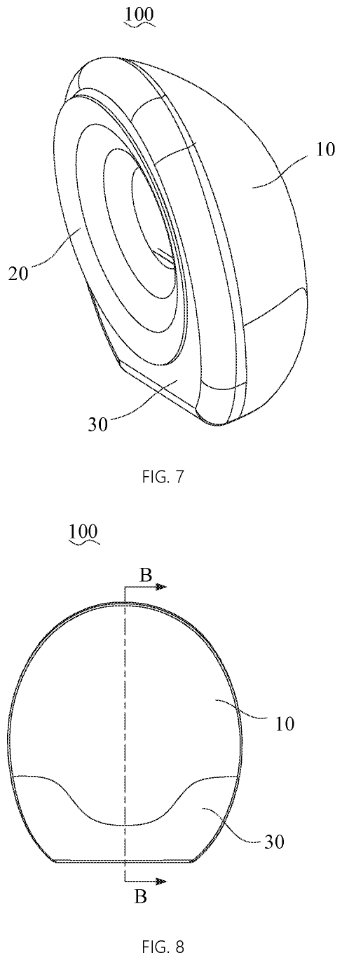

Further, referring to FIGS. 7-9, an embodiment of the present disclosure further provides a breast pump 100, including: a breast shield 20, a main pump module 30, and the breast pump assembly 10. A breast receptacle 21 is formed in the breast shield 20. The main pump module 30 is provided with the negative pressure suction port 311 configured to transfer a negative pressure. The nipple tunnel 141 communicates with the breast receptacle 21. The negative pressure chamber 131 communicates with the negative pressure suction port 311. With the above breast pump assembly 10, the assembly efficiency of the breast pump is improved, and the situation where the airtightness is poor due to assembly errors of the negative pressure part 13 and the nipple tunnel cap element 14 is reduced.

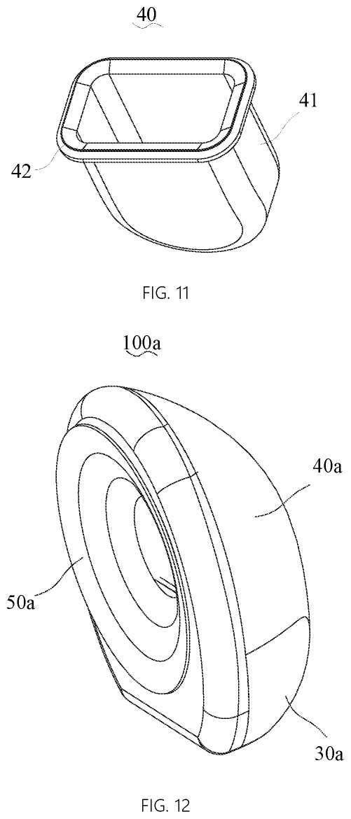

As an implementation, referring to FIG. 4 and FIGS. 9-11, the first perforation 121 is formed in the container lid 12. The negative pressure part 13 is connected to the first perforation 121 and located on the side of the container lid 12 facing the milk storage chamber 111. The breast pump 100 further includes a liquid separation device, specifically a flexible diaphragm 40. The flexible diaphragm 40 includes a diaphragm body 41 and a ring 42 connected to the diaphragm body 41. A circumferential direction of the main pump module 30 around the negative pressure suction port 311 and/or a circumferential direction of the container lid 12 around the first perforation 121 is provided with a second mounting groove 123. The ring 42 is disposed in the second mounting groove 123. The diaphragm body 41 is accommodated in the negative pressure chamber 131. In this way, while the flexible diaphragm 40 is mounted, the thickness of the breast pump 100 is not increased, facilitating thinning of the breast pump 100.

For example, in this embodiment, the circumferential direction of the container lid 12 around the first perforation 121 is provided with the second mounting groove 123. The ring 42 of the flexible diaphragm 40 is disposed in the second mounting groove 123. The diaphragm body 41 of the flexible diaphragm 40 is accommodated in the negative pressure chamber 131. In this way, the flexible diaphragm 40 is mounted more conveniently.

As an implementation, referring to FIG. 3, FIG. 8, and FIG. 9, the negative pressure part 13 further has a first through hole 132 and a second through hole 133. The first through hole 132 is configured to communicate the negative pressure chamber 131 with the nipple tunnel 141. The second through hole 133 is configured to communicate the negative pressure chamber 131 with the milk storage chamber 111. When the breast pump 100 is placed against the breast, the negative pressure chamber 131 is located below the nipple tunnel 141. The flexible diaphragm 40 has a contracted state and an expanded state. When the flexible diaphragm 40 is in the expanded state, at least a portion of the flexible diaphragm 40 corresponding to the first through hole 132 is spaced apart from a wall of the negative pressure chamber 131. In this way, when the flexible diaphragm 40 is in the expanded state, there is a certain space between the at least portion of the flexible diaphragm 40 corresponding to the first through hole 132 and the wall of the negative pressure chamber 131. This space can accommodate the milk, thereby preventing the milk from flowing back to the nipple tunnel 141 through the first through hole 132 when the flexible diaphragm 40 is in the expanded state. A check valve (not shown in the figure) is disposed at the second through hole 133, so as to prevent backflow of the milk to the negative pressure chamber 131 from the milk storage chamber 111.

For example, in this embodiment, when the flexible diaphragm 40 is in the expanded state, a portion, corresponding to the first through hole 132, of a side of the flexible diaphragm 40 facing the first through hole 132 inclines to form a certain space.

As an implementation, referring to FIGS. 8-10, the main pump module 30 includes a first support arm 31 and a second support arm 32. The first support arm 31 is clamped between the breast shield 20 and the breast pump assembly 10. The second support arm 32 is located on one side of the breast pump assembly 10. The negative pressure suction port 311 is formed in a side of the first support arm 31 close to the second support arm 32. The second support arm 32 includes the pumping power device 321. The container lid 12 is disposed on a side of the container body 11 close to the first support arm 31. Specifically, when the breast pump 100 is located on the breast, the second support arm 32 is located below the breast pump assembly 10. Considering that the first perforation 121 is located below the second perforation 122, and the negative pressure part 13 is located below the nipple tunnel cap element 14, a negative pressure generated by the pumping power device 321 can be quickly transferred to the negative pressure chamber 131. Meanwhile, this arrangement can reduce the thickness of the breast pump 100, such that the breast pump 100 is thinner.

As shown in FIGS. 12-14, an embodiment of the present disclosure provides a breast pump 100a, including: a nipple tunnel cap element 10a, a negative pressure part 20a, and a main pump module 30a. The nipple tunnel cap element 10a is made of an optically transparent material and has a nipple tunnel 11a. The nipple tunnel 11a is at least configured to guide milk from a breast. The negative pressure part 20a has a negative pressure chamber 21a. The negative pressure chamber 21a at least communicates with the nipple tunnel 11a. The main pump module 30a is provided with a negative pressure suction port 31a. The negative pressure suction port 31a communicates with the negative pressure chamber 21a. The main pump module 30a is configured to generate a negative pressure at the negative pressure suction port 31a. The negative pressure chamber 21a communicates with the negative pressure suction port 31a and the nipple tunnel 11a, such that a negative pressure can be generated in the nipple tunnel 11a. When the breast pump 100a is placed against the breast, the negative pressure part 20a is located below the nipple tunnel cap element 10a, and the main pump module 30a is located at a position other than on the nipple tunnel cap element 10a.

According to the above technical solution, the nipple tunnel cap element 10a having the nipple tunnel 11a is provided, and configured to deliver the milk from the breast, and the nipple tunnel cap element 10a is made of the optically transparent material, such that the user can directly view a condition in the nipple tunnel 11a. Since the negative pressure part 20a is disposed under the nipple tunnel cap element 10a, and the main pump module 30a is disposed at the position other than on the nipple tunnel cap element 10a, when the breast pump 100a is used, the user views the breast pump 100a from the position above the breast pump 100a, and neither the negative pressure part 20a nor the main pump module 30a shields the nipple tunnel cap element 10a. Thus, the user can view a flowing condition of the milk in the nipple tunnel 11a in real time. In case of an excessively fast or slow flow rate, an operation mode can be adjusted at any time, so as to slow down or speed up flowing of the milk. Therefore, when the breast pump 100a provided by this embodiment is used, the user can quickly and effectively get the problem of the breast pump 100a to immediately adjust the operation mode, greatly improving the use effect of the breast pump 100a.

As an implementation, at least a portion of the main pump module 30a is disposed on a side of the negative pressure part 20a away from the nipple tunnel cap element 10a. That is, when the breast pump 100a is placed against the breast, at least a portion of the main pump module 30a is located below the negative pressure part 20a. The nipple tunnel 11a is typically disposed in a middle of the breast pump 100a because it receives the milk from the breast. That is, the nipple tunnel cap element 10a is disposed in the middle of the breast pump 100a. By disposing the negative pressure part 20a and at least a portion of the main pump module 30a under the nipple tunnel cap element 10a, a center of gravity of the breast pump 100a is lowered, enabling the breast pump 100a to be tightly worn on the breast.

For example, in this embodiment, a portion of the main pump module 30a is disposed on the side of the negative pressure part 20a away from the nipple tunnel cap element 10a, and a portion of the main pump module 30a is disposed aside the negative pressure part 20a and the nipple tunnel cap element 10a.

As an implementation, the negative pressure part 20a is made of the optically transparent material. In specific applications, when being disposed under the nipple tunnel cap element 10a, the negative pressure part 20a serves as a three-way structure, and communicates with the negative pressure suction port 31a, the nipple tunnel 11a, and a following milk storage chamber 43a. The negative pressure chamber 21a also serves to guide the milk. Since the negative pressure part 20a is made of the optically transparent material, the user can view the interior of the negative pressure chamber 21a, thereby viewing a condition of the milk in the negative pressure chamber 21a.

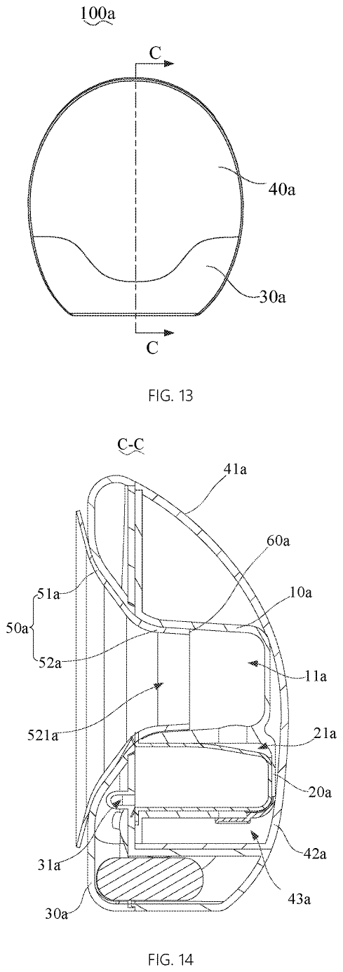

As an implementation, referring to FIG. 13 and FIG. 14, the breast pump 100a further includes a milk storage container 40a. Both the nipple tunnel cap element 10a and the negative pressure part 20a are connected to an inner side of the milk storage container 40a, and the main pump module 30a is disposed on an outer side of the milk storage container 40a. Certainly, in specific applications, as an alternative implementation, the breast pump 100a may also not include the milk storage container 40a. In this case, the breast pump 100a may be externally connected to a container such as a milk storage bag or a milk bottle to store the milk.

On the basis of the above implementation, when the breast pump 100a is placed against the breast, at least a portion of the milk storage container 40a located above the nipple tunnel cap element 10a is made of the optically transparent material. In this way, the user can further view the interior of the milk storage container 40a, and can view the interior of the nipple tunnel 11a through the milk storage container 40a from above in use.

As an implementation, the milk storage container 40a is entirely made of the optically transparent material. In this way, the user can view the interior of the milk storage container 40a from all directions. Considering that both the nipple tunnel cap element 10a and the negative pressure part 20a are made of the transparent material, the user can clearly know an entire delivery condition of the milk expressed from the nipple to the milk storage container 40a.

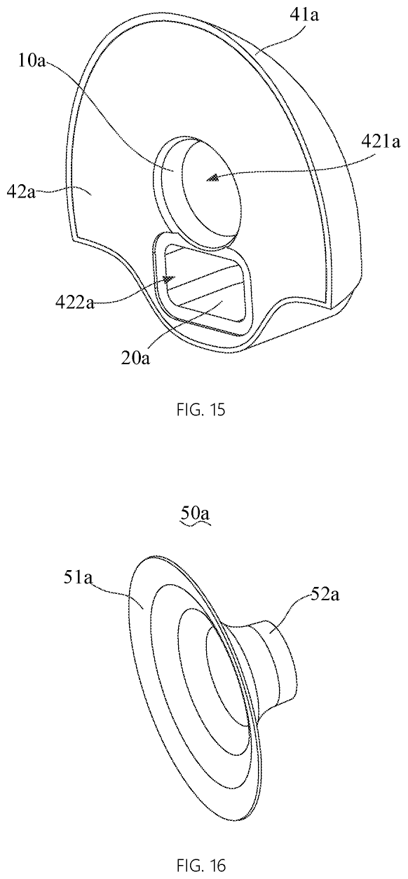

As an implementation, referring to FIGS. 13-15, the milk storage container 40a includes a first container wall 41a and a second container wall 42a. The first container wall 41a and the second container wall 42a enclose the milk storage chamber 43a. Both the negative pressure part 20a and the nipple tunnel cap element 10a are connected to a side of the second container wall 42a facing the milk storage chamber 43a. When the breast pump 100a is placed against the breast, the first container wall 41a is located above the nipple tunnel cap element 10a. Specifically, each of the first container wall 41a and the second container wall 42a has a hollow cavity. After the first container wall 41a and the second container wall 42a are connected, their cavities form the milk storage chamber 43a. A first perforation 421a and a second perforation 422a are formed in the second container wall 42a. Both the first perforation 421a and the second perforation 422a communicate with the milk storage chamber 43a. The nipple tunnel cap element 10a is connected to the first perforation 421a. The first perforation 421a is configured to communicate the nipple tunnel 11a with an exterior of the milk storage container 40a, such that the milk can be guided to the nipple tunnel 11a. The negative pressure part 20a is connected to the second perforation 422a. The second perforation 422a is configured to communicate the negative pressure chamber 21a with the negative pressure suction port 31a.

On the basis of the above implementation, transparency of the first container wall 41a is greater than or equal to transparency of the second container wall 42a. In this way, the user can clearly view the interior of the milk storage container 40a when using the breast pump 100a.

As an implementation, referring to FIG. 12 and FIGS. 14-16, the breast pump 100a further includes a breast shield 50a. The breast shield 50a includes a breast attachment portion 51a and a channel portion 52a connected to the breast attachment portion 51a. The breast attachment portion 51a is configured to be adsorbed to the breast. The channel portion 52a is provided with a nipple channel 521a. At least a portion of the channel portion 52a extends into the nipple tunnel cap element 10a, such that the nipple channel 521a communicates with the nipple tunnel 11a. Specifically, the channel portion 52a extends through the first perforation 421a into the nipple tunnel 11a of the nipple tunnel cap element 10a. A nipple is accommodated in the nipple channel 521a. The milk expressed from the nipple flows from the nipple channel 521a to the nipple tunnel 11a.

As an implementation, the channel portion 52a is made of the optically transparent material. In this way, the user can view the interior of the nipple channel 521a. When the breast pump 100a is worn on the breast, the user can check whether the nipple is aligned with the nipple channel 521a and whether the nipple is accurately accommodated in the nipple channel 521a, thereby improving the wearing efficiency of the breast pump 100a on the breast.

As an implementation, the breast attachment portion 51a and the channel portion 52a are integrally formed. This reduces the assembly steps of the breast shield 50a, and effectively ensures the sealing performance of connection between the breast attachment portion 51a and the channel portion 52a. Certainly, in specific applications, as an alternative implementation, the breast attachment portion 51a and the channel portion 52a may also be manufactured individually for assembly.

On the basis of the above implementation, the breast attachment portion 51a is made of the optically transparent material.

As an implementation, the channel portion 52a is in an interference fit with the nipple tunnel cap element 10a. In this way, the channel portion 52a is connected to the nipple tunnel cap element 10a conveniently and quickly.

As an implementation, the breast shield 50a is detachably connected to the nipple tunnel cap element 10a. Upon completion of milk expression, the breast shield 50a can be detached from the nipple tunnel cap element 10a, facilitating the cleaning of the breast shield 50a and the nipple tunnel cap element 10a.

As an implementation, an outer surface of the channel portion 52a is smooth, which is structurally simple, and easy to manufacture.

As an implementation, referring to FIG. 14, an end of the channel portion 52a away from the breast attachment portion 51a and an inner surface of the nipple tunnel cap element 10a define a step 60a, so as to prevent backflow of the milk. Specifically, the milk comes into contact with the step 60a when flowing back, and the step 60a obstructs the milk, preventing further flowing of the milk to the breast attachment portion 51a.

As shown in FIG. 17 and FIG. 22, an embodiment of the present disclosure provides a breast pump assembly 10b for a breast pump 100b. The breast pump 100b can be worn on a breast, which frees the user's hands and allows the user to perform other tasks while using the breast pump 100b for milk expression. The breast pump assembly 10b is at least configured to express the milk.

Referring to FIG. 17 and FIG. 20, the breast pump assembly 10b includes a container body 11b and a container lid 12b. The container body 11b and the container lid 12b enclose a milk storage chamber 111b. In this way, the milk can be temporarily stored in the milk storage chamber 111b well.

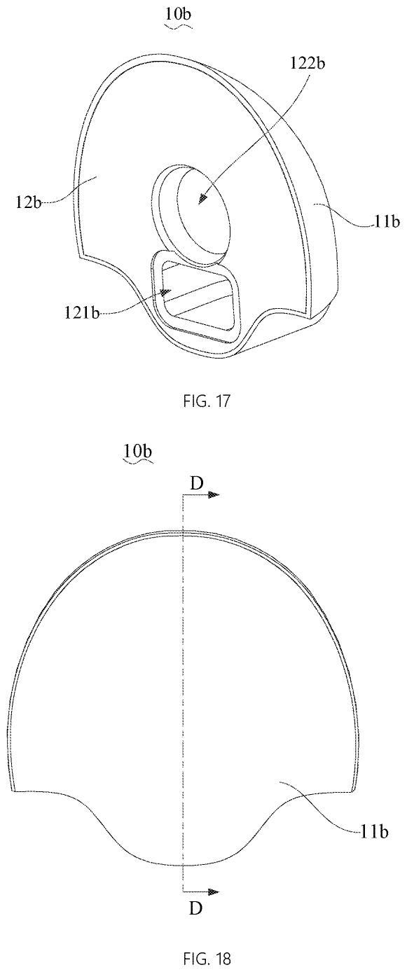

As an implementation, referring to FIGS. 18-20, FIG. 23, and FIG. 24, the breast pump assembly 10b further includes a negative pressure part 13b and a nipple tunnel cap element 14b. The negative pressure part 13b has a negative pressure chamber 131b. The negative pressure chamber 131b is configured to communicate with a negative pressure suction port 311b of the breast pump 100b. The negative pressure suction port 311b is configured to provide a negative pressure for the negative pressure chamber 131b. The nipple tunnel cap element 14b has a nipple tunnel 141b. The nipple tunnel 141b communicates with the negative pressure chamber 131b. The nipple tunnel 141b is at least configured to guide milk from a breast. The nipple tunnel cap element 14b and the negative pressure part 13b are arranged side by side in the breast pump 100b.

According to the above technical solution, the nipple tunnel cap element 14b having the nipple tunnel 141b is provided. The nipple tunnel 141b communicates with a breast receptacle 21b that is configured to accommodate the breast, and can be configured to guide the milk expressed from the breast. The negative pressure part 13b having the negative pressure chamber 131b is provided. The negative pressure chamber 131b communicates with the nipple tunnel 141b. The negative pressure chamber 131b can be configured to provide the negative pressure for the nipple tunnel 141b, thereby providing a negative pressure for the breast receptacle 21b, and thus expressing the milk. The nipple tunnel cap element 14b and the negative pressure part 13b are arranged side by side in the breast pump 100b, i.e., the nipple tunnel cap element 14b and the negative pressure part 13b have a same extension direction, so the breast pump 100b only needs to provide an enough space in a direction same as the extension direction of the nipple tunnel cap element 14b and the negative pressure part 13b to accommodate the nipple tunnel cap element 14b and the negative pressure part 13b. In contrast to the arrangement where the nipple tunnel cap element 14b and the negative pressure part 13b are crossed, and the breast pump 100b provides enough accommodating spaces in two different extension directions for the nipple tunnel cap element 14b and the negative pressure part 13b, the occupied space of the breast pump assembly 10b is effectively reduced. Therefore, the breast pump assembly 10b provided by this embodiment has a small occupied space, thereby reducing the overall size of the breast pump 100b.

As an implementation, the nipple tunnel cap element 14b and the negative pressure part 13b are connected side by side to a same surface of the container lid 12b and extend toward one side of the milk storage chamber 111b. This can effectively utilize the space of the milk storage chamber 111b, reducing assembly of external components for the breast pump assembly 10b.

As an implementation, when the breast pump 100b is placed against the breast, the negative pressure part 13b is located below the nipple tunnel cap element 14b. By this time, the negative pressure chamber 131b is located below the nipple tunnel 141b. In specific applications, the nipple tunnel 141b is located in a middle of the breast pump 100b. In this way, the negative pressure chamber 131b is located on a middle lower portion of the breast pump 100b. Therefore, the following pumping power device 321b is disposed on a lower portion of the breast pump 100b, thereby lowering a center of gravity of the breast pump 100b and further enabling the breast pump 100b to be tightly worn on the breast. Certainly, in specific applications, when the breast pump 100b is placed against the breast, the negative pressure part 13b may also be located at other positions, such as a left side, a right side, and an upside, of the nipple tunnel cap element 14b.

On the basis of the above implementation, when the breast pump 100b is placed against the breast, the negative pressure part 13b is located directly under the nipple tunnel cap element 14b, and the negative pressure chamber 131b communicates with the milk storage chamber 111b. This reduces the occupied space for left and right sides of the container body 11b, and makes an external contour of the container body 11b more aesthetically-pleasing. By this time, the negative pressure part 13b serves as a three-way structure, which communicates with the negative pressure suction port 311b, the nipple tunnel 141b, and the milk storage chamber 111b.

As an implementation, referring to FIG. 20, the nipple tunnel cap element 14b and the negative pressure part 13b share a first side wall 132b. Two opposite sides of the first side wall 132b face the negative pressure chamber 131b and the nipple tunnel 141b, respectively. A first through hole 1321b configured to communicate the negative pressure chamber 131b with the nipple tunnel 141b is formed in the first side wall 132b. This simplifies the communication structure between the nipple tunnel cap element 14b and the negative pressure part 13b, improving the production efficiency. Certainly, in specific applications, as an alternative implementation, the nipple tunnel cap element 14b and the negative pressure part 13b may also be spaced apart, and the negative pressure chamber 131b and the nipple tunnel 141b may also communicate via a communication tube.

As an implementation, when the breast pump 100b is placed against the breast, the first through hole 1321b is located at a lowest position of the nipple tunnel cap element 14b. The milk in the nipple tunnel 141b flows out from the first through hole 1321b under the action of gravity. By forming the first through hole 1321b at the lowest position of the nipple tunnel cap element 14b, the milk in the nipple tunnel 141b flows out from the first through hole 1321b as much as possible, thereby reducing milk accumulation in the nipple tunnel 141b.

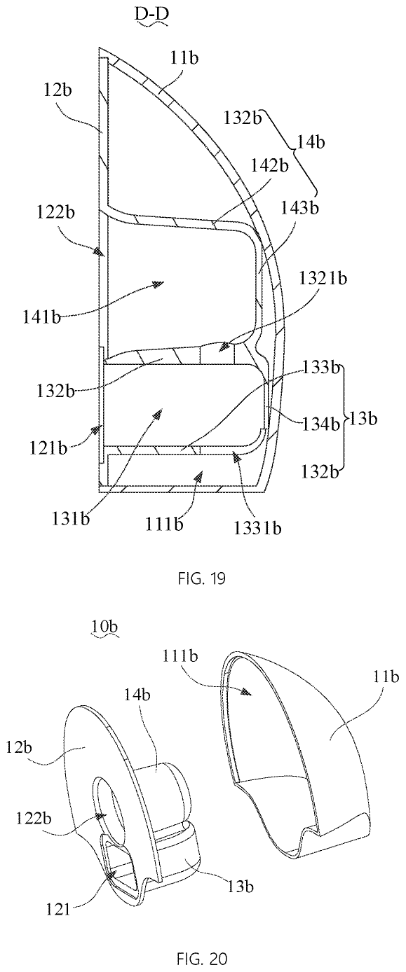

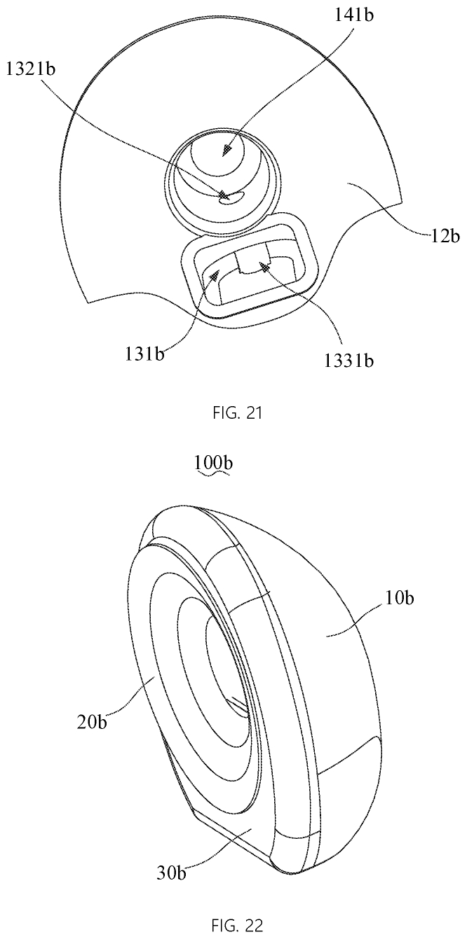

As an implementation, referring to FIG. 19, FIG. 21, FIG. 22, and FIG. 24, the negative pressure part 13b further includes a second side wall 133b and a first bottom wall 134b. The first side wall 132b, the second side wall 133b, and the first bottom wall 134b enclose the negative pressure chamber 131b. A second through hole 1331b configured to communicate the negative pressure chamber 131b with the milk storage chamber 111b is formed in the second side wall 133b. When the breast pump 100b is placed against the breast, the second through hole 1331b is located below the first through hole 1321b. In this way, the milk flows from the negative pressure chamber 131b to the milk storage chamber 111b. The first bottom wall 134b is opposite to an opening of the negative pressure chamber 131b. The opening of the negative pressure chamber 131b communicates with the negative pressure suction port 311b.

As an implementation, when the breast pump 100b is placed against the breast, the second through hole 1331b is located at a lowest position of the negative pressure part 13b. The milk in the negative pressure chamber 131b flows out from the second through hole 1331b under the action of gravity. By forming the second through hole 1331b at the lowest position of the negative pressure part 13b, the milk in the negative pressure chamber 131b flows out from the second through hole 1331b as much as possible, thereby reducing milk accumulation in the negative pressure chamber 131b.

As an implementation, when the breast pump 100b is placed against the breast, the second through hole 1331b directly faces the first through hole 1321b along a vertical direction. In this way, the milk entering the negative pressure chamber 131b through the first through hole 1321b can directly enter the milk storage chamber 111b through the second through hole 1331b, reducing a flow path of the milk in the negative pressure chamber 131b, and shortening retention time of the milk in the negative pressure chamber 131b.

In another implementation, when the breast pump 100b is placed against the breast, the second through hole 1331b is formed in an end of the second side wall 133b close to the first bottom wall 134b. In specific applications, a diaphragm 40b is disposed in the negative pressure chamber 131b. The diaphragm 40b has a contracted state and an expanded state. When the diaphragm 40b located in the negative pressure chamber 131b is in the expanded state, the diaphragm 40b expands from the opening of the negative pressure chamber 131b to the first bottom wall 134b. By forming the second through hole 1331b in the end of the second side wall 133b close to the first bottom wall 134b, the diaphragm 40b can push the milk to the second through hole 1331b during expansion, such that the milk flows out from the second through hole 1331b, reducing accumulation of the milk in the negative pressure chamber 131b.

As an implementation, the first bottom wall 134b is in smooth transition with the first side wall 132b and the second side wall 133b. This prevents dead angles at junctions of the first bottom wall 134b with the first side wall 132b and the second side wall 133b, facilitating the cleaning of the negative pressure chamber 131b.

As an implementation, referring to FIG. 19, the nipple tunnel cap element 14b further includes a third side wall 142b and a second bottom wall 143b. The first side wall 132b, the third side wall 142b, and the second bottom wall 143b enclose the nipple tunnel 141b. The second bottom wall 143b is opposite to an entrance of the nipple tunnel 141b.

On the basis of the above implementation, the second bottom wall 143b is in smooth transition with the first side wall 132b and the third side wall 142b. This prevents dead angles at junctions of the second bottom wall 143b with the first side wall 132b and the third side wall 142b, facilitating the cleaning of the nipple tunnel 141b.

As an implementation, a length of the nipple tunnel cap element 14b is the same as a length of the negative pressure part 13b. In specific applications, the nipple tunnel cap element 14b and the negative pressure part 13b extend along a thickness direction of the breast pump 100b, with the same length. This reduces the thickness of the breast pump 100b, and avoids the situation where either member is excessively long to cause the overlarge thickness of the breast pump 100b.

As an implementation, an opening of the negative pressure chamber 131b for communicating with the negative pressure suction port 311b and an opening of the nipple tunnel 141b for receiving the milk are arranged side by side and have a same orientation. In this way, for the breast pump 100b, the main pump module 30b provided with the negative pressure suction port 311b and the breast shield 20b configured to deliver the milk to the nipple tunnel 141b are located on a same side of the breast pump assembly 10b, such that the main pump module 30b, the breast shield 20b, and the breast pump assembly 10b are arranged more compactly, thereby reducing the overall size.

As an implementation, referring to FIG. 20, FIG. 21, and FIG. 24, a first perforation 121b and a second perforation 122b are formed in the container lid 12b. A central axis of the first perforation 121b and a central axis of the second perforation 122b are parallel or approximately parallel, with an included angle not greater than 20°. The negative pressure part 13b is connected to the first perforation 121b. The first perforation 121b is configured to communicate the negative pressure chamber 131b with the negative pressure suction port 311b. The nipple tunnel cap element 14b is connected to the second perforation 122b. The second perforation 122b communicates with the nipple tunnel 141b, and is configured to allow the milk to pass through and flow to the nipple tunnel 141b. In this way, the negative pressure part 13b and the nipple tunnel cap element 14b are connected to the container lid 12b, the milk can flow to the nipple tunnel 141b, and the negative pressure suction port 311b communicates with the negative pressure chamber 131b. Specifically, both the central axis of the first perforation 121b and the central axis of the second perforation 122b are perpendicular or approximately perpendicular to a plane where the container lid 12b is located, and form an included angle of 80-100° with the plane where the container lid 12b is located. Both the negative pressure part 13b and the nipple tunnel cap element 14b are perpendicular or approximately perpendicular to the container lid 12b. The negative pressure part 13b and the nipple tunnel cap element 14b each form an included angle of 80-100° with the container lid 12b.

Further, referring to FIGS. 22-24, an embodiment further provides a breast pump 100b, including: a breast shield 20b, a main pump module 30b, and the breast pump assembly 10b. A breast receptacle 21b is formed in the breast shield 20b. The main pump module 30b is provided with a negative pressure suction port 311b configured to transfer a negative pressure. The nipple tunnel 141b communicates with the breast receptacle 21b. The negative pressure chamber 131b communicates with the negative pressure suction port 311b. With the above breast pump assembly 10b, the size of the breast pump 100b is reduced, which is convenient for the user to carry and use.

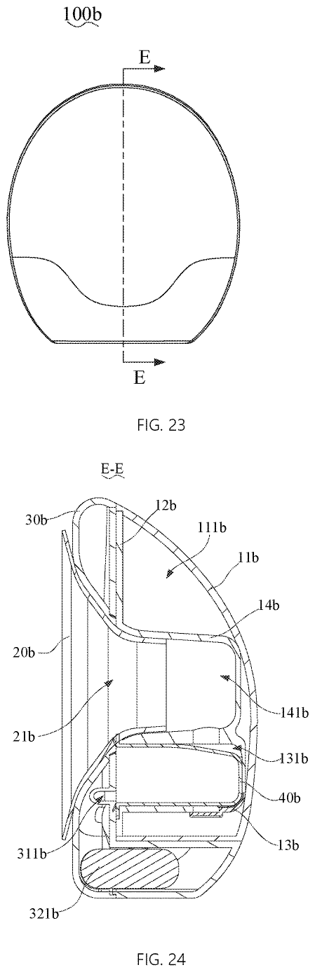

As an implementation, referring to FIGS. 23-25, the main pump module 30b includes a first support arm 31b and a second support arm 32b. The first support arm 31b is clamped between the breast shield 20b and the breast pump assembly 10b. The second support arm 32b is located on one side of the breast pump assembly 10b. The negative pressure suction port 311b is formed in a side of the first support arm 31b close to the second support arm 32b. The second support arm 32b includes a pumping power device 321b. Specifically, when the breast pump 100b is located on the breast, the second support arm 32b is located below the breast pump assembly 10b. Considering that the negative pressure part 13b is located below the nipple tunnel cap element 14b, a negative pressure generated by the pumping power device 321b can be quickly transferred to the negative pressure chamber 131b. Meanwhile, this arrangement can reduce the thickness of the breast pump 100b, such that the breast pump 100b is thinner.

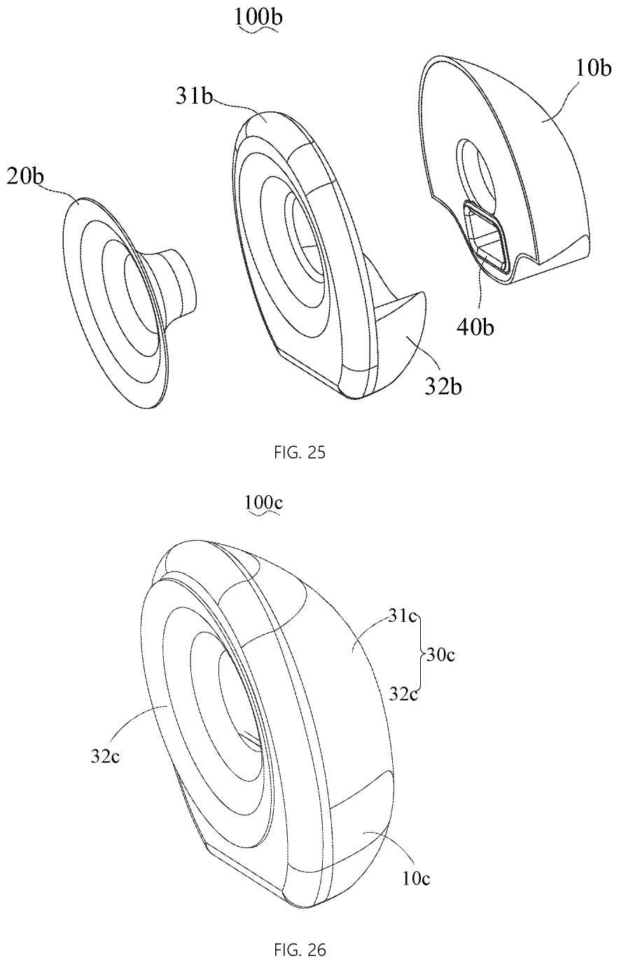

As an implementation, referring to FIG. 24 and FIG. 25, the breast pump 100b further includes a diaphragm 40b. The diaphragm 40b is connected to the container lid 12b and accommodated in the negative pressure chamber 131b. The diaphragm 40b is at least configured to prevent the milk from flowing to the negative pressure suction port 311b.

The main pump module of the breast pump is mainly configured to provide the negative pressure. In the related art, the structure of the main pump module is designed unreasonably with the large occupied space, such that the breast pump has an overlarge overall size and is inconvenient to carry and use, affecting the user experience.

In view of this, an embodiment of the present disclosure provides a breast pump. By reasonably arranging a structure of the main pump module, this embodiment effectively reduces the occupied space.

As shown in FIGS. 26-30, the breast pump 100c provided by this embodiment of the present disclosure includes: a main pump module housing 10c, a pumping power device 20c, and a milk pumping assembly 30c. The main pump module housing 10c includes a first shell 12c and a second shell 13c extending relative to the first shell 12c in a bending manner, so as to form a step 14c on the main pump module housing 10c. An accommodating chamber 11c is formed in the main pump module housing 10c. The pumping power device 20c is disposed in the accommodating chamber 11c, and configured to generate a negative pressure. The milk pumping assembly 30c is at least partially located on the step 14c.

The pumping power device 20c is disposed in the accommodating chamber 11c of the main pump module housing 10c, so the main pump module housing 10c serves to support and protect the pumping power device 20c.

According to the above technical solution, the second shell 13c extends relative to the first shell 12c in the bending manner, the step 14c is formed on the main pump module housing 10c, and the milk pumping assembly 30c is at least partially located on the step 14c, such that the main pump module housing 10c and the milk pumping assembly 30c are closely assembled together. This arrangement is reasonable, and greatly reduces the occupied space of the main pump module housing 10c and the overall size of the breast pump 100c, thereby making the breast pump 100c convenient to carry and use, and enhancing the user experience.

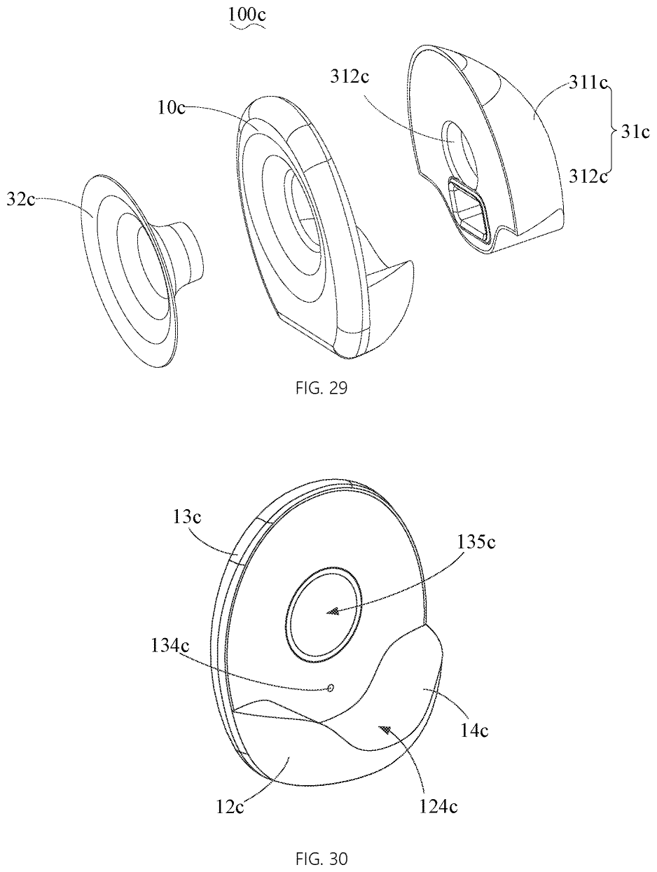

As an implementation, referring to FIGS. 29-31, the main pump module housing 10c is L-shaped, and an L-shaped step 14c is formed on the main pump module housing 10c. The L-shaped main pump module housing 10c features a neat and regular structure with a small occupied space, and the L-shaped step 14c facilitates the assembly of a breast shield 32c and a milk collection unit 31c, such that all components of the breast pump 100c can be tightly assembled, thereby achieving a reasonable arrangement, and effectively reducing the overall size of the breast pump 100c.

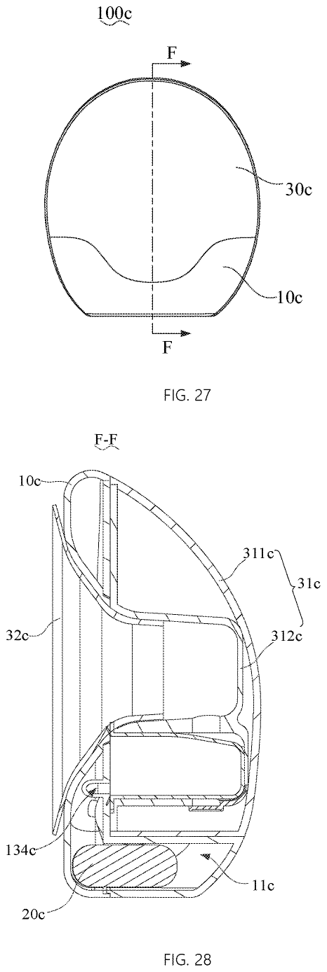

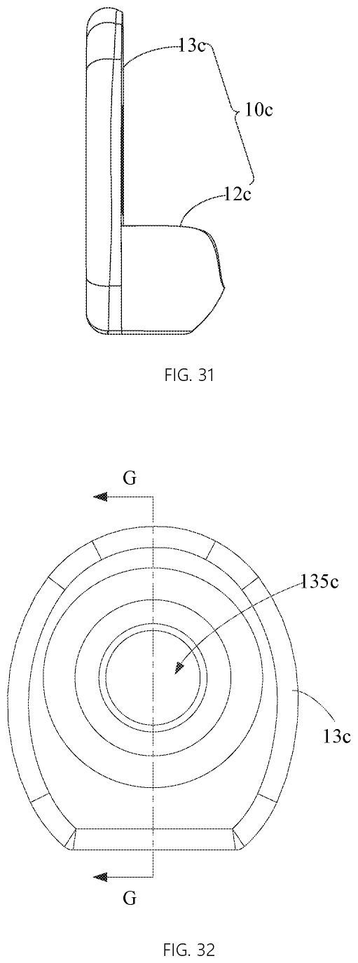

As an implementation, referring to FIG. 27, FIG. 28 and FIG. 31, the milk pumping assembly 30c includes the milk collection unit 31c and the breast shield 32c. The milk collection unit 31c is disposed on one side of the first shell 12c. The second shell 13c is disposed between the breast shield 32c and the milk collection unit 31c. At least one of the first shell 12c and the second shell 13c has a cavity, so as to form the accommodating chamber 11c. By clamping the second shell 13c between the breast shield 32c and the milk collection unit 31c, the breast shield 32c is connected to the milk collection unit 31c. The first shell 12c is disposed on one side, such as an upside, an underside, a left side or a right side, of the milk collection unit 31c. Terms such as the upside, the underside, the left side, and the right side are positions when the breast pump 100c is in an operating state.

On the basis of the above implementation, the milk collection unit 31c is supported on the first shell 12c. That is, the first shell 12c is disposed on the underside of the milk collection unit 31c, i.e., when the breast pump 100c is used, the first shell 12c is located below the milk collection unit 31c. In this way, the first shell 12c can support the milk collection unit 31c, improving stability of the milk collection unit 31c. As an implementation, referring to FIGS. 31-34, the first shell 12c has a first cavity 121c. The second shell 13c has a second cavity 131c. The accommodating chamber 11c includes the first cavity 121c and the second cavity 131c. In this way, both the first cavity 121c and the second cavity 131c can accommodate the component, achieving reasonable arrangement of the pumping power device 20c, and minimizing the occupied space of the main pump module housing 10c. Certainly, in specific applications, it is also appreciated that the second cavity 131c is not provided, but the second cavity 121c forms the accommodating chamber 11c, or the first cavity 121c is not provided, but the second cavity 131c forms the accommodating chamber 11c.

On the basis of the above implementation, at least a portion of the pumping power device 20c is accommodated in the first cavity 121c. This reduces the size of the second cavity 131c, the thickness of the second shell 13c, and the overall thickness of the breast pump 100c, thereby making the breast pump 100c thinner.

As an implementation, referring to FIG. 26, FIG. 31 and FIG. 36, the pumping power device 20c includes a negative pressure pump 21c, a solenoid valve 22c, a battery 23c, and a circuit board (not shown in the figure). The negative pressure pump 21c, the solenoid valve 22c, and the battery 23c are accommodated in the first cavity 121c. The circuit board is accommodated in the second cavity 131c. In this way, the weight of the pumping power device 20c is largely concentrated in the first cavity 121c. When the breast pump 100c is used, and the first shell 12c is located below the milk collection unit 31c, an overall center of gravity of the breast pump 100c is lowered, thereby achieving better sealing performance when the breast pump 100c is worn. Moreover, most components of the pumping power device 20c are disposed in the first cavity 121c, which can reduce the size of the second cavity 131c and the thickness of the second shell 13c, thereby making the overall breast pump 100c thinner.

In specific applications, any one or two of the negative pressure pump 21c, the solenoid valve 22c, and the battery 23c are accommodated in the second cavity 131c, or the circuit board is accommodated in the first cavity 121c.

The battery 23c is electrically connected to the circuit board. The circuit board is electrically connected to the negative pressure pump 21c and the solenoid valve 22c. When the negative pressure pump 21c is in operation, the solenoid valve 22c is idle, and the negative pressure pump 21c draws air out to generate a negative pressure. When the solenoid valve 22c is in operation, the negative pressure pump 21c is idle, and the solenoid valve 22c is opened, such that the air can be charged to a breast pump assembly 312c of the milk collection unit 31c.

On the basis of the above implementation, the negative pressure pump 21c, the battery 23c, and the solenoid valve 22c are arranged side by side. The battery 23c is located between the negative pressure pump 21c and the solenoid valve 22c. Both the negative pressure pump 21c and the solenoid valve 22c generate heat in operation. The negative pressure pump 21c and the solenoid valve 22c are spaced apart by the battery 23c, so as to prevent an excessive temperature in a local region. Certainly, in specific applications, as an alternative implementation, it is appreciated that the negative pressure pump 21c is disposed between the battery 23c and the solenoid valve 22c, or the solenoid valve 22c is disposed between the battery 23c and the negative pressure pump 21c.

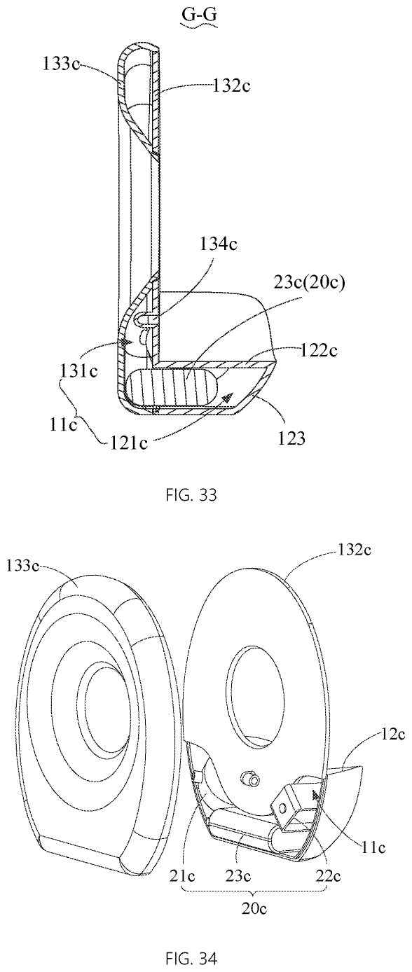

As an implementation, referring to FIG. 29, FIG. 31, and FIG. 33, the first shell 12c includes a first top shell 122c and a first bottom shell 123c. The first top shell 122c and the first bottom shell 123c enclose the first cavity 121c. The second shell 13c includes a second top shell 132c and a second bottom shell 133c. The second top shell 132c and the second bottom shell 133c enclose the second cavity 131c. The first top shell 122c and the second top shell 132c are perpendicular or approximately perpendicular and are respectively located on two sides of the milk collection unit 31c.

As an implementation, referring to FIG. 29, FIG. 30, FIG. 33, and FIG. 34, a negative pressure suction port 134c is formed in a side of the second shell 13c provided with the first shell 12c. The negative pressure suction port 134c communicates with the second cavity 131c. That is, a side of the second shell 13c facing the milk collection unit 31c is provided with the negative pressure suction port 134c, i.e., the second shell 132c is provided with the negative pressure suction port 134c. In specific applications, the negative pressure pump 21c and the solenoid valve 22c communicate with one end of an air guide tube. Another end of the air guide tube communicates with the negative pressure suction port 134c or penetrates into the negative pressure suction port 134c. The negative pressure suction port 134c can be configured to provide a negative pressure for the breast pump assembly 312c of the milk collection unit 31c or transfer air to it. By forming the negative pressure suction port 134c in the second top shell 132c, the negative pressure is provided for the breast pump assembly 312c of the milk collection unit 31c or the air is transferred to it.

On the basis of the above implementation, the negative pressure suction port 134c is formed in an end of the second shell 13c close to the first shell 12c. Both the negative pressure pump 21c and the solenoid valve 22c are disposed in the first cavity 121c of the first shell 12c. One end of the air guide tube communicates with the negative pressure pump 21c and the solenoid valve 22c. Another end of the air guide tube communicates with the negative pressure suction port 134c or penetrates into the negative pressure suction port 134c. The negative pressure suction port 134c is formed in the end of the second shell 13c close to the first shell 12c. This can shorten the air guide tube, and enhance the neatness in the accommodating chamber 11c.

As an implementation, the main pump module of the breast pump 100c further includes a key unit. The key unit is disposed on a circumferential side, other than an end close to the first shell 12c, of the second shell 13c. Disposing the key unit on the circumferential side of the second shell 13c facilitates the key operation of the user. The key unit is electrically connected to the circuit board. By operating different keys on the key unit, such parameters as a pumping frequency, a single pumping duration, and a pumping force in pumping operation can be adjusted or the on-off state of the breast pump can be operated, thereby meeting different milk pumping requirements of different users.

On the basis of the above implementation, the key unit is disposed on an end of the second shell 13c away from the first shell 12c. When the breast pump 100c is used, the first shell 12c is located on a bottom of the breast pump 100c, and the end of the second shell 13c away from the first shell 12c is located on a top of the breast pump 100c. By disposing the key unit on the end of the second shell 13c away from the first shell 12c, i.e., disposing the key unit on the end of the breast pump 100c, the key operation of the user is more convenient.

As an implementation, referring to FIG. 29 and FIG. 30, the second shell 13c is further provided with a through hole 135c. The through hole 135c extends from a side of the second shell 13c away from the first shell 12c to a side of the second shell 13c close to the first shell 12c. The through hole 135c is configured to allow the breast shield 32c to penetrate through. With the through hole 135c, the breast shield 32c and the milk collection unit 31c respectively located on two sides of the second shell 13c are connected. In specific applications, the breast shield 32c includes a breast attachment portion and a nipple channel. The through hole 135c corresponds to the nipple channel, so as to allow the nipple channel to penetrate through.

As an implementation, referring to FIG. 29, FIG. 30, and FIG. 33, a side of the first shell 12c provided with the second shell 13c is provided with a limiting recess 124c. That is, a side of the first shell 12c facing the milk collection unit 31c is provided with the limiting recess 124c, i.e., the first top shell 122c forms the limiting recess 124c. The limiting recess 124c is configured to limit the milk collection unit 31c, so as to enhance mounting stability of the milk collection unit 31c.

As an implementation, referring to FIG. 28 and FIG. 29, the milk collection unit 31c includes a milk storage container 311c and the breast pump assembly 312c extending into the milk storage container 311c. The pumping power device 20c is configured to provide the negative pressure for the breast pump assembly 312c. The breast pump assembly 312c extends into the milk storage container 311c, such that the milk collection unit 31c is arranged compactly, reducing the overall size of the breast pump 100c.

In the related art, heat sinks are used by the heat pump to dissipate heat of the main pump module. The heat sinks are mostly sheet-like structures made of aluminum alloy, brass or bronze. In order to improve the heat dissipation effect, a plurality of heat sinks are to be assembled, resulting in relatively large size and weight of the breast pump to greatly compromise the user experience.

In view of this, an embodiment of the present disclosure provides a breast pump. The breast pump makes use of milk to dissipate heat of the main pump module, and eliminates the heat sinks.

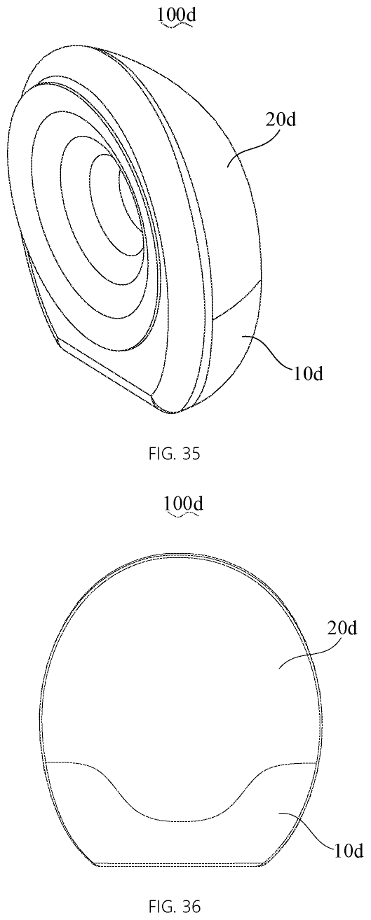

As shown in FIG. 35 and FIG. 36, the breast pump 100d provided by this embodiment of the present disclosure includes a main pump module 10d and a milk storage container 20d. The main pump module 10d is configured to provide a negative pressure. The milk storage container 20d is configured to store expressed milk.

In use, the main pump module 10d generates the negative pressure, and a breast pump assembly of the breast pump 100d expresses milk from a breast of a user through the negative pressure, and guide the milk to the milk storage container 20d for temporary storage.

As an implementation, referring to FIG. 36 and FIG. 37, the milk storage container 20d includes a support wall 21d and an enclosure wall 22d. The support wall 21d and the enclosure wall 22d enclose a milk storage chamber (not shown in the figure). The support wall 21d is configured to support milk flowing to the milk storage chamber. In specific applications, the expressed milk flows to the milk storage chamber of the milk storage container 20d for temporary storage. When the breast pump 100d is used, the support wall 21d is located below the enclosure wall 22d. The milk entering the milk storage chamber flows to the support wall 21d under the action of gravity, and is supported on the support wall 21d.

As an implementation, referring to FIG. 36 and FIG. 37, at least a portion of the main pump module 10d is attached to a side of the support wall 21d away from the milk storage chamber. That is, when the breast pump 100d is used, at least a portion of the main pump module 10d is located below the support wall 21d, and attached to the support wall 21d. In this way, when the milk flows to the milk storage chamber, travels downward to the support wall 21d, and is supported on the support wall 21d, since a temperature of the milk is less than a temperature of the main pump module 10d (the milk has the temperature of about 50-60° C., and the main pump module 10d has the temperature of far greater than 60° C.), the milk can absorb heat of the main pump module 10d, and serve to dissipate the heat of the main pump module 10d. In contrast to the heat dissipation method using the heat sinks, this omits the heat sinks, greatly reducing the overall size and weight of the device and thus enhancing the user experience. In addition, the main pump module 10d is disposed under the milk storage container 20d, serving to support the milk storage container 20d.

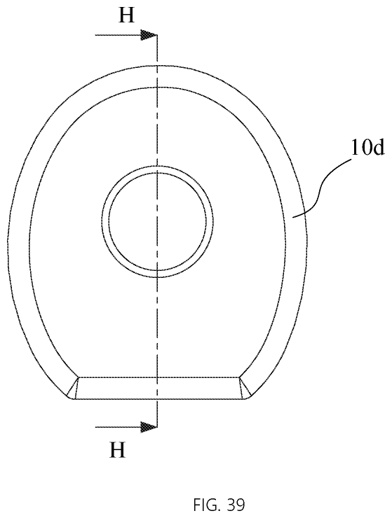

As an implementation, referring to FIGS. 37-40, the main pump module 10d includes a first bottom shell 11d, a first top shell 12d, and a negative pressure pump 13d. The first bottom shell 11d and the first top shell 12d enclose a first accommodating chamber 14d. The negative pressure pump 13d is accommodated in the first accommodating chamber 14d. The first top shell 12d is attached to the side of the support wall 21d away from the milk storage chamber. The negative pressure pump 13d generates a large amount of heat during operation. In this way, the milk can absorb the heat of the negative pressure pump 13d, and serve to dissipate the heat of the negative pressure pump 13d, effectively prolonging the service life of the negative pressure pump 13d.

The first top shell 12d is made of a material with a certain strength and an excellent thermal conductivity, such as thermal conductive plastic. In this way, the first top shell 12d can not only support the milk storage container 20d well, but also transfer heat efficiently.

As an implementation, a projected contour of the first top shell 12d along a direction toward the support wall 21d falls within a contour of the support wall 21d. In this way, the entire first top shell 12d is attached to the support wall 21d, maximizing a heat transfer area of the first top shell 12d to improve heat transfer efficiency. In this embodiment, the projected contour of the first top shell 12d along the direction toward the support wall 21d coincides with the contour of the support wall 21d.

As an implementation, a thickness of the first top shell 12d is less than a thickness of the first bottom shell 11d. The first top shell 12d with the smaller thickness increases the heat transfer efficiency of the first top shell 12d, while the first bottom shell 11d with the larger thickness greatly enhances the support performance of the first bottom shell 11d. Certainly, in specific applications, as an alternative, the thickness of the first top shell 12d may also be the same as the thickness of the first bottom shell 11d.

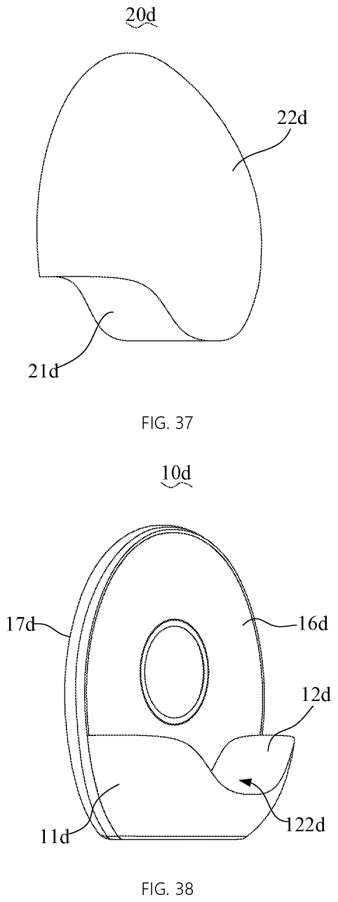

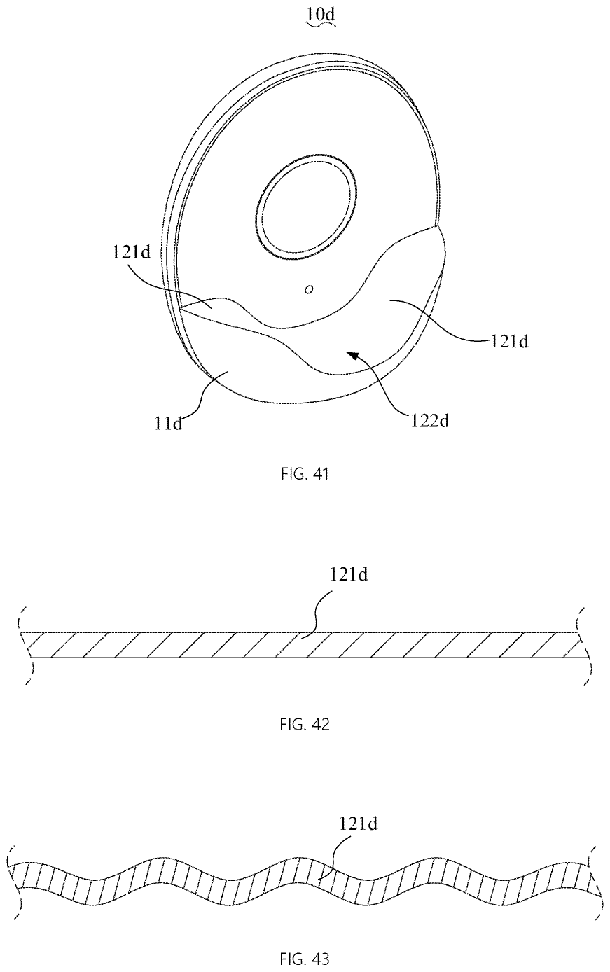

As an implementation, referring to FIG. 35, FIG. 38, and FIG. 41, the first top shell 12d includes at least two panels 121d. Any two adjacent ones of the panels 121d are disposed at an included angle. The included angle is less than 180°. In this way, the surface area of the first top shell 12d per unit lateral area of the breast pump 100d is increased, i.e., the heat transfer area per unit lateral area of the breast pump 100d is increased, thereby improving the heat transfer efficiency.

On the basis of the above embodiment, the included angle ranges from 90° to 170°. In specific applications, the included angle may be set as required. For example, it may be set to 100° or 130° or 150° or the like. For example, in this embodiment, the included angle is set to 120°.

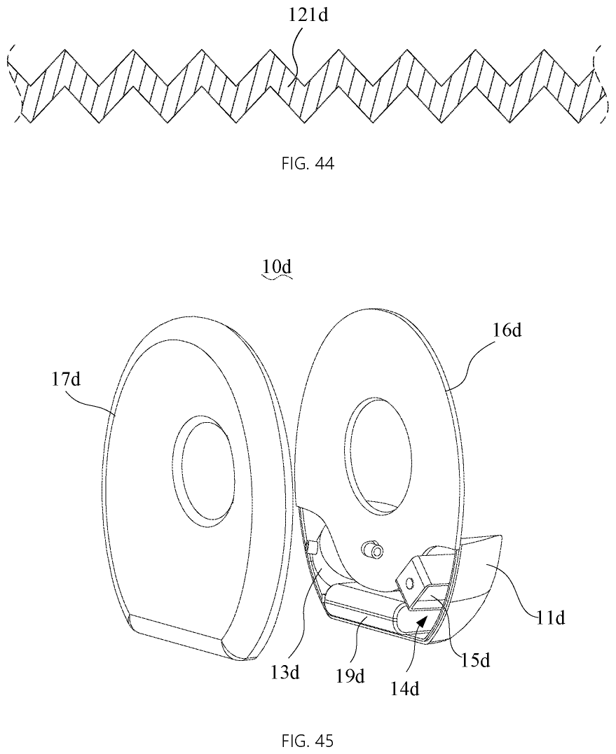

Referring to FIG. 42, each of the panels 121d is planar, which is simple in process, and easy to manufacture. In another embodiment, the panel 121d may also be non-planar. This further increases the surface area of the first top shell 12d to improve the heat transfer efficiency. Referring to FIG. 43 and FIG. 44, the panel 121d may be waved, and may also be in other shapes. The shape of the panel 121d is not limited herein.

As an implementation, two adjacent panels 121d are in smooth transition. This prevents a dead corner between the panels 121d, such that the first accommodating chamber 14d can effectively accommodate components, expanding an accommodation rate of the first accommodating chamber 14d. In addition, the support wall 21d is attached to the first top shell 12d, which also prevents a dead corner of the support wall 21d to facilitate the cleaning of the milk storage container 20d.

As an implementation, referring to FIG. 36 and FIG. 41, the panels 121d form a limiting recess 122d. The limiting recess 122d is configured to limit the milk storage container 20d. In specific applications, the limiting recess 122d is configured to limit a left-right direction of the milk storage container 20d.

As an implementation, the negative pressure pump 13d corresponds to a bottom wall of the limiting recess 122d. That is, a position of the first accommodating chamber 14d for accommodating the negative pressure pump 13d corresponds to the bottom wall of the limiting recess 122d. The milk flows to a lowest position of the support wall 21d under the action of gravity, and the lowest position of the support wall 21d is attached to the bottom wall of the limiting recess 122d, ensuring that the milk can still effectively dissipate the heat of the negative pressure pump 13d, even at an early stage of the milk flowing into the milk storage chamber or when the amount of the milk is small.

As an implementation, one of the first top shell 12d and the support wall 21d is provided with an anti-disengagement snap groove, while the other is provided with an anti-disengagement snap protrusion. The anti-disengagement snap protrusion is snap-fitted into the anti-disengagement snap groove. With snap-fitted assembly between the anti-disengagement snap protrusion and the anti-disengagement snap groove, the connection strength between the first top shell 12d and the support wall 21d can be enhanced, ensuring that the milk storage container 20d is not prone to loosening or even falling off the main pump module 10d when being accidentally pulled by an external force.

For example, in this embodiment, a side of the first top shell 12d away from the first accommodating chamber 14d is provided with the anti-disengagement snap protrusion, and a side of the support wall 21d away from the milk storage chamber is provided with the anti-disengagement snap groove.

It is to be noted that, optionally, the anti-disengagement snap protrusion is a spherical protrusion, and the correspondingly matched anti-disengagement snap groove is a spherical groove with a spherical bottom wall. This effectively prevents the milk storage container 20d from moving along a horizontal plane. Alternatively, optionally, the anti-disengagement snap protrusion may be a slide rail structure with a T-shaped cross-section, and the correspondingly matched anti-disengagement snap groove is a slide groove structure with a T-shaped cross-section. In this way, the milk storage container 20d can be slidably disposed on the main pump module 10d through the slide groove structure and the slide rail structure, and the milk storage container 20d is limited in an up-down direction.

As an implementation, the main pump module 10d further includes a solenoid valve 15d. The negative pressure pump 13d and the solenoid valve 15d are accommodated side by side in the first accommodating chamber 14d. The solenoid valve 15d generates heat during operation. By disposing the solenoid valve 15d in the first accommodating chamber 14d, the milk can also serve to dissipate heat of the solenoid valve 15d.

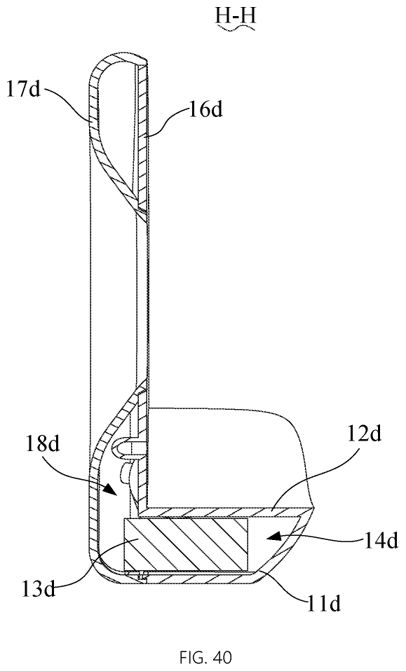

As an implementation, referring to FIG. 40 and FIG. 45, the main pump module 10d further includes a second top shell 16d and a second bottom shell 17d. The second top shell 16d and the second bottom shell 17d enclose a second accommodating chamber 18d. The first accommodating chamber 14d communicates with the second accommodating chamber 18d. The main pump module 10d further includes a battery 19d and a control panel (not shown in the figure). The battery 19d is accommodated in the first accommodating chamber 14d. The control panel is accommodated in the second accommodating chamber 18d. The battery 19d is electrically connected to the control panel. Both the negative pressure pump 13d and the solenoid valve 15d are electrically connected to the control panel.

The battery 19d, the negative pressure pump 13d, and the solenoid valve 15d are arranged side by side in the first accommodating chamber 14d. Referring to FIG. 40, FIG. 41, and FIG. 45, the negative pressure pump 13d may be disposed between the battery 19d and the solenoid valve 15d, and correspond to the bottom wall of the limiting recess 122d. The battery 19d may also be disposed between the negative pressure pump 13d and the solenoid valve 15d, thereby spacing two heat-producing components apart.

According to a second aspect, in view of shortcomings in the prior art, the present disclosure provides a breast pump, to solve the problems of milk accumulation and low milk expression efficiency.

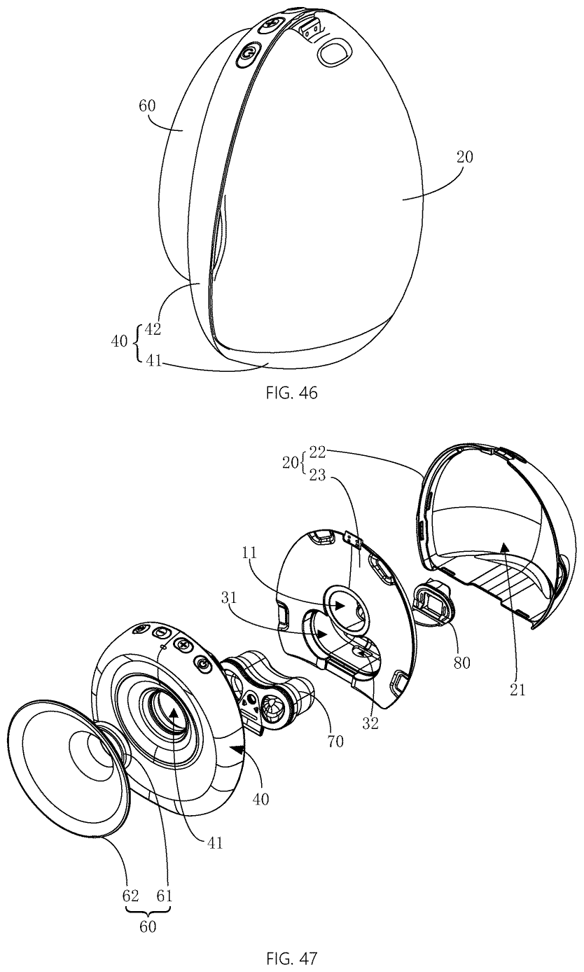

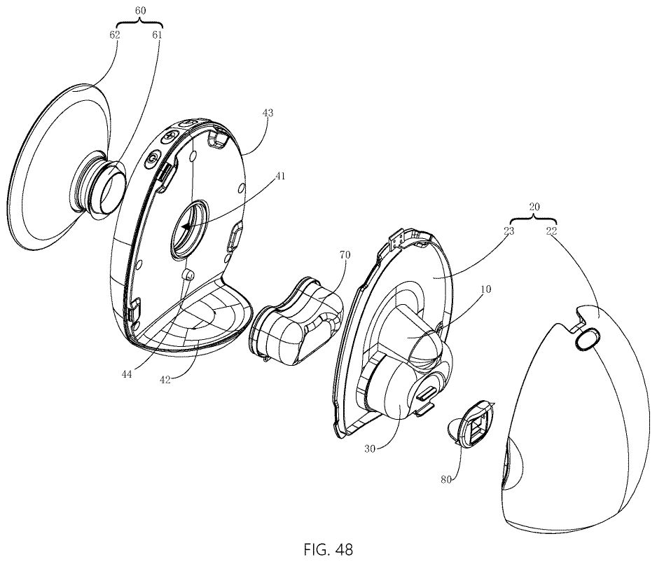

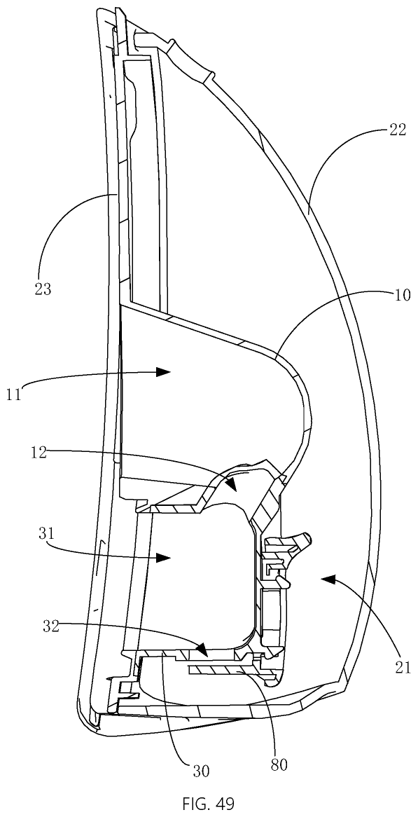

Referring to FIG. 46, FIG. 47, FIG. 48, FIG. 49, and FIG. 54, in an embodiment, a breast pump may include: a milk pumping conduit 10 and a milk storage container 20.

A nipple tunnel 11 is formed in the milk pumping conduit 10. The nipple tunnel 11 is configured to receive milk from a breast. A milk outlet 12 is formed in the milk pumping conduit 10. A negative pressure is transferred to the nipple tunnel 11 through the milk outlet 12.

A milk storage chamber 21 is formed in the milk storage container 20. The milk in the nipple tunnel 11 flows out of the nipple tunnel 11 through the milk outlet 12 and flows to the milk storage chamber 21. As used herein, the term “flows” means directly flowing to the milk storage chamber 21 or indirectly flowing to the milk storage chamber 21 (for example, via a negative pressure chamber), which is not limited in this embodiment of the present disclosure.

The transfer of the negative pressure and the outflow of the milk are realized through the milk outlet 12. The milk flows along a direction of an airflow formed by the negative pressure, thereby preventing the milk from remaining in the nipple tunnel 11 and improving the milk expression efficiency.

In an implementation, the milk storage container 20 is a milk bowl assembly 20. The milk pumping conduit 10 is connected to the milk bowl assembly 20. In some implementations, the milk storage container may also be a feeding bottle. When the feeding bottle is used, a bottle mouth of the feeding bottle may be directly or indirectly docked with the milk outlet 12. In some implementations, the milk pumping conduit 10 may be integrally connected to the milk bowl assembly 20. In some other implementations, the milk pumping conduit 10 may be detachably connected to the milk bowl assembly 20, which is not limited in this embodiment of the present disclosure. The milk pumping conduit 10 may extend into the milk storage chamber 21.

In an implementation, the milk bowl assembly 20 includes a bowl body 22 and a cover plate 23. The bowl body 22 and the cover plate 23 enclose the milk storage chamber 21. The milk pumping conduit 10 is connected to the cover plate 23. In some implementations, the milk pumping conduit 10 may be detachably or integrally connected to the cover plate 23, which is not limited in this embodiment of the present disclosure.

In an implementation, the breast pump further includes a diaphragm bracket 30. A negative pressure chamber 31 is formed in the diaphragm bracket 30. The negative pressure chamber 31 communicates with the nipple tunnel 11 through the milk outlet 12. The diaphragm bracket 30 may be located under the milk pumping conduit 10. The diaphragm bracket 30 may extend side by side with the milk pumping conduit 10. The diaphragm bracket 30 may extend into the milk storage chamber 21.

In an implementation, the diaphragm bracket 30 is provided with a milk flow port 32. After flowing out of the nipple tunnel 11 through the milk outlet 12, the milk in the nipple tunnel 11 flows through the negative pressure chamber 31 and flows to the milk storage chamber 21 through the milk flow port 32.

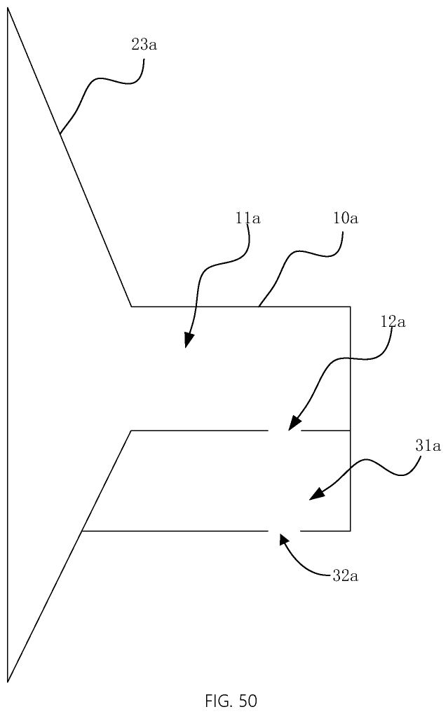

As shown in FIG. 50, in another implementation, the breast pump includes a breast shield 60. A milk pumping conduit 10a is connected to the breast shield 60. A nipple tunnel 11a is formed in the milk pumping conduit 10a. The nipple tunnel 11a is configured to receive milk from a breast. A milk outlet 12a is formed in the milk pumping conduit 10a. A negative pressure is transferred to the nipple tunnel 11a through the milk outlet 12a. The breast pump may further include a diaphragm bracket 30a. A negative pressure chamber 31a is formed in the diaphragm bracket 30a. The negative pressure chamber 31a communicates with the nipple tunnel 11a through the milk outlet 12a. The diaphragm bracket 30a is provided with a milk flow port 32a. After flowing out of the nipple tunnel 11a through the milk outlet 12a, the milk in the nipple tunnel 11a flows through the negative pressure chamber 31a and flows to a milk storage chamber through the milk flow port 32a.

In an implementation, the breast pump further includes a main pump module 40. The main pump module 40 is configured to provide the negative pressure. The main pump module 40 may include a housing and such components located in the housing as a negative pressure pump, a solenoid valve, a battery, and a circuit board. The main pump module 40 may form an integrated breast pump with the milk bowl assembly 20, or may be connected to the milk bowl assembly 20 via a conduit to form a split breast pump. It should be understood that the negative pressure is not limited to an electric negative pressure pump, and may also be provided by a manual negative pressure mechanism, which is not limited by this embodiment of the present disclosure. In some implementations, the breast pump may not include a negative pressure mechanism, but the negative pressure is provided by an external power.

In an implementation, the breast pump further includes a main pump module 40. The main pump module 40 includes a negative pressure port 44. The negative pressure port 44 may communicate with the negative pressure pump. The main pump module 40 communicates with the negative pressure chamber 31 through the negative pressure port 44. The main pump module 40 is configured to form the negative pressure in the negative pressure chamber 31. The negative pressure is transferred to the nipple tunnel 11 through the milk outlet 12.

In an implementation, the negative pressure chamber 31 is at least partially lower than the nipple tunnel 11 when the breast pump is worn. In some implementations, the negative pressure chamber 31 is completely located under the nipple tunnel 11. As used herein, the term “worn” means a worn state by a human body with an upper body in a normal upright posture, i.e., a state in which the wearing surface is substantially along a vertical direction.

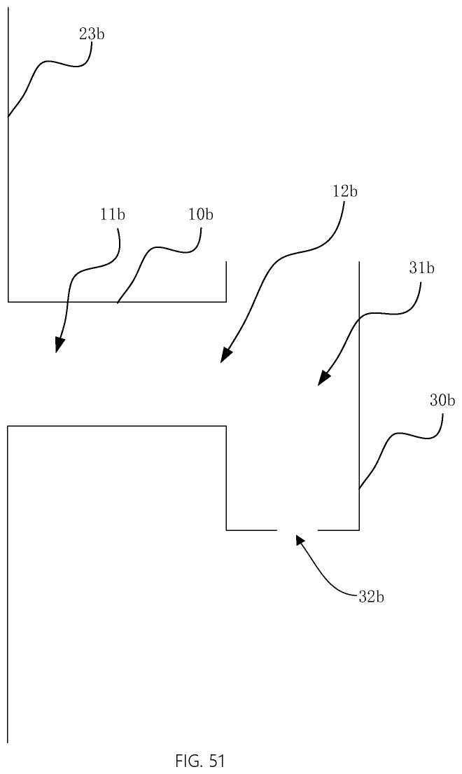

As shown in FIG. 51, in some other implementations, a negative pressure chamber 31b may also only have a portion located at a position lower than a nipple tunnel 11b. A milk pumping conduit 10b is connected to a cover plate 23b. The nipple tunnel 11b is formed in the milk pumping conduit 10b. The nipple tunnel 11b is configured to receive milk from a breast. A milk outlet 12b is formed in the milk pumping conduit 10b. The milk outlet 12b is formed in an end of the milk pumping conduit 10b. A negative pressure is transferred to the nipple tunnel 11b through the milk outlet 12b. The breast pump further includes a diaphragm bracket 30b. A negative pressure chamber 31b is formed in the diaphragm bracket 30b. The negative pressure chamber 31b communicates with the nipple tunnel 11b through the milk outlet 12b. The diaphragm bracket 30b is provided with a milk flow port 32b. After flowing out of the nipple tunnel 11b through the milk outlet 12b, the milk in the nipple tunnel 11b flows through the negative pressure chamber 31b and flows to the milk storage chamber through the milk flow port 32b.

In this way, the negative pressure chamber is at least partially located under the nipple tunnel, which can improve the milk expression efficiency, and prevent the milk accumulation.

In an implementation, the breast pump further includes a diaphragm 70. The diaphragm 70 is disposed in the negative pressure chamber 31. When the breast pump is worn, at least a portion of the diaphragm 50 is actuated below the nipple tunnel 11.

In this way, the negative pressure chamber is at least partially located under the nipple tunnel, which can improve the milk expression efficiency, and prevent the milk accumulation.

In an implementation, the milk outlet 12 is located at an underside of the milk pumping conduit 10. By forming the milk outlet 12 in the underside of the milk pumping conduit 10, the milk accumulation can further be reduced, preventing the milk from remaining in the milk pumping conduit 10. Meanwhile, the expressed milk in the milk pumping conduit 10 can flow out from the milk pumping conduit 10 in part by means of gravity, thereby further reducing the power consumption and improving the milk expression efficiency.

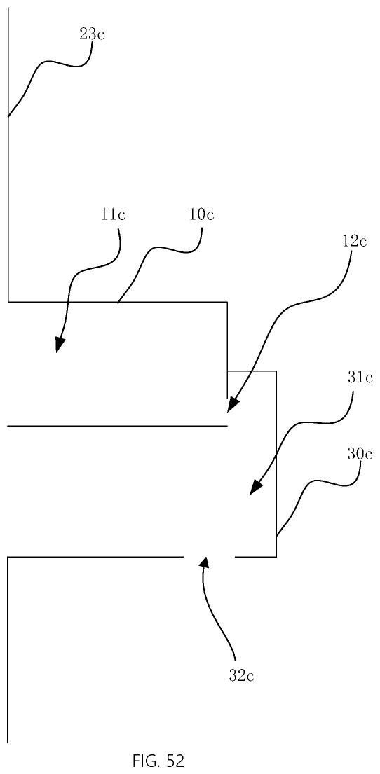

As shown in FIG. 52, in another implementation, a milk outlet 12c is formed at a lower position of an end of a milk pumping conduit 10c. A milk pumping conduit 10c is connected to a cover plate 23c. A nipple tunnel 11c is formed in the milk pumping conduit 10c. The nipple tunnel 11c is configured to receive milk from a breast. The milk outlet 12c is formed in the milk pumping conduit 10c. The milk outlet 12c is formed in an end of the milk pumping conduit 10c. A negative pressure is transferred to the nipple tunnel 11c through the milk outlet 12c. The breast pump may further include a diaphragm bracket 30c. A negative pressure chamber 31c is formed in the diaphragm bracket 30c. The negative pressure chamber 31c communicates with the nipple tunnel 11c through the milk outlet 12c. The diaphragm bracket 30c is provided with a milk flow port 32c. After flowing out of the nipple tunnel 11c through the milk outlet 12c, the milk in the nipple tunnel 11c flows through the negative pressure chamber 31c and flows to a milk storage chamber through the milk flow port 32c.

In an implementation, the milk outlet 12 is located between an end of the milk pumping conduit 10 and the cover plate 23. The milk in the nipple tunnel 11 flows out of the milk pumping conduit 10 through the milk outlet 12 under the action of the negative pressure. The milk pumping conduit 10 includes a distal end away from the cover plate 23. The milk outlet 12 is located between the cover plate 23 and the distal end. Since the milk outlet 12 is located between the end of the milk pumping conduit 10 and the cover plate 23, the overall structure of the breast pump can be more compact, the milk expression efficiency is improved, and the milk accumulation is prevented.

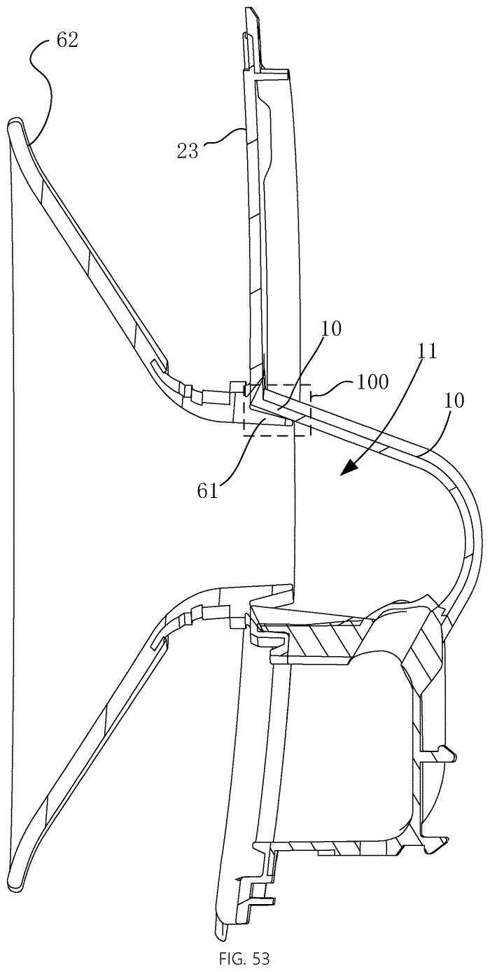

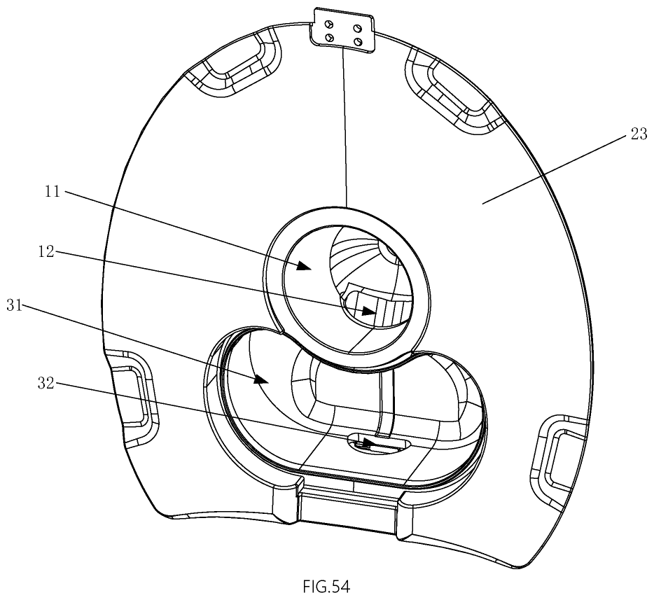

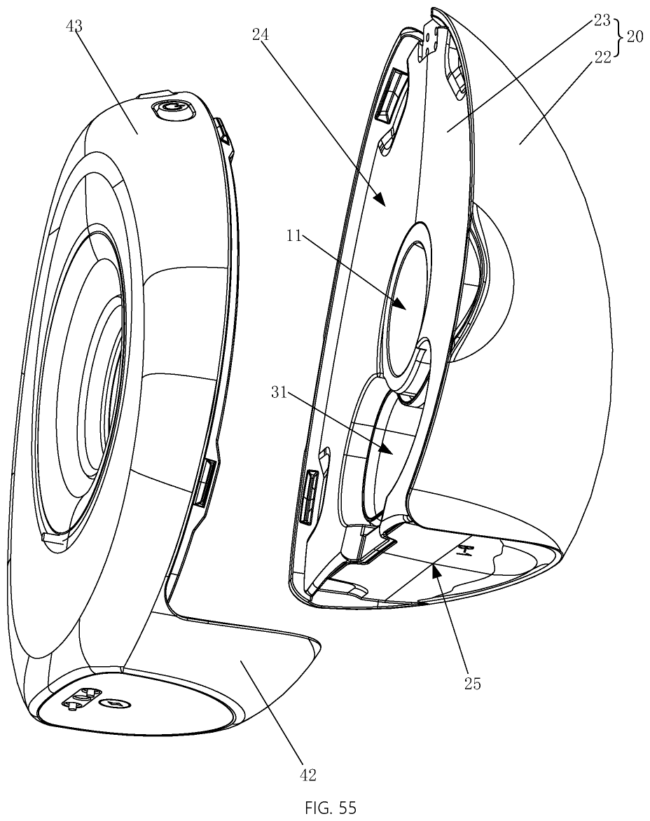

As shown in FIG. 48, FIG. 49, and FIG. 53, in an implementation, the distal end of the milk pumping conduit 10 is a sealed end. In an implementation, an end of the milk pumping conduit 10 away from a human body is sealed when the breast pump is worn. When the breast pump is worn, the end of the milk pumping conduit 10 away from the human body is sealed to form the sealed end, and the milk outlet 12 is located between the sealed end and the cover plate 23.

As shown in FIG. 52, in another implementation, a distal end of the milk pumping conduit 10c is an unsealed end. The milk outlet 12c is located at a lower position of the distal end.

In an implementation, the breast pump further includes a breast shield 60. An internal space of the breast shield 60 communicates with the nipple tunnel 11. One end of the breast shield 60 at least partially overlaps with the milk pumping conduit 10.

In an implementation, a position where the one end of the breast shield 60 overlaps with the milk pumping conduit 10 forms a double-layered structure 100. The double-layered structure 100 is at least partially made of a light-transmissive material, such that an interior of the nipple tunnel 11 or the breast shield 60 can be observed through the double-layered structure 100. The double-layered structure 100 includes at least two layers, one layer being a portion of the breast shield 60, and another layer being a portion of the milk pumping conduit 10.

Optionally, a narrow end of the breast shield 60 at least partially overlaps with the milk pumping conduit 10 to form the double-layered structure 100.

In an implementation, a region of the double-layered structure 100 made of the light-transmissive material is located on the nipple tunnel 11.

In an implementation, the end of the breast shield 60 is inserted into the milk pumping conduit 10. In some other implementations, the milk pumping conduit may also be inserted into the end of the breast shield, which is not limited in this embodiment of the present disclosure. In this embodiment of the present disclosure, the breast shield 60 may be connected to the main pump module 40. There is no connection or contact between the breast shield 60 and the milk pumping conduit 10. The narrow end 61 of the breast shield 60 is only inserted into the milk pumping conduit 10 without any interference fit, threaded connection, or the like. The breast shield 60 is fixed by the main pump module 40.

In an implementation, the milk bowl assembly 20 is at least partially made of the light-transmissive material, such that the interior of the nipple tunnel 11 or the breast shield 60 can be observed through the milk bowl assembly 20 and the double-layered structure 100, or an interior of the milk storage chamber 21 can be observed through the milk bowl assembly 20.

In an implementation, the breast pump further includes the breast shield 60. A through hole 41 is formed in a thickness direction of the main pump module 40. The breast shield 60 has the narrow end 61 and a wide end 62. The narrow end of the breast shield 60 penetrates into the through hole 41. The main pump module 40 is at least partially located between the wide end 62 of the breast shield 60 and the milk bowl assembly 20.

In an implementation, the milk storage container 20 is the milk bowl assembly 20. The main pump module 40 includes a transverse portion 42 and a longitudinal portion 43 connected to the transverse portion 42 in a bending manner. A first side 24 of the milk bowl assembly 20 contacts the longitudinal portion 43 of the main pump module 40. A second side 25 of the milk bowl assembly 20 contacts the transverse portion 42 of the main pump module 40. The first side 24 and the second side 25 are two adjacent sides of the milk bowl assembly 20.

In an implementation, the breast pump further includes a diaphragm 70. The diaphragm 70 is configured to be placed in a negative pressure chamber 31. The diaphragm 70 divides an internal space of a diaphragm bracket 30 into a first subspace and a second subspace. The first subspace communicates with a negative pressure port 44 of the main pump module 40. The second subspace communicates with an internal space of the milk pumping conduit 10. Driven by the negative pressure provided by the main pump module 40, the diaphragm 70 can deform in the internal space of the diaphragm bracket 30 to transfer the negative pressure.

In an implementation, the breast pump further includes a one-way valve 80. The one-way valve 80 is disposed at the milk flow port 32. The one-way valve 80 is configured to allow the milk to flow to the milk storage chamber 21 unidirectionally. The one-way valve 80 may be a disc-type one-way valve or a duckbill valve, which is not limited by this embodiment of the present disclosure.

According to this embodiment of the present disclosure, the breast pump includes the milk pumping conduit and the milk storage container. The nipple tunnel is formed in the milk pumping conduit. The nipple tunnel is configured to receive the milk from the breast. The milk outlet is formed in the milk pumping conduit. The negative pressure is transferred to the nipple tunnel through the milk outlet. The milk storage chamber is formed in the milk storage container. The milk in the nipple tunnel flows out of the nipple tunnel through the milk outlet and flows to the milk storage chamber. The transfer of the negative pressure and the outflow of the milk are realized through the milk outlet, preventing the milk from remaining in the nipple tunnel, and improving the milk expression efficiency.

According to the structural features of the first through hole 132, the second through hole 133, the milk outlet 12, and the milk flow port 32, etc., the correspondence between the first through hole 132 and the milk outlet 12, as well as the correspondence between the second through hole 133 and the milk flow port 32, can be clearly identified by referring to FIG. 9 and FIG. 49. In other words, the first through hole 132 is essentially the milk outlet 12, and the second through hole 133 is essentially the milk flow port 32. They are technically identical features, in only different ways of expression in naming. Other similar portions of the present application may also be compared with reference to the accompanying drawings.

According to a third aspect, in view of shortcomings in the prior art, the present disclosure provides a breast pump, to solve the problems of the uncompact structure and large size of the electric breast pump.

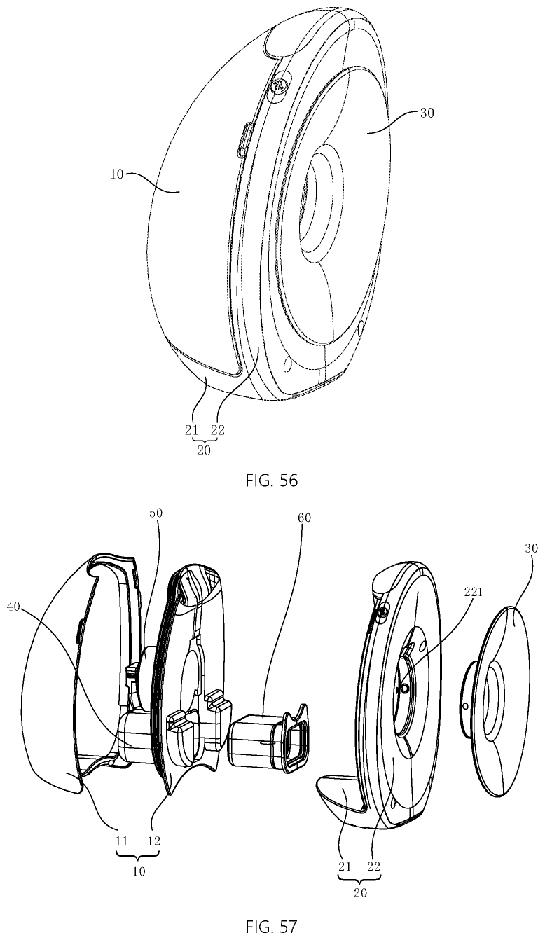

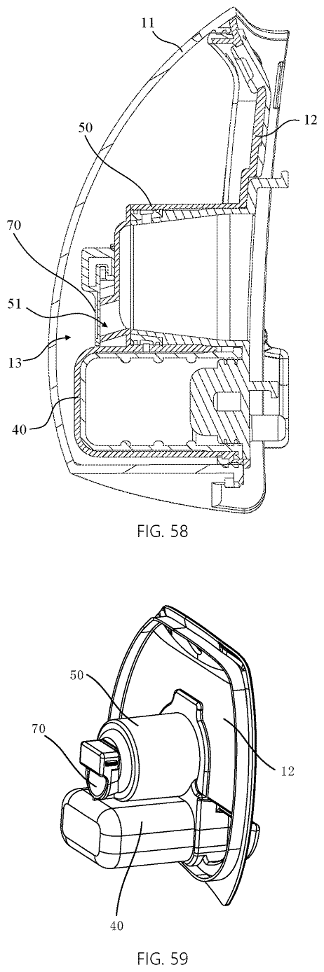

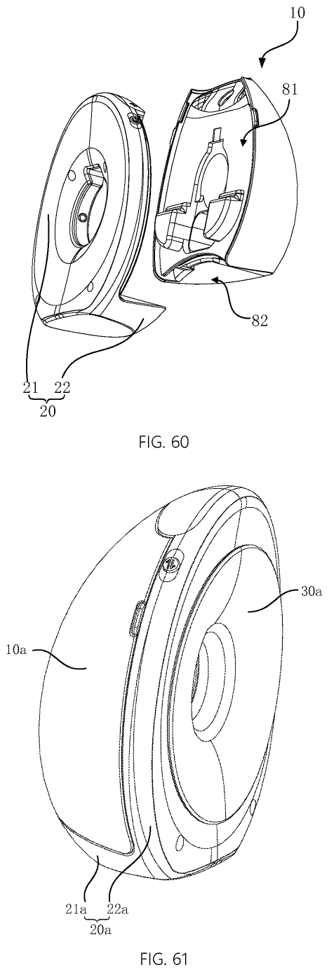

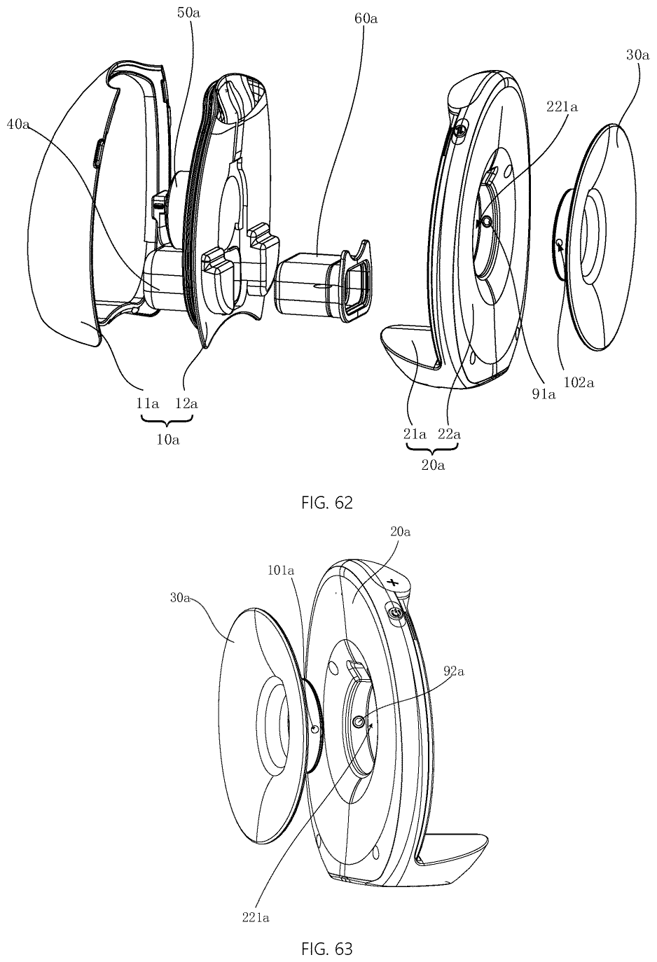

Referring to FIGS. 56-60, FIG. 56 is a schematic structural view of a breast pump according to an embodiment of the present disclosure. FIG. 57 is a schematic exploded view of a breast pump according to an embodiment of the present disclosure. FIG. 58 is a schematic sectional view of a breast pump after a breast shield and a main pump module are removed according to an embodiment of the present disclosure. FIG. 59 is a schematic structural view of a cover plate, a milk pumping conduit, and a diaphragm bracket of a breast pump according to an embodiment of the present disclosure. FIG. 61 is a schematic exploded view of a milk bowl assembly and a main pump module of a breast pump according to an embodiment of the present disclosure.

In this embodiment, the breast pump may include: a milk bowl assembly 10, a main pump module 20, a breast shield 30, and a diaphragm bracket 40. A milk storage chamber 13 is formed in the milk bowl assembly 10. A through hole 221 is formed in a thickness direction of the longitudinal portion of the main pump module 20. The breast shield 30 has a narrow end and a wide end. The narrow end of the breast shield 30 penetrates into the through hole 221. The main pump module 20 is at least partially located between the wide end of the breast shield 30 and the milk bowl assembly 10. The diaphragm bracket 40 is connected to the milk bowl assembly 10 and extends into the milk storage chamber 13.

In this embodiment, the milk bowl assembly 10 serves as a milk storage unit. The milk bowl assembly 10 is internally provided with the special milk storage chamber 13, and is configured to collect and temporarily store milk expressed from a mother. The through hole 221 is formed in the thickness direction of the main pump module 20. The design of the through hole 221 not only reduces the weight of the main pump module 20 and saves materials, but also can provide a space for a layout of other components, thereby facilitating the miniaturization of the overall structure.

In this embodiment, the main pump module 20 serves as a core power and control unit of the breast pump. Such components as a negative pressure pump, a control circuit, a battery, and a solenoid valve are integrated in the main pump module 20. The through hole 221 is formed in the thickness direction of the main pump module 20. The design of the through hole 221 not only reduces the weight of the main pump module 20 and saves materials, but also can provide a space for a layout of other components, thereby facilitating the miniaturization of the overall structure.

The wide end of the breast shield 30 comes into contact with a breast of a user. The breast shield 30 has the narrow end and the wide end, and from the narrow end to the wide end, the narrow end at least partially penetrates into the through hole 221 of the main pump module 20 to achieve a close fit with the main pump module 20. Meanwhile, the main pump module 20 is at least partially located between the wide end of the breast shield 30 and the milk bowl assembly 10. This layout keeps the overall structure of the breast pump stable, and ensures effective connection and functional coordination of the main pump module 20 with the breast shield 30 and the milk bowl assembly 10 during milk expression.

The diaphragm bracket 40 is connected to the milk bowl assembly 10 and extends into the milk storage chamber 13. A negative pressure chamber is formed in the diaphragm bracket. The negative pressure chamber of the diaphragm bracket 40 is configured to place a diaphragm 60. For details, reference may be made to the descriptions below.

In this embodiment of the present disclosure, the above configuration can reduce the overall occupied thickness space of the main pump module 20, the milk bowl assembly 10, and the breast shield 30. Moreover, the diaphragm bracket 40 is connected to the milk bowl assembly 10 and extends into the internal milk storage chamber 13 of the milk bowl assembly 10, such that the diaphragm bracket 40 does not additionally occupy the thickness space, further reducing the overall size of the breast pump, and facilitating portability.

In this embodiment, the breast pump may further include a milk pumping conduit 50 connected to the milk bowl assembly 10 and extending into the milk storage chamber 13. The milk pumping conduit 50 communicates with an internal space of the breast shield 30. A length of the milk pumping conduit extending into the milk storage chamber 13 is less than a length of the diaphragm bracket 40.

In this embodiment, the milk pumping conduit 50 is connected to the milk bowl assembly 10, extends into the milk storage chamber 13, and communicates with the internal space of the breast shield 30. The milk pumping conduit 50 serves as a delivery channel of the milk from the breast shield 30 to the milk storage chamber 13, ensuring that the milk can smoothly and effectively flow from the breast to the milk storage chamber 13 of the milk bowl assembly 10 under the action of a negative pressure, and preventing leakage or blockage of the milk during delivery. Since the milk pumping conduit 50 communicates with the internal space of the breast shield 30, the negative pressure generated during the milk expression can be directly applied to the breast shield 30, ensuring that the milk flows to the milk storage chamber 13 through the milk pumping conduit 50. This design ensures continuity and stability of the milk expression. The length of the milk pumping conduit 50 extending into the milk storage chamber 13 is less than the length of the diaphragm bracket 40, such that the overall structure is more compact, reducing the occupied space, and enhancing the portability. With the shorter milk pumping conduit 50, the risk of remaining the milk in the conduit can be lowered, facilitating cleaning, and reducing the risk of bacteria breeding.

Optionally, an end of the milk pumping conduit 50 away from the breast shield 30 is provided with a milk outlet 51. The milk outlet 51 is configured to allow milk in the milk pumping conduit 50 to flow to the milk storage chamber 13. By forming the milk outlet 51 in the end of the milk pumping conduit 50, the milk outlet 51 can be conveniently cleaned. The position of the milk outlet 51 is more prominent, which facilitates observation of the user on a milk outflow condition and effectively feeds back a milk discharge condition to the user.

In an implementation, a sum of a length of the milk pumping conduit 50 and a length of the milk outlet 51 is less than or equal to the length of the diaphragm bracket 40. By setting a total length of the milk pumping conduit 50 and the milk outlet 51 to be not greater than the length of the diaphragm bracket 40, the overall structure of the breast pump is compact, and particularly for the portable breast pump, the size of the device can be reduced, enhancing the portability and the use convenience.

In an implementation, the milk bowl assembly 10 includes a bowl body 11 and a cover plate 12. The bowl body 11 and the cover plate 12 enclose the milk storage chamber 13. Both the milk pumping conduit 50 and the diaphragm bracket 40 are connected to the cover plate 12. The bowl body 11 may be at least partially transparent, such that the user can observe the condition of milk in the milk pumping conduit 50 through the bowl body 11, or observe the outflow of milk at the milk outlet 51, or conveniently align a nipple when the breast pump is worn. In some implementations, a sealing ring may be disposed between the bowl body 11 and the cover plate 12, thereby ensuring sealing performance of connection between the bowl body 11 and the cover plate 12, and preventing leakage of the milk in the milk storage chamber 13.

In an implementation, the milk pumping conduit 50 and the diaphragm bracket 40 are integrally connected with the cover plate 12. In some implementations, the milk pumping conduit 50 may also be detachably connected to the cover plate 12. Likewise, the diaphragm bracket 40 may also be detachably connected to the cover plate 12.

In an implementation, the milk pumping conduit 50 and the diaphragm bracket 40 are arranged side by side. Arranging the milk pumping conduit 50 and the diaphragm bracket 40 side by side can further reduce their occupied thickness space. Both share the thickness space, such that the breast pump can be lighter and thinner.

In an implementation, the milk pumping conduit 50 is located on the diaphragm bracket 40. By disposing the milk pumping conduit 50 on the diaphragm bracket 40, the condition in the milk pumping conduit 50 can be conveniently observed, and the milk pumping conduit 50 is not shielded by the diaphragm bracket 40. The milk pumping conduit 50 is at least partially made of a light-transmissive material, such that the condition in the milk pumping conduit 50, like the milk flowing condition and whether the nipple channel is aligned in wearing, can be conveniently observed.

In an implementation, the main pump module 20 includes a transverse portion 21 and a longitudinal portion 22 connected to the transverse portion 21 in a bending manner. The through hole 221 is formed in the longitudinal portion 22. The longitudinal portion 22 is located between the wide end of the breast shield 30 and the milk bowl assembly 10. A first side S1 of the milk bowl assembly 10 comes into contact with the longitudinal portion 22 of the main pump module 20. A second side S2 of the milk bowl assembly 10 comes into contact with the transverse portion of the main pump module 20. The first side S1 and the second side S2 are two adjacent sides of the milk bowl assembly 10. Through the above configuration, an L-shaped step is formed on the main pump module 20. The milk bowl assembly 10 is disposed on the step of the main pump module 20, such that internal components in the main pump module 20 can be conveniently arranged, and the milk bowl assembly 10 is fixed more conveniently. This configuration can also achieve a large contact area between the main pump module 20 and the milk bowl assembly 10, facilitating heat dissipation of the main pump module 20.

In an implementation, the breast pump further includes a diaphragm 60. The diaphragm 60 is configured to be placed in the diaphragm bracket 40. The diaphragm 60 divides an internal space of the diaphragm bracket 40 into a first subspace and a second subspace. The first subspace communicates with a negative pressure port of the main pump module 20. The second subspace communicates with an internal space of the milk pumping conduit 50. Driven by a negative pressure provided by the main pump module 20, the diaphragm 60 can deform in the internal space of the diaphragm bracket 40 to transfer the negative pressure to the milk pumping conduit 50, thereby generating a negative pressure for milk expression in the milk pumping conduit 50. The diaphragm 60 is made of a flexible material, and mainly configured to prevent backflow of the milk to the main pump module 20 because of the negative pressure to cause damage to the main pump module 20.

In an embodiment, the breast pump further includes a one-way valve 70. The one-way valve 70 is disposed at the milk outlet 51. The one-way valve 70 is configured to allow the milk in the milk pumping conduit 50 to unidirectionally flow to the milk storage chamber 13. For example, the one-way valve may be a disc-type one-way valve, and may be a duckbill valve in other implementations. For example, in case of the negative pressure in the milk pumping conduit 50, the one-way valve 70 is closely attached to the milk outlet 51 under the action of a suction force, such that the milk in the milk storage chamber 13 cannot flow back. When the suction force is removed, the milk pushes the one-way valve 70 open under the action of gravity, thereby flowing to the milk storage chamber 13.

According to this embodiment of the present disclosure, the breast pump includes the milk bowl assembly, the main pump module, and the breast shield. The milk storage chamber is formed in the milk bowl assembly. The through hole is formed in the thickness direction of the main pump module. The breast shield has the narrow end and the wide end. The narrow end of the breast shield penetrates into the through hole. The main pump module is at least partially located between the wide end of the breast shield and the milk bowl assembly. The diaphragm bracket is connected to the milk bowl assembly and extends into the milk storage chamber. This configuration can reduce the overall occupied thickness space of the main pump module, the milk bowl assembly, and the breast shield. Moreover, the diaphragm bracket is connected to the milk bowl assembly and extends into the internal milk storage chamber of the milk bowl assembly, such that the diaphragm bracket does not additionally occupy the thickness space, further reducing the overall size of the breast pump, and facilitating portability.

Referring to FIG. 61, FIG. 62, and FIG. 67, FIG. 61 is a schematic structural view of a breast pump according to an embodiment of the present disclosure. FIG. 62 is a schematic exploded view of a breast pump according to an embodiment of the present disclosure. FIG. 67 is a schematic sectional view of a breast pump after a breast shield and a main pump module are removed according to an embodiment of the present disclosure.

In this embodiment, the breast pump may include: a milk bowl assembly 10a, a main pump module 20a, and a breast shield 30a.

The main pump module 20a is configured to provide a negative pressure for milk expression. The milk bowl assembly 10a is configured to accommodate milk. The milk bowl assembly 10a is disposed on a first side of the main pump module 20a. The breast shield 30a has a narrow end and a wide end. An inner diameter of the breast shield 30a from the narrow end to the wide end gradually increases. The wide end of the breast shield 30a is disposed on a second side of the main pump module 20a away from the first side. The breast shield 30a is connected to the main pump module 20a.

The breast shield 30a and the milk bowl assembly 10a are respectively disposed on two opposite sides, and the breast shield 30a is connected to the main pump module 20a, such that the overall size of the breast pump can be smaller. Meanwhile, the breast pump is connected to the main pump module, such that the connecting structure therebetween can be designed more stably and reliably compared with a solution in which the main pump module is fixed with the milk bowl assembly 10a.

In an implementation, the breast shield 30a is detachably connected to the main pump module 20a. The breast shield 30a and the main pump module 20a may be connected through detachable connecting structures complementary to each other. For example, one of the breast shield 30a and the main pump module 20a is provided with a snap fastener, while the other is provided with a snap groove. Alternatively, one is provided with an internal thread, while the other is provided with an external thread. Alternatively, one is provided with a ball plunger, while the other is provided with a snap groove. For details, reference may be made to the descriptions below.

In an embodiment, the breast pump further includes a milk pumping conduit 50a connected to the milk bowl assembly 10a. The milk pumping conduit 50a extends into a milk storage chamber 13a of the milk bowl assembly 10a. The narrow end of the breast shield 30a communicates with an end of the milk pumping conduit 50a. The breast shield 30a may be inserted into the milk pumping conduit 50a. Alternatively, an end surface of the narrow end abuts against an end surface of the milk pumping conduit 50a.

In an implementation, a through hole 221a is formed in a thickness direction of the main pump module 20a. The narrow end of the breast shield 30a is inserted into the through hole 221a. The narrow end of the breast shield 30a cooperates with the through hole to realize a connection between the breast shield 30a and the main pump module 20a.

In an embodiment, one of an outer wall of the narrow end of the breast shield 30a and an inner wall of the through hole 221a is provided with a groove, while the other is provided with a ball plunger. The groove cooperates with the ball plunger to realize the connection between the breast shield 30a and the main pump module 20a.

Referring to FIG. 63, FIG. 63 is a schematic exploded view of a main pump module and a breast shield according to an embodiment of the present disclosure. In this embodiment, a ball plunger 91a and a ball plunger 92a are disposed on the inner wall of the through hole 221a. A groove 101a and a groove 102a are formed in the outer wall of the narrow end of the breast shield 30a. The ball plunger 91a cooperates with the groove 101a, and the ball plunger 92a cooperates with the groove 102a. The ball plunger 91a and the ball plunger 92a are opposite to each other, and are located on two opposite sides of the inner wall of the through hole 221a, respectively. Accordingly, the groove 101a and the groove 102a are located on two opposite sides of the narrow end of the breast shield 30a, respectively. By providing two ball plungers and two corresponding grooves, the main pump module 20a and the breast shield 30a can be connected more stably, and detached conveniently and smoothly.

In another implementation, the narrow end of the breast shield 30a may be in an interference fit with the through hole 221a to realize the connection between the breast shield 30a and the main pump module 20a. That is, an outer diameter of the narrow end of the breast shield 30a may be slightly greater than an inner diameter of the through hole 221a, and the breast shield 30a is inserted into the through hole 221a through an interference fit, thereby realizing the connection between the breast shield 30a and the main pump module 20a. Alternatively, the outer diameter of the narrow end is less than the inner diameter of the through hole 221a, and an outer diameter of the breast shield 30a from the narrow end to the wide end gradually increases, so as to realize the interference fit for fixation during insertion.

In some implementations, one of an outer wall of the narrow end of the breast shield 30a and an inner wall of the through hole 221a is provided with a snap fastener, while the other is provided with a snap groove. The snap groove cooperates with the snap fastener to realize the connection between the breast shield 30a and the main pump module 20a.

In some implementations, the breast shield 30a and the main pump module 20a may be further fixed by providing a snap-fit connecting structure at any position of the main pump module 20a. The fixation is achieved by combining a variety of fixing methods with different structures and different positions, and is not limited in this embodiment of the present disclosure.

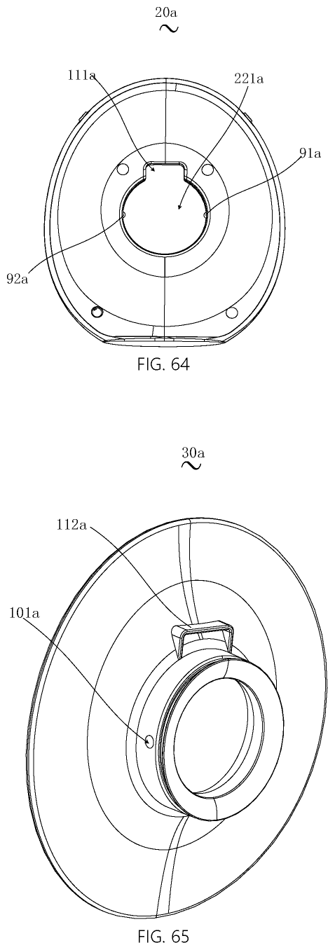

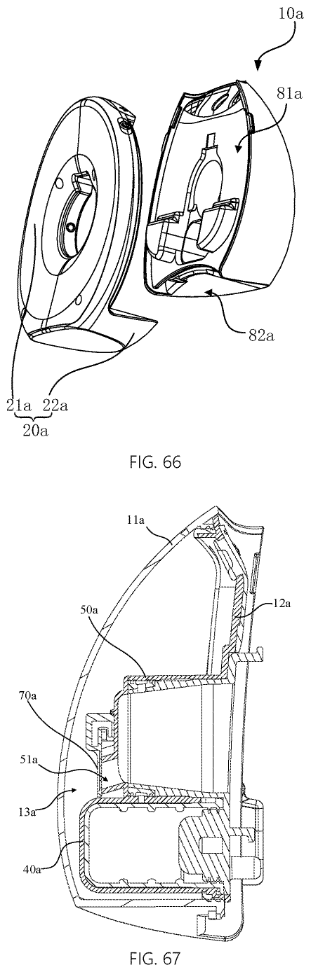

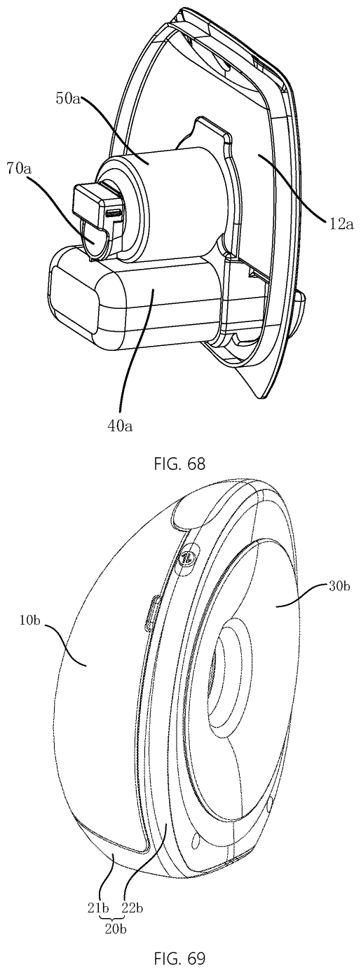

Referring to FIG. 64 and FIG. 65, FIG. 64 is a schematic front view of a main pump module according to an embodiment of the present disclosure. FIG. 65 is a schematic structural view of a breast shield according to an embodiment of the present disclosure.

Optionally, one of the main pump module 20a and the breast shield 30a is provided with a locating groove, while the other is provided with a locating protrusion. In this embodiment, the locating groove 111a is formed in the main pump module 20a, and may specifically formed in a periphery of the through hole 221a. The locating groove 111a communicates with the through hole 221a to form the notched locating groove 111a. The locating protrusion 112a is disposed on the breast shield 30a. Since connecting structures on the main pump module 20a and the breast shield 30a are provided at special positions, the locating groove 111a and the locating protrusion 112a can be more convenient for locating and mounting of the user.

Referring to FIG. 61, FIG. 61 is a schematic exploded view of a main pump module and a milk bowl assembly according to an embodiment of the present disclosure.

In an implementation, the main pump module 20a includes a transverse portion 21a and a longitudinal portion 22a connected to the transverse portion 21a in a bending manner. The through hole 221a is formed in the longitudinal portion 22a. The longitudinal portion 22a is located between the wide end of the breast shield 30a and the milk bowl assembly 10a. A first side 81a of the milk bowl assembly 10a comes into contact with the longitudinal portion 22a of the main pump module 20a. A second side 82a of the milk bowl assembly 10a comes into contact with the transverse portion of the main pump module 20a. The first side 81a and the second side 82a are two adjacent sides of the milk bowl assembly 10a. Through the above configuration, an L-shaped step is formed on the main pump module 20a. The milk bowl assembly 10a is disposed on the step of the main pump module 20a, such that internal components in the main pump module 20a can be conveniently arranged, and the milk bowl assembly 10 is fixed more conveniently. This configuration can also achieve a large contact area between the main pump module 20a and the milk bowl assembly 10a, facilitating heat dissipation of the main pump module 20a.

Optionally, an end surface of the narrow end of the breast shield 30a abuts against an end surface of the milk pumping conduit 50a, such that the narrow end communicates with the milk pumping conduit 50a in a sealing manner. The end surface of the narrow end may be provided with a soft rubber portion, or the end surface of the milk pumping conduit 50a may be provided with a soft rubber portion. Specifically, the soft rubber portion and the hard rubber portion may be integrally formed, or the soft rubber portion may be sleeved on the hard rubber portion. In this way, when the end surface of the narrow end abuts against the end surface of the milk pumping conduit 50a, the sealing communication is realized to prevent milk leakage.

As shown in FIG. 62, the milk storage chamber 13a is formed in the milk bowl assembly 10a. The through hole 221a is formed in the thickness direction of the main pump module 20a. The breast shield 30a has the narrow end and the wide end. The narrow end of the breast shield 30a penetrates into the through hole 221a. The main pump module 20a is at least partially located between the wide end of the breast shield 30a and the milk bowl assembly 10a. The diaphragm bracket 40a is connected to the milk bowl assembly 10a and extends into the milk storage chamber 13a.

In this embodiment, the milk bowl assembly 10a serves as a milk storage unit. The milk bowl assembly 10a is internally provided with the special milk storage chamber 13a, and is configured to collect and temporarily store milk expressed from a mother. The through hole 221a is formed in the thickness direction of the main pump module 20a. The design of the through hole 221a not only reduces the weight of the main pump module 20 and saves materials, but also can provide a space for a layout of other components, thereby facilitating the miniaturization of the overall structure.

In this embodiment, the main pump module 20a serves as a core power and control unit of the breast pump. Such components as a negative pressure pump, a control circuit, a battery, and a solenoid valve are integrated in the main pump module 20a. The through hole 221a is formed in the thickness direction of the main pump module 20a. The design of the through hole 221a not only reduces the weight of the main pump module 20a and saves materials, but also can provide a space for a layout of other components, thereby facilitating the miniaturization of the overall structure.

The wide end of the breast shield 30a comes into contact with a breast of the user. The breast shield 30a has the narrow end and the wide end, and from the narrow end to the wide end, the narrow end at least partially penetrates into the through hole 221a of the main pump module 20a to achieve a close fit with the main pump module 20a. Meanwhile, the main pump module 20a is at least partially located between the wide end of the breast shield 30a and the milk bowl assembly 10a. This layout keeps the overall structure of the breast pump stable, and ensures effective connection and functional coordination of the main pump module 20a with the breast shield 30a and the milk bowl assembly 10a during milk expression.

As shown in FIG. 63, FIG. 63 is a schematic structural view of a cover plate, a milk pumping conduit, and a diaphragm bracket according to an embodiment of the present disclosure. The diaphragm bracket 40a is connected to the milk bowl assembly 10a and extends into the milk storage chamber 13a. A negative pressure chamber is formed in the diaphragm bracket 40a. The negative pressure chamber of the diaphragm bracket 40a is configured to place a diaphragm 60a. For details, reference may be made to the descriptions below.

In this embodiment of the present disclosure, the above configuration can reduce the overall occupied thickness space of the main pump module 20a, the milk bowl assembly 10a, and the breast shield 30a. Moreover, the diaphragm bracket 40a is connected to the milk bowl assembly 10a and extends into the internal milk storage chamber 13a of the milk bowl assembly 10a, such that the diaphragm bracket 40a does not additionally occupy the thickness space, further reducing the overall size of the breast pump, and facilitating portability.

In this embodiment, the breast pump may further include a milk pumping conduit 50a connected to the milk bowl assembly 10a and extending into the milk storage chamber 13a. The milk pumping conduit 50a communicates with an internal space of the breast shield 30a. A length of the milk pumping conduit 50a extending into the milk storage chamber 13a is less than a length of the diaphragm bracket 40a.

In this embodiment, the milk pumping conduit 50a is connected to the milk bowl assembly 10a, extends into the milk storage chamber 13a, and communicates with the internal space of the breast shield 30a. The milk pumping conduit 50a serves as a delivery channel of the milk from the breast shield 30a to the milk storage chamber 13a, ensuring that the milk can smoothly and effectively flow from the breast to the milk storage chamber 13a of the milk bowl assembly 10a under the action of a negative pressure, and preventing leakage or blockage of the milk during delivery. Since the milk pumping conduit 50a communicates with the internal space of the breast shield 30a, the negative pressure generated during the milk expression can be directly applied to the breast shield 30a, ensuring that the milk flows to the milk storage chamber 13a through the milk pumping conduit 50a. This design ensures continuity and stability of the milk expression. Since the length of the milk pumping conduit 50a extending into the milk storage chamber 13a is less than the length of the diaphragm bracket 40a, such that the overall structure is more compact, reducing the occupied space, and enhancing the portability. With the shorter milk pumping conduit 50a, the risk of remaining the milk in the conduit can be lowered, facilitating cleaning, and reducing the risk of bacteria breeding.

Optionally, an end of the milk pumping conduit 50a away from the breast shield 30a is provided with a milk outlet 51a. The milk outlet 51a is configured to allow milk in the milk pumping conduit 50a to flow to the milk storage chamber 13a. By forming the milk outlet 51a in the end of the milk pumping conduit 50a, the milk outlet 51a can be conveniently cleaned. The position of the milk outlet 51a is more prominent, which facilitates observation of the user on a milk outflow condition and effectively feeds back the milk discharge condition to the user.

In an implementation, a sum of a length of the milk pumping conduit 50a and a length of the milk outlet 51a is less than or equal to the length of the diaphragm bracket 40a. By setting a total length of the milk pumping conduit 50a and the milk outlet 51a to be not greater than the length of the diaphragm bracket 40a, the overall structure of the breast pump is compact, and particularly for the portable breast pump, the size of the device can be reduced, enhancing the portability and the use convenience.

In an implementation, the milk bowl assembly 10a includes a bowl body 11a and a cover plate 12a. The bowl body 11a and the cover plate 12a enclose the milk storage chamber 13a. Both the milk pumping conduit 50a and the diaphragm bracket 40a are connected to the cover plate 12a. The bowl body 11a may be at least partially transparent, such that the user can observe the condition of milk in the milk pumping conduit 50a through the bowl body 11a, or observe the outflow of milk at the milk outlet 51a, or conveniently align a nipple when the breast pump is worn. In some implementations, a sealing ring may be disposed between the bowl body 11a and the cover plate 12a, thereby ensuring sealing performance of connection between the bowl body 11a and the cover plate 12a, and preventing leakage of the milk in the milk storage chamber 13a.

In an implementation, the milk pumping conduit 50a and the diaphragm bracket 40a are integrally connected with the cover plate 12a. In some implementations, the milk pumping conduit 50a may also be detachably connected to the cover plate 12a. Likewise, the diaphragm bracket 40a may also be detachably connected to the cover plate 12a.

In an implementation, the milk pumping conduit 50a and the diaphragm bracket 40a are arranged side by side. Arranging the milk pumping conduit 50a and the diaphragm bracket 40a side by side can further reduce their occupied thickness space. Both share the thickness space, such that the breast pump can be lighter and thinner.

In an implementation, the milk pumping conduit 50a is located on the diaphragm bracket 40a. By disposing the milk pumping conduit 50a on the diaphragm bracket 40a, the condition in the milk pumping conduit 50a can be conveniently observed, and the milk pumping conduit 50a is not shielded by the diaphragm bracket 40a. The milk pumping conduit 50a is at least partially made of a light-transmissive material, such that the condition in the milk pumping conduit 50a, like the milk flowing condition and whether the nipple channel is aligned in wearing, can be conveniently observed.

In an implementation, the breast pump further includes a diaphragm 60a. The diaphragm 60a is configured to be placed in the diaphragm bracket 40a. The diaphragm 60a divides an internal space of the diaphragm bracket 40a into a first subspace and a second subspace. The first subspace communicates with a negative pressure port of the main pump module 20a. The second subspace communicates with an internal space of the milk pumping conduit 50a. Driven by a negative pressure provided by the main pump module 20a, the diaphragm 60a can deform in the internal space of the diaphragm bracket 40a to transfer the negative pressure to the milk pumping conduit 50a, thereby generating a negative pressure for milk expression in the milk pumping conduit 50a. The diaphragm 60a is made of a flexible material, and mainly configured to prevent backflow of the milk to the main pump module 20a because of the negative pressure to cause damage to the main pump module 20a.

In an embodiment, the breast pump further includes a one-way valve 70a. The one-way valve 70a is disposed at the milk outlet 51a. The one-way valve 70a is configured to allow the milk in the milk pumping conduit 50a to unidirectionally flow to the milk storage chamber 13a. For example, the one-way valve may be a disc-type one-way valve, and may be a duckbill valve in other implementations. For example, in case of the negative pressure in the milk pumping conduit 50a, the one-way valve 70a is closely attached to the milk outlet 51a under the action of a suction force, such that the milk in the milk storage chamber 13a cannot flow back. When the suction force is removed, the milk pushes the one-way valve 70a open under the action of gravity, thereby flowing to the milk storage chamber 13a.

According to this embodiment of the present disclosure, the breast pump includes the main pump module, the milk bowl assembly, and the breast shield. The main pump module is configured to provide the negative pressure for the milk expression. The milk bowl assembly is configured to accommodate the milk. The milk bowl assembly is disposed on the first side of the main pump module. The breast shield has the narrow end and the wide end. The inner diameter of the breast shield from the narrow end to the wide end gradually increases. The wide end of the breast shield is disposed on the second side of the main pump module away from the first side. The breast shield is connected to the main pump module. The breast shield and the milk bowl assembly are respectively disposed on two opposite sides of the main pump module, and the breast shield is connected to the main pump module, such that the overall size of the breast pump can be smaller. Meanwhile, the breast pump is connected to the main pump module, such that the connecting structure therebetween can be designed more stably and reliably.

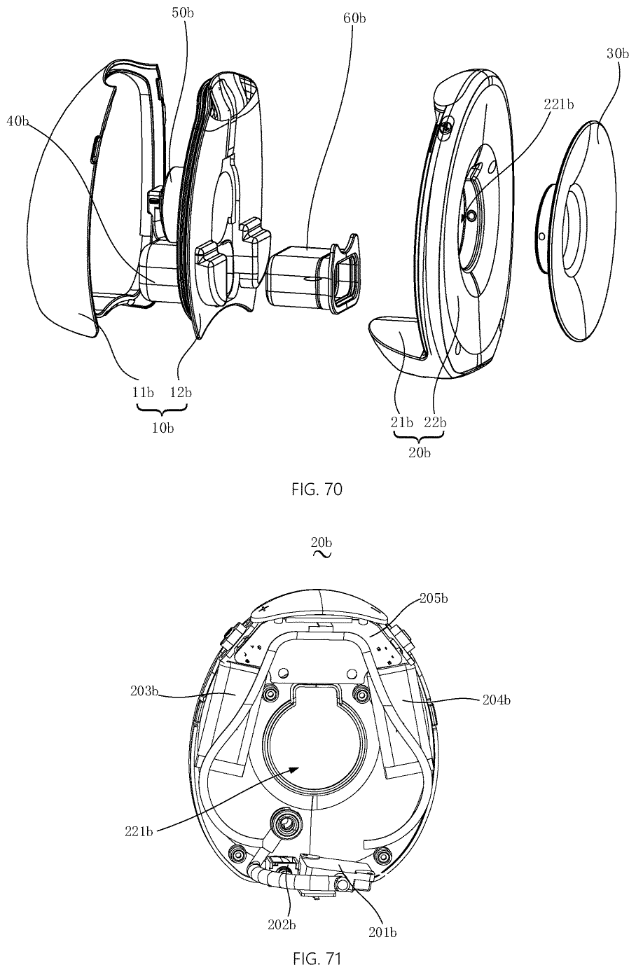

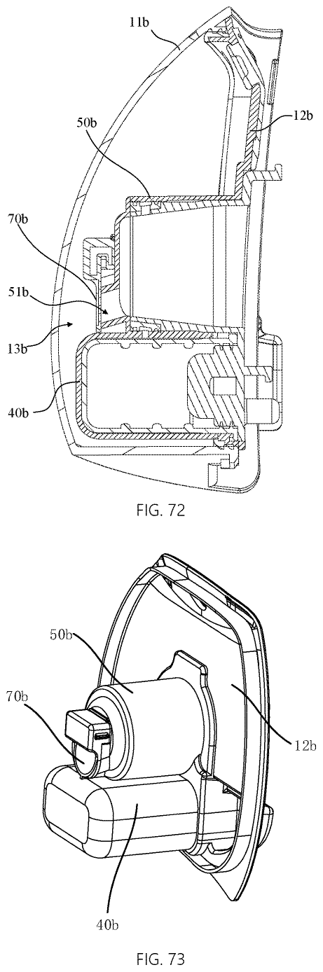

Referring to FIG. 69, FIG. 70, FIG. 72, and FIG. 76, FIG. 73 is a schematic structural view of a breast pump according to an embodiment of the present disclosure. FIG. 70 is a schematic exploded view of a breast pump according to an embodiment of the present disclosure. FIG. 72 is a schematic sectional view of a breast pump after a main pump module and a breast shield are removed according to an embodiment of the present disclosure. FIG. 73 is a schematic structural view of a cover plate, a milk pumping conduit, and a diaphragm bracket according to an embodiment of the present disclosure.

In this embodiment, the breast pump may include: a milk bowl assembly 10b, a main pump module 20b, a breast shield 30b, a diaphragm bracket 40b, and a milk pumping conduit 50b.

The breast shield 30b has a wide end and a narrow end. An inner diameter of the breast shield 30b from the narrow end to the wide end gradually increases. A milk storage chamber 13b is formed in the milk bowl assembly 10b. The milk pumping conduit 50b is connected to the milk bowl assembly 10b and extends into the milk storage chamber 13b. The milk pumping conduit 50b communicates with the breast shield 30b. The milk storage chamber 13b is configured to receive milk flowing out of the milk pumping conduit 50b. The main pump module 20b is configured to provide the milk pumping conduit 50b with a negative pressure for milk expression. When the milk storage chamber 13b is empty, a center of gravity of the breast pump is located within the milk pumping conduit 50b or the narrow end of the breast shield 30b.

The breast shield 30b includes a flange and a conduit located on the narrow end. The expression that the center of gravity of the breast pump is located within the narrow end means that the center of gravity of the breast pump is located within the conduit of the narrow end. With the narrow end and the milk pumping conduit 50b, a communicating nipple channel is formed in the narrow end and the milk pumping conduit 50b. The nipple channel is configured to receive a nipple of a user when the breast pump is used for milk expression. The design that the center of gravity is located within the milk pumping conduit 50b or the narrow end of the breast shield 30b may be achieved by adjusting a spatial layout of each component of the main pump module 20b. For details, reference may be made to the descriptions below.

By disposing the center of gravity of the breast pump within the milk pumping conduit 50b or the narrow end of the breast shield 30b, and close to the central position of the nipple, the breast pump is worn on the human body more stably and comfortably, with a more uniform suction force.

In an implementation, a through hole 221b is formed in the main pump module 20b. The breast shield 30b penetrates into the through hole 221b. The main pump module 20b is located between the wide end of the breast shield 30b and the milk bowl assembly 10b.

Referring to FIG. 71, FIG. 71 is a schematic internal view of a main pump module of a breast pump after a housing is removed according to an embodiment of the present disclosure. Optionally, a through hole 221b is formed in the main pump module 20b. A center of gravity of the main pump module 20b is located within the through hole 221b.

As shown in FIG. 71, in an implementation, the main pump module 20b includes a housing as well as a negative pressure pump 201b, a solenoid valve 202b, a first battery 203b, and a second battery 204b that are disposed in the housing. The negative pressure pump 201b and the solenoid valve 202b are located below the through hole 221b. The first battery 203b and the second battery 204b are located at a left side and a right side of the through hole 221b, respectively.

In some implementations, an internal spatial layout of the main pump module 20b is not limited to this, provided that the center of gravity can be located within the milk pumping conduit 50b or the narrow end of the breast shield 30b. For example, the negative pressure pump and the solenoid valve are respectively disposed on lower positions of left and right sides of the through hole 221b, and the first battery and the second battery are respectively disposed on upper positions of the left and right sides of the through hole 221b. Also for example, the negative pressure pump and the solenoid valve are respectively disposed on the upper positions of the left and right sides of the through hole 221b, and the first battery and the second battery are respectively disposed on the lower positions of the left and right sides of the through hole 221b. This embodiment of the present disclosure is not limited in this regard.

In an implementation, the main pump module 20b further includes a circuit board 205b located in the housing. The circuit board 205b is located above the through hole 221b.

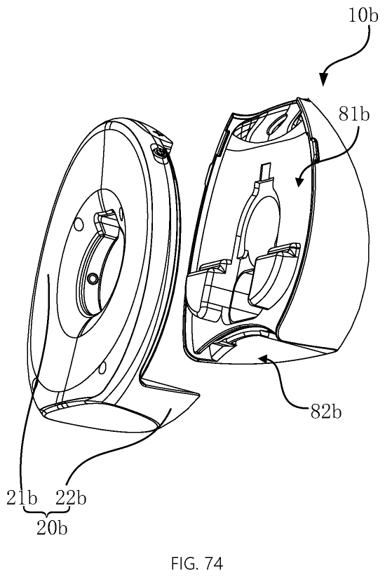

Referring to FIG. 74, FIG. 74 is a schematic exploded view of a main pump module and a milk bowl assembly according to an embodiment of the present disclosure. In an implementation, the main pump module 20b includes a transverse portion 21b and a longitudinal portion 22b connected to the transverse portion 21b in a bending manner. The through hole 221b is formed in the longitudinal portion 22b. The longitudinal portion 22b is located between the wide end of the breast shield 30b and the milk bowl assembly 10b. A first side 81b of the milk bowl assembly 10b comes into contact with the longitudinal portion 22b of the main pump module 20b. A second side 82b of the milk bowl assembly 10b comes into contact with the transverse portion 21b of the main pump module 20b. The first side 81b and the second side 82b are two adjacent sides of the milk bowl assembly 10b. The negative pressure pump 201b and the solenoid valve 202b are disposed in the transverse portion 21b. By disposing the main pump module 20b in an L shape including the transverse portion 21b and the longitudinal portion 22b, an internal space of the transverse portion 21b can be made larger. For example, such larger components as the negative pressure pump 201b and the solenoid valve 202b can be disposed in the transverse portion 21b, which can facilitate the layout of each large component in the main pump module 20b, and facilitates the configuration of the center of gravity within the milk pumping conduit 50b or the narrow end of the breast shield 30b.

In an implementation, the negative pressure pump 201b is a piezoelectric pump. Specifically, the piezoelectric pump may specifically be a piezoelectric ceramic pump, and may certainly be a piezoelectric pump using other media. This embodiment of the present disclosure is not limited in this regard. The piezoelectric pump is substantially flat, with its height being less than a half of its width.

In an implementation, the milk bowl assembly 10b includes a bowl body 11b and a cover plate 12b. The bowl body 11b and the cover plate 12b enclose the milk storage chamber 13b. The milk pumping conduit 50b is connected to the cover plate 12b.

In an implementation, the milk pumping conduit 50b and the cover plate 12b are integrally formed.

In an implementation, the breast pump further includes a diaphragm 60b. The diaphragm 60b is configured to be placed in the diaphragm bracket 40b. The diaphragm 60b divides an internal space of the diaphragm bracket 40b into a first subspace and a second subspace. The first subspace communicates with a negative pressure port of the main pump module 20b. The second subspace communicates with an internal space of the milk pumping conduit 50b. Driven by a negative pressure provided by the main pump module 20b, the diaphragm 60b can deform in the internal space of the diaphragm bracket 40b to transfer the negative pressure to the milk pumping conduit 50b, thereby generating a negative pressure for milk expression in the milk pumping conduit 50b. The diaphragm 60b is made of a flexible material, and mainly configured to prevent backflow of the milk to the main pump module 20b because of the negative pressure to cause damage to the main pump module 20b.

In an implementation, the milk bowl assembly 10b includes a bowl body 11b and a cover plate 12b. The bowl body 11b and the cover plate 12b enclose the milk storage chamber 13b. The diaphragm bracket 40b is connected to the milk bowl assembly 10b and extends into the milk storage chamber 13b. Both the milk pumping conduit 50b and the diaphragm bracket 40b are connected to the cover plate 12b. The bowl body 11b may be at least partially transparent, such that the user can observe the condition of milk in the milk pumping conduit 50b through the bowl body 11b, or observe the outflow of milk at the milk outlet 51b, or conveniently align a nipple when the breast pump is worn. In some implementations, a sealing ring may be disposed between the bowl body 11b and the cover plate 12b, thereby ensuring sealing performance of connection between the bowl body 11b and the cover plate 12b, and preventing leakage of the milk in the milk storage chamber 13b.

In an implementation, the milk pumping conduit 50b and the diaphragm bracket 40b are integrally connected with the cover plate 12b. In some implementations, the milk pumping conduit 50b may also be detachably connected to the cover plate 12b. Likewise, the milk pumping conduit 50b may also be detachably connected to the cover plate 12b.

In an implementation, the milk pumping conduit 50b and the diaphragm bracket 40b are arranged side by side. Arranging the milk pumping conduit 50b and the diaphragm bracket 40b side by side can further reduce their occupied thickness space. Both share the thickness space, such that the breast pump can be lighter and thinner.

In an implementation, the milk pumping conduit 50b is located on the diaphragm bracket 40b. By disposing the milk pumping conduit 50b on the diaphragm bracket 40b, the condition in the milk pumping conduit 50b can be conveniently observed, and the milk pumping conduit 50b is not shielded by the diaphragm bracket 40b. The milk pumping conduit 50b is at least partially made of a light-transmissive material, such that the condition in the milk pumping conduit 50b, like the milk flowing condition and whether the nipple channel is aligned in wearing, can be conveniently observed.

In an embodiment, the breast pump further includes a one-way valve 70b. The one-way valve 70b is disposed at the milk outlet 51b. The one-way valve 70b is configured to allow the milk in the milk pumping conduit 50b to unidirectionally flow to the milk storage chamber 13b. For example, the one-way valve may be a disc-type one-way valve, and may be a duckbill valve in other implementations. For example, in case of the negative pressure in the milk pumping conduit 50b, the one-way valve 70b is closely attached to the milk outlet 51b under the action of a suction force, such that the milk in the milk storage chamber 13b cannot flow back. When the suction force is removed, the milk pushes the one-way valve 70b open under the action of gravity, thereby flowing to the milk storage chamber 13b.

According to this embodiment of the present disclosure, the breast pump includes the breast shield, the milk bowl assembly, the milk pumping conduit, and the main pump module. The breast shield has the wide end and the narrow end. The inner diameter of the breast shield from the narrow end to the wide end gradually increases. The milk storage chamber is formed in the milk bowl assembly. The milk pumping conduit is connected to the milk bowl assembly and extends into the milk storage chamber. The milk pumping conduit communicates with the breast shield. The milk storage chamber is configured to receive the milk flowing out of the milk pumping conduit. The main pump module is configured to provide the milk pumping conduit with the negative pressure for the milk expression. When the milk storage chamber is empty, the center of gravity is located within the milk pumping conduit or the narrow end of the breast shield. By disposing the center of gravity of the breast pump within the milk pumping conduit or the narrow end of the breast shield, and close to the central position of the nipple, the breast pump is worn on the human body more stably and comfortably, with a more uniform suction force.

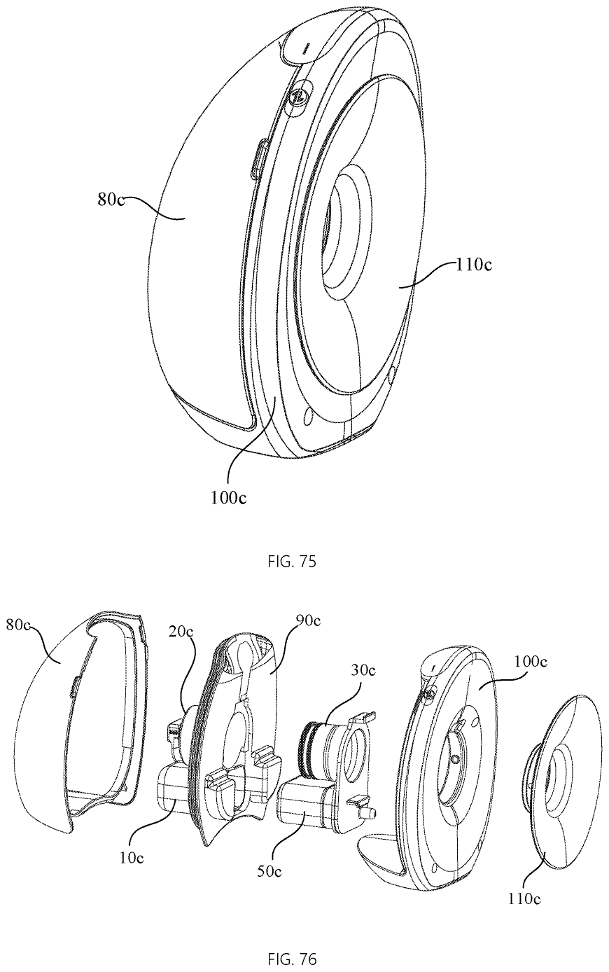

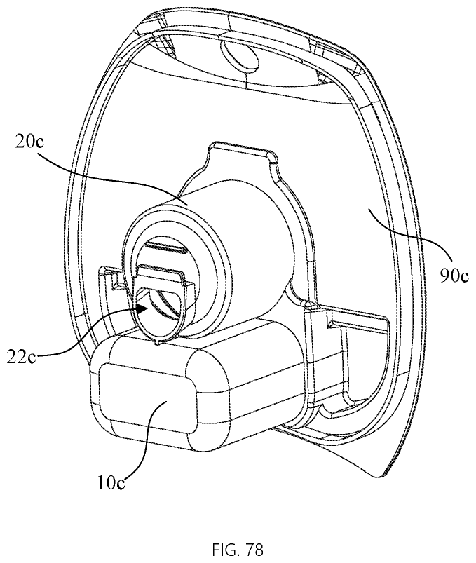

As shown in FIGS. 75-79, an embodiment of the present disclosure provides a breast pump assembly, applied to a breast pump, and including: a fixing member, a diaphragm bracket 10c, and an external nipple tunnel cap element 20c. Both the diaphragm bracket 10c and the external nipple tunnel cap element 20c are connected to the fixing member. When the breast pump is placed against a breast, the external nipple tunnel cap element 20c is located on the diaphragm bracket 10c. In other words, when the breast pump is worn by a user for milk expression, the external nipple tunnel cap element 20c is located on the diaphragm bracket 10c. The diaphragm bracket 10c has a negative pressure chamber 11c. The external nipple tunnel cap element 20c is provided with a first nipple tunnel 21c, a milk flow hole 22c configured to communicate the first nipple tunnel 21c with a milk storage chamber 81c of the breast pump, and a first air guide hole 23c configured to communicate the first nipple tunnel 21c with the negative pressure chamber 11c. By disposing the external nipple tunnel cap element 20c on the diaphragm bracket 10c, the diaphragm bracket 10c does not shield the external nipple tunnel cap element 20c, thereby facilitating observation of the user on a condition in the first nipple tunnel 21c.

During use, a negative pressure suction force enters the first nipple tunnel 21c through the first air guide hole 23c. Expressed milk flows to the first nipple tunnel 21c, and then to the milk storage chamber 81c through the milk flow hole 22c for temporary storage.

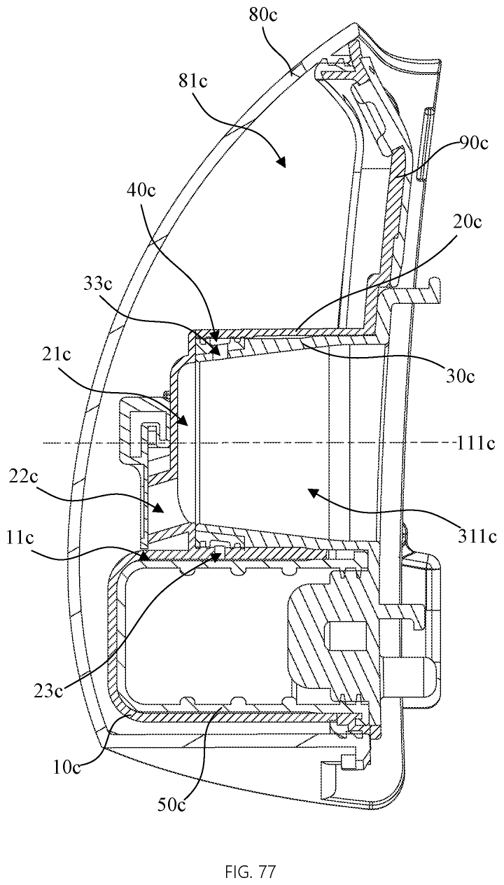

Referring to FIG. 76 and FIG. 77, the breast pump assembly further includes an internal nipple tunnel cap element 30c. At least a portion of the internal nipple tunnel cap element 30c extends into the first nipple tunnel 21c. The internal nipple tunnel cap element 30c is provided with a second nipple tunnel 311c and a second air guide hole 33c communicating with the second nipple tunnel 311c. The second nipple tunnel 311c communicates with the first nipple tunnel 21c. The internal nipple tunnel cap element 30c and an inner side wall of the external nipple tunnel cap element 20c enclose an air guide channel 40c. Both the first air guide hole 23c and the second air guide hole 33c communicate with the air guide channel 40c. The second air guide hole 33c is located above a central axis 111c of the second nipple tunnel 311c when the breast pump is placed against the breast. Since at least the portion of the internal nipple tunnel cap element 30c extends into the first nipple tunnel 21c, the internal nipple tunnel cap element 30c is located on the diaphragm bracket 10c. That is, the second nipple tunnel 311c is located on the diaphragm bracket 10e, such that the diaphragm bracket 10c does not shield the internal nipple tunnel cap element 30c, thereby facilitating observation of the user on a condition in the second nipple tunnel 311c. When the breast pump is placed against the breast, the second air guide hole 33c is located above the first air guide hole 23c. That is, when the breast pump is worn by the user for the milk expression, the second air guide hole 33c is located above the first air guide hole 23c. Optionally, the first air guide hole 23c is located below the central axis 111c of the second nipple tunnel 311c. An extension direction of the central axis 111c is the same as an extension direction of the second nipple tunnel 311c. When the breast pump is placed against the breast, the extension direction of the second nipple tunnel 311c is horizontal or substantially horizontal, and the central axis 111c is also horizontal or substantially horizontal.

During use, the nipple is aligned at or extended into the second nipple tunnel 311c, a negative pressure suction force enters the air guide channel 40c through the first air guide hole 23c, and then enters the second nipple tunnel 311c through the second air guide hole 33c. Expressed milk flows to the second nipple tunnel 311c, then to the first nipple tunnel 21c, and to the milk storage chamber 81c through the milk flow hole 22c for temporary storage.

According to the above technical solution, by forming the air guide channel 40c, and providing the first air guide hole 23c and the second air guide hole 33c to communicate with the air guide channel 40c, the negative pressure chamber 11c communicates with the second nipple tunnel 311c, and a negative pressure can be generated in the second nipple tunnel 311c, thereby expressing the milk. By forming the second air guide hole 33c above the central axis 111c of the second nipple tunnel 311c, the milk in the second nipple tunnel 311c does not easily flow to the air guide channel 40c and the negative pressure chamber 11c through the second air guide hole 33c, thereby preventing contamination to the negative pressure chamber 11c, and degradation of suction performance of the breast pump. Therefore, the breast pump assembly provided by this embodiment can prevent the milk from flowing to the negative pressure chamber 11c from the nipple tunnel (the first nipple tunnel 21c and/or the second nipple tunnel 311c), thereby preventing contamination to the negative pressure chamber 11c, and degradation of suction performance of the breast pump.

As an implementation, when the breast pump is placed against the breast, a line connecting the second air guide hole 33c and the central axis 111c of the second nipple tunnel 311c forms an included angle of 80-100° with a horizontal plane. Optionally, the line connecting the second air guide hole 33c and the central axis 111c of the second nipple tunnel 311 forms the included angle of 90° with the horizontal plane. That is, the second air guide hole 33c is located on an uppermost side of the second nipple tunnel 311c, preventing the milk from flowing to the air guide channel 40c through the second air guide hole 33c.

As an implementation, the external nipple tunnel cap element 20c and the diaphragm bracket 10c are arranged side by side and share a wall. That is, the nipple tunnel cap element 20c and the diaphragm bracket 10c share a same side wall. The side wall includes one side facing the first nipple tunnel 21c, and another side facing the negative pressure chamber 11c. The first air guide hole 23c is formed in the side wall.

As an implementation, the external nipple tunnel cap element 20c is approximately of a cylindrical structure with one end provided with an opening. It includes a cavity forming the first nipple tunnel 21c, a bottom wall provided with the milk flow hole 22c, and the side wall provided with the first air guide hole 23c. When the breast pump is placed against the breast, the external nipple tunnel cap element 20c is substantially horizontal, with the opening facing the breast. The internal nipple tunnel cap element 30c is approximately of a tubular structure, with a cavity forming the second nipple tunnel 311c. When the internal nipple tunnel cap element 30c extends into the external nipple tunnel cap element 20c, the second nipple tunnel 311c communicates with the first nipple tunnel 21c through an opening in its one end.

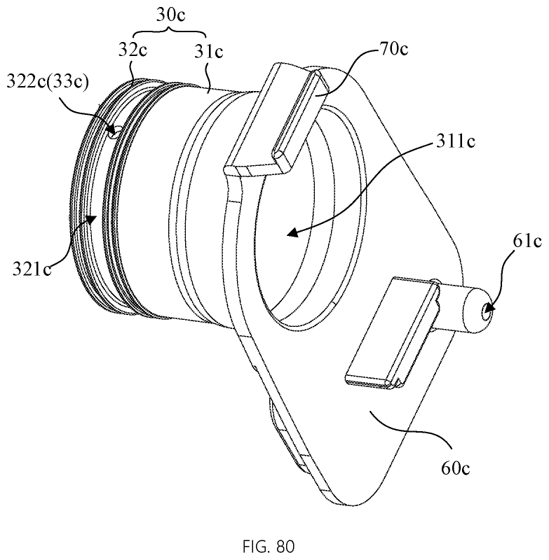

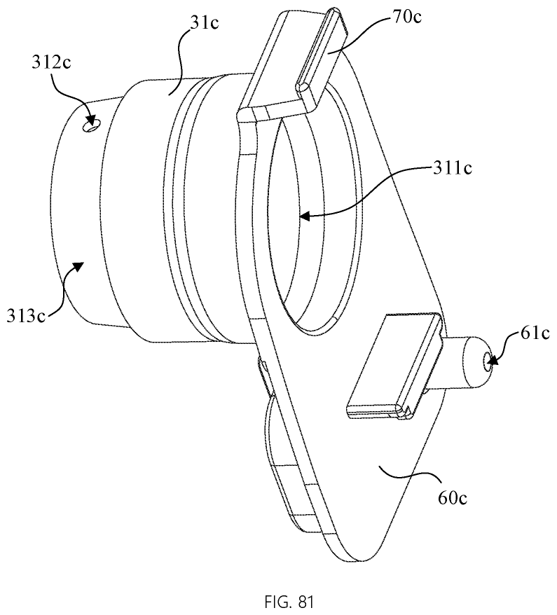

Referring to FIG. 77, FIG. 80, and FIG. 81, the internal nipple tunnel cap element 30c includes a milk guide body 31c and a soft rubber ring 32c. The milk guide body 31c extends into the first nipple tunnel 21c, and is provided with the second nipple tunnel 311c and a first perforation 312c communicating with the second nipple tunnel 311c. The soft rubber ring 32c is sleeved on a periphery of the milk guide body 31c and located between the milk guide body 31c and the external nipple tunnel cap element 20c. The soft rubber ring 32c is provided with a first annular groove 321c and a second perforation 322c communicating with the first annular groove 321c. The external nipple tunnel cap element 20c is configured to seal the first annular groove 321c to form the air guide channel 40c. The first perforation 312c and the second perforation 322c correspondingly form the second air guide hole 33c. The milk guide body 31c is approximately of a tubular structure. The soft rubber ring 32c is sleeved on the periphery of the milk guide body 31c, and clamped between the milk guide body 31c and the external nipple tunnel cap element 20c, so the enclosed air guide channel 40c has desirable sealing performance to ensure no leakage of the air guide channel 40c.

Referring to FIG. 80 and FIG. 81, an outer side wall of the milk guide body 31c is provided with a second annular groove 313c. The soft rubber ring 32c is nested in the second annular groove 313c. This configuration enhances stability of connection between the soft rubber ring 32c and the milk guide body 31c. Moreover, the soft rubber ring 32c does not entirely protrude from the milk guide body 31c, such that a gap between the milk guide body 31c and the external nipple tunnel cap element 20c can be reduced, thereby enhancing stability of assembly between the milk guide body 31c and the external nipple tunnel cap element 20c.

As an implementation, the second annular groove 313c is formed in an end of the milk guide body 31c extending into the first nipple tunnel 21c, i.e., the second annular groove 313c is formed in an end of the milk guide body 31c close to the milk flow hole 22c. By forming the air guide channel 40c in the end of the milk guide body 31c close to the milk flow hole 22c, the distance from the air guide channel 40c to the milk flow hole 22c is small, facilitating flowing of the milk to the milk flow hole 22c. In addition, compared with a solution in which the second annular groove 313c is formed in a middle of the milk guide body 31c, the soft rubber ring 32c can be quickly assembled.

As an implementation, the internal nipple tunnel cap element 30c is detachably connected to the external nipple tunnel cap element 20c. When the breast pump is used, the internal nipple tunnel cap element 30c is assembled in the external nipple tunnel cap element 20c, preventing the milk from flowing to the negative pressure chamber 11c. Upon completion of milk expression, when the breast pump is not used, the internal nipple tunnel cap element 30c can be detached from the external nipple tunnel cap element 20c, facilitating cleaning of the internal nipple tunnel cap element 30c and the external nipple tunnel cap element 20c. Certainly, in other embodiments, the internal nipple tunnel cap element 30c may also be fixedly connected to the external nipple tunnel cap element 20c.

Referring to FIG. 76, the breast pump assembly further includes a flexible diaphragm 50c. The flexible diaphragm 50c is circumferentially connected to the diaphragm bracket 10c and extends into the negative pressure chamber 11c. In specific application, the flexible diaphragm 50c may deform, changing an air pressure in the second nipple tunnel 311c to promote milk secretion.

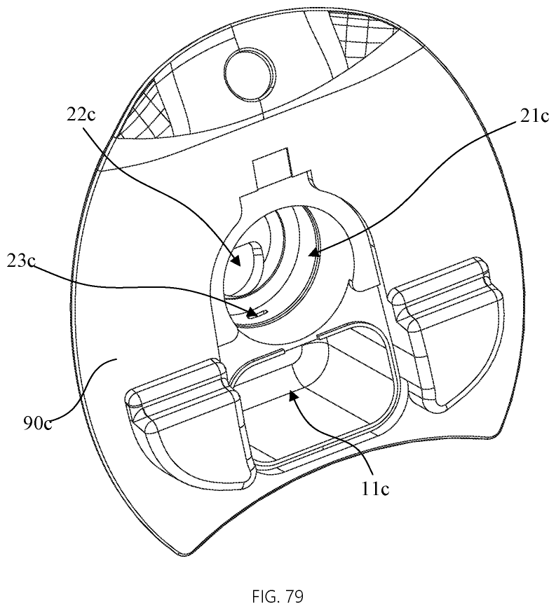

Referring to FIG. 79 and FIG. 80, the breast pump assembly further includes a negative pressure chamber cover 60c. The negative pressure chamber cover 60c is configured to cover an opening of the negative pressure chamber 11c and seal the negative pressure chamber 11c. In specific applications, a negative pressure suction port 61c is formed in the negative pressure chamber cover 60c, such that the negative pressure chamber 11c communicates with a pump body of the breast pump.

As an implementation, the negative pressure chamber cover 60c is connected to the milk guide body 31c, and integrally formed with the milk guide body 31c. This configuration reduces the number of components, prevents the components from losing, and improves the assembly efficiency of the breast pump.

Referring to FIG. 80, the breast pump assembly further includes a handle 70c. The handle 70c is connected to the internal nipple tunnel cap element 30c. The handle 70c offers a force application point for the user, facilitating the disassembly and assembly of the inner nipple tunnel cap element by the user.

As an implementation, the handle 70c is connected to the milk guide body 31c, and integrally formed with the milk guide body 31c. This configuration reduces the number of components, prevents the components from losing, and improves the assembly efficiency of the breast pump. On the other hand, this configuration reduces fabrication steps, and improves the fabrication efficiency.

Referring to FIG. 76 and FIG. 77, the breast pump assembly further includes a container body 80c, a container lid 90c, a main pump module housing 100c, and a breast shield 110c. The container lid 90c is configured to cover and seal the container body 80c, and encloses the milk storage chamber 81c with the container body 80c. The main pump module housing 100c is disposed between the container lid 90c and the breast shield 110c. The breast shield 110c is configured to be placed against the breast. The fixing member is the container lid 90c or the main pump module housing 100c or the breast shield 110c. Both the external nipple tunnel cap element 20c and the diaphragm bracket 10c extend toward the milk storage chamber 81c from the fixing member. In this embodiment, the fixing member is the container lid 90c. Both the external nipple tunnel cap element 20c and the diaphragm bracket 10c are connected to a side of the container lid 90c facing the milk storage chamber 81c, and extend toward the milk storage chamber 81c.

Further, the embodiment of the present disclosure further provides a breast pump, including the above breast pump assembly. With the above breast pump assembly, the milk cannot enter the negative pressure chamber 11c to contaminate the negative pressure chamber 11c or even affect pumping performance of the breast pump, greatly improving the quality of the breast pump.

As an implementation, the breast pump further includes a pump body and a solenoid valve. Both the pump body and the solenoid valve are disposed in the main pump module housing 100c. When the pump body is in operation, the solenoid valve is idle, and the pump body draws air out to generate a negative pressure. When the solenoid valve is in operation, the pump body is idle, and the solenoid valve is opened, such that the air can be charged to the negative pressure chamber 11.

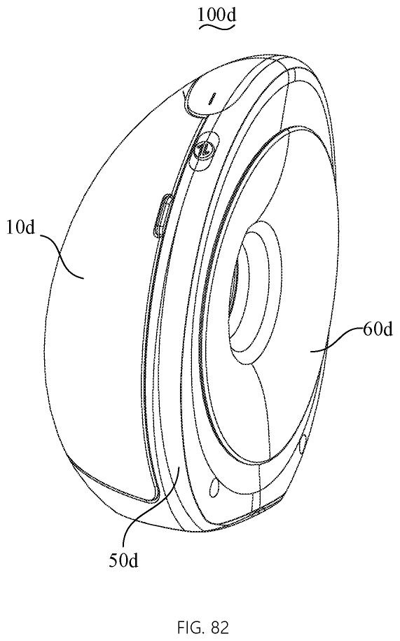

As shown in FIGS. 82-84, an embodiment of the present disclosure provides a breast pump assembly, applied to a breast pump 100d, and including: a milk storage container 10d, a nipple tunnel cap element 20d, and a sealing member 30d. The milk storage container 10d includes a container body 11d and a container lid 12d. The container lid 12d and the container body 11d enclose a milk storage chamber 13d. The nipple tunnel cap element 20d is connected to a side of the container lid 12d facing the milk storage chamber 13d or connected to a fixing member of the breast pump 100d and extends into the milk storage chamber 13d through the container lid 12d. The nipple tunnel cap element 20d has a nipple tunnel 21d. The container lid 12d is provided with a milk pouring port 121d communicating with the milk storage chamber 13d. The sealing member 30d is configured to seal the milk pouring port 121d.

When the breast pump 100d is used for milk expression, milk is expressed to the nipple tunnel 21d, and then flows to the milk storage chamber 13d for temporary storage. When the breast pump 100d has expressed the milk from one or both breasts, the milk in the milk storage chamber 13d is poured out through the milk pouring port 121d.

According to the above technical solution, the nipple tunnel cap element 20d is provided, and the nipple tunnel cap element 20d is provided with the nipple tunnel 21d, such that the milk can be guided. Since the nipple tunnel cap element 20d is connected to the side of the container lid 12d facing the milk storage chamber 13d or connected to the fixing member of the breast pump 100d and extends into the milk storage chamber 13d through the container lid 12d, when the breast pump 100d is used for the milk expression, the container lid 12d faces one side of the breast, and expressed milk can flow to the milk storage chamber 13d through the nipple tunnel 21d, thereby facilitating observation of the user on a milk flowing condition in the nipple tunnel 21d through the container body 11d and the nipple tunnel cap element 20d during the milk expression. By providing the milk pouring port 121d and the sealing member 30d, the milk temporarily stored in the milk storage chamber 13d can be poured out through the milk pouring port 121d, and the milk pouring port 121d is sealed by the sealing member 30d. When the milk is to be poured out from the milk storage chamber 13d, the sealing member 30d is detached to pour out the milk. When the breast pump 100d is used for the milk expression, the milk pouring port 121d is sealed by the sealing member 30d, so as to prevent the milk from flowing out of the milk pouring port 121d. By forming the milk pouring port 121d in the container lid 12d, since the container lid 12d faces the breast and is not located on a path of the user for viewing the nipple tunnel 21d during the milk expression of the breast pump 100d, the milk pouring port 121d is also not located on the path of the user for viewing the nipple tunnel 21d, either. Thus, the sealing member 30d for sealing the milk pouring port 121d does not shield a sight of the user, such that the user can quickly and effectively know the problem of the breast pump 100d according to the milk flowing condition in use.

Therefore, by forming the milk pouring port 121d in the container lid 12d, the breast pump assembly provided by this embodiment prevents the sealing member 30d from shielding the sight of the user for viewing the nipple tunnel 21d, such that the user can quickly and effectively know the problem of the breast pump 100d in use, improving the user experience.

As an implementation, when the breast pump 100d is placed against the breast, the milk pouring port 121d is located above the nipple tunnel cap element 20d. When the milk flows from the nipple tunnel 21d to the milk storage chamber 13d, the milk flows to a lower side of the nipple tunnel 21d under the action of gravity. When the milk pouring port 121d is located below the nipple tunnel cap element 20d (i.e., below the nipple tunnel 21d), the milk quickly rises to a level where the milk pouring port 121d is located. If the sealing member 30d is not tight, the milk may easily leak out through the milk pouring port 121d, which not only affects the user experience but also results in a milk waste. In this embodiment, forming the milk pouring port 121d above the nipple tunnel cap element 20d can effectively prevent the above condition.

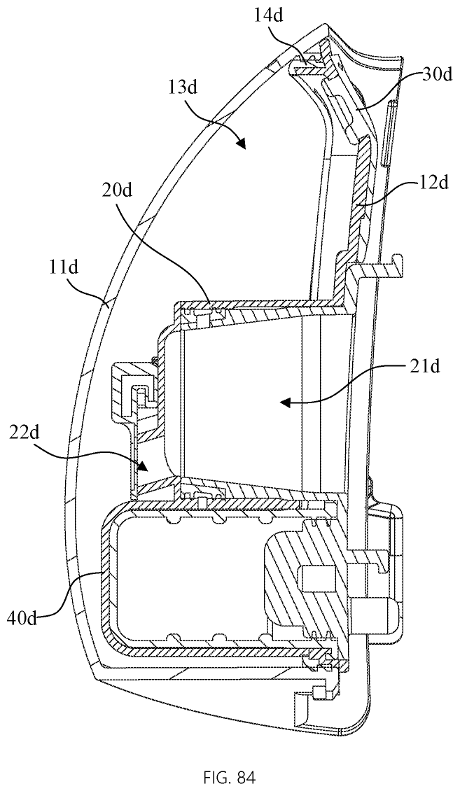

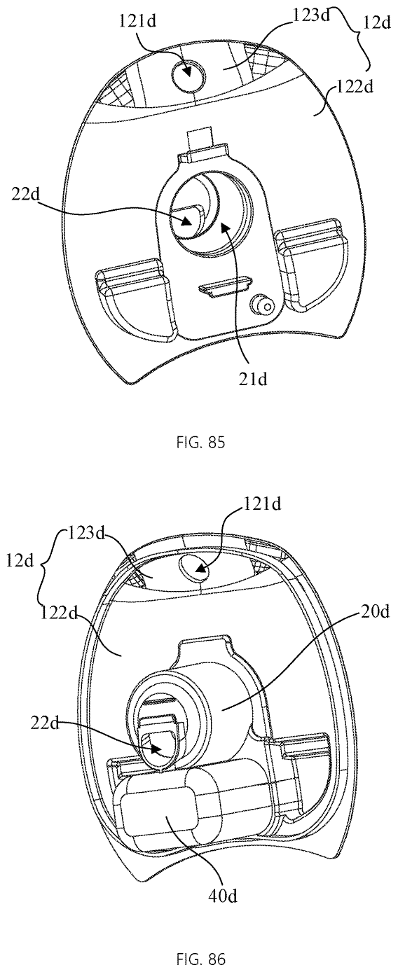

Referring to FIGS. 84-86, the container lid 12d includes a first plate 122d and a second plate 123d. The second plate 123d gradually extends toward the milk storage chamber 13d from the first plate 122d, and forms an included angle of greater than 90° and less than 180° with the first plate 122d. The nipple tunnel cap element 20d is connected to the first plate 122d. The milk pouring port 121d is formed in the second plate 123d. When the breast pump 100d is placed against the breast, the second plate 123d is located on the first plate 122d. Through this configuration, when the breast pump 100d is placed against the breast, the milk pouring port 121d is located above the nipple tunnel 21d, and the milk pouring port 121d inclines upward, which can effectively prevent milk leakage when the sealing member 30d is not tight. In addition, in this configuration, the milk can be accommodated below the milk pouring port 121d more concentratedly, and the milk can be poured out more easily through the milk pouring port 121d.

As an implementation, the sealing member 30d includes one end configured to detachably seal the milk pouring port 121d, and another end connected to the container lid 12d. Specifically, the another end of the sealing member 30d is connected to a side of the container lid 12d away from the milk storage chamber 13d. In this configuration, the sealing member 30d can be used according to an actual condition to seal the milk pouring port 121d or open the milk pouring port 121d. In addition, when the sealing member 30d is detached to open the milk pouring port 121d, the another end of the sealing member 30d is connected to the container lid 12d, preventing the sealing member 30d from losing. Certainly, in other embodiments, the sealing member 30d is configured to detachably seal the milk pouring port 121d. When the sealing member 30d is detached from the milk pouring port 121d, the sealing member 30d may also be separated from the container lid 12d.

As an implementation, the sealing member 30d and the container lid 12d are integrally injection-molded. This can not only prevent the sealing member 30d from losing, but also improves the production efficiency of the breast pump assembly. Specifically, the sealing member 30d is made of soft rubber, and the container lid 12d is made of hard rubber. The sealing member 30d and the container lid 12d are integrally injection-molded with the soft rubber and the hard rubber. It is to be understood that in other embodiments, the sealing member 30d and the container lid 12d may also be fabricated independently and then assembled.

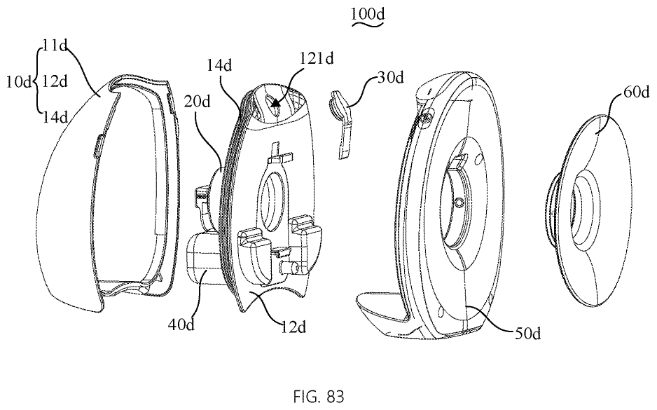

Referring to FIG. 83 and FIG. 84, the milk storage container 10d further includes a sealing ring 14d. The sealing ring 14d is disposed between the container body 11d and the container lid 12d. With the sealing ring 14d, the sealing performance of the milk storage chamber 13d is enhanced, preventing leakage of the milk from a gap between the container body 11d and the container lid 12d. Preferably, the sealing ring 14d and the container lid 12d are integrally injection-molded, which reduces the number of components, lowers the probability of losing the components, and improves the production efficiency and assembly efficiency of the breast pump assembly.

Referring to FIGS. 84-86, an end of the nipple tunnel cap element 20d facing the milk storage chamber 13d is provided with a milk flow port 22d. The milk flow port 22d is configured to communicate the nipple tunnel 21d with the milk storage chamber 13d. This facilitates observation of the user on a milk flowing condition in the whole nipple tunnel 21d.

As an implementation, when the breast pump 100d is placed against the breast, at least an upper half of the container body 11d and at least an upper half of the nipple tunnel cap element 20d are made of an optically transparent material. In this way, when the breast pump 100d is used for milk expression, the user can view the condition in the nipple tunnel 21d. Preferably, both the container body 11d and the nipple tunnel cap element 20d are made of a transparent material.

Referring to FIG. 83, FIG. 84, and FIG. 86, the breast pump assembly further includes a diaphragm bracket 40d. The diaphragm bracket 40d is connected to a side of the container lid 12d facing the milk storage chamber 13d or connected to a fixing member of the breast pump 100d and extends into the milk storage chamber 13d through the container lid 12d. When the breast pump 100d is placed against the breast, the diaphragm bracket 40d is located under the nipple tunnel cap element 20d. This configuration can effectively prevent the diaphragm bracket 40d from shielding the user to view the nipple tunnel 21d. The diaphragm bracket 40d has a negative pressure chamber communicating with the nipple tunnel 21d. When a negative pressure is generated in the negative pressure chamber, the negative pressure is conducted to the nipple tunnel 21d through a pump body of the breast pump 100d to express the milk.

As an implementation, the diaphragm bracket 40d and the nipple tunnel cap element 20d are arranged side by side. This effectively utilizes a space in the milk storage container 10d, such that the milk storage chamber 13d has a sufficiently large space as much as possible for containing the milk.

Referring to FIG. 82 and FIG. 83, the breast pump assembly further includes a main pump module housing 50d and a breast shield 60d. The main pump module housing 50d is disposed between the container lid 12d and the breast shield 60d. The breast shield 60d is configured to be placed against the breast. When the nipple tunnel cap element 20d and the diaphragm bracket 40d are connected to the fixing member of the breast pump 100d, the fixing member may be the main pump module housing 50d or the breast shield 60d, i.e., the nipple tunnel cap element 20d and the diaphragm bracket 40d may be connected to the main pump module housing 50d or the breast shield 60d.

Further, referring to FIGS. 82-84, the embodiment of the present disclosure further provides a breast pump 100d, including the above breast pump assembly. With the above breast pump assembly, when the breast pump 100d is used for the milk expression, the sealing member 30d does not shield the sight of the user for viewing the nipple tunnel 21d, such that the user can quickly and effectively know the problem of the breast pump 100d in use.

As an implementation, the breast pump 100d further includes a pump body and a solenoid valve. Both the pump body and the solenoid valve are disposed in the main pump module housing 50d. When the pump body is in operation, the solenoid valve is idle, and the pump body draws air out to generate a negative pressure. When the solenoid valve is in operation, the pump body is idle, and the solenoid valve is opened, such that the air can be charged to the negative pressure chamber.

The breast pump assembly and the breast pump 100d provided in this embodiment differ from those in the above embodiment in the arrangement manner of the sealing member 30d. Specifically, in an embodiment, the sealing member 30d and the container lid 12d are integrally injection-molded. However, in this embodiment, the sealing member 30d, the sealing ring 14d, and the container lid 12d are integrally injection-molded.

In this embodiment, the sealing member 30d includes one end configured to detachably seal the milk pouring port 121d, and another end connected to the sealing ring 14d through the container lid 12d. The sealing member 30d, the sealing ring 14d, and the container lid 12d are integrally injection-molded. This configuration reduces the number of components, lowers the probability of losing the components, and improves the production efficiency and assembly efficiency of the breast pump assembly.

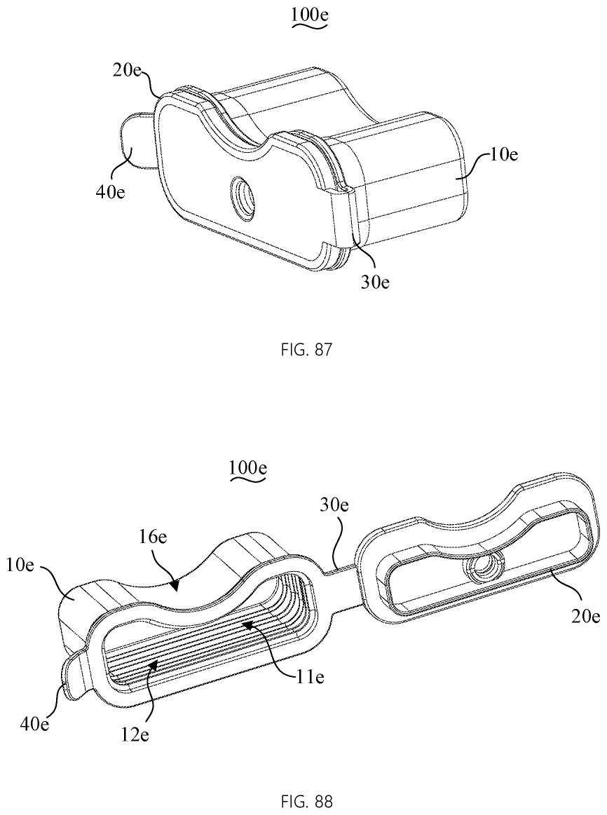

As shown in FIGS. 87-89, an embodiment of the present disclosure provides a diaphragm assembly 100e, applied to a breast pump, and including a flexible diaphragm 10e, a diaphragm cover 20e, and a connector 30e. The flexible diaphragm 10e has a cavity 11e and an opening 12e communicating with the cavity 11e. The diaphragm cover 20e is configured to detachably cover the opening 12e, and provided with a first through hole 21e. The connector 30e includes one end fixedly connected to the flexible diaphragm 10e, and another end fixedly connected to the diaphragm cover 20e.

According to the above technical solution, since the diaphragm cover 20e is configured to detachably cover the opening 12e, the diaphragm cover 20e can cover the opening 12e to seal the cavity 11e, and can also be detached from the opening 12e of the flexible diaphragm 10e, thereby facilitating cleaning of the flexible diaphragm 10e. Since the connector 30e is provided, and the connector 30e includes one end fixedly connected to the flexible diaphragm 10e, and another end fixedly connected to the diaphragm cover 20e, the diaphragm cover 20e detached from the opening 12e of the flexible diaphragm 10e is still connected to the flexible diaphragm 10e through the connector 30e. Therefore, the diaphragm cover 20e and the flexible diaphragm 10e are not separated into two independent components, preventing the diaphragm cover 20e (with a relatively small size) from losing, and reducing the number of independent components of the breast pump. Therefore, the diaphragm assembly 100e provided by this embodiment reduces the number of components, accelerates the disassembly and assembly speeds, and lowers the probability of losing components.

As an implementation, the connector 30e is configured to allow the diaphragm cover 20e to pivotally cover the opening 12e. It is to be understood that in other embodiments, the diaphragm cover 20e may also be configured to translationally cover the opening 12e.

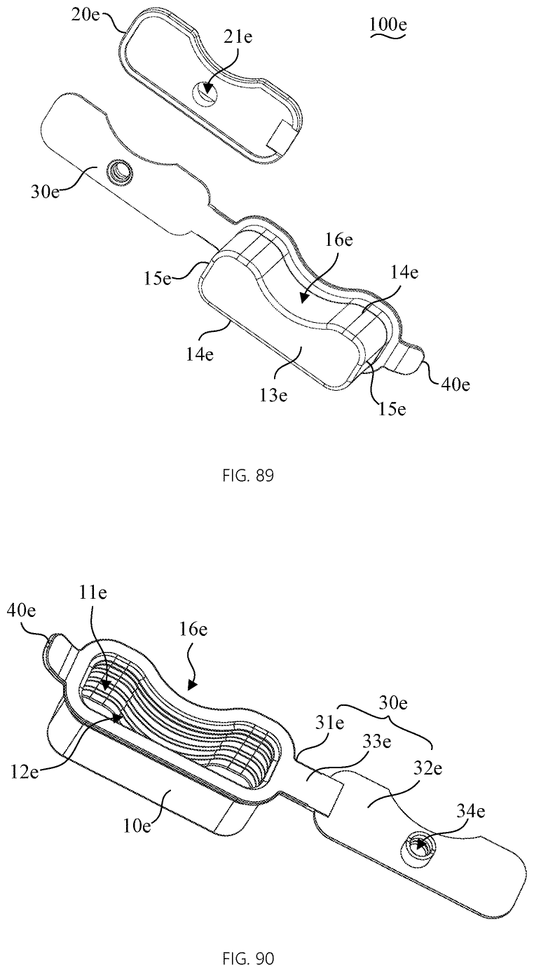

Referring to FIGS. 89-91, the connector 30e includes a connecting portion 31e, an embedding portion 32e, and a bendable portion 33e. The bendable portion 33e is connected between the connecting portion 31e and the embedding portion 32e and is bendable. The connecting portion 31e is fixedly connected to the flexible diaphragm 10e. A side of the diaphragm cover 20e away from the cavity 11e is provided with a mounting groove 22e. The embedding portion 32e is nested in the mounting groove 22e. The connector 30e is structurally simple, with the embedding portion 32e nested in the mounting groove 22e. This improves the firmness of connection between the connector 30e and the diaphragm cover 20e. On the other hand, compared with a solution in which the embedding portion 32e protrudes, the thickness of the diaphragm assembly 100e can be reduced, facilitating assembly of the diaphragm assembly 100e with other components of the breast pump.

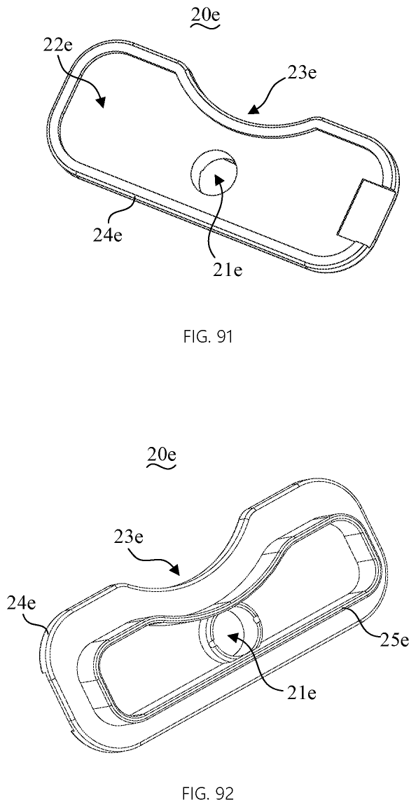

Referring to FIG. 90 and FIG. 91, the first through hole 21e is formed in a bottom wall of the mounting groove 22e. The embedding portion 32e is provided with a second through hole 34e. The first through hole 21e corresponds to the second through hole 34e to form a negative pressure suction port. The negative pressure suction port is configured to communicate the cavity 11e with a suction pump of the breast pump. In this embodiment, the mounting groove 22e has a large area, and nearly occupies the whole side of the diaphragm cover 20e away from the cavity 11e. Certainly, in specific applications, as an alternative implementation, the first through hole 21e may also be formed in the diaphragm cover 20e outside a region where the mounting groove 22e is located. When this technical solution is used, the first through hole 21e forms the negative pressure suction port, and the mounting groove 22e can be made smaller, such as it occupies ⅓ or ¼ of the side of the diaphragm cover 20e away from the cavity 11e.

As an implementation, both the connector 30e and the flexible diaphragm 10e are made of soft rubber. The diaphragm cover 20e is made of hard rubber. The diaphragm cover 20e, the connector 30e, and the flexible diaphragm 10e are integrally formed. This configuration greatly improves the fabrication efficiency of the diaphragm assembly 100e. It is to be understood that in other embodiments, the diaphragm cover 20e and the connector 30e are integrally formed, or the flexible diaphragm 10e and the connector 30e are integrally formed, or the diaphragm cover 20e, the connector 30e, and the flexible diaphragm 10e are fabricated independently and then assembled.

Referring to FIG. 88, FIG. 89 and FIG. 94, the flexible diaphragm 10e includes a bottom wall 13e, two long side walls 14e, and two short side walls 15e. The two short side walls 15e are spaced-apart and opposite to each other, and are connected between the two long side walls 14e. The bottom wall 13e, the two long side walls 14e, and the two short side walls 15e enclose the cavity 11e. Ends of the two long side walls 14e and the two short side walls 15e away from the bottom wall 13e enclose the opening 12e. The bottom wall 13e is opposite to the opening 12e. In this configuration, the flexible diaphragm 10e is cuboid or substantially cuboid. In specific applications, the flexible diaphragm 10e is disposed in a negative pressure chamber 2031e of the breast pump. The negative pressure chamber 2031e is also cuboid or substantially cuboid and is horizontally disposed on the breast pump, thereby reducing the height of the breast pump while ensuring the spatial size of the negative pressure chamber 2031e.

As an implementation, the connector 30e is fixedly connected to an end of one of the two short side walls 15e close to the opening 12e. Certainly, in other embodiments, the connector 30e may also be fixedly connected to an end of one of the two long side walls 14e close to the opening 12e, or an end of a junction between adjacent short side wall 15e and long side wall 14e close to the opening 12e.

Referring to FIG. 88, FIG. 89, and FIG. 94, the diaphragm assembly 100e further includes a lug 40e. The lug 40e is connected to an end of the other short side wall 15e close to the opening 12e. That is, the lug 40e is opposite to the connector 30e. By providing the lug 40e, when the flexible diaphragm 10e is to be taken out from the negative pressure chamber 2031e, the lug 40e offers a force application point to take out the flexible diaphragm 10e conveniently. Certainly, in other embodiments, the lug 40e may also be fixedly connected to an end of one long side wall 14e close to the opening 12e, or an end of a junction between adjacent short side wall 15e and long side wall 14e close to the opening 12e.

Referring to FIG. 89 and FIG. 94, one long side wall 14e is provided with an avoidance groove 16e. The avoidance groove 16e is configured to avoid a nipple tunnel 2041e of the breast pump. Correspondingly, the negative pressure chamber 2031e is provided with an avoidance space that is configured to avoid the nipple tunnel 2041e. The nipple tunnel 2041e and the negative pressure chamber 2031e are arranged side by side along an up-down direction. By providing the avoidance groove 16e for avoiding the nipple tunnel 2041e, an occupied space by the nipple tunnel 2041e and the negative pressure chamber 2031e in a height direction can be reduced as much as possible, shortening a height of the breast pump.

As an implementation, an outer contour of the diaphragm cover 20e matches with a shape of the opening 12e, i.e., the diaphragm cover 20e is of a rectangle, with a long side provided with an avoidance recess 23e corresponding to the avoidance groove 16e. The avoidance recess 23e is configured to avoid the nipple tunnel 2041e.

Referring to FIG. 88 and FIG. 92, the diaphragm cover 20e includes a cover body 24e and a limiting protrusion 25e. The limiting protrusion 25e is connected to one side of the cover body 24e. The cover body 24e is configured to cover the opening 12e. The limiting protrusion 25e extends into the cavity 11e and abuts against the flexible diaphragm 10e, so as to limit the cover body 24e. The connector 30e is fixedly connected to a side of the cover body 24e away from the limiting protrusion 25e. This improves the firmness of the cover body 24e for covering the opening 12e, and prevents movement of the cover body 24e. Moreover, by fixedly connecting the connector 30e to the side of the cover body 24e away from the limiting protrusion 25e, the connector 30e cannot disturb extension of the limiting protrusion 25e into the cavity 11e.

As an implementation, the limiting protrusion 25e is annular. In this way, the limiting protrusion 25e circumferentially abuts against the flexible diaphragm 10e to limit the cover body 24e desirably. It is to be understood that in other embodiments, the limiting protrusion 25e includes a plurality of tabs that are arranged annularly at intervals.

Further, the embodiment of the present disclosure further provides a breast pump, including the above diaphragm assembly 100e. With the above diaphragm assembly 100e, this embodiment reduces the number of components, accelerates the disassembly and assembly speeds, and lowers the probability of losing components.

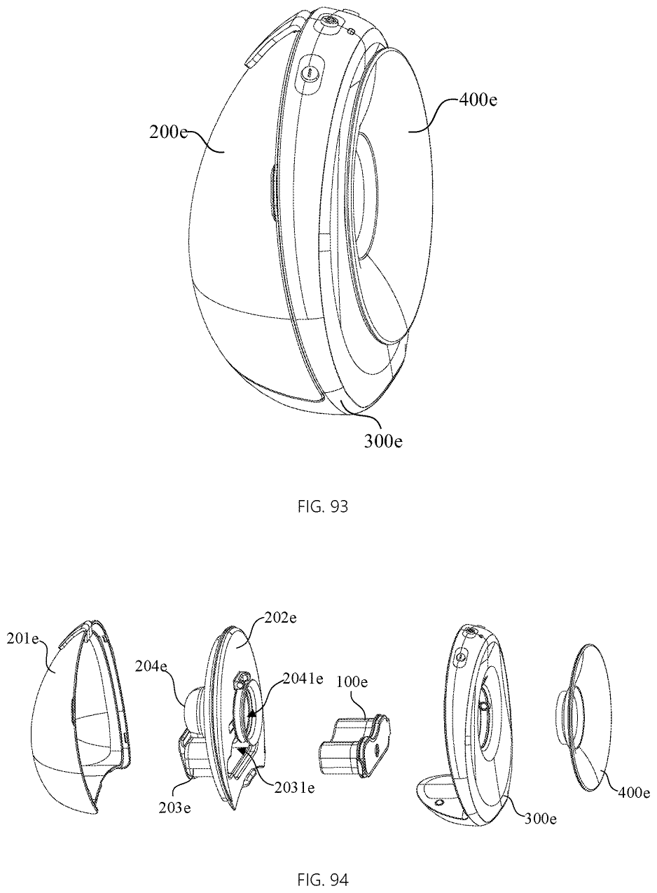

Referring to FIG. 89, FIG. 93, and FIG. 94, the breast pump further includes a breast pump assembly 200e, a main pump module 300e, and a breast shield 400e. The breast pump assembly 200e is provided with the negative pressure chamber 2031e and the nipple tunnel 2041e communicating with the negative pressure chamber 2031e. The flexible diaphragm 10e is disposed in the negative pressure chamber 2031e. The nipple tunnel 2041e is configured to guide milk. The main pump module 300e is disposed between the breast pump assembly 200e and the breast shield 400e. The breast shield 400e is configured to be placed against a breast. The first through hole 21e is configured to communicate the cavity 11e with a suction pump of the main pump module 300e. The breast pump has a simple structure, is miniaturized, and is easy and quick to assemble.

Referring to FIG. 93 and FIG. 94, the breast pump assembly 200e includes the container body 201e, the container lid 202e, the negative pressure part 203e, and the nipple tunnel cap element 204e. The container lid 202e is configured to cover and seal the container body 201e, and enclose the milk storage chamber with the container body 201e. The main pump module 300e is disposed between the container lid 202e and the breast shield 400e. Both the negative pressure part 203e and the nipple tunnel cap element 204e are connected to the side of the cover plate 202e facing the milk storage chamber. The negative pressure part 203e is provided with the negative pressure chamber 2031e. The nipple tunnel cap element 204e is provided with the nipple tunnel 2041e.

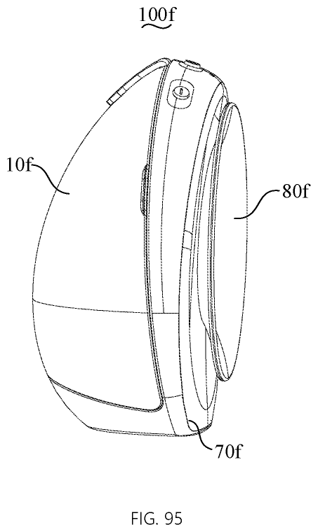

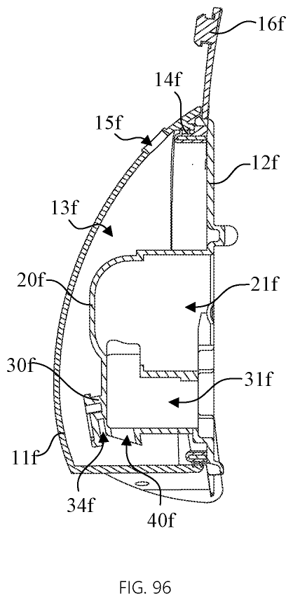

As shown in FIG. 95 and FIG. 96, an embodiment of the present disclosure provides a breast pump assembly, applied to a breast pump 100f, and including a milk storage container 10f. The milk storage container 10f includes a container body 11f and a container lid 12f. The container lid 12f and the container body 11f enclose a milk storage chamber 13f. The milk storage chamber 13f is configured to temporarily store milk expressed from a breast.

Referring to FIG. 95 and FIG. 96, the breast pump assembly further includes a nipple tunnel cap element 20f and a negative pressure part 30f. Both the nipple tunnel cap element 20f and the negative pressure part 30f are connected to a side of the container lid 12f facing the milk storage chamber 13f or penetrate through the container lid 12f and extend into the milk storage chamber 13f. The nipple tunnel cap element 20f has a nipple tunnel 21f. The negative pressure part 30f has a negative pressure chamber 31f communicating with the nipple tunnel 21f. The nipple tunnel cap element 20f or the negative pressure part 30f is further provided with a milk flow port 40f communicating with the milk storage chamber 13f. The milk flow port 40f is configured to allow the milk to flow to the milk storage chamber 13f from the nipple tunnel 21f or the negative pressure chamber 31f. In specific applications, a nipple is extended into or aligned at the nipple tunnel 21f. A suction pump in the breast pump 100f is configured to generate a negative pressure suction force in the negative pressure chamber 31f. Through communication between the negative pressure chamber 31f and the nipple tunnel 21f, a negative pressure suction force is generated in the nipple tunnel 21f, thereby expressing the milk. Since the nipple tunnel cap element 20f and the negative pressure part 30f are connected to the side of the container lid 12f facing the milk storage chamber 13f or penetrate through the container lid 12f and extend into the milk storage chamber 13f, by forming the milk flow port 40f in the nipple tunnel cap element 20f or the negative pressure part 30f, the milk flow port 40f communicates with the milk storage chamber 13f.

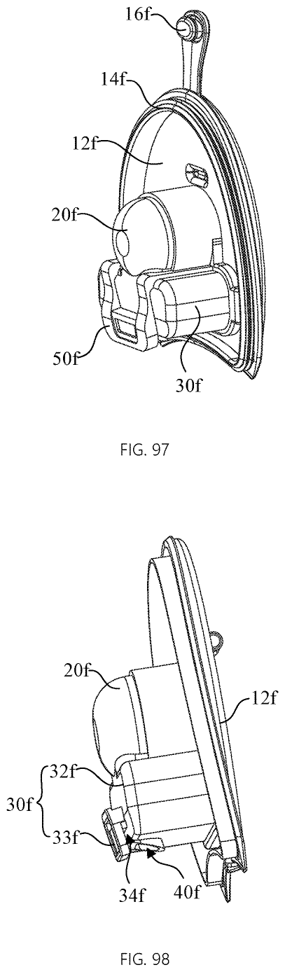

Referring to FIG. 96 and FIG. 97, the breast pump further includes a one-way valve 50f. The one-way valve 50f is connected to at least one of the container lid 12f, the nipple tunnel cap element 20f, and the negative pressure part 30f, extends to the milk flow port, and is configured to allow the milk in the nipple tunnel 21f to unidirectionally flow to the milk storage chamber 13f. In specific applications, the one-way valve 50f is located in the milk storage chamber 13f and configured to shield the milk flow port 40f. When the suction pump of the breast pump 100f generates the negative pressure suction force in the negative pressure chamber 31f, the one-way valve 50f is closely attached to the milk flow port 40f under the action of the negative pressure suction force, such that the milk flow port 40f is sealed, and the milk cannot pass through the milk flow port 40f. When the suction pump of the breast pump 100f is idle, and a pressure in the negative pressure chamber 31f is normal, the one-way valve 50f shields the milk flow port 40f, and the milk flows from the negative pressure chamber 31f or the nipple tunnel 21f to the milk storage chamber 13f under the action of gravity, pushes the one-way valve 50f open, and flows to the milk storage chamber 13f through the milk flow port 40f.

According to the above technical solution, by connecting the one-way valve 50f to at least one of the container lid 12f, the nipple tunnel cap element 20f, and the negative pressure part 30f, when the breast pump assembly is disassembled (for example, various components of the breast pump are disassembled for cleaning), the one-way valve 50f is still connected to at least one of the container lid 12f, the nipple tunnel cap element 20f, and the negative pressure part 30f. This greatly reduces the probability of losing the one-way valve 50f, reduces the number of independent components of the breast pump assembly, and can accelerate the disassembly and assembly speeds. Therefore, the breast pump assembly provided by this embodiment of the present disclosure reduces the number of components, accelerates the disassembly and assembly speeds, and lowers the probability of losing components.

As an implementation, the one-way valve 50f is made of a soft material. The container lid 12f, the nipple tunnel cap element 20f, and the negative pressure part 30f are made of a hard material. The one-way valve 50f is fixedly connected to at least one of the container lid 12f, the nipple tunnel cap element 20f, and the negative pressure part 30f via integrated injection molding. In specific applications, the fixed connection using the integrated injection molding of soft rubber and hard rubber effectively ensures the connection stability.

As an implementation, the nipple tunnel cap element 20f and the negative pressure part 30f are arranged side by side and share a wall. When the breast pump 100f is placed against the breast, the negative pressure part 30f is located under the nipple tunnel cap element 20f. This configuration reduces the occupied space of the nipple tunnel cap element 20f and the negative pressure part 30f, thereby ensuring a spatial size of the milk storage chamber 13f. On the other hand, this configuration facilitates observation of the user on a milk flowing condition in the nipple channel, thereby selecting an operating mode of the breast pump 100f. The expression “share a wall” means that the nipple tunnel cap element 20f and the negative pressure part 30f share a side wall. The side wall includes one side facing the nipple tunnel 21f, and another side facing the negative pressure chamber 31f. Certainly, in specific applications, as an alternative implementation, the nipple tunnel cap element 20f and the negative pressure part 30f may also not share the wall. For example, a connecting tube is disposed therebetween, so as to communicate the nipple tunnel 21 with the negative pressure chamber 31. As another alternative implementation, when the breast pump 100f is placed against the breast, the negative pressure part 30f may also be located on or aside the nipple tunnel cap element 20f. Preferably, the nipple tunnel cap element 20f and the negative pressure part 30f are integrally injection-molded, effectively reducing the number of components of the breast pump assembly, and accelerating the disassembly and assembly speeds of the breast pump assembly.

Referring to FIG. 95, FIG. 96, and FIG. 98, the milk flow port 40f is formed in a side of the negative pressure part 30f away from the nipple tunnel cap element 20f. When the breast pump 100f is placed against the breast, the negative pressure part 30f is located under the nipple tunnel 21f, and the milk flow port 40f is formed in the side of the negative pressure part 30f away from the nipple tunnel cap element 20f, i.e., the milk flow port 40f is formed in an underside of the negative pressure part 30f. Since liquid flows downward under the action of gravity, by forming the milk flow port 40f in the underside of the negative pressure part 30f, the milk flows out of the negative pressure chamber 31f as much as possible, thereby preventing the milk from remaining in the negative pressure chamber 31f and avoiding waste. Certainly, in specific applications, as an alternative implementation, the milk flow port 40f may also be formed in other positions of the negative pressure part 30f, and may, for example, be formed in an end of the negative pressure part 30f away from the container lid 12f. As another alternative implementation, the milk flow port 40f may also be formed in the nipple tunnel cap element 20f.

As an implementation, the one-way valve 50f is fixedly connected to the nipple tunnel cap element 20f and the negative pressure part 30f. This configuration reduces the number of components of the breast pump assembly. Preferably, the one-way valve 50f, the negative pressure part 30f, and the nipple tunnel cap element 20f are integrally injection-molded. It is to be understood that in other embodiments, the one-way valve 50f may also be fixedly connected to other components, and may, for example, be fixedly connected to the nipple tunnel cap element 20f or the negative pressure part 30f or the container lid 12f.

Referring to FIGS. 95-99, the one-way valve 50f includes one end fixedly connected to a junction between the negative pressure part 30f and the nipple tunnel cap element 20f, and another end extending to the milk flow port 40f. This not only achieves the connection of the one-way valve 50f, but also enables the one-way valve 50f to control opening and closing of the milk flow port 40f. In specific applications, in order to adapt to the miniaturized design of the breast pump 100f, the milk storage container 10f has a small size, which results in a limited space under the nipple tunnel cap element 20f. To ensure the spatial size of the negative pressure chamber 31f, the negative pressure part 30f has a rectangular cross-section, with a long side connected to the nipple tunnel cap element 20f. Moreover, to match the nipple tunnel cap element 20f with the nipple, the nipple tunnel cap element 20f generally has a circular cross-section, a groove 60f is formed in the junction between the negative pressure part 30f and the nipple tunnel cap element 20f, and one end of the one-way valve 50f is fixedly connected to the junction between the negative pressure part 30f and the nipple tunnel cap element 20f, i.e., one end of the one-way valve 50f is fixedly disposed in the groove 60f, thereby enhancing the connection stability of the one-way valve 50f.

Referring to FIGS. 96-98, the negative pressure part 30f includes a body portion 32f and a snap-fit portion 33f. The body portion 32f and the nipple tunnel cap element 20f are arranged side by side and share a wall. The body portion 32f is provided with the negative pressure chamber 31f and the milk flow port 40f. The snap-fit portion 33f is connected to a side of the body portion 32f facing away from the negative pressure chamber 31f and disposed close to the milk flow port 40f. The snap-fit portion 33f and the body portion 32f enclose a snap groove 34f, so as to allow the one-way valve 50f to be snap-inserted for limiting. Specifically, the milk flow port 40f is formed in a side of the body portion 32f away from the nipple tunnel cap element 20f and disposed away from the container lid 12f. The snap groove 34f is formed in an end of the body portion 32f away from the container lid 12f.

As an implementation, the nipple tunnel cap element 20f and the negative pressure part 30f are connected to a side of the container lid 12f facing the milk storage chamber 13f. This configuration reduces the number of components of the breast pump assembly. Preferably, the container lid 12f, the nipple tunnel cap element 20f, and the negative pressure part 30f are integrally injection-molded. It is to be understood that in other embodiments, the nipple tunnel cap element 20f and the negative pressure part 30f may also penetrate through the container lid 12f and extend into the milk storage chamber 13f.

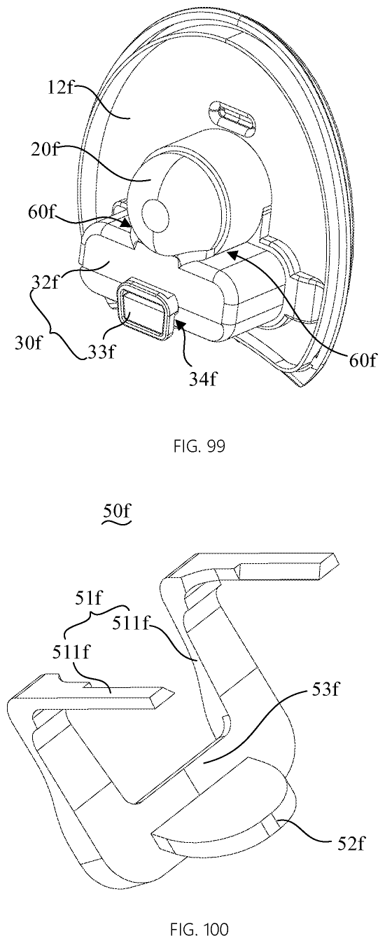

Referring to FIG. 96, FIG. 99, and FIG. 100, the one-way valve 50f includes a fixing portion 51f and a shielding portion 52f. The shielding portion 52f is configured to shield the milk flow port 40f. The fixing portion 51f is fixedly connected to at least one of the container lid 12f, the nipple tunnel cap element 20f and the negative pressure part 30f. This is structurally simple, and is manufactured conveniently and quickly. In specific applications, when the milk flows from the negative pressure chamber 31f to the milk storage chamber 13f, the shielding portion 52f opens the milk flow port 40f to allow the milk to pass through. When the milk flows from the milk storage chamber 13f to the negative pressure chamber 31f, the shielding portion 52f shields the milk flow port 40f, such that the milk cannot enter the negative pressure chamber 31f through the milk flow port 40f, thereby preventing backflow of the milk. In addition, in this embodiment, the fixing portion 51f is fixedly connected to a junction between the nipple tunnel cap element 20f and the negative pressure part 30f, i.e., the fixing portion 51f is fixedly disposed in the groove 60f, thereby greatly enhancing the connection stability of the one-way valve 50f.

Referring to FIG. 98 and FIG. 100, the one-way valve 50f further includes a snap insertion portion 53f. The snap insertion portion 53f is connected between the fixing portion 51f and the shielding portion 52f, and configured to be inserted into a portion of the negative pressure part 30f or the nipple tunnel cap element 20f close to the milk flow port 40f to limit the shielding portion 52f. Specifically, the snap insertion portion 53f is inserted into the snap groove 34f. In this configuration, the snap insertion portion 53f can limit and fix the shielding portion 52f, such that the shielding portion 52f can be closely attached to the milk flow port 40f to shield the milk flow port 40f.

Referring to FIG. 99 and FIG. 100, the fixing portion 51f includes two support arms 511f. The two support arms 511f are connected to two ends of the snap insertion portion 53f, respectively. Specifically, two grooves 60f are formed in the junction between the nipple tunnel cap element 20f and the negative pressure part 30f. The two grooves 60f are respectively located on two sides of the side wall shared by the nipple tunnel cap element 20f and the negative pressure part 30f. One support arm 511f is fixedly disposed in one groove 60f. This enhances stability of fixed connection of the fixing portion 51f.

As an implementation, the one-way valve 50f is a single-disc one-way valve. The single-disc one-way valve has a small size and a small occupied space, thereby ensuring that the milk storage chamber 13f has a sufficiently large space.

Referring to FIG. 96 and FIG. 97, the breast pump assembly further includes a sealing ring 14f. The sealing ring 14f is disposed between the container body 11f and the container lid 12f, and configured to seal the milk storage chamber 13f. This can prevent milk leakage of the milk storage chamber 13f.

Referring to FIG. 95 and FIG. 96, the container body 11f is provided with a milk pouring port 15f communicating with the milk storage chamber 13f. The breast pump assembly further includes a sealing plug 16f. The sealing plug 16f includes one end configured to detachably seal the milk pouring port 15f, and another end connected to the sealing ring 14f through the container lid 12f. In specific applications, when the breast pump 100f is used for milk expression, the sealing plug 16f seals the milk pouring port 15f. Expressed milk flows to the milk storage chamber 13f for temporary storage, and does not flow out from the milk pouring port 15f. When the breast pump 100f has expressed the milk from one or both breasts, the sealing plug 16f is separated from the milk pouring port 15f, and the milk in the milk storage chamber 13f is poured out through the milk pouring port 15f. It is to be understood that in other embodiments, the milk pouring port 15f may also be formed in the container lid 12f.

As an implementation, both the sealing ring 14f and the sealing plug 16f are made of a soft material, and the sealing ring 14f and the sealing plug 16f are at least integrally injection-molded with the container lid 12f. This reduces the number of components of the breast pump assembly, and lowers the probability of losing the components, particularly small components such as the sealing plug 16f. In this embodiment, the sealing ring 14f, the sealing plug 16f, the container lid 12f, the nipple tunnel cap element 20f, and the negative pressure part 30f are integrally injection-molded.

Referring to FIG. 95, FIG. 96, and FIG. 101, the breast pump assembly further includes a main pump module housing 70f and a breast shield 80f. The breast shield 80f includes a first side portion 81f and a second side portion 82f opposite to the first side portion 81f. The first side portion 81f is configured to be placed against the breast. The main pump module housing 70f is disposed between the container lid 12f and the second side portion 82f. When the nipple tunnel cap element 20f and the negative pressure part 30f penetrate through the container lid 12f and extend into the milk storage chamber 13f, both the nipple tunnel cap element 20f and the negative pressure part 30f are connected to the main pump module housing 70f or the breast shield 80f.

Further, the embodiment of the present disclosure further provides a breast pump 100f, including the above breast pump assembly. With the above breast pump assembly, this embodiment reduces the number of components, accelerates the disassembly and assembly speeds, and lowers the probability of losing components.

As an implementation, the breast pump 100f further includes a pump body and a solenoid valve. Both the pump body and the solenoid valve are disposed in the main pump module housing 70f. When the pump body is in operation, the solenoid valve is idle, and the pump body draws air out to generate a negative pressure. When the solenoid valve is in operation, the pump body is idle, and the solenoid valve is opened, such that the air can be charged to the negative pressure chamber 31f.

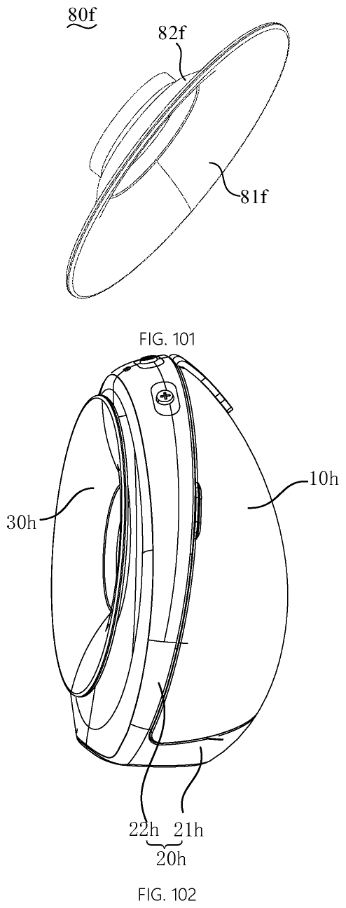

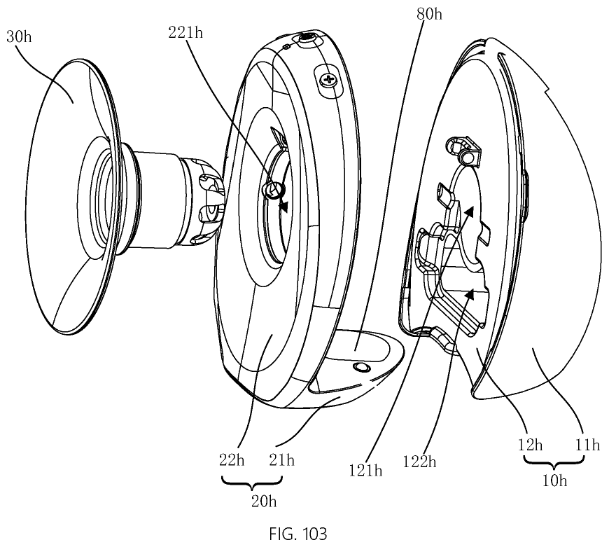

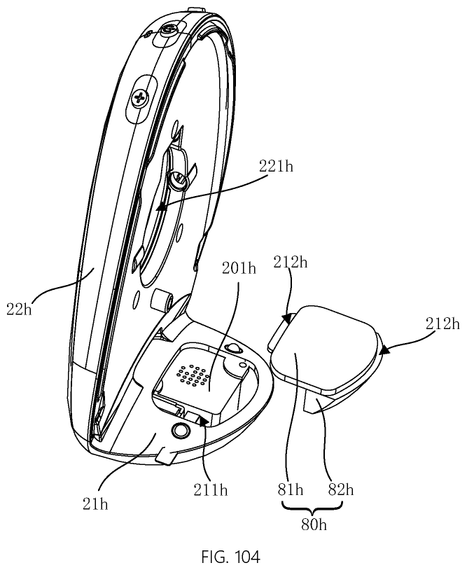

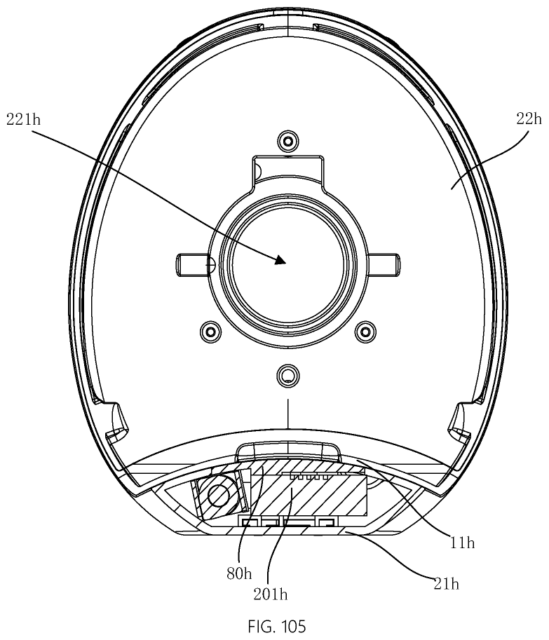

Referring to FIGS. 102-105, FIG. 102 is a schematic structural view of a breast pump according to an embodiment of the present disclosure. FIG. 103 is a schematic exploded view of a breast pump according to an embodiment of the present disclosure. FIG. 104 is a schematic exploded view of a main pump module and a heat sink of a breast pump according to an embodiment of the present disclosure. FIG. 105 is a schematic sectional view of a transverse portion of a main pump module of a breast pump according to an embodiment of the present disclosure.

In this embodiment, the breast pump may include: a milk bowl assembly 10h, a main pump module 20h, a breast shield 30h, and a heat sink 80h.

A milk storage chamber is formed in the milk bowl assembly 10h. The milk bowl assembly 10h includes a milk bowl 11h and a cover plate 12h. The milk bowl 11h and the cover plate 12h enclose the milk storage chamber. Optionally, a groove 121h and a negative pressure chamber 122h that extend into the milk storage chamber are formed in the cover plate 12h. The negative pressure chamber 122h is configured to place a diaphragm. The diaphragm is configured to transfer a negative pressure and achieve gas-liquid isolation between the main pump module 20h and a milk pumping side, so as to prevent reverse osmosis of the milk to the main pump module 20h.

The breast shield 30h has a wide end and a narrow end. An inner diameter of the breast shield 30h from the narrow end to the wide end gradually increases. The narrow end of the breast shield 30h passes through a through hole 221h, is inserted into the groove 121h, and communicates with an internal space of the groove 121h.

The main pump module 20h includes a negative pressure pump 201h. The negative pressure pump 201h is configured to provide the breast pump with a negative pressure for milk expression.

The heat sink 80h is at least partially disposed between the negative pressure pump 201h and the milk bowl assembly 10h. For example, the heat sink 80h may be further disposed between a solenoid valve and the milk bowl assembly 10h.

By disposing the heat sink 80h between the negative pressure pump 201h and the milk bowl assembly 10h, heat of the negative pressure pump 201h is conducted to a gap between the milk bowl assembly 10h and the main pump module 20h, thereby dispersing to the air. Alternatively, the heat is conducted to the milk bowl assembly 10h, and the milk in the milk bowl assembly 10h is used for heat dissipation, thereby effectively mitigating the problem of heating of the main pump module 20h.

Optionally, the main pump module 20h includes a transverse portion 21h and a longitudinal portion 22h connected to the transverse portion 21h in a bending manner. A first side of the milk bowl assembly 10h comes into contact with the longitudinal portion 22h of the main pump module 20h. A second side of the milk bowl assembly 10h comes into contact with the transverse portion 21h of the main pump module 20h. The first side and the second side are two adjacent sides of the milk bowl assembly 10h. The main pump module 20h is configured in an L shape, and the milk bowl assembly 10h is disposed on an L-shaped step. While reducing the overall size of the breast pump, this configuration increases a contact area between the main pump module 20h and the milk bowl assembly 10h, and fully utilizes the milk bowl assembly 10h to dissipate the heat of the main pump module 20h.

In an implementation, the negative pressure pump 201h is located in the transverse portion 21h of the main pump module 20h. A side of the transverse portion 21h close to the milk bowl assembly 10h is provided with a mounting hole 211h. The heat sink 80h is at least partially disposed in the mounting hole 211h. While the main pump module 20h is configured in the L shape, the negative pressure pump 201h can be disposed in the transverse portion 21h of the main pump module 20h with a sufficient space. Moreover, the mounting hole 211h for providing the heat sink 80h is formed in the side of the transverse portion 21h facing the milk bowl assembly 10h, which enhances the heat conduction efficiency of the main pump module 20h and the outside.

In an implementation, the heat sink 80h includes a first portion 81h and a second portion 82h connected to the first portion 81h in a bending manner. The first portion 81h is disposed between the negative pressure pump 201h and the milk bowl assembly 10h. The first portion 81h and the second portion 82h are disposed on two adjacent sides of the negative pressure pump 201h, respectively. By enclosing the two adjacent sides of the negative pressure pump 201h with the heat sink 80h, the heat conduction efficiency between the heat sink 80h and the negative pressure pump 201h can be enhanced, thereby enhancing the heat dissipation effect.

In an implementation, a side of the heat sink 80h close to the milk bowl assembly 10h is provided with an assembly step 212h. The assembly step 212h cooperates with the mounting hole 211h of the transverse portion 21h to limit the heat sink 80h in the transverse portion 21h. With the assembly step 212h, the heat sink 80h is limited in the transverse portion 21h, thereby ensuring that the heat sink 80h can be stably mounted without falling.

In an implementation, the heat sink 80h is exposed through the mounting hole 211h. The heat sink 80h comes into contact with the milk bowl assembly 10h when the milk bowl assembly 10h and the main pump module 20h are well-assembled. By exposing the heat sink 80h through the mounting hole 211h, the problem of low heat dissipation efficiency caused by isolation of the housing of the main pump module 20h can be further reduced. It should be understood that in some implementations, the heat sink 80h may be a cooling fin attached to an outer surface of the main pump module 20h and located between the main pump module 20h and the milk bowl assembly 10h. This embodiment of the present disclosure is not limited to the solution of exposing the heat sink 80h through the mounting hole 211h.

In an implementation, the heat sink 80h is made of a metal material. For example, the heat sink 80h may be made of copper, aluminum, or a copper-aluminum composite. In some implementations, the heat sink 80h may also be made of a non-metal material with good thermal conductivity, such as ceramic and graphene.

In an implementation, the through hole 221h is formed in a thickness direction of the main pump module 20h. The narrow end of the breast shield 30h penetrates into the through hole 221h. The main pump module 20h is at least partially located between the wide end of the breast shield 30 and the milk bowl assembly 10. In this embodiment of the present disclosure, since the through hole 221h is formed in the thickness direction of the main pump module 20, the narrow end of the breast shield 30h penetrates into the through hole 221h, and the main pump module 20h is at least partially located between the wide end of the breast shield 30h and the milk bowl assembly 10h, the overall thickness space of the breast pump can be reduced.

Optionally, the through hole 221h is formed in the longitudinal portion 22h. The longitudinal portion 22h is located between the wide end of the breast shield 30h and the milk bowl assembly 10h.

Optionally, the negative pressure pump 201h may be a piezoelectric pump. The negative pressure pump in this embodiment of the present disclosure may, for example, be an electric piston pump, an electric vortex pump, an electric diaphragm pump, an air pump-type negative pressure pump, a piezoelectric pump, and the like. This is not limited in this embodiment of the present disclosure. These negative pressure pumps suffer from heating problems in different degrees. The problems in the piezoelectric pump are particularly prominent. The piezoelectric pump is a type of fluid actuator. Without an additional drive motor, a piezoelectric vibrator deforms through an inverse piezoelectric effect of the piezoelectric medium. The volume of the pump chamber changes during deformation, thereby outputting the fluid. Alternatively, through movement of the piezoelectric vibrator, the fluid is driven to form a negative pressure. The piezoelectric pump has the small size, low noise and a fast response speed. However, some problems such as heating of the piezoelectric pump are more severe than those of the traditional air pump. In an implementation, the piezoelectric pump may be a piezoelectric ceramic pump.

According to this embodiment of the present disclosure, the breast pump includes the milk bowl assembly, the breast shield, the main pump module, and the heat sink. The milk storage chamber is formed in the milk bowl assembly. The breast shield has the wide end and the narrow end. The inner diameter of the breast shield from the narrow end to the wide end gradually increases. The main pump module includes the negative pressure pump. The negative pressure pump is configured to provide the breast pump with the negative pressure for milk expression. The heat sink is at least partially disposed between the negative pressure pump and the milk bowl assembly. By disposing the heat sink between the negative pressure pump and the milk bowl assembly, heat of the negative pressure pump is conducted to a gap between the milk bowl assembly and the main pump module, thereby dispersing to the air. Alternatively, the heat is conducted to the milk bowl assembly, thereby effectively mitigating the problem of heating of the main pump module.

According to a fourth aspect, in view of shortcomings in the prior art, the present disclosure provides a breast pump, to solve the problem of the unstable center of gravity of the breast pump during wearing.

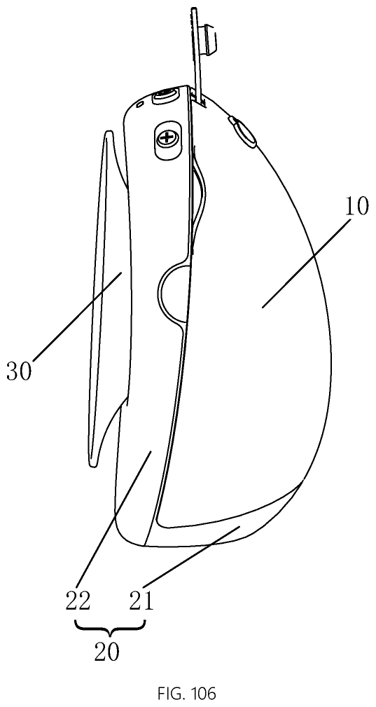

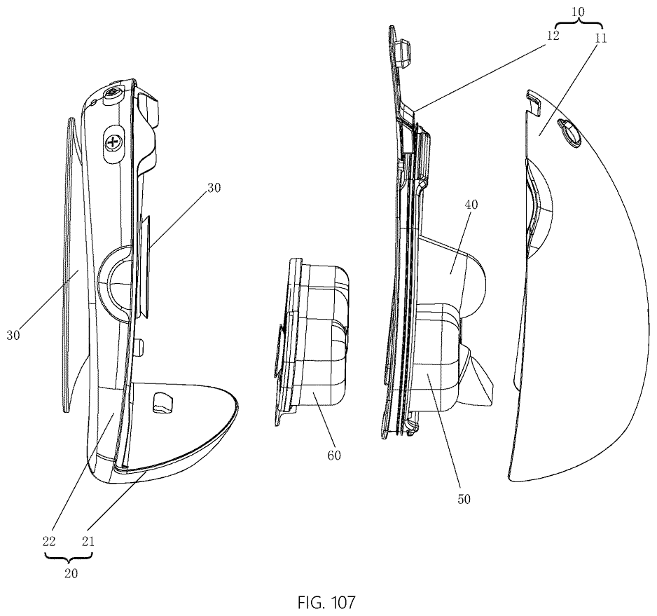

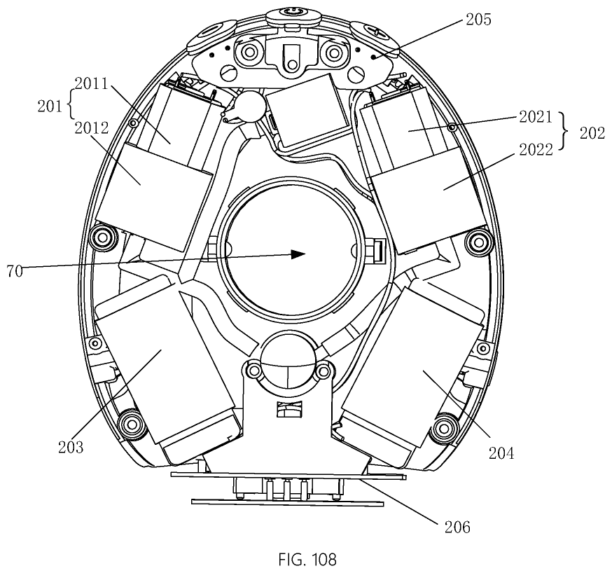

Referring to FIGS. 106-108, FIG. 106 is a schematic structural view of a breast pump according to a first embodiment of the present disclosure. FIG. 107 is a schematic exploded view of a breast pump according to a first embodiment of the present disclosure. FIG. 108 is a schematic view of an internal structure of a main pump module according to a first embodiment of the present disclosure.

In this embodiment, the breast pump includes: a milk bowl assembly 10, a main pump module 20, a breast shield 30, and a milk pumping conduit 40.

A milk storage chamber is formed in the milk bowl assembly 10.

A through hole 70 is formed in a thickness direction of the main pump module 20.

The breast shield 30 has a narrow end and a wide end. The narrow end of the breast shield 30 penetrates into the through hole 70. The main pump module 20 is at least partially located between the wide end of the breast shield 30 and the milk bowl assembly 10. The breast pump according to this embodiment of the present disclosure adopts a three-piece structure with the main pump module 20 clamped between the breast shield 30 and the milk bowl assembly 10, and this structure contributes to the miniaturized design of the breast pump. In this structure, the through hole 70 is formed in the main pump module 20, and a portion of the main pump module 20 clamped between the breast shield 30 and the milk bowl assembly 10 is approximately annular.

The milk pumping conduit 40 communicates with an internal space of the breast shield 30. When the milk storage chamber is empty, a center of gravity of the breast pump is located in the through hole 70 of the main pump module 20 or the milk pumping conduit 40. The main pump module 20 includes a housing as well as a first pumping power device 201 and a second pumping power device 202 that are located in the housing. The first pumping power device 201 and the second pumping power device 202 are located at two opposite sides of the through hole 70, respectively. In this embodiment, with two pumping power assemblies, while a suction force is met, each pumping power device can be made smaller. The two pumping power assemblies can be respectively disposed at the two opposite sides of the through hole 70, which facilitates the layout of the center of gravity, making an internal layout of the main pump module 20 more compact, and contributing to the miniaturized design of the main pump module 20 and even the overall breast pump.

In an implementation, the main pump module 20 further includes a first battery 203 and a second battery 204 that are located in the housing. The first battery 203 and the second battery 204 are located at the two opposite sides of the through hole, respectively. In some implementations, positions of the pumping power device and the battery are interchangeable, provided that the center of gravity is located in the through hole 70 or the milk pumping conduit 40.

In an implementation, the first pumping power device 201 is located at a left upper side of the through hole 70. The second pumping power device 202 is located at a right upper side of the through hole 70. The first battery 203 is located at a left lower side of the through hole 70. The second battery 204 is located at a right lower side of the through hole 70.

In an implementation, the main pump module 20 further includes a first circuit board 205. The first circuit board 205 is located above the through hole 70. The first circuit board 205 may be configured for electrical connection of components such as a button, a display screen or a touch screen.

In an implementation, the main pump module 20 further includes a second circuit board 206. The second circuit board 206 is located below the through hole 70. The second circuit board 206 may be configured for electrical connection of components such as a sensor or a chip.

In an implementation, the first pumping power device 201 includes a first negative pressure pump 2011 and a first solenoid valve 2012. The second pumping power device 202 includes a second negative pressure pump 2021 and a second solenoid valve 2022.

The first negative pressure pump 2011 is an air diaphragm pump or a piezoelectric pump. The second negative pressure pump 2021 is an air diaphragm pump or a piezoelectric pump.

The main pump module 20 includes a transverse portion 21 and a longitudinal portion 22 connected to the transverse portion 21 in a bending manner. The through hole 70 is formed in the longitudinal portion 22. The longitudinal portion 22 is located between the wide end of the breast shield 30 and the milk bowl assembly 10. A first side of the milk bowl assembly 10 contacts the longitudinal portion 22 of the main pump module 20. A second side of the milk bowl assembly 10 contacts the transverse portion of the main pump module 20. The first side and the second side are two adjacent sides of the milk bowl assembly 10.

The second circuit board 206 is disposed in the transverse portion 21.

The milk bowl assembly 10 includes a bowl body 11 and a cover plate 12. The bowl body 11 and the cover plate 12 enclose the milk storage chamber. The milk pumping conduit 40 is connected to the cover plate 12.

In an implementation, the milk bowl assembly 10 includes a bowl body 11 and a cover plate 12. The breast pump further includes a diaphragm bracket 50. A negative pressure chamber is formed in the diaphragm bracket 50. Both the milk pumping conduit 40 and the diaphragm bracket 50 are connected to the cover plate 12.

In an implementation, the milk pumping conduit 40 is located above the diaphragm bracket 50.

In an implementation, the breast pump further includes a diaphragm 60. The diaphragm 60 is configured to be placed in the negative pressure chamber. The diaphragm 60 divides an internal space of the diaphragm bracket 50 into a first subspace and a second subspace. The first subspace communicates with a negative pressure port of the main pump module 20. The second subspace communicates with an internal space of the milk pumping conduit 40. Driven by a negative pressure provided by the main pump module, the diaphragm 60 can deform in the internal space of the diaphragm bracket to transmit the negative pressure.

According to this embodiment of the present disclosure, the breast pump includes the milk bowl assembly, the main pump module, the breast shield, and the milk pumping conduit. The milk storage chamber is formed in the milk bowl assembly. The through hole is formed in the thickness direction of the main pump module. The breast shield has the narrow end and the wide end. The narrow end of the breast shield penetrates into the through hole. The main pump module is at least partially located between the wide end of the breast shield and the milk bowl assembly. The milk pumping conduit communicates with the internal space of the breast shield. When the milk storage chamber is empty, the center of gravity of the breast pump is located in the through hole of the main pump module or the milk pumping conduit. The main pump module includes the housing as well as the first pumping power device and the second pumping power device that are located in the housing. The first pumping power device and the second pumping power device are located at the two opposite sides of the through hole, respectively. The breast pump can be worn stably and more comfortably, and is not prone to falling off.

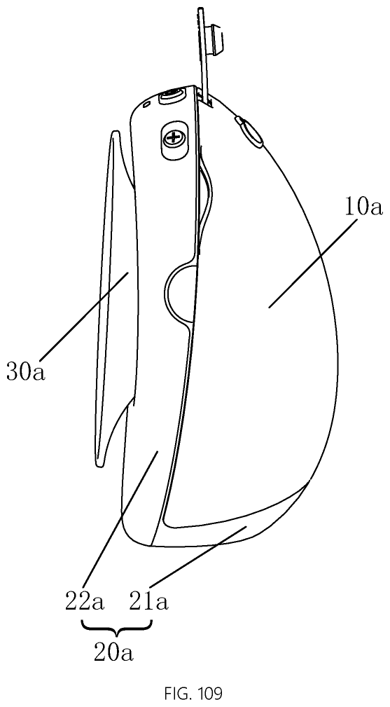

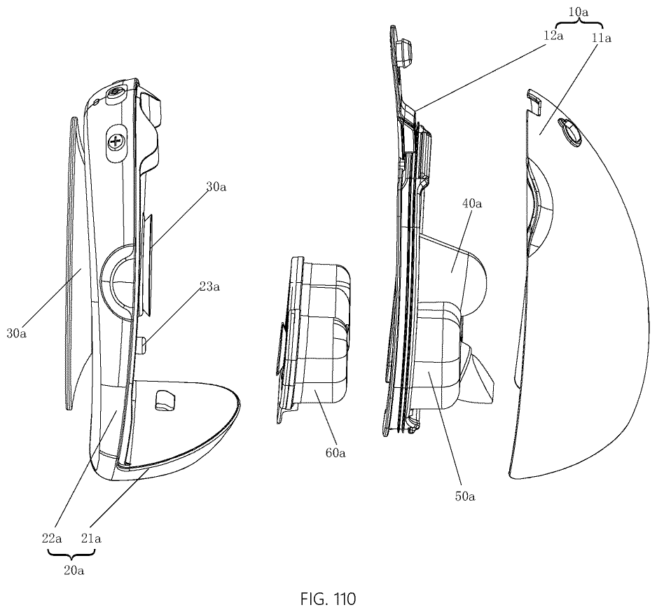

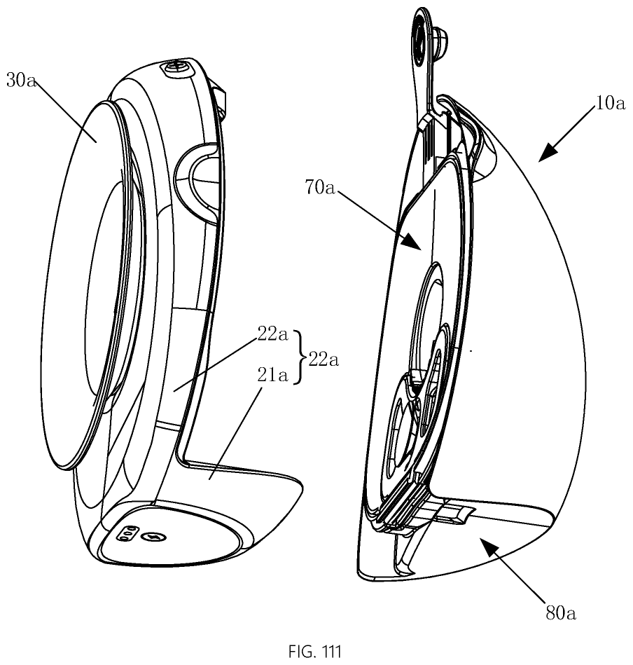

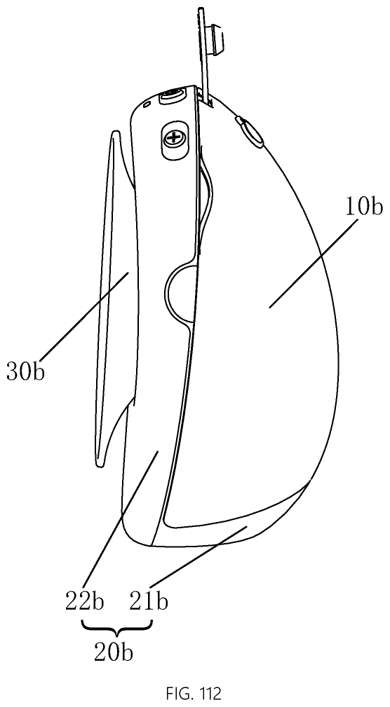

Referring to FIGS. 109-111, FIG. 109 is a schematic structural view of a breast pump according to an embodiment of the present disclosure. FIG. 110 is a first schematic exploded view of a breast pump according to an embodiment of the present disclosure. FIG. 111 is a second schematic exploded view of a breast pump according to an embodiment of the present disclosure.

In this embodiment, the breast pump includes a main pump module 20a and a milk bowl assembly 10a.

The main pump module 20a includes a transverse portion 21a and a longitudinal portion 22a connected to the transverse portion 21a in a bending manner. An included angle between an extension direction of the transverse portion 21a and an extension direction of the longitudinal portion 22a is approximately 90°, which is not limited in this embodiment of the present disclosure, provided that both extend in the bending manner.

A first side 70a of the milk bowl assembly 10a contacts the longitudinal portion 22a of the main pump module 20a. A second side 80a of the milk bowl assembly 10a contacts the transverse portion 21a of the main pump module 20a. The first side 70a and the second side 80a are two adjacent sides of the milk bowl assembly 10a.

In this way, the main pump module 20a is L-shaped as a whole to form an L-shaped step, and the milk bowl assembly 10a is disposed on the L-shaped step of the main pump module 20a. This layout contributes to the miniaturization, lightness and thinness of the breast pump and facilitates the spatial layout in the main pump module. All components of the breast pump can be closely assembled together with a reasonable layout, such that the overall size of the breast pump is effectively reduced to be convenient to carry and use, thereby enhancing the user experience.

In this embodiment, a thickness of an upper half of the longitudinal portion 22a of the main pump module 20a is greater than a thickness of a lower half of the longitudinal portion 22a of the main pump module 20a. In some implementations, the thickness of the upper half may refer to an average thickness of the upper half at various positions in a height direction, and the thickness of the lower half may refer to an average thickness of the lower half at various positions in the height direction.

This facilitates the layout of components in the main pump module 20a. The components in the main pump module, such as a negative pressure pump, a solenoid valve, a battery, and a circuit board, typically have different sizes. The larger-sized components can be arranged in an upper portion, while the smaller-sized ones can be arranged in a lower portion. On the other hand, without changing the overall thickness of the breast pump, the lower half of the milk bowl assembly can be made thicker to increase the capacity of the lower half of the milk bowl assembly. This reduces the probability that the components such as the one-way valve or the milk outlet in the milk bowl assembly are submerged by milk, improves the milk expression efficiency, lowers the energy consumption for milk expression, and reduces the backflow risk. Furthermore, this makes the breast shield assembly and the main pump module staggered with each other, limiting the milk bowl assembly and the main pump module more conveniently in an up-down direction and a left-right direction.

In an implementation, a thickness of the longitudinal portion 22a of the main pump module 20a gradually decreases from top to bottom.

In an implementation, a thickness of an upper half of the milk bowl assembly 10a is less than a thickness of a lower half of the milk bowl assembly 10a.

In an implementation, a thickness of the milk bowl assembly 10a gradually increases from top to bottom.

In an implementation, a thickness of the transverse portion 21a of the main pump module 20a gradually decreases along a direction toward a junction between the transverse portion 21a and the longitudinal portion 22a.

In an implementation, a surface of the transverse portion 21a of the main pump module 20a that contacts the milk bowl assembly 10a is non-planar. For example, a top surface of the transverse portion 21a of the main pump module 20a protrudes toward the second side 80a of the milk bowl assembly 10a. The top surface of the transverse portion 21a of the main pump module 20a is a curved surface, which can increase a contact area between the main pump module 20a and the milk bowl assembly 10a, facilitating heat dissipation of the main pump module 20a via the milk in the milk bowl assembly 10a. The curved shape is not limited to a smoothly transitioning curved surface, but may also be a non-smoothly transitioning curved surface, such as a surface bending and transitioning at an included angle, a zigzag-shaped surface, and a wavy-shaped surface. The second side 80a of the milk bowl assembly 10a is approximately consistent with the top surface of the transverse portion 21a in shape. On the other hand, such a non-planar design also facilitates the limited design between the main pump module 20a and the milk bowl assembly 10a in the transverse direction, and makes locating easier during mounting.

In an implementation, a through hole is formed in a thickness direction of the longitudinal portion 22a of the main pump module 20a. The breast pump further includes a breast shield 30a. The breast shield 30a has a narrow end and a wide end. The narrow end of the breast shield 30a penetrates into the through hole. The main pump module 20a is at least partially located between the wide end of the breast shield 30a and the milk bowl assembly 10a.

In an implementation, the milk bowl assembly 10a includes a bowl body 11a and a cover plate 12a. The bowl body 11a and the cover plate 12a enclose a milk storage chamber. The breast pump further includes a milk pumping conduit 40a and a diaphragm bracket 50a. A negative pressure chamber is formed in the diaphragm bracket 50a. Both the milk pumping conduit 40a and the diaphragm bracket 50a are connected to the cover plate 12a.

In an implementation, the milk pumping conduit 40a is located above the diaphragm bracket 50a.

In an implementation, the breast pump further includes a diaphragm 60a. The diaphragm 60a is configured to be placed in the negative pressure chamber. The diaphragm 60a divides an internal space of the diaphragm bracket 50a into a first subspace and a second subspace. The first subspace communicates with a negative pressure port 23a of the main pump module 20a. The second subspace communicates with an internal space of the milk pumping conduit 40a. Driven by a negative pressure provided by the main pump module, the diaphragm 60a can deform in the internal space of the diaphragm bracket to transmit the negative pressure.

According to this embodiment of the present disclosure, the breast pump includes the main pump module and the milk bowl assembly. The main pump module includes the transverse portion and the longitudinal portion connected to the transverse portion in the bending manner. The thickness of the upper half of the longitudinal portion of the main pump module is greater than the thickness of the lower half of the longitudinal portion of the main pump module. The first side of the milk bowl assembly contacts the longitudinal portion of the main pump module. The second side of the milk bowl assembly contacts the transverse portion of the main pump module. The first side and the second side are two adjacent sides of the milk bowl assembly. This can increase a capacity of the lower half of the milk bowl, without additionally increasing the overall thickness of the breast pump. Moreover, this is more convenient for layout of components in the main pump module.

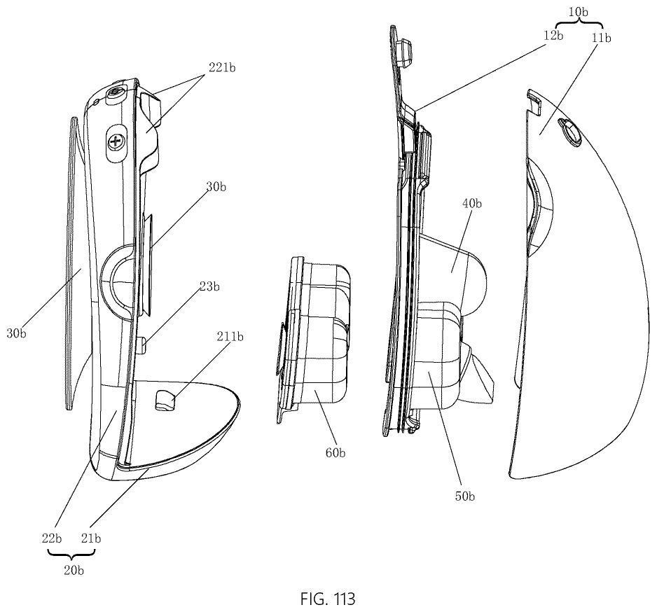

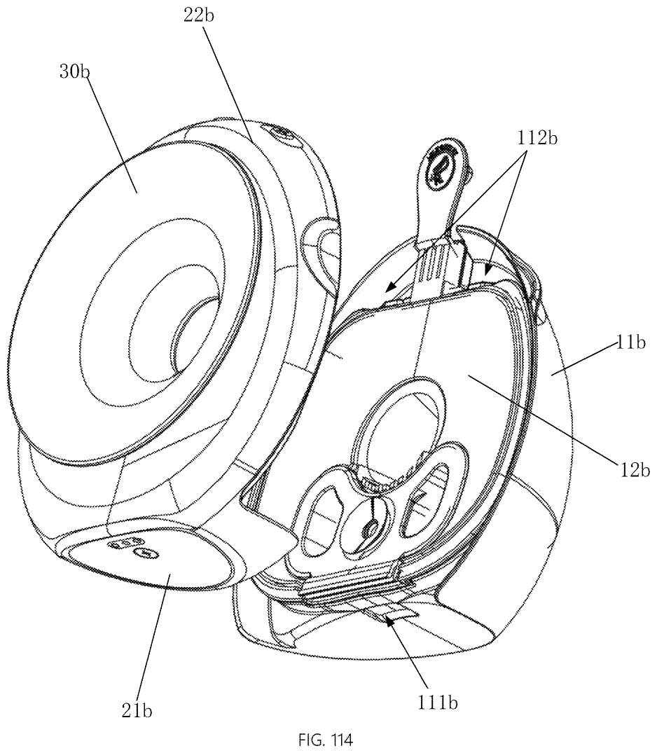

Referring to FIGS. 112-114, FIG. 112 is a schematic structural view of a breast pump according to a third embodiment of the present disclosure. FIG. 113 is a first schematic exploded view of a breast pump according to a third embodiment of the present disclosure. FIG. 114 is a second schematic exploded view of a breast pump according to a third embodiment of the present disclosure.

In this embodiment, the breast pump includes a main pump module 20b and a milk bowl assembly 10b.

The main pump module 20b includes a transverse portion 21b and a longitudinal portion 22b connected to the transverse portion 21b in a bending manner.

A first side of the milk bowl assembly 10b contacts the longitudinal portion 22b of the main pump module 20b. A second side of the milk bowl assembly 10b contacts the transverse portion 21b of the main pump module 20b. The first side and the second side are two adjacent sides of the milk bowl assembly 10b. By bending the main pump module 20b into an approximately L shape, the stability of assembly of the milk bowl assembly 10b and the main pump module 20b can be improved.

A first coupling structure 211b is disposed on the transverse portion 21b. A second coupling structure 111b matching with the first coupling structure 211b is disposed on the second side of the milk bowl assembly 10b. The first coupling structure 211b and the second coupling structure 111b are configured to be detachably coupled with each other, so as to realize mounting or positioning of the main pump module 20b and the milk bowl assembly 10b. By respectively disposing the first coupling structure 211b and the second coupling structure 111b on the transverse portion 21b of the main pump module 20b and on the corresponding position of the milk bowl assembly 10b, the stability of assembly of the milk bowl assembly 10b and the main pump module 20b can further be improved. The first coupling structure 211b and the second coupling structure 111b may be complementary coupling structures, for example, one is a snap groove and the other is a snap fastener. See the following description for details.

In an implementation, a third coupling structure 221b is disposed on the longitudinal portion 22b. A fourth coupling structure 112b matching with the third coupling structure 221b is disposed on the first side of the milk bowl assembly 10b. The third coupling structure 221b and the fourth coupling structure 112b are configured to be detachably coupled with each other, so as to realize mounting or positioning of the main pump module 20b and the milk bowl assembly 10b. The first coupling structure 211b and the second coupling structure 111b may be complementary coupling structures, for example, one is a snap groove and the other is a snap fastener.

Optionally, two third coupling structures 221b are disposed on the longitudinal portion 22b. Two fourth coupling structures 112b are correspondingly disposed on the first side of the milk bowl assembly 10b. One first coupling structure 211b is disposed on the transverse portion 21b. By providing one coupling point on the transverse portion 21b, and two coupling points on the longitudinal portion, the stability of mounting and locating can be improved, and the removal and mounting are smoother.

In an implementation, lines connecting positions where the two third coupling structures 221b and the one first coupling structure 211b are disposed enclose a triangle. By setting positions of the three coupling points as a triangular positional relationship, the stability of mounting and locating can be improved.

In an implementation, one of the first coupling structure 211b and the second coupling structure 111b is an elastic snap fastener, while the other is a snap groove.

In an implementation, the first coupling structure 211b is the elastic snap fastener. The second coupling structure 111b is the snap groove. A surface of the elastic snap fastener 211b facing the milk bowl assembly 10b has an oblique plane that inclines upward along a direction toward the longitudinal portion 22b. By providing the oblique surface on the elastic snap fastener 211b, the main pump module 20b and the milk bowl assembly 10b can be mounted and located smoothly.

One of the third coupling structure 221b and the fourth coupling structure 112b is a protrusion, while the other is a recess.

Optionally, the third coupling structure 221b is the protrusion 221b, and the fourth coupling structure 112b is the recess.

A surface of the protrusion 221b facing the transverse portion 21b of the main pump module 20b is provided with a ball plunger.

The milk bowl assembly 10b includes a bowl body 11b and a cover plate 12b. The bowl body 11b and the cover plate 12b enclose a milk storage chamber. The second coupling structure 111b is disposed on the bowl body 11b. The breast pump further includes a milk pumping conduit 40b and a diaphragm bracket 50b. A negative pressure chamber is formed in the diaphragm bracket 50b. Both the milk pumping conduit 40b and the diaphragm bracket 50b are connected to the cover plate 12b.

In an implementation, the milk pumping conduit 40b is located above the diaphragm bracket 50b.

In an implementation, the breast pump further includes a diaphragm 60b. The diaphragm 60b is configured to be placed in the negative pressure chamber. The diaphragm 60b divides an internal space of the diaphragm bracket 50b into a first subspace and a second subspace. The first subspace communicates with a negative pressure port 23b of the main pump module 20b. The second subspace communicates with an internal space of the milk pumping conduit 40b. Driven by a negative pressure provided by the main pump module, the diaphragm 60b can deform in the internal space of the diaphragm bracket to transmit the negative pressure.

According to this embodiment of the present disclosure, the breast pump includes the main pump module and the milk bowl assembly. The main pump module includes the transverse portion and the longitudinal portion connected to the transverse portion in the bending manner. The first side of the milk bowl assembly contacts the longitudinal portion of the main pump module. The second side of the milk bowl assembly contacts the transverse portion of the main pump module. The first side and the second side are two adjacent sides of the milk bowl assembly. The first coupling structure is disposed on the transverse portion. The second coupling structure matching with the first coupling structure is disposed on the second side of the milk bowl assembly. The first coupling structure and the second coupling structure are configured to be coupled with each other, so as to realize mounting or positioning of the main pump module and the milk bowl assembly. This can improve the stability of mounting or positioning the milk bowl assembly and the main pump module.

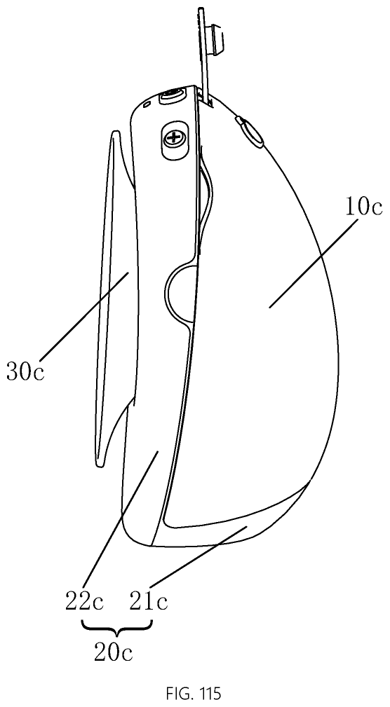

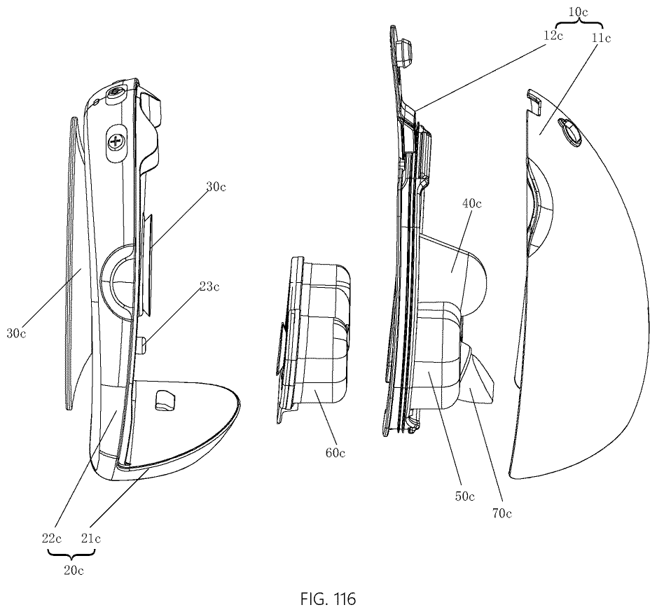

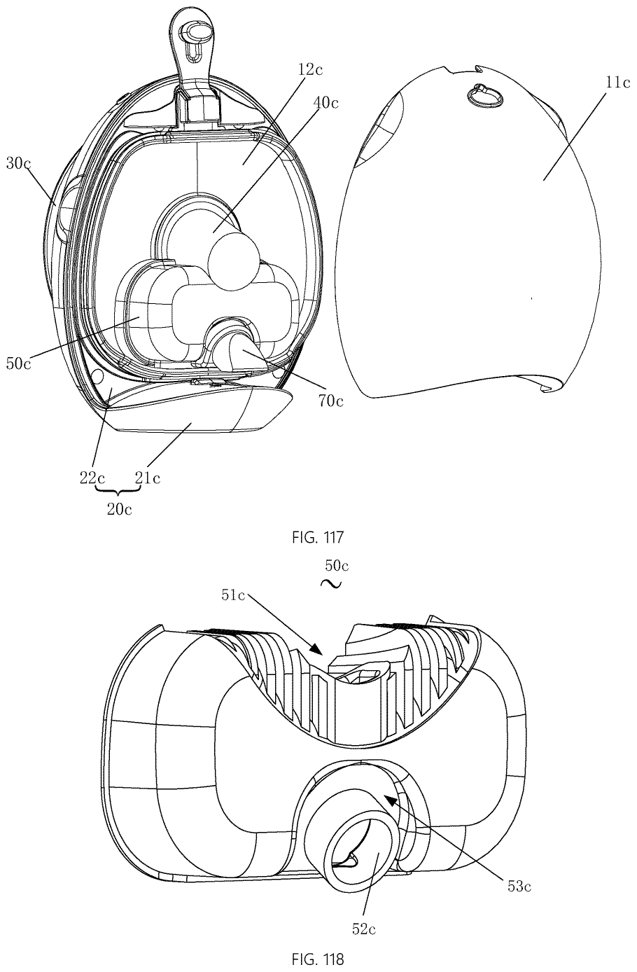

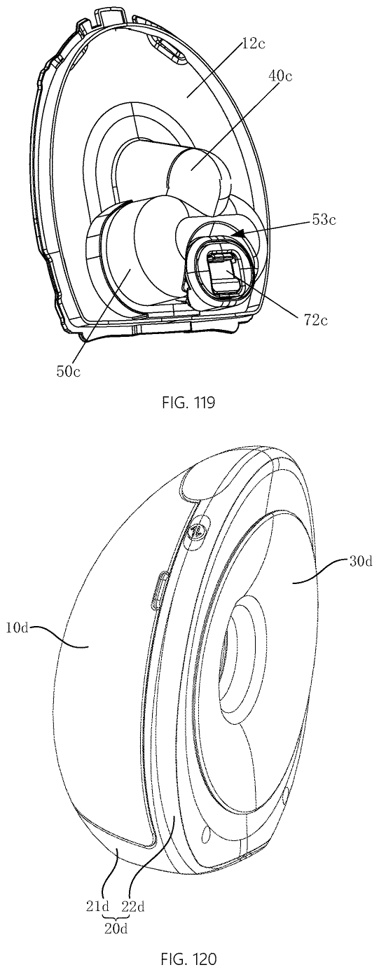

Referring to FIGS. 115-119, FIG. 115 is a schematic structural view of a breast pump according to a fourth embodiment of the present disclosure. FIG. 116 is a first schematic exploded view of a breast pump according to a fourth embodiment of the present disclosure. FIG. 117 is a second schematic exploded view of a breast pump according to a fourth embodiment of the present disclosure. FIG. 118 is a schematic structural view of a diaphragm bracket according to a fourth embodiment of the present disclosure. FIG. 119 is a schematic structural view of a disc-type one-way valve disposed at a milk flow port according to a fourth embodiment of the present disclosure.

In this embodiment, the breast pump includes: a diaphragm bracket 40c and a milk pumping conduit 50c.

A negative pressure chamber is formed in the diaphragm bracket 40c. A nipple tunnel is formed in the milk pumping conduit 50c. The milk pumping conduit 50c communicates with the negative pressure chamber. The nipple tunnel is at least configured to guide milk from a breast. The milk pumping conduit 50c and the diaphragm bracket 40c are arranged side by side in the breast pump. A notch 51c configured to avoid the milk pumping conduit 50c is formed in the diaphragm bracket 40c. The milk pumping conduit 50c is at least partially embedded into the notch 51c of the diaphragm bracket 40c.

In an implementation, the notch 51c is formed in an upper side or a lower side of the diaphragm bracket 40c, such that spaces respectively occupied by the diaphragm bracket 40c and the milk pumping conduit 50c in a vertical direction at least partially overlap.

In an implementation, a milk flow port 52c is formed in the diaphragm bracket 40c. The milk flow port 52c is configured to flow the milk in the negative pressure chamber to a milk storage chamber of the breast pump.

In an implementation, a recess 53c that at least partially accommodates the milk flow port 52c is formed in the diaphragm bracket 40c.

In an implementation, a one-way valve 70c is disposed at the milk flow port 52c. The one-way valve 70c is at least partially accommodated in the recess 53c. The one-way valve 70c is configured to allow the milk to flow to the milk storage chamber unidirectionally.

In some implementations, the one-way valve 70c may use a duckbill valve 70c. In other implementations, the one-way valve 70c may use a disc-type one-way valve 71c. When the disc-type one-way valve 71c is used, the disc-type one-way valve 71c can be completely accommodated in the recess 53c, contributing to the light and thin design without interfering with other components to cause blockage of the one-way valve.

In some implementations, the breast pump includes a milk bowl assembly 10c. The milk storage chamber is formed in the milk bowl assembly 10c. The diaphragm bracket 40c is connected to the milk bowl assembly 10c and extends into the milk storage chamber.

In an implementation, the milk pumping conduit 40c is connected to the milk bowl assembly 10c and extends into the milk storage chamber.

In an implementation, the milk bowl assembly 10c includes a bowl body 11c and a cover plate 12c. The bowl body 11c and the cover plate 12c enclose the milk storage chamber. Both the milk pumping conduit 40c and the diaphragm bracket 50c are connected to the cover plate 12c.

In an implementation, the milk pumping conduit 40c and the diaphragm bracket 50c are integrally connected with the cover plate 12c.

In an implementation, the milk pumping conduit 40c is located above the diaphragm bracket 50c.

In an implementation, the breast pump further includes a main pump module 20c and a breast shield 30c. A through hole is formed in a thickness direction of the main pump module 20c. The breast shield 30c has a narrow end and a wide end. The narrow end of the breast shield 30c penetrates into the through hole. The main pump module 20c is at least partially located between the wide end of the breast shield 30c and the milk bowl assembly 10c.

Optionally, the main pump module 20c includes a transverse portion 21c and a longitudinal portion 22c connected to the transverse portion 21c in a bending manner. The through hole is formed in the longitudinal portion 22c. The longitudinal portion 22c is located between the wide end of the breast shield 30c and the milk bowl assembly 10c. A first side of the milk bowl assembly 10c contacts the longitudinal portion 22c of the main pump module 20c. A second side of the milk bowl assembly 10c contacts the transverse portion 21c of the main pump module 20c. The first side and the second side are two adjacent sides of the milk bowl assembly 10c.

Optionally, the breast pump further includes a diaphragm 60c. The diaphragm 60c is configured to be placed in the negative pressure chamber. The diaphragm 60c divides an internal space of the diaphragm bracket 50c into a first subspace and a second subspace. The first subspace communicates with a negative pressure port 23c of the main pump module 20c. The second subspace communicates with an internal space of the milk pumping conduit 40c. Driven by a negative pressure provided by the main pump module 20c, the diaphragm 60c can deform in the internal space of the diaphragm bracket 50c to transmit the negative pressure.

According to this embodiment of the present disclosure, the breast pump includes the diaphragm bracket and the milk pumping conduit. The negative pressure chamber is formed in the diaphragm bracket. The nipple tunnel is formed in the milk pumping conduit. The milk pumping conduit communicates with the negative pressure chamber. The nipple tunnel is at least configured to guide the milk from the breast. The milk pumping conduit and the diaphragm bracket are arranged side by side in the breast pump. The notch configured to avoid the milk pumping conduit is formed in the diaphragm bracket. The milk pumping conduit is at least partially embedded into the notch of the diaphragm bracket. Since the diaphragm bracket and the milk pumping conduit are arranged side by side, and the notch configured to avoid the milk pumping conduit is formed in the diaphragm bracket, such that the milk pumping conduit is at least partially embedded into the notch of the diaphragm bracket, and spaces occupied by the milk pumping conduit in transverse and longitudinal directions overlap. This effectively reduces the overall size of the breast pump, such that the breast pump is carried and stored more conveniently, meeting requirements of modern mothers on miniaturization and portability of the breast pump.

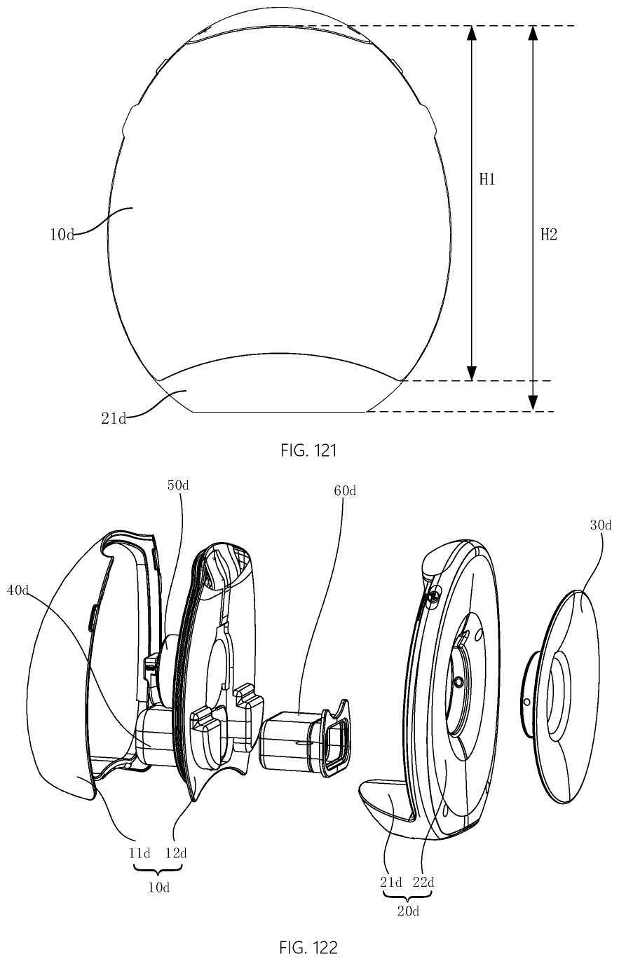

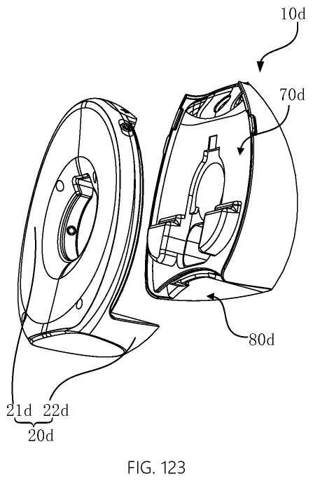

Referring to FIGS. 120-123, FIG. 120 is a schematic structural view of a breast pump from a first viewing angle according to a fifth embodiment of the present disclosure. FIG. 121 is a schematic structural view of a breast pump from a second viewing angle according to a fifth embodiment of the present disclosure. FIG. 122 is a first schematic exploded view of a breast pump according to a fifth embodiment of the present disclosure. FIG. 123 is a second schematic exploded view of a breast pump according to a fifth embodiment of the present disclosure.

In an implementation, the breast pump includes a main pump module 20d and a milk bowl assembly 10d.

The main pump module 20d includes a transverse portion 21d and a longitudinal portion 22d connected to the transverse portion 21d in a bending manner.

A first side 70d of the milk bowl assembly 10d contacts the longitudinal portion 22d of the main pump module 20d. A second side 80d of the milk bowl assembly 10d contacts the transverse portion 21d of the main pump module 20d. The first side 70d and the second side 80d are two adjacent sides of the milk bowl assembly 10d. A height H1 of the milk bowl assembly 10d accounts for 70% or more of a height H2 of the breast pump.

The main pump module is approximately L-shaped, the milk bowl assembly is placed on an L-shaped step, the height of the milk bowl assembly accounts for 70% or more of the total height of the breast pump, and a height of the transverse portion of the main pump module accounts for 30% or less of the total height of the breast pump. This can greatly increase the visible area of the milk bowl assembly, and facilitate observation of the user on a milk condition in the milk bowl, improving the visualization degree, and enhancing expression feedback experience of the user.

As an implementation, at least a portion of the milk bowl assembly 10d not shielded by the main pump module 20d is made of a light-transmissive material.

As an implementation, an area of a portion of the milk bowl assembly 10d not shielded by the main pump module 20d accounts for 60% or more of a surface area of the breast pump. This can greatly increase the visible area of the milk bowl assembly, and facilitate observation of the user on the milk condition in the milk bowl, improving the visualization degree, and enhancing expression feedback experience of the user.

As an implementation, the breast pump further includes a milk pumping conduit 40d extending into the milk bowl assembly 10d. The milk pumping conduit 50d is made of a light-transmissive material. The milk pumping conduit 50d is typically configured to accommodate a nipple, facilitating observation and adjustment of the user to place the nipple at an appropriate position in the milk pumping conduit 50d during wearing.

As an implementation, the breast pump further includes a diaphragm bracket 50d extending into the milk bowl assembly 10d. The diaphragm bracket 40d is located below the milk pumping conduit 50d.

As an implementation, the milk bowl assembly 10d includes a bowl body 11d and a cover plate 12d. The bowl body 11d and the cover plate 12d enclose a milk storage chamber. The breast pump further includes the milk pumping conduit 50d and the diaphragm bracket 40d. A negative pressure chamber is formed in the diaphragm bracket 40d. Both the milk pumping conduit 50d and the diaphragm bracket 40d are connected to the cover plate 12d.

In an implementation, the milk pumping conduit 50d and the diaphragm bracket 40d are integrally connected with the cover plate 12d.

As an implementation, the diaphragm bracket 40d is made of a light-transmissive material. The light-transmissive material of the diaphragm bracket 40d can facilitate observation of the user on the whole milk expression process through the milk bowl assembly 10d and the diaphragm bracket 40d, providing a desirable milk expression feedback for the user.

As an implementation, a through hole is formed in a thickness direction of the longitudinal portion 22d of the main pump module 20d.

The breast pump further includes a breast shield 30d. The breast shield 30d has a narrow end and a wide end. The narrow end of the breast shield 30d penetrates into the through hole. The main pump module 20d is at least partially located between the wide end of the breast shield 30d and the milk bowl assembly 10d.

As an implementation, the breast pump further includes a diaphragm 60d. The diaphragm 60d is configured to be placed in the negative pressure chamber. The diaphragm 60d divides an internal space of the diaphragm bracket 40d into a first subspace and a second subspace. The first subspace communicates with a negative pressure port of the main pump module 20d. The second subspace communicates with an internal space of the milk pumping conduit 50d. Driven by a negative pressure provided by the main pump module 20d, the diaphragm 60d can deform in the internal space of the diaphragm bracket 40d to transmit the negative pressure.

Another technical solution adopted by the present disclosure is as follows: A breast pump is provided, including: a main pump module, where the main pump module includes a transverse portion and a longitudinal portion connected to the transverse portion in a bending manner; and a milk bowl assembly, where a first side of the milk bowl assembly contacts the longitudinal portion of the main pump module, a second side of the milk bowl assembly contacts the transverse portion of the main pump module, the first side and the second side are two adjacent sides of the milk bowl assembly, and an area of a portion of the milk bowl assembly not shielded by the main pump module accounts for 60% or more of a surface area of the breast pump.

According to this embodiment of the present disclosure, the breast pump includes the main pump module and the milk bowl assembly. The main pump module includes the transverse portion and the longitudinal portion connected to the transverse portion in the bending manner. The first side of the milk bowl assembly contacts the longitudinal portion of the main pump module. The second side of the milk bowl assembly contacts the transverse portion of the main pump module. The first side and the second side are two adjacent sides of the milk bowl assembly. The height of the milk bowl assembly accounts for 70% or more of the height of the breast pump. The main pump module is approximately L-shaped, the milk bowl assembly is placed on an L-shaped step of the milk bowl assembly, the height of the milk bowl assembly accounts for 70% or more of the total height of the breast pump, and a height of the transverse portion of the main pump module accounts for 30% or less of the total height of the breast pump. This can greatly increase the visible area of the milk bowl assembly, and facilitate observation of the user on a milk condition in the milk bowl, improving the visualization degree, and enhancing expression feedback experience of the user.

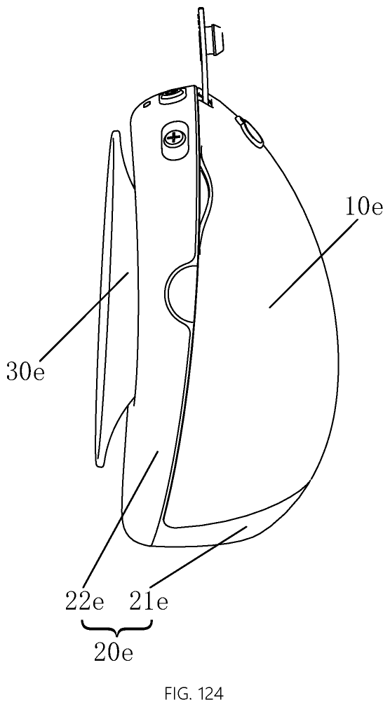

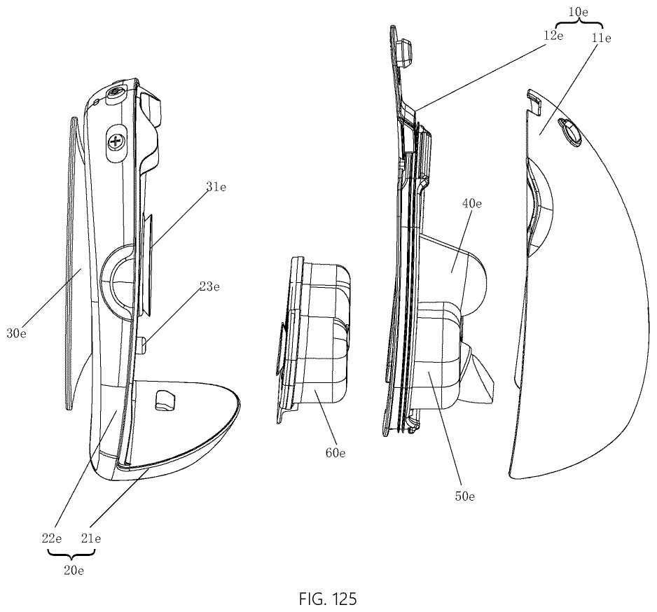

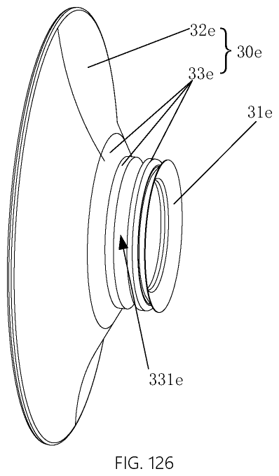

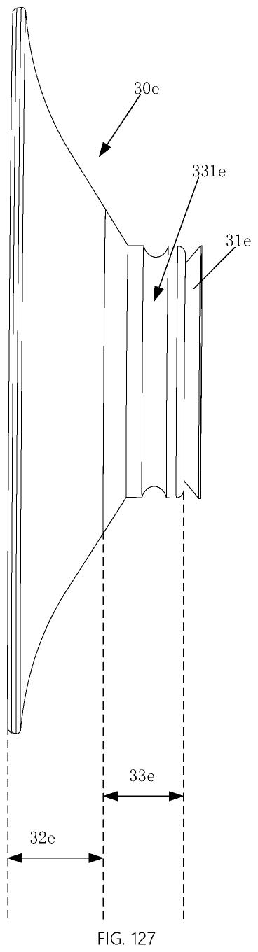

Referring to FIGS. 124-127, FIG. 124 is a schematic structural view of a breast pump according to a sixth embodiment of the present disclosure. FIG. 125 is a schematic exploded view of a breast pump according to a sixth embodiment of the present disclosure. FIG. 126 is a schematic structural view of a breast shield and a sealing structure from a first viewing angle according to a sixth embodiment of the present disclosure. FIG. 127 is a schematic structural view of a breast shield and a sealing structure from a second viewing angle according to a sixth embodiment of the present disclosure.

In this embodiment, the breast pump includes: a breast shield 30e, a milk pumping conduit 40e, and a sealing structure 31e.

The breast shield 30e has a narrow end and a wide end. An inner diameter of the breast shield 30e gradually increases from the narrow end to the wide end.

An end surface of one end of the milk pumping conduit 40e abuts against an end surface of the narrow end of the breast shield 30e. The milk pumping conduit 40e communicates with an internal space of the breast shield 30e. Since the end surface of one end of the milk pumping conduit 40e abuts against the end surface of the narrow end of the breast shield 30e, the docking manner through which the narrow end of the breast shield 30e is inserted into the milk pumping conduit 40e can be omitted, and the internal space of the milk pumping conduit 40e having the same size can be made larger.

In this embodiment, the sealing structure 31e is disposed around a port of the narrow end. In some implementations, the sealing structure may also be disposed around a port of an end of the milk pumping conduit 40e close to the breast shield 30e.

In this embodiment, the sealing structure 31e is disposed at the port of the narrow end of the breast shield.

In this embodiment, the sealing structure 31e is horn-shaped, with an inner diameter gradually increasing along a direction toward the milk pumping conduit 40e. In this way, when the narrow end of the breast shield 30e is docked with the milk pumping conduit 40e, the sealing structure 31e can be attached to the end surface of the milk pumping conduit 40e well to achieve a desirable sealing effect.

In an implementation, the sealing structure 31e is made of a soft material. The narrow end of the breast shield 30e is at least partially made of a rigid material.

In an implementation, more than 50% of the breast shield 30e is made of the soft material. For example, as shown in FIG. 126 and FIG. 127, the breast shield 30e includes a soft portion 32e and a rigid portion 33e. The soft portion 32e is close to the wide end of the breast shield 30e, while the rigid portion 33e is close to the narrow end. The soft portion 32e contacts the user during wearing, being soft and comfortable to the user. The rigid portion 33e facilitates connection between the breast shield 30e and the main pump module 20e. For example, a snap groove 331e is formed in the rigid portion of the breast shield 30e, and a ball plunger is disposed at the through hole of the main pump module 20e. When the narrow end of the breast shield 30e is inserted into the through hole, the ball plunger is snapped into the snap groove 331e to realize detachable connection between the main pump module 20e and the breast shield 30e. Optionally, the snap groove 331e is annular, i.e., the snap groove 331e is formed on an outer wall of the narrow end. In this way, the breast shield 30e can be snapped into the through hole in any direction to match with the ball plunger.

In an implementation, the sealing structure 31e and the breast shield 30e are integrally formed. Specifically, they are formed by integral injection molding with hard and soft rubber.

In an implementation, the breast pump further includes a milk bowl assembly 10e. A milk storage chamber is formed in the milk bowl assembly 10e. The milk pumping conduit 40e is connected to the milk bowl assembly 10e and extends into the milk storage chamber.

As an implementation, the breast pump further includes a diaphragm bracket 50e extending into the milk bowl assembly 10e. The diaphragm bracket 50e is located below the milk pumping conduit 40e.

As an implementation, the milk bowl assembly 10e includes a bowl body 11e and a cover plate 12e. The bowl body 11e and the cover plate 12e enclose a milk storage chamber. The breast pump further includes the milk pumping conduit 40e and the diaphragm bracket 50e. A negative pressure chamber is formed in the diaphragm bracket 50e. Both the milk pumping conduit 40e and the diaphragm bracket 50e are connected to the cover plate 12e.

In an implementation, the milk pumping conduit 40e and the diaphragm bracket 50e are integrally formed with the cover plate 12e.

In an implementation, the breast pump further includes a main pump module 20e. A through hole is formed in a thickness direction of the main pump module 20e. The narrow end of the breast shield 30e penetrates into the through hole. The main pump module 20e is at least partially located between the wide end of the breast shield 30e and the milk bowl assembly 10e.

In an implementation, the main pump module 20e includes a transverse portion 21e and a longitudinal portion 22e connected to the transverse portion 21e in a bending manner. A first side of the milk bowl assembly 10e contacts the longitudinal portion of the main pump module 20e. A second side of the milk bowl assembly 10e contacts the transverse portion 21e of the main pump module 20e. The first side and the second side are two adjacent sides of the milk bowl assembly 10e.

In an implementation, the breast shield 30e is detachably connected to the main pump module 20e.

Optionally, the breast pump further includes a diaphragm 60e. The diaphragm 60e is configured to be placed in the negative pressure chamber. The diaphragm 60e divides an internal space of the diaphragm bracket 50e into a first subspace and a second subspace. The first subspace communicates with a negative pressure port 23e of the main pump module 20e. The second subspace communicates with an internal space of the milk pumping conduit 40e. Driven by a negative pressure provided by the main pump module 20e, the diaphragm 60e can deform in the internal space of the diaphragm bracket 50e to transmit the negative pressure.

According to this embodiment of the present disclosure, the breast pump includes the breast shield, the milk pumping conduit, and the sealing structure. The breast shield has the narrow end and the wide end. The inner diameter of the breast shield gradually increases from the narrow end to the wide end. The end surface of one end of the milk pumping conduit abuts against the end surface of the narrow end of the breast shield. The milk pumping conduit communicates with the internal space of the breast shield. The sealing structure is disposed around the port of the narrow end or around the port of the end of the milk pumping conduit close to the breast shield. In this way, the present disclosure can improve the airtightness of the milk pumping channel of the breast pump.

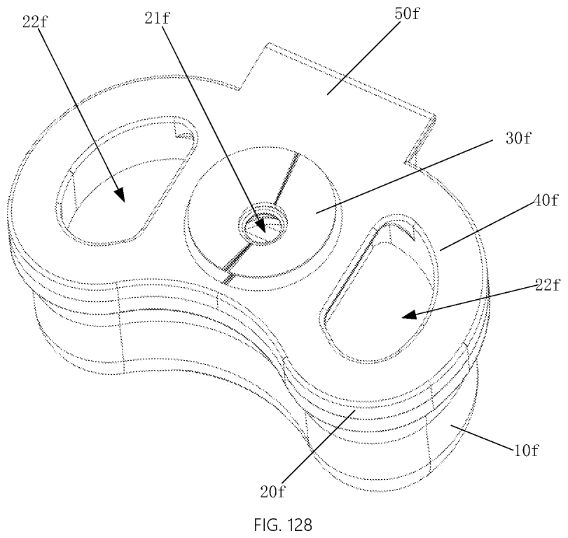

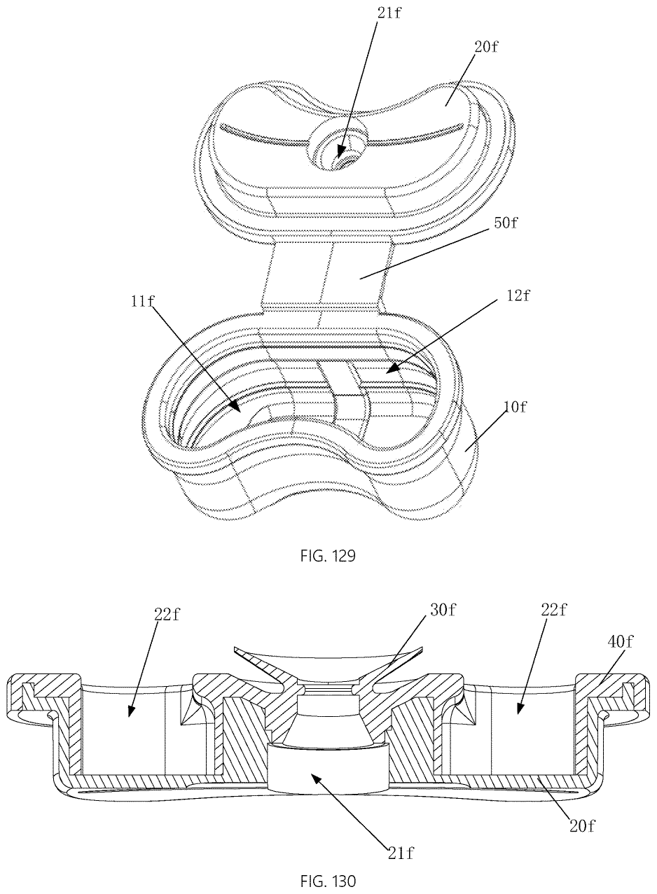

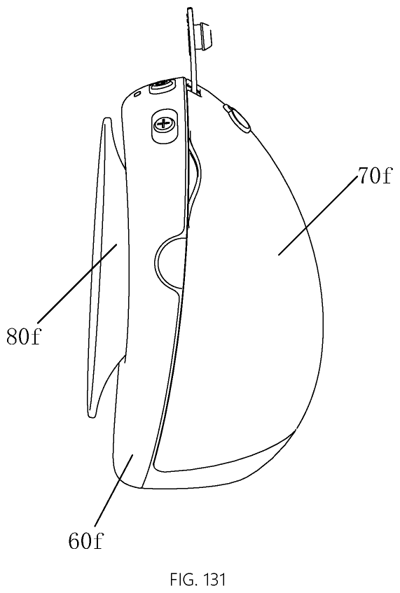

Referring to FIGS. 128-130, FIG. 128 is a schematic structural view of a diaphragm assembly in a closed state of a diaphragm cover according to a seventh embodiment of the present disclosure. FIG. 129 is a schematic structural view of a diaphragm assembly in an open state of a diaphragm cover according to a seventh embodiment of the present disclosure. FIG. 130 is a schematic sectional view of a diaphragm cover, a sealing structure, and a sealing layer according to a seventh embodiment of the present disclosure.

In this embodiment, the diaphragm assembly for a breast pump includes a diaphragm 10f, a diaphragm cover 20f, a sealing structure 30f, and a sealing layer 40f.

The diaphragm 10f has a cavity 11f and an opening 12f communicating with the cavity 11f.

The diaphragm cover 20f is configured to cover the opening 12f, and provided with a through hole 21f.

The sealing structure 30f is disposed around the through hole 21f. The sealing structure 30f is configured to form a seal with the main pump module when the diaphragm cover 20f abuts against the main pump module of the breast pump.

By providing the sealing structure 30f on the contact portion between the diaphragm cover 20f and the main pump module of the breast pump, when the breast pump operates, the main pump module is closely attached to the diaphragm cover 20f to form the reliable sealing effect, thereby effectively preventing leakage and contamination.

As an implementation, the sealing layer 40f is disposed on a surface of the diaphragm cover 20f facing away from the diaphragm 10f. The sealing layer 40f forms a secondary seal, further ensuring the sealing performance.

As an implementation, the sealing layer 40f and the sealing structure 30f are integrally formed. The sealing layer 40f on the diaphragm cover 20f does not cover the through hole 21f. The sealing layer 40f extends to a periphery of the through hole 21f to form the sealing structure 30f. A height that the sealing structure 30f protrudes from the diaphragm cover 20f is greater than a thickness of the sealing layer 40f.

As an implementation, the diaphragm cover 20f is made of a rigid material. The sealing layer 40f is made of a soft material.

As an implementation, the sealing structure 30f is a horn-shaped suction cup. A narrow end of the suction cup communicates with the through hole 21f. A wide end of the suction cup is configured to form a seal with the main pump module when the diaphragm cover 20f abuts against the main pump module of the breast pump.

As an implementation, a recess 22f is formed in the diaphragm cover 20f. The recess 22f is configured to facilitate removal of the diaphragm cover 20f from the opening 12f by the user.

As an implementation, there are two recesses 22f. The two recesses 22f are located at two opposite sides of the through hole 21f, respectively. For example, the recess 22f is configured to accommodate a finger of the user, such that the user can pinch the diaphragm cover 20f through the recess 22f to open the diaphragm cover 20f.

As an implementation, the diaphragm assembly further includes a connector 50f. The connector 50f includes one end connected to the diaphragm 10f, and another end connected to the diaphragm cover 20f. In an implementation, the diaphragm cover 20f may be integrally formed with the diaphragm 10f and the connector 50f, and the connector 50f is bendable to enable the diaphragm cover 20f to flip open or close.

The integral connection of the diaphragm cover 20f and the diaphragm 10f can reduce the number of components of the breast pump and prevent the components from being lost.

As an implementation, the connector 50f is disposed on an edge of a long side of an opening 12f of the diaphragm 10f. By disposing the connector 50f on the edge of the long side of an opening 12f of the diaphragm 10f, the stability of connection between the diaphragm 10f and the diaphragm cover 20f can be improved, prolonging the service life of the diaphragm assembly.

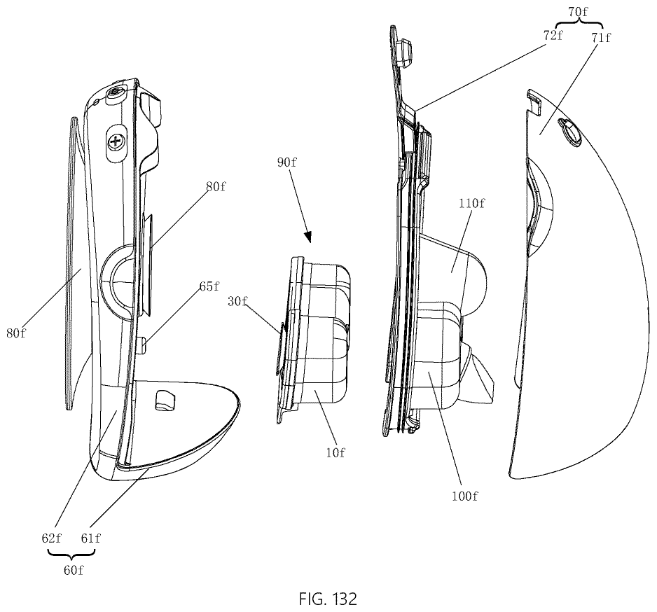

Referring to FIG. 131 and FIG. 132, FIG. 131 is a schematic structural view of a breast pump according to a seventh embodiment of the present disclosure. FIG. 132 is a schematic exploded view of a breast pump according to a seventh embodiment of the present disclosure.

This embodiment of the present disclosure further provides the breast pump. The breast pump includes the diaphragm assembly 90f in any of the foregoing embodiments. The breast pump includes a main pump module 60f. An air vent communicating with the through hole 21f of the diaphragm assembly is formed in the main pump module 60f. A negative pressure pump is disposed in the main pump module 60f. The negative pressure pump communicates with the air vent. Specifically, an air tube joint 65f is disposed on the main pump module 60f. The air vent is formed in the air tube joint 65f. The air tube joint 65f is inserted into the through hole 21f.

In some implementations, the diaphragm 10f may be made of an elastic material, and may deform under a negative pressure of the negative pressure pump of the main pump module 60f to transmit the negative pressure. The diaphragm 10f is configured for gas-liquid isolation between a negative pressure port side of the main pump module 60f and a milk expression side of the breast pump, preventing milk from flowing back to the main pump module 60f and causing damage or contamination to the main pump module 60f. Meanwhile, the diaphragm 10f is further configured to prevent entry of air into the milk pumping side to contaminate the milk.

Specifically, in this embodiment, the breast pump includes the main pump module 60f, a milk bowl assembly 70f, a breast shield 80f, and the diaphragm assembly 90f in any of the foregoing embodiments.

A milk storage chamber is formed in the milk bowl assembly 70f. A mounting hole is formed in a thickness direction of the main pump module 60f. The breast shield 80f has a narrow end and a wide end. The narrow end of the breast shield 80f penetrates through the mounting hole. The main pump module 60f is at least partially located between the wide end of the breast shield 80f and the milk bowl assembly 70f. A diaphragm bracket 100f is connected to the milk bowl assembly 70f and extends into the milk storage chamber.

The breast pump further includes a milk pumping conduit 110f connected to the milk bowl assembly 70f and extending into the milk storage chamber. The milk pumping conduit 110f communicates with an internal space of the breast shield 80f.

The milk bowl assembly 70f includes a bowl body 71f and a cover plate 72f. The bowl body 71f and the cover plate 72f enclose the milk storage chamber. Both the milk pumping conduit 110f and the diaphragm bracket 100f are connected to the cover plate 72f.

The milk pumping conduit 110f and the diaphragm bracket 100f are integrally connected with the cover plate 72f.

The milk pumping conduit 110f and the diaphragm bracket 100f are arranged side by side.

The milk pumping conduit 110f is located above the diaphragm bracket 100f.

The main pump module 60f includes a transverse portion 61f and a longitudinal portion 62f connected to the transverse portion 61f in a bending manner. The mounting hole is formed in the longitudinal portion 62f. The longitudinal portion 62f is located between the wide end of the breast shield 80f and the milk bowl assembly 70f. A first side of the milk bowl assembly 70f contacts the longitudinal portion of the main pump module 60f. A second side of the milk bowl assembly 70f contacts the transverse portion of the main pump module 60f. The first side and the second side are two adjacent sides of the milk bowl assembly.

The diaphragm 10f of the diaphragm assembly 90f is configured to be placed in the diaphragm bracket 100f. The diaphragm 10f divides an internal space of the diaphragm bracket 100f into a first subspace and a second subspace. The first subspace communicates with an internal space of the air tube joint 65f of the main pump module 60f. The second subspace communicates with an internal space of the milk pumping conduit 110f. Driven by a negative pressure provided by the main pump module 60f, the diaphragm 10f can deform in the internal space of the diaphragm bracket 100f to transmit the negative pressure.

According to the embodiments of the present disclosure, the diaphragm assembly includes the diaphragm, the diaphragm cover, and the sealing structure. The diaphragm has the cavity and the opening communicating with the cavity. The diaphragm cover is configured to cover the opening, and provided with the through hole. The sealing structure is disposed around the through hole. The sealing structure is configured to form the seal with the main pump module when the diaphragm cover abuts against the main pump module of the breast pump. By providing the sealing structure on the contact portion between the diaphragm cover and the main pump module of the breast pump, when the breast pump operates, the main pump module is closely attached to the diaphragm cover to form the reliable sealing effect, thereby effectively preventing leakage and contamination.

According to a fifth aspect, in view of shortcomings in the prior art, the present disclosure provides a breast pump, to solve the problem of low visibility.

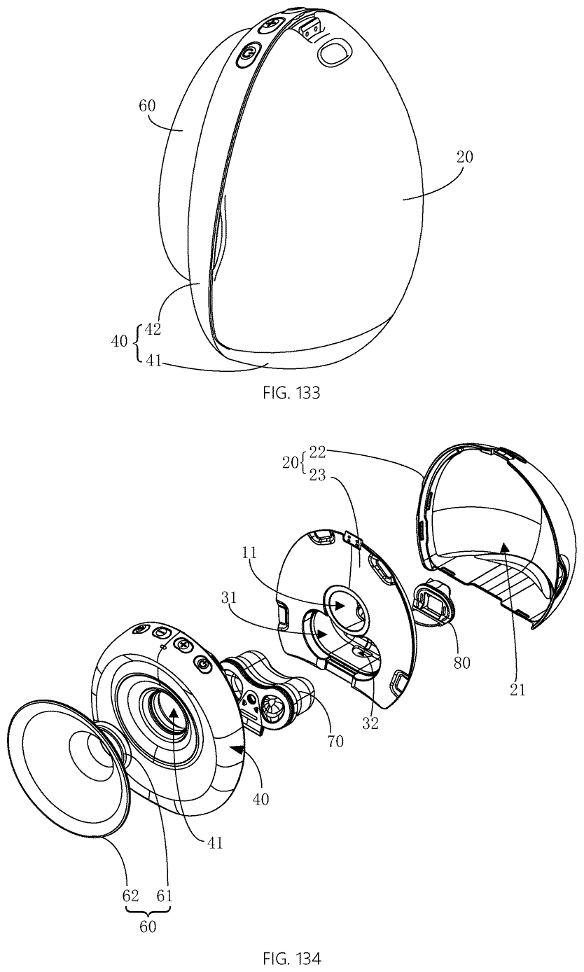

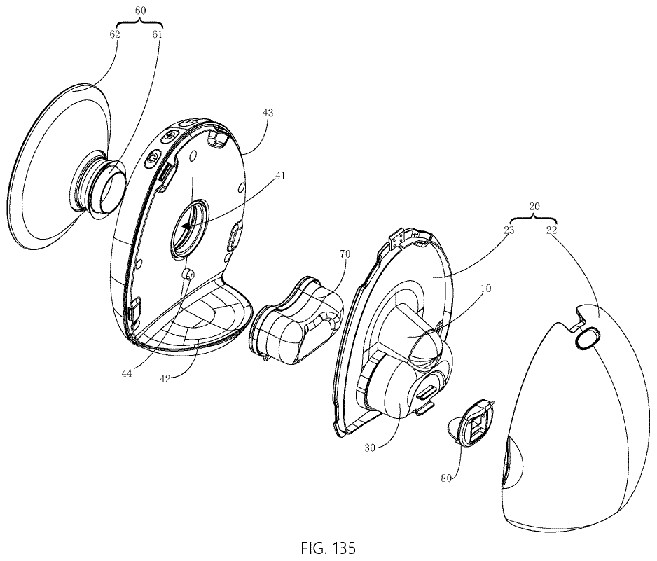

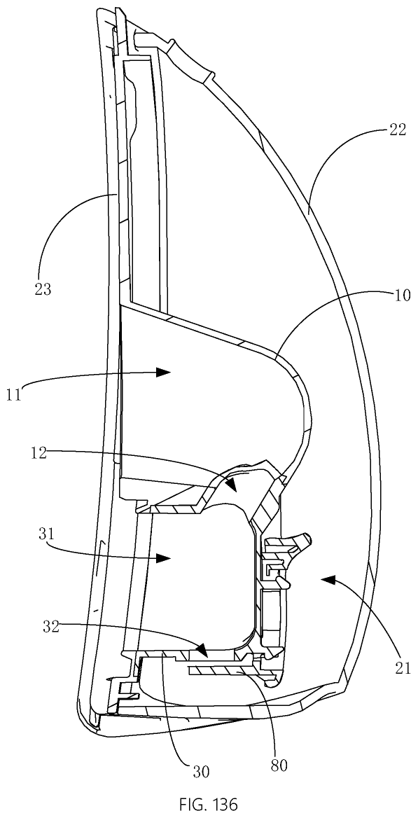

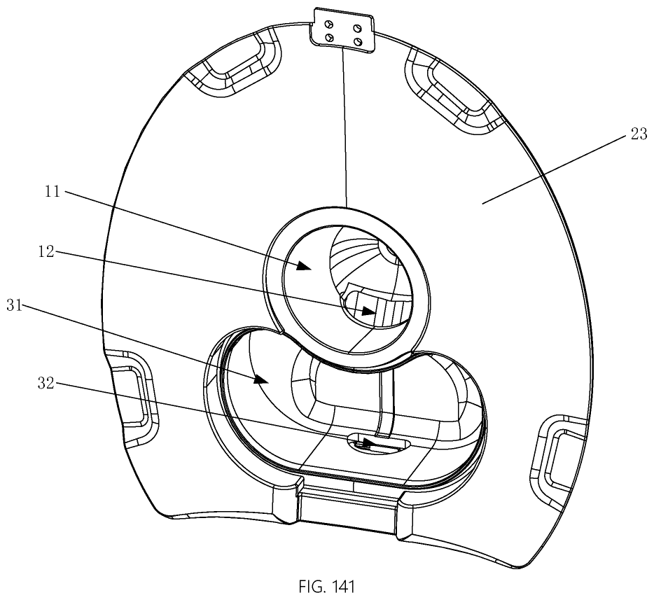

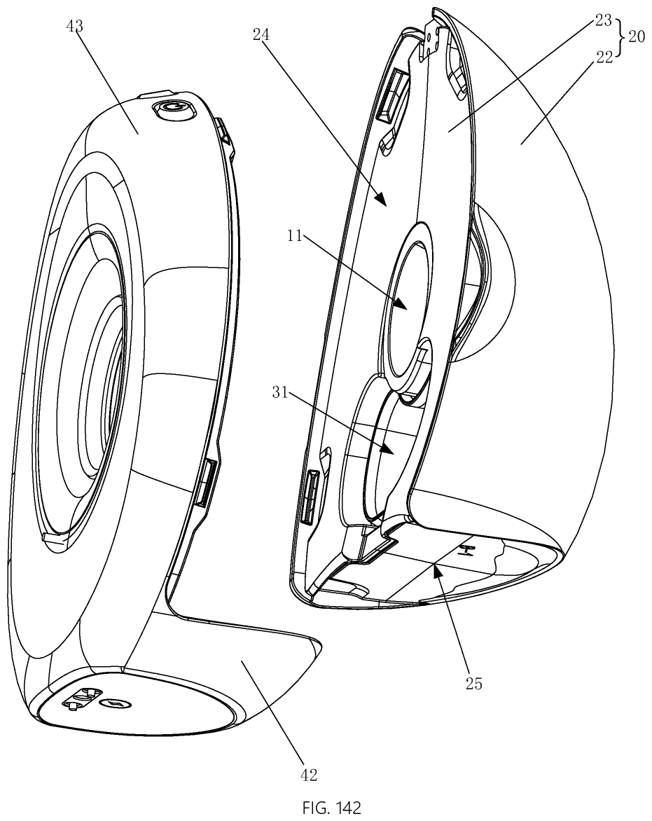

Referring to FIG. 133, FIG. 134, FIG. 135, FIG. 136, FIG. 141, and FIG. 142, in an embodiment, a breast pump may include: a milk pumping conduit 10 and a milk storage container 20.

A nipple tunnel 11 is formed in the milk pumping conduit 10. The nipple tunnel 11 is configured to receive milk from a breast. A milk outlet 12 is formed in the milk pumping conduit 10. A negative pressure is transferred to the nipple tunnel 11 via the milk outlet 12.

A milk storage chamber 21 is formed in the milk storage container 20. The milk in the nipple tunnel 11 flows out of the nipple tunnel 11 via the milk outlet 12 and flows to the milk storage chamber 21. As used herein, the term “flows” means directly flowing to the milk storage chamber 21 or indirectly flowing to the milk storage chamber 21 (for example, via a negative pressure chamber), which is not limited in this embodiment of the present disclosure.

The transfer of the negative pressure and the outflow of the milk are realized via the milk outlet 12. The milk flows along a direction of an airflow formed by the negative pressure, thereby preventing the milk from remaining in the nipple tunnel 11 and improving the milk expression efficiency.

In an implementation, the milk storage container 20 serves as a milk bowl assembly 20. The milk pumping conduit 10 is connected to the milk bowl assembly 20. In some implementations, the milk storage container may also be a feeding bottle. When the feeding bottle is used, a bottle mouth of the feeding bottle may be directly or indirectly docked with the milk outlet 12. In some implementations, the milk pumping conduit 10 may be integrally connected to the milk bowl assembly 20. In some other implementations, the milk pumping conduit 10 may be detachably connected to the milk bowl assembly 20, which is not limited in this embodiment of the present disclosure. The milk pumping conduit 10 may extend into the milk storage chamber 21.

In an implementation, the milk bowl assembly 20 includes a bowl body 22 and a cover plate 23. The bowl body 22 and the cover plate 23 enclose the milk storage chamber 21. The milk pumping conduit 10 is connected to the cover plate 23. In some implementations, the milk pumping conduit 10 may be detachably or integrally connected to the cover plate 23, which is not limited in this embodiment of the present disclosure.

In an implementation, the breast pump further includes a diaphragm bracket 30. A negative pressure chamber 31 is formed in the diaphragm bracket 30. The negative pressure chamber 31 communicates with the nipple tunnel 11 via the milk outlet 12. The diaphragm bracket 40 may be located under the milk pumping conduit 10. The diaphragm bracket 40 may extend side by side with the milk pumping conduit 10. The diaphragm bracket 40 may extend into the milk storage chamber 21.

In an implementation, the diaphragm bracket 30 is provided with a milk flow port 32. The milk in the nipple tunnel 11 flows out of the nipple tunnel 11 via the milk outlet 12, passes through the negative pressure chamber 31, and flows to the milk storage chamber 21 via the milk flow port 32.

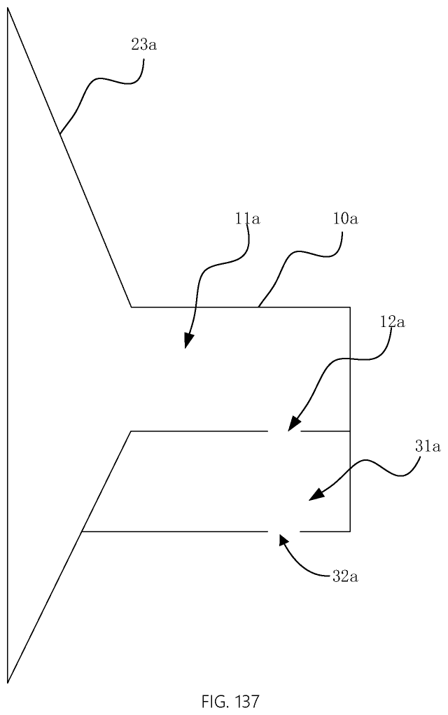

As shown in FIG. 137, in another implementation, the breast pump includes a breast shield 23a. A milk pumping conduit 10a is connected to the breast shield 23a. A nipple tunnel 11a is formed in the milk pumping conduit 10a. The nipple tunnel 11a is configured to receive milk from a breast. A milk outlet 12a is formed in the milk pumping conduit 10a. A negative pressure is transferred to the nipple tunnel 11a via the milk outlet 12a. The breast pump may further include a diaphragm bracket 30a. A negative pressure chamber 31a is formed in the diaphragm bracket 30a. The negative pressure chamber 31a communicates with the nipple tunnel 11a via the milk outlet 12a. The diaphragm bracket 30a is provided with a milk flow port 32a. The milk in the nipple tunnel 11a flows out of the nipple tunnel 11a via the milk outlet 12a, passes through the negative pressure chamber 31a, and flows to a milk storage chamber via the milk flow port 32a.

In an implementation, the breast pump further includes a main pump module 40. The main pump module 40 is configured to provide a negative pressure. The main pump module 40 may include a housing and such components located in the housing as a negative pressure pump, a solenoid valve, a battery, and a circuit board. The main pump module 40 may form an integrated breast pump with the milk bowl assembly 20, or may be connected to the milk bowl assembly 20 via a conduit to form a split breast pump. It should be understood that the negative pressure is not limited to an electric negative pressure pump, and may also be provided by a manual negative pressure mechanism, which is not limited in this embodiment of the present disclosure. In some implementations, the breast pump may not include a negative pressure mechanism, but the negative pressure is provided by an external power.

In an implementation, the breast pump further includes a main pump module 40. The main pump module 40 includes a negative pressure interface 44. The negative pressure interface 44 may communicate with a negative pressure pump. The main pump module 40 communicates with the negative pressure chamber 31 via the negative pressure interface 44. The main pump module 40 is configured to form a negative pressure in the negative pressure chamber 31. The negative pressure is transferred to the nipple tunnel 11 via the milk outlet 12.

In an implementation, the negative pressure chamber 31 is at least partially lower than the nipple tunnel 11 when the breast pump is worn. In some implementations, the negative pressure chamber 31 is completely located under the nipple tunnel 11. As used herein, the term “worn” means a worn state by a human body with an upper body in a normal upright posture, i.e., a state in which the wearing surface is substantially along a vertical direction.

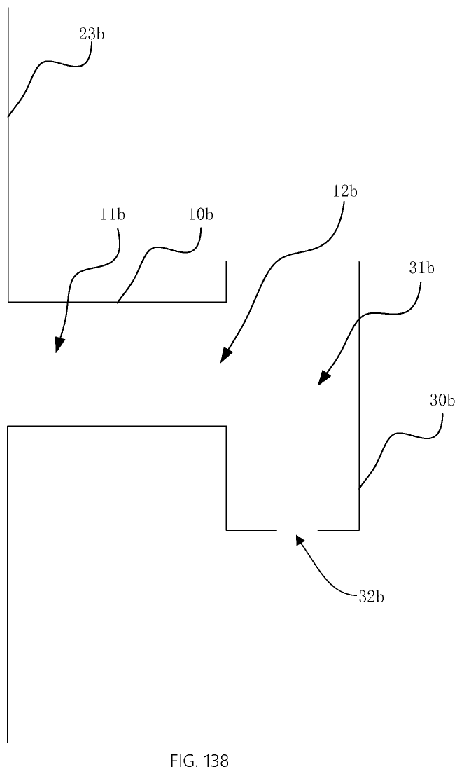

As shown in FIG. 138, in some other implementations, a negative pressure chamber 31b may also only have a portion located at a position lower than a nipple tunnel 11b. A milk pumping conduit 10b is connected to a cover plate 23b. A nipple tunnel 11b is formed in the milk pumping conduit 10b. The nipple tunnel 11b is configured to receive milk from a breast. A milk outlet 12b is formed in the milk pumping conduit 10b. The milk outlet 12b is formed in an end of the milk pumping conduit 10b. A negative pressure is transferred to the nipple tunnel 11b via the milk outlet 12b. The breast pump may further include a diaphragm bracket 30b. The negative pressure chamber 31b is formed in the diaphragm bracket 30b. The negative pressure chamber 31b communicates with the nipple tunnel 11b via the milk outlet 12b. The diaphragm bracket 30b is provided with a milk flow port 32b. The milk in the nipple tunnel 11b flows out of the nipple tunnel 11b via the milk outlet 12b, passes through the negative pressure chamber 31b, and flows to a milk storage chamber via the milk flow port 32b.

In this way, the negative pressure chamber is at least partially located under the nipple tunnel, which can improve the milk expression efficiency, and prevent milk accumulation.

In an implementation, the breast pump further includes a diaphragm 50. The diaphragm 50 is disposed in the negative pressure chamber 31. When the breast pump is worn, the diaphragm 50 is at least partially driven at a position lower than the nipple tunnel 11.

In this way, the negative pressure chamber is at least partially located under the nipple tunnel, which can improve the milk expression efficiency, and prevent milk accumulation.

In an implementation, the milk outlet 12 is formed in an underside of the milk pumping conduit 10. By forming the milk outlet 12 in the underside of the milk pumping conduit 10, the milk accumulation can be further reduced, preventing the milk from remaining in the milk pumping conduit 10. Meanwhile, the expressed milk in the milk pumping conduit 10 can flow out from the milk pumping conduit 10 partially by means of gravity, thereby further reducing the power consumption and improving the milk expression efficiency.

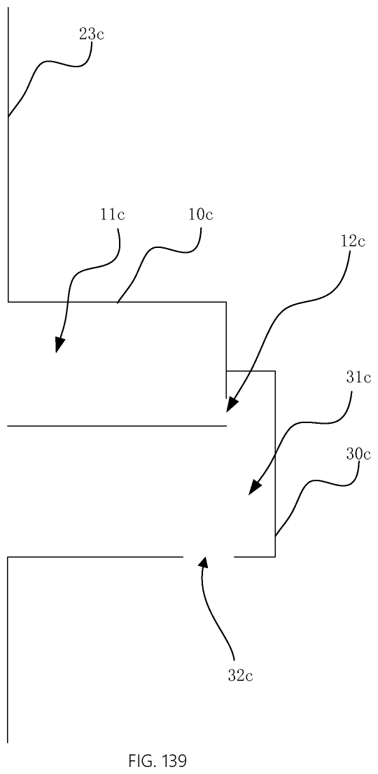

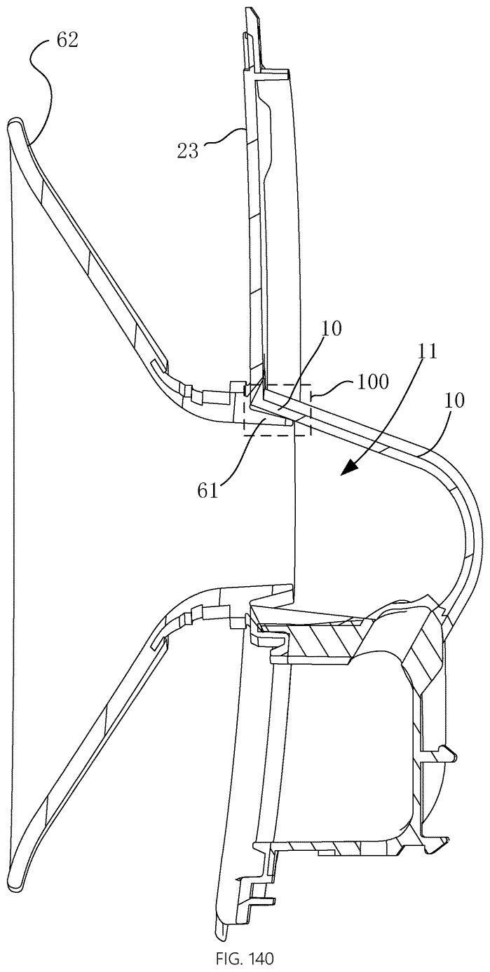

As shown in FIG. 139, in another implementation, a milk outlet 12c is formed at an end and lower position of a milk pumping conduit 10c. A milk pumping conduit 10c is connected to a cover plate 23c. A nipple tunnel 11c is formed in the milk pumping conduit 10c. The nipple tunnel 11c is configured to receive milk from a breast. The milk outlet 12c is formed in the milk pumping conduit 10c. The milk outlet 12c is formed in an end of the milk pumping conduit 10c. A negative pressure is transferred to the nipple tunnel 11c via the milk outlet 12c. The breast pump may further include a diaphragm bracket 30c. A negative pressure chamber 31c is formed in the diaphragm bracket 30c. The negative pressure chamber 31c communicates with the nipple tunnel 11c via the milk outlet 12c. The diaphragm bracket 30c is provided with a milk flow port 32c. The milk in the nipple tunnel 11c flows out of the nipple tunnel 11c via the milk outlet 12c, passes through the negative pressure chamber 31c, and flows to a milk storage chamber via the milk flow port 32c.

In an implementation, the milk outlet 12 is located between an end of the milk pumping conduit 10 and the cover plate 23. The milk in the nipple tunnel 11 flows out of the milk pumping conduit 10 via the milk outlet 12 under the action of the negative pressure. The milk pumping conduit 10 includes a distal end away from the cover plate 23. The milk outlet 12 is located between the cover plate 23 and the distal end. Since the milk outlet 12 is located between the end of the milk pumping conduit 10 and the cover plate 23, the overall structure of the breast pump can be more compact, the milk expression efficiency is improved, and the milk accumulation is prevented.

As shown in FIG. 135, FIG. 136, and FIG. 140, in an implementation, the distal end of the milk pumping conduit 10 is a sealed end. In an implementation, an end of the milk pumping conduit 10 away from the human body is sealed when the breast pump is worn. When the breast pump is worn, the end of the milk pumping conduit 10 away from the human body is sealed to form the sealed end, and the milk outlet 12 is located between the sealed end and the cover plate 23.

As shown in FIG. 139, in another implementation, a distal end of the milk pumping conduit 10c is an unsealed end. The milk outlet 12c is located at a lower position of the distal end.

In an implementation, the breast pump further includes a breast shield 60. An internal space of the breast shield 60 communicates with the nipple tunnel 11. An end of the breast shield 60 at least partially overlaps with the milk pumping conduit 10.

In an implementation, a position where the end of the breast shield 60 overlaps with the milk pumping conduit 10 forms a double-layered structure 100. The double-layered structure 100 is at least partially made of a light-transmissive material, such that an interior of the nipple tunnel 11 or the breast shield 60 can be observed through the double-layered structure 100. The double-layered structure 100 includes at least two layers, one layer being a portion of the breast shield 60, and another layer being a portion of the milk pumping conduit 10.

Optionally, a narrow end of the breast shield 60 at least partially overlaps with the milk pumping conduit 10 to form the double-layered structure 100.

In an implementation, a region of the double-layered structure 100 made of the light-transmissive material is located on the nipple tunnel 11.

In an implementation, the end of the breast shield 60 is inserted into the milk pumping conduit 10. In some other implementations, the milk pumping conduit may also be inserted into the end of the breast shield, which is not limited in this embodiment of the present disclosure. In this embodiment of the present disclosure, the breast shield 60 may be connected to the main pump module 40. There is no connection or contact between the breast shield 60 and the milk pumping conduit 10. The narrow end 61 of the breast shield 60 is only inserted into the milk pumping conduit 10 without any interference fit or threaded connection. The breast shield 60 is fixed by the main pump module 40.

In an implementation, the milk bowl assembly 20 is at least partially made of the light-transmissive material, such that the interior of the nipple tunnel 11 or the breast shield 60 can be observed through the milk bowl assembly 20 and the double-layered structure 100, or an interior of the milk storage chamber 21 can be observed through the milk bowl assembly 20.

In an implementation, the breast pump further includes a breast shield 60. A through hole 41 is formed in a thickness direction of the main pump module 40. The breast shield 60 has a narrow end 61 and a wide end 62. The narrow end of the breast shield 60 penetrates into the through hole 41. The main pump module 40 is at least partially located between the wide end 62 of the breast shield 60 and the milk bowl assembly 20.

In an implementation, the milk storage container 20 serves as a milk bowl assembly 20. A main pump module 40 includes a transverse portion 42 and a longitudinal portion 43 connected to the transverse portion 42 in a bending manner. A first side 24 of the milk bowl assembly 20 comes into contact with the longitudinal portion 43 of the main pump module 40. A second side 25 of the milk bowl assembly 20 comes into contact with the transverse portion 42 of the main pump module 40. The first side 24 and the second side 25 are two adjacent sides of the milk bowl assembly 20.

In an implementation, the breast pump further includes a diaphragm 70. The diaphragm 70 is configured to be placed in the negative pressure chamber 31. The diaphragm 70 divides an internal space of the diaphragm bracket 30 into a first subspace and a second subspace. The first subspace communicates with a negative pressure interface 44 of the main pump module 40. The second subspace communicates with an internal space of the milk pumping conduit 10. Driven by the negative pressure provided by the main pump module 40, the diaphragm 70 can deform in the internal space of the diaphragm bracket 30 to transfer the negative pressure.

In an implementation, the breast pump further includes a one-way valve 80. The one-way valve 80 is disposed at the milk flow port 32. The one-way valve 80 is configured to allow the milk to flow to the milk storage chamber 21 unidirectionally. The one-way valve 80 may be a disc-type one-way valve or a duckbill valve, which is not limited in this embodiment of the present disclosure.

According to this embodiment of the present disclosure, the breast pump includes the milk pumping conduit and the breast shield. The nipple tunnel is formed in the milk pumping conduit. The nipple tunnel is configured to receive the milk from the breast. An end of the breast shield at least partially overlaps with the milk pumping conduit. The position where the end of the breast shield overlaps with the milk pumping conduit forms the double-layered structure. The double-layered structure is at least partially made of the light-transmissive material, such that the interior of the nipple tunnel or the breast shield can be observed through the double-layered structure. The present disclosure can enable the user to observe the internal condition of the milk pumping conduit or the breast shield.

To make the objectives, technical solutions, and advantages of the present disclosure clearer, the present disclosure is further described in detail below with reference to the drawings and embodiments. It should be understood that the specific embodiments described herein are merely intended to explain the present application, rather than to limit the present application.

The foregoing is merely an alternative example of the present application and does not constitute a limitation on the scope of the present patent application. Any equivalent structure or equivalent process change made under the application concept of the present application by using the description and the accompanying drawings of the present application, or direct or indirect application thereof in other related technical fields, shall still fall in the protection scope of the patent of this application.

1. A breast pump, comprising a main pump module, a breast shield, a liquid separation device, and a breast pump assembly, wherein the main pump module is configured to generate negative pressure, and the breast shield is provided with a breast receptacle;
the breast pump assembly comprises:
a container body and a container lid, wherein the container body and the container lid enclose a milk storage chamber;
a negative pressure part, wherein the negative pressure part is connected to the container lid, the negative pressure part is provided with a negative pressure chamber; and
a nipple tunnel cap element, wherein the nipple tunnel cap element is connected to the container lid, the nipple tunnel cap element is provided with a nipple tunnel, the nipple tunnel communicates with the negative pressure chamber, and the nipple tunnel is at least configured to guide milk from a breast;
the liquid separation device is at least partially accommodated within the negative pressure chamber, and the liquid separation device is configured to transfer the negative pressure generated by the main pump module to the negative pressure chamber;
the nipple tunnel communicates with the breast receptacle; and
both the nipple tunnel cap element and the negative pressure part extend to an interior of the milk storage chamber; the negative pressure part is further provided with a first through hole and a second through hole; the first through hole is configured to communicate the negative pressure chamber with the nipple tunnel; and the second through hole is configured to communicate the negative pressure chamber with the milk storage chamber.

2. The breast pump according to claim 1, wherein the negative pressure part, the nipple tunnel cap element, and the container lid are integrally formed.

3. The breast pump according to claim 1, wherein the container lid is provided with a first perforation and a second perforation;
the negative pressure part is connected to the first perforation;
the nipple tunnel cap element is connected to the second perforation; and the second perforation communicates with the nipple tunnel, and is configured to allow the milk to pass through and flow to the nipple tunnel.

4. The breast pump according to claim 3, wherein the first perforation and the second perforation meet at least one of following conditions:
when the breast pump is placed against the breast, the first perforation is located below the second perforation;
a central axis of the first perforation is coaxial with a central axis of the negative pressure chamber, and a central axis of the second perforation is coaxial with a central axis of the nipple tunnel;
the central axis of the first perforation is parallel to the central axis of the second perforation; or
the first perforation is closely adjacent to the second perforation.

5. The breast pump according to claim 4, wherein when the breast pump is placed against the breast, the first perforation is located directly below the second perforation, and the negative pressure chamber communicates with the milk storage chamber.

6. The breast pump according to claim 1, wherein the nipple tunnel cap element and the negative pressure part share a first side wall, and two opposite sides of the first side wall face the negative pressure chamber and the nipple tunnel, respectively.

7. The breast pump according to claim 6, wherein the first through hole penetrates the first side wall and communicates the negative pressure chamber with the nipple tunnel.

8. The breast pump according to claim 7, wherein the negative pressure part further comprises a second side wall that is arranged opposite to the first side wall, and the second through hole penetrates the second side wall and communicates with the negative pressure chamber.

9. The breast pump according to claim 8, wherein the first through hole and the second through hole meet at least one of following conditions:
when the breast pump is placed against the breast, the second through hole is located below the first through hole;
when the breast pump is placed against the breast, the first through hole is located at a lowest position of the nipple tunnel cap element;
when the breast pump is placed against the breast, the second through hole is located at a lowest position of the negative pressure part;
when the breast pump is placed against the breast, the second through hole is aligned with the first through hole along a vertical direction;
the negative pressure part further comprises a first bottom wall; the first side wall, the second side wall, and the first bottom wall enclose the negative pressure chamber; and the second through hole is formed in an end of the second side wall close to the first bottom wall;
when the breast pump is placed against the breast, the first through hole is located at a bottom of the nipple tunnel cap element; or
when the breast pump is placed against the breast, the second through hole is located at a bottom of the negative pressure part.

10. The breast pump according to claim 9, wherein the first bottom wall is in smooth transition with both the first side wall and the second side wall.

11. The breast pump according to claim 7, wherein a length of the nipple tunnel cap element is equal to or substantially the same as a length of the negative pressure part.

12. The breast pump according to claim 1, wherein when the breast pump is placed against the breast, the negative pressure chamber is located below the nipple tunnel.

13. The breast pump according to claim 12, wherein when the breast pump is placed against the breast, the first through hole extends in a vertical direction to communicate the negative pressure chamber with the nipple tunnel; and when the breast pump is placed against the breast, the second through hole extends in the vertical direction, and the second through hole is located below the first through hole.

14. The breast pump according to claim 1, wherein the nipple tunnel is a circular structure, and the negative pressure chamber is a non-circular structure.

15. The breast pump according to claim 1, wherein the breast pump assembly further meets at least one of following conditions:
the container body is provided with a first mounting groove; and the container lid is accommodated in the first mounting groove, so as to enclose the milk storage chamber with the container body; or
the nipple tunnel cap element and the negative pressure part are arranged side by side.

16. The breast pump according to claim 1, wherein the container lid is provided with a first perforation; and the negative pressure part is connected to the first perforation and is located on a side of the container lid toward the milk storage chamber;
the liquid separation device comprises a liquid separation device body and a ring connected to the liquid separation device body; and
a circumferential direction of the main pump module around a negative pressure suction port and/or a circumferential direction of the container lid around the first perforation is provided with a second mounting groove; the ring is disposed in the second mounting groove; and the liquid separation device body is accommodated in the negative pressure chamber.

17. The breast pump according to claim 16, wherein when the breast pump is placed against the breast, the negative pressure chamber is located below the nipple tunnel; and
the liquid separation device has a contracted state and an expanded state; and when the liquid separation device is in the expanded state, at least a portion of the liquid separation device corresponding to the first through hole is spaced apart from a wall of the negative pressure chamber.

18. The breast pump according to claim 1, wherein the nipple tunnel cap element is at least partially made of a transparent material; and at least a portion of the container body that is located above the nipple tunnel cap element is made of an optically transparent material, such that a user is able to directly view an internal situation of the nipple tunnel through the container body and the nipple tunnel cap element.

19. The breast pump according to claim 1, wherein a one-way valve is provided at the second through hole, so as to prevent backflow of the milk to the negative pressure chamber from the milk storage chamber.

20. A breast pump assembly, applied to a breast pump and comprising:
a container body and a container lid, wherein the container body and the container lid enclose a milk storage chamber;
a negative pressure part, wherein the negative pressure part is connected to the container lid, and the negative pressure part is provided with a negative pressure chamber;
and a nipple tunnel cap element, wherein the nipple tunnel cap element is connected to the container lid, the nipple tunnel cap element is provided with a nipple tunnel, the nipple tunnel communicates with the negative pressure chamber, and the nipple tunnel is at least configured to guide milk from a breast;
wherein, the negative pressure chamber is at least configured to accommodate at least a portion of a liquid separation device of a main pump module of the breast pump;
and the liquid separation device is configured to transfer negative pressure generated by the main pump module to the negative pressure chamber; and
both the nipple tunnel cap element and the negative pressure part extend to an interior of the milk storage chamber; the negative pressure part is further provided with a first through hole and a second through hole; the first through hole is configured to communicate the negative pressure chamber with the nipple tunnel; and the second through hole is configured to communicate the negative pressure chamber with the milk storage chamber.
